(19) United States
(12) Patent Application Publication
(10) Pub. No.: US 2024/0417719 A1
(43) Pub. Date: Dec. 19, 2024

(54) METHODS AND COMPOSITIONS FOR EDITING A GENOME WITH PRIME EDITING AND A RECOMBINASE

(71) Applicants: The Broad Institute, Inc., Cambridge, MA (US); President and Fellows of Harvard College, Cambridge, MA (US)

(72) Inventors: David R. Liu, Cambridge, MA (US); Andrew Vito Anzalone, Cambridge, MA (US); Christopher J. Podracky, Cambridge, MA (US); Xin Gao, Cambridge, MA (US)

(73) Assignees: The Broad Institute, Inc., Cambridge, MA (US); President and Fellows of Harvard College, Cambridge, MA (US)

(21) Appl. No.: 18/704,328

(22) PCT Filed: Oct. 25, 2022
(86) PCT No.: PCT/US2022/078655

§ 371 Date: Apr. 24, 2024

Related U.S. Application Data
(60) Provisional application No. 63/271,700, filed on Oct. 25, 2021.

(51) Int. Cl. C12N 15/11; C12N 9/12; C12N 15/90

(52) U.S. Cl. CPC C12N 15/11; C12N 9/1276; C12N 15/907; C12Y 207/07049; C12N 2310/20

(57) ABSTRACT

Disclosed are constructs, systems, and methodologies using prime editing (PE), twin prime editing (twinPE), or multi-flap prime editing to carry out site-specific and large-scale genetic modification, such as, but not limited to, insertions, deletions, inversions, replacements, and chromosomal translocations of whole or partial genes (e.g., whole gene, gene exons and/or introns, and gene regulatory regions). In certain embodiments, the disclosure provides constructs, systems, and methods using prime editing (PE), e.g., single flap or “classical” PE or twinPE or multi-flap PE, to install one or more target sites for site specific recombination in a target genomic locus (e.g., a specific gene, exon, intron, or regulatory sequence), which may then be acted on by one or more site-specific recombinases to effectuate a large-scale genetic modification, such as an insertions, deletions, inversions, replacements, and chromosomal translocations.

### RELATED APPLICATIONS

This application claims priority under 35 U.S.C. § 119(e) to U.S. Provisional Application, U.S. Ser. No. 63/271,700, filed Oct. 25, 2021, the contents of which is incorporated herein by reference.

### GOVERNMENT SUPPORT

This invention was made with government support under grant numbers U01 AI142756, RM1 HG009490, and R35 GM118062 awarded by the National Institutes of Health. The government has certain rights in the invention.

### BACKGROUND OF THE INVENTION

Efficient, programmable, and site-specific genome modification remains a longstanding goal of genetics and medicine. In particular, the ability to efficiently and accurately render large-scale genomic changes, such as gene-level or exon-level insertions, inversions, translocations, and deletions has long been sought. Such methods would greatly advance the state of the art. Indeed, numerous genetic diseases caused by large-scale genomic defects, such as gene loss, gene inversion, or gene duplication, and chromosomal translocations could be treated with gene editing technologies that are capable of rendering such large-scale genetic modifications.

Early attempts at making such large-scale changes in the genome were focused on harnessing the power of homologous recombination. For instance, previous methods involved directing recombination to loci of interest (e.g., a disease-associated inverted or duplicated gene) based on available endogenous sites of recombination. This strategy was hampered by poor efficiency. More recent efforts have exploited the ability of double-stranded DNA breaks (DSBs) to induce homology-directed repair (HDR). See e.g., International PCT Application, PCT/US2017/046144, the contents of which are incorporated by reference. Homing endonucleases and programmable endonucleases, such as, zinc finger nucleases, TALE nucleases, and Cas9 nucleases, have been used to introduce targeted DSBs and induce HDR in the presence of donor DNA. In most post-mitotic cells, however, DSB-induced HDR is strongly down-regulated and generally inefficient. Moreover, repair of DSBs by error-prone repair pathways, such as non-homologous end-joining (NHEJ) or single-strand annealing (SSA), causes random insertions or deletions (indels) of nucleotides at the DSB site at a higher frequency than HDR. The efficiency of HDR can be increased if cells are subjected to conditions forcing cell-cycle synchronization, or if the enzymes involved in NHEJ are inhibited. However, such conditions can cause many random and unpredictable events, limiting potential applications.

Additional methods, compositions, and systems capable of efficiently and accurately introducing large-scale genomic changes, such as gene-level or exon-level insertions, inversions, translocations, and deletions would significantly advance the art.

The instant disclosure provides constructs, systems, and methodologies based on prime editing (PE), e.g., single-flap or “classical” PE or twinPE or multi-flap PE, in combination with recombinases to carry out site-specific and large-scale genetic modifications. The instant disclosure significantly advances the state of the art of genome editing as it provides for large-scale genetic modifications, including, but not limited to modifications of chromosomal regions, chromosomal loci containing one or multiple genes, or modifications of a single gene or portions thereof, such as exons, introns, and regulatory regions of a gene

### SUMMARY OF THE INVENTION

Recently, the inventors, led by David R. Liu et al., developed prime editing (PE), a highly versatile and precise genome editing method that directly writes new genetic information into a specified DNA site using a catalytically impaired Cas9 fused to an engineered reverse transcriptase, programmed with an engineered prime editing guide RNA (PEgRNA) that both specifies the target site and encodes the desired edit. See Anzalone et al., “Search-and-replace genome editing without double-strand breaks or donor DNA,” Nature 576, 149-157 (2019), the entire contents of which are incorporated herein by reference. In addition, prime editing is disclosed by the inventors in International PCT Application Nos.: PCT/US20/23721; PCT/US20/23730; PCT/US20/23713; PCT/US20/23712; PCT/US20/23727; PCT/US20/23724; PCT/US20/23725; PCT/US20/23728; PCT/US20/23732; PCT/US20/23723; PCT/US20/23553; and PCT/US20/23583, each filed on Mar. 19, 2020 and the contents each of which are incorporated herein by reference in their entireties. In another embodiment of prime editing (PE), the inventors recently developed twin prime editing (twinPE) or otherwise referred to as “multi-flap prime editing.” In various embodiments, twinPE and multi-flap PE involves forming pairs or multiple pairs of 3′ flaps on different strands, which form duplexes comprising desired edits and which can become incorporated into target nucleic acid molecules, e.g., at specific loci or edit sites in a genome. See International PCT Application No. PCT/US2021/31439, filed May 7, 2021, the contents of which are incorporated herein by reference. Thus, prime editing (PE) used herein may include prime editing (PE) which forms a single 3′ flap, twinPE which forms a pair of 3′ flaps, and multi-flap PE which forms multiple sets of pairs of 3′ flaps. The instant disclosure now provides compositions, constructs, systems, and methodologies based on prime editing (PE), including twin prime editing (twinPE) or multi-flap PE, to carry out site-specific and large-scale genetic modifications. The instant disclosure significantly advances the state of the art of genome editing as it provides for large-scale genomic changes, such as, insertions, deletions, inversions, replacements, and chromosomal translocations of one or more chromosomal regions, including one or more loci, one or more genes, or one or more portions of genes (e.g., gene exons, introns, and gene regulatory regions).

The instant disclosure provides compositions, constructs, nucleic acid molecules, fusion proteins, systems, and methods that leverage the power of prime editing (PE), e.g., single-flap or “classical” PE or twinPE or multi-flap PE, to carry out site-specific and large-scale genetic modifications, such as, but not limited to, insertions, deletions, inversions, replacements, and chromosomal translocations of chromosomes or portions thereof, chromosomal loci, or one or more genes or regions thereof, such as exons, introns, or gene regulatory regions of a gene. In certain embodiments, the disclosure provides compositions, constructs, systems, and methods using prime editing (PE), e.g., single-flap or “classical” PE or twinPE or multi-flap PE, to install a target site for site-specific recombination in a target genomic locus (e.g., a specific gene, exon, intron, or regulatory sequence). In certain other embodiments, the disclosure provides compositions, constructs, systems, and methods using prime editing (PE), e.g., single-flap or “classical” PE or twinPE or multi-flap PE, to install one or more target sites for site-specific recombination in a target genomic locus (e.g., a specific gene, exon, intron, or regulatory sequence). In still other embodiments, the disclosure provides compositions, constructs, systems, and methods using prime editing (PE), e.g., single-flap or “classical” PE or twinPE or multi-flap PE, to install one or more target sites for site-specific recombination in one or more target genomic loci (e.g., a specific gene, exon, intron, or regulatory sequence).

As referenced throughout, this disclosure combines the use of prime editing (PE), twinPE, or multi-flap prime editing with site-specific recombination. The term “site-specific recombination” refers a type of genetic recombination also known as “conservative site-specific recombination.” Site-specific recombination is a type of genetic recombination in which DNA strand exchange takes place between segments possessing at least a certain degree of sequence homology. Enzymes known as site-specific recombinases (“SSRs”) perform rearrangements of DNA segments by recognizing and binding to short, specific DNA sequence (“SSR recognition sequences”), at which they cleave the DNA backbone, exchange the two DNA helices involved, and rejoin the DNA strands. In some cases the presence of a recombinase enzyme and the recombination sites is sufficient for the reaction to proceed; in other systems a number of accessory proteins and/or accessory sites are required. Many different genome modification strategies, among these recombinase-mediated cassette exchange (RMCE), an advanced approach for the targeted introduction of transcription units into predetermined genomic loci, rely on SSRs. Site-specific recombination systems are highly specific, fast, and efficient, even when faced with complex eukaryotic genomes. They are employed naturally in a variety of cellular processes, including bacterial genome replication, differentiation and pathogenesis, and movement of mobile genetic elements. Recombination sites are typically between about 30 and 200 nucleotides in length and generally consist of two motifs with a partial inverted-repeat symmetry, to which the recombinase binds, and which flank a central crossover sequence at which the recombination takes place. The pairs of sites between which the recombination occurs are usually identical, but there are exceptions (e.g. attP and attB of λ integrase). Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. Recognition sequences for each of these recombinases are known in the art, but also are provided herein at Tables B and C, for example.

Once an SSR recognition sequence is installed in the genome, a cognate SSR which recognizes the installed SSR recognition sequence may be used to catalyze the precise cleavage, strand exchange, and rejoining of DNA fragments at the defined SSR recombination sites. This is accomplished without relying on endogenous repair mechanisms in a cell for repairing double-strand breaks which otherwise can induce indels and other undesirable DNA rearrangements. The reactions catalyzed by SSRs and SSR recognition sequences result in large-scale genomic changes, such as, insertions, deletions, inversions, replacements, and chromosomal translocations of one or more chromosomal regions, including one or more loci, one or more genes, or one or more portions of genes (e.g., gene exons, introns, and gene regulatory regions).

In certain embodiments, the one or more SSR recognition sites can be inserted or introduced anywhere within genome. In some organisms, a genome is organized as a single chromosome (e.g., bacteria) and the SSR recognition site may be inserted at any locus within the chromosome. The insertion site may be within a gene or within an intergenic region of a chromosome. The insertion may be within an exon, intron, or therebetween, or within a regulatory sequence, such as a promoter, enhancer, or transcription binding sequence. In other organisms, e.g., humans, the genome is organized into more than one chromosome and the SSR recognition site may be inserted at any locus within the chromosome. For instance, in humans, the genome comprises 23 pairs of chromosomes. In addition, the genome also may be mitochondrial DNA. The insertion site may be within a gene or within an intergenic region of a chromosome. The insertion may be within an exon, intron, or therebetween, or within a regulatory sequence, such as a promoter, enhancer, or transcription binding sequence.

As used herein “inserting in a genome” in any organism can include inserting one or more SSR recognition sites in any one or more chromosomes of a given genome (depending upon the number of chromosomes making up the genome) and at any chromosomal locus or loci. Where a genome comprises more than one chromosome, reference to “inserting in a genome” may include inserting the one or more SSRs into the one or more chromosomes of the genome. For example, in humans-which have 23 pairs of chromosomes-reference to “inserting in a genome” refers to inserting one or more SSR recognition sites in any one of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, or chromosome 23 (aka, XX chromosome or XY chromosome), or insertion into any combination of said chromosomes, or in a mitochondria genome.

In various embodiments, the site-specific recombination recognition sites are inserted by PE or twinPE upstream of a gene. For instance, the site-specific recombination recognition sites may be inserted upstream by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs from the 5′ end of gene. In other embodiments, the site-specific recombination recognition sites are inserted upstream by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs from the 5′ end of the transcription start site of a gene. In still another embodiment, the site-specific recombination recognition sites are inserted upstream by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs from the 5′ end of a promoter element.

In various embodiments, the site-specific recombination recognition sites are inserted by PE or twinPE downstream of a gene. For instance, the site-specific recombination recognition sites are inserted downstream by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs from the 3′ end of gene. In other embodiments, the site-specific recombination recognition sites are inserted downstream by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs from the 3′ end of the transcription termination site of a gene.

In still another embodiment, the site-specific recombination recognition sites are inserted within an exon, within an intron, or at the junction between an intron and exon, or upstream or downstream of an exon or intron. In various embodiments, the site-specific recombination recognition sites may be inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs upstream from the 5′ end of an exon.

In various embodiments, the site-specific recombination recognition sites are inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs downstream from the 3′ end of an exon.

In various embodiments, the site-specific recombination recognition sites are inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs upstream from the 5′ end of an intron.

In various embodiments, the site-specific recombination recognition sites are inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs downstream from the 3′ end of an intron.

In other embodiments, the site-specific recombination recognition sites are inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs upstream from the 5′ end of a regulatory sequence or element (e.g., a promoter, transcription binding site, or enhancer element).

In various embodiments, the site-specific recombination recognition sites may be inserted at a position which is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or up to 150 base pairs downstream from the 3′ end of a regulatory sequence or element (e.g., a cis-promoter element, transcription binding site, or enhancer element).

In various embodiments, the disclosure provides (i) a prime editor system comprising a prime editor (PE) comprising a nucleic acid programmable DNA binding protein (“napDNAbp”) and a polymerase (e.g., reverse transcriptase) and a prime editing guide RNA (PEgRNA) for targeting the prime editor to a target DNA sequence and (ii) a site-specific recombinase, wherein the PEgRNA comprises (a) a spacer sequence that comprises a region of complementarity that hybridizes to a first strand of a target DNA sequence, (b) an extension arm that comprises a DNA synthesis template and a primer binding site in a 5′ to 3′ orientation, and (c) a gRNA core that associates with the napDNAbp, and wherein the DNA synthesis template codes for one or more site-specific recombinase recognition sequences which become integrated into the target DNA sequence by inserting into or replacing the endogenous sequence at the target DNA sequence. The integrated one or more site-specific recombinase recognition sequences may then undergo site-specific recombination in the presence of the recombinase, which may be provided along with the PE or dual PE systems on the same nucleic acid molecules (e.g., expression vectors), or provided on separate nucleic acid molecules (e.g., expression vectors), or otherwise delivered separately by any means to a cell. In various embodiments, the disclosure also provides isolated prime editor systems describe herein which comprise the above-indicated PEgRNAs with DNA synthesis templates encoding the necessary site-specific recombination sequences. In various other embodiments, the disclosure provides complexes comprising the prime editor and a PEgRNA, wherein the PEgRNAs comprise the necessary site-specific recombination sequences. In still other embodiments, the disclosure provides one or more nucleic acid molecules encoding the prime editor systems, PEgRNAs, and recombinases. In various embodiments, the prime editor systems, PEgRNAs, and recombinases may be encoded on the same nucleic acid molecule (e.g., an expression vector), or they may be encoded on different nucleic molecule (e.g., a separate expression vector). In some embodiments (e.g., integration, cassette exchange recombination events), the disclosure also provides for one or more donor DNA molecules comprising one or more site-specific recombination sequences that are capable of undergoing recombination with the site-specific recombination sequences installed in the genome by PE, twinPE, or multi-flap PE.

In various embodiments, the disclosure provides (i) a prime editor system comprising a twin prime editor (twinPE) comprising a nucleic acid programmable DNA binding protein (“napDNAbp”) and a polymerase (e.g., reverse transcriptase) and a pair of prime editing guide RNAs (PEgRNA) for targeting the prime editor to opposite strands of a target DNA sequence and (ii) a site-specific recombinase. The PE system comprises a first PEgRNA comprising (a) a spacer sequence that comprises a region of complementarity that hybridizes to a first strand of a target DNA sequence, (b) an extension arm that comprises a DNA synthesis template and a primer binding site in a 5′ to 3′ orientation, and (c) a gRNA core that associates with the napDNAbp, and wherein the DNA synthesis template codes for one or more site-specific recombinase recognition sequences. In this embodiment, the PE system comprises a second PEgRNA comprising (a) a spacer sequence that comprises a region of complementarity that hybridizes to a first strand of a target DNA sequence, (b) an extension arm that comprises a DNA synthesis template and a primer binding site in a 5′ to 3′ orientation, and (c) a gRNA core that associates with the napDNAbp, and wherein the DNA synthesis template codes for one or more site-specific recombinase recognition sequences, wherein the recombinase recognition sequences of the first and second extension arms comprise a region of complementarity to one another. In operation, once the polymerase component synthesizes 3′ DNA flaps based on the sequence of the DNA synthesis templates, the 3′ DNA flaps are capable of forming a duplex comprising the one or more site-specific recombinase recognition sequences. This duplex then replaces the endogenous and corresponding strands of the target DNA sequence, such that after replacement and then ligation, the one or more recombinase recognition sequences become permanently installed into the target DNA sequence.

In various embodiments, the disclosure provides a prime editor system for installing one site-specific recombinase recognition sequence at a target DNA locus, or multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one target DNA locus or multiple target DNA loci.

In various embodiments, the disclosure provides a prime editor system comprising a PE for installing one site-specific recombinase recognition sequence at a target DNA locus, or multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one target DNA locus or multiple target DNA loci.

In various embodiments, the disclosure provides a prime editor system comprising a twinPE for installing one site-specific recombinase recognition sequence at a target DNA locus, or multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one target DNA locus or multiple target DNA loci.

The integrated one or more site-specific recombinase recognition sequences may then undergo site-specific recombination in the presence of the recombinase. In various embodiments, the disclosure also provides isolated prime editor systems describe herein. In various other embodiments, the disclosure provides complexes comprising the prime editor and a PEgRNA. In still other embodiments, the disclosure provides one or more nucleic acid molecules encoding the prime editor systems, PEgRNAs, and recombinases. In various embodiments, the prime editor systems, PEgRNAs, and recombinase may be encoded on the same nucleic acid molecule, or they may be encoded on different nucleic molecule.

In some embodiments, the disclosure provides a prime editor system having a recombinase for introducing a single recombinase recognition site in the target DNA, target gene, target genome, or target cell, and results in an intended edit in the target DNA, target gene, target genome, or target cell.

In some embodiments, a prime editor system with a recombinase component can result in insertion of an exogenous DNA sequence in a target DNA or target gene. In some embodiments, a single installed recombinase recognition site can be used as a landing site for a recombinase mediated reaction between the landing site installed in the target DNA and a second recombinase recognition site in a donor polynucleotide, for example, an exogenous donor DNA.

Insertion of a single recombinase recognition site can be accomplished with either PE having a single PEgRNA or twinPE. For example, in some embodiments, a prime editor system comprises a single PEgRNA comprising a DNA synthesis template comprising a single recombinase recognition site, which then directs the prime editor system to introduce the single recombinase recognition sites into a target DNA.

In some embodiments, a prime editor system comprises a pair of PEgRNAs each comprising a DNA synthesis template, wherein the first DNA synthesis template and the second DNA synthesis template comprises a region of complementarity to each other, and wherein the sequence (first DNA synthesis template and second DNA synthesis template have a region of complementarity between one another) comprises a single recombinase recognition site, and the prime editor system introduces the single recombinase recognition site in the target DNA.

For example, in some embodiments, a PEgRNA directs the prime editor system to introduce a recombinase recognition site in a target DNA. In some embodiments, a first PEgRNA and a second PEgRNA having a region of complementarity to each other introduces a recombinase recognition site in a target DNA. In some embodiments, the prime editor system further comprises a donor polynucleotide, e.g., a donor DNA, wherein the donor polynucleotide comprises one or more recombinase recognition site. In some embodiments, the recombinase component of the prime editor system results in recombination between the donor polynucleotide and the target DNA at the recombinase recognition sites, thereby inserting the sequence of the donor polynucleotide in the target DNA.

In some embodiments, the recombinase is a serine recombinase.

In some embodiments, the recombinase is a Bxb1 recombinase. In some embodiments, the recombinase is a phiC31 recombinase. In some embodiments, the recombinase is a serine recombinase as described herein, or any serine recombinase known in the art, or any functional variant thereof. In some embodiments, the recombinase recognition site introduced in the target DNA is an attP sequence, and the second recombinase recognition site in the donor polynucleotide is an attB sequence.

In some embodiments, a prime editor system having a recombinase component introduces two or more recombinase recognition sites in the target DNA, target gene, target genome, or target cell, and results in an intended edit in the target DNA, target gene, target genome, or target cell.

In certain embodiments, insertion of a two or more recombinase recognition sites can be accomplished with PE, including twinPE or multi-flap PE. For example, in some embodiments, a prime editor system comprising a single PEgRNA directs the prime editor system to introduce two or more recombinase recognition sites in a target DNA (for example, wherein the PEgRNA's DNA synthesis template strand comprises two or more site-specific recombinase recognition sequences).

In some embodiments, a prime editor system for PE comprises two or more PEgRNAs, wherein each of the two or more PEgRNAs comprises a DNA synthesis template that independently comprises a recombinase recognition site. For example, in some embodiments, a prime editor system for PE comprises a first PEgRNA and a second PEgRNA, wherein the first PEgRNA comprises a first spacer that is complementary to a first target region in a target DNA, and a first DNA synthesis template that comprises a first recombinase recognition site, and wherein the second PEgRNA comprises a second spacer that is complementary to a second target region in a target DNA, and a second DNA synthesis template that comprises a second recombinase recognition site, and wherein the first target region and the second target region are in different positions in the target DNA. In embodiments, the first and second recombinase recognition sites are the same. In other embodiments, the first and second recombinase recognition sites are different.

In some embodiments, a prime editor system for PE comprises a pair of PEgRNAs each comprising a DNA synthesis template, wherein the first DNA synthesis template and the second DNA synthesis template comprises a region of complementarity to each other, and wherein the sequence (first DNA synthesis template+second DNA synthesis template—region of complementarity between the first DNA synthesis template and second DNA synthesis template) comprises two or more recombinase recognition sites.

In some embodiments, a prime editor system for PE comprises at least two pair of PEgRNAs each comprising a DNA synthesis template, wherein the first pair comprises a PEgRNA comprising a first DNA synthesis template and a second PEgRNA comprising a second DNA synthesis template, wherein the first DNA synthesis template and the second DNA synthesis template comprises a region of complementarity to each other, and wherein the sequence (first DNA synthesis template+second DNA synthesis template—region of complementarity between the first DNA synthesis template and second DNA synthesis template) comprises a recombinase recognition sites; and wherein the second pair comprises a third DNA a third PEgRNA comprising a third DNA synthesis template and a fourth PEgRNA comprising a fourth DNA synthesis template, wherein the third and the fourth DNA synthesis template comprise a region of complementarity to each other, and wherein the sequence (third DNA synthesis template+fourth DNA synthesis template—region of complementarity between the third DNA synthesis template and fourth DNA synthesis template) comprises a recombinase recognition site.

In some embodiments, the recombinase is a tyrosine recombinase. In other embodiments, the recombinase is a Cre recombinase. In some embodiments, the recombinase is a Flp recombinase. In some embodiments, the recombinase is a tyrosine recombinase disclosed herein, or any tyrosine recombinase known in the art. In some embodiments, the two or more recombinase recognition sites introduced in the target DNA each comprises a Lox sequence. In some embodiments, the two or more recombinase recognition sites introduced in the target DNA each individually comprises a different (orthogonal) Lox sequence, e.g., a LoxP sequence, a Lox511 sequence, a Lox66 sequence, a Lox71 sequence, or a Lox2272 sequence.

In some embodiments, the recombinase is a serine recombinase. In some embodiments, the recombinase is a Bxb1 recombinase. In some embodiments, the two or more recombinase recognition sites each independently comprises an attB sequence or an attP sequence. In some embodiments, the two or more recombinase recognition sites each independently comprises an attB sequence or an attP sequence, wherein the central dinucleotide of the two recombinase recognition sites are the same, e.g., both recombinase recognition sites have GT central dinucleotide or both recombinase recognition sites have GA central dinucleotide. In some embodiments, the central dinucleotide of the two recombinase recognition sites are different, e.g., a first recombinase recognition site has GT central dinucleotide and a second recombinase recognition site has GA central dinucleotide, or vice versa. In some embodiments, the central dinucleotide of attB sequence or the attB sequence is GT. In some embodiments, the central dinucleotide of attB sequence or the attB sequence is GA. In some embodiments, the central dinucleotide of attB sequence or the attB sequence is GC. In some embodiments, the central dinucleotide of attB sequence or the attB sequence is CT.

Recombinase recognition sites introduced by prime editing can be used to generate an intended edit, including deletions, insertions, integrations, and replacement by donor sequences.

In some embodiments, a prime editor system for PE with a recombinase component can result in deletion of one or more nucleotides in a target DNA or target gene. For example, in some embodiments, a prime editor system for PE can result in integration of a first recombinase recognition site and a second recombinase recognition site in the target DNA, wherein the first and the second recombinase recognition sites are in the same orientation, and wherein the recombinase component mediates recombination between the two recombinase recognition sites, thereby resulting in deletion of the sequence in between the first and the second recombinase recognition sites.

In some embodiments, a prime editor system for PE with a recombinase component can result in replacement of an endogenous sequence in a target DNA or a target gene by an exogenous DNA sequence. For example, in some embodiments, a prime editor system for PE can result in a first recombinase recognition site and a second recombinase recognition site in the target DNA. In some embodiments, the prime editor system for PE further comprises a donor DNA, wherein the donor DNA comprises a third and a forth recombinase recognition sites, and wherein the recombinase component mediates recombination between the first recombinase recognition site and the third recombinase recognition site and recombination between the second recombinase recognition site and the fourth recombinase recognition site, thereby resulting in replacement of the sequence between the first and the second recombinase recognition sites in the target DNA by the sequence between the third and the fourth recombinase recognition sites in the donor DNA.

The replacement of an endogenous sequence by a sequence in a donor DNA can be done with either a serine recombinase or a tyrosine recombinase and corresponding recombinase recognition sequences. In some embodiments, the recombinase is a tyrosine recombinase, e.g., a tyrosine recombinase disclosed herein or any tyrosine recombinase known in the art. In some embodiments, the recombinase is a Cre recombinase. In some embodiments, the recombinase is a Flp recombinase. In some embodiments, the two or more recombinase recognition sites introduced by the prime editor system (e.g., twinPE or multi-flap PE) into the target DNA each comprises a Lox sequence. In some embodiments, the two or more recombinase recognition sites introduced in the target DNA each individually comprises a different (orthogonal) Lox sequence, for example, a first recombinase recognition site being a LoxP sequence, and the second one being a Lox2272 sequence.

In some embodiments, the recombinase is a serine recombinase, e.g., a serine recombinase disclosed herein or any serine recombinase known in the art. In some embodiments, the recombinase is a Bxb recombinase, and the two recombinase recognition sites introduced into the target DNA by the prime editor system (e.g., PE, or twinPE, or multi-flap PE) are orthogonal recombinase recognition sites, e.g., an attB-GT sequence and an attB-GA sequence. In such embodiments, the donor DNA sequence comprises two recombinase recognition sites, e.g., an attP-GT sequence and an attP-GA sequence, that can each individually recombine with to the two recombinase recognition sites introduced into the target DNA, wherein the central dinucleotide (GA or GT) controls the recombination between the attB-GA sequence with the attP-GA sequence and the recombination between the attB-GT sequence and the attP-GA sequence.

In some embodiments, a prime editor system (e.g., PE, or twinPE, or multi-flap PE) with a recombinase component can result in an inversion of a DNA fragment between two nucleotides in a target DNA or target gene. For example, in some embodiments, a prime editor system can result in a first recombinase recognition site and a second recombinase recognition site in a target DNA, wherein the first and the second recombinase recognition sites are in opposite directions, and wherein the recombinase component mediates recombination between the first and the second recombinase recognition sites, thereby resulting in inversion of the sequence in the target DNA between the first and the second recombinase recognition sites.

In some embodiments, a prime editor system with a recombinase component can result in an insertion of a DNA fragment between two nucleotides in a target DNA or target gene. For example, in some embodiments, a prime editor system can result in integration of a first recombinase recognition site, a second recombinase recognition site, and a linker sequence between the first and the second recombinase recognition sites in the target DNA. In some embodiments, the linker sequence is an exogenous DNA sequence, e.g., a expression tag or reporter tag. In some embodiments, the prime editor system further comprises a donor DNA, wherein the donor DNA comprises a third and a forth recombinase recognition sites, and wherein the recombinase component mediates recombination between the first recombinase recognition site and the third recombinase recognition site and recombination between the second recombinase recognition site and the fourth recombinase recognition site, thereby resulting in replacement of the sequence between the first and the second recombinase recognition sites in the target DNA by the sequence between the third and the fourth recombinase recognition sites in the donor DNA. Because the linker sequence is exogenous to the target DNA, the effect of the recombination is insertion of the sequence between the third and the fourth recombinase recognition sites in the target DNA.

In some embodiments, a prime editor system with a recombinase component introduces two or more recombinase recognition sites in the target DNA, target gene, target genome, or target cell, and results in two or more intended edits in the target DNA, target gene, target genome, or target cell.

In some embodiments, the two or more intended edits are in the same gene.

In some embodiments, the two or more intended edits are in different genes.

In some embodiments, the two or more intended edits are both insertions, deletions, inversions, or replacement by exogenous sequences.

In some embodiments, the two or more intended edits is different from each other, and is each independently an insertion, a deletion, an inversion, or a replacement by an exogenous sequence.

### BRIEF DESCRIPTION OF THE DRAWINGS

FIGS. 1A-1F provide schematics showing the introduction of various site-specific recombinase (SSR) targets into the genome using PE. (FIG. 1A) provides a general schematic of the insertion of a recombinase target sequence by a prime editor. (FIG. 1B) shows how a single SSR target inserted by PE can be used as a site for genomic integration of a DNA donor template. (FIG. 1C) shows how a tandem insertion of SSR target sites can be used to delete a portion of the genome. (FIG. 1D) shows how a tandem insertion of SSR target sites can be used to invert a portion of the genome. (FIG. 1E) shows how the insertion of two SSR target sites at two distal chromosomal regions can result in chromosomal translocation. (FIG. 1F) shows how the insertion of two different SSR target sites in the genome can be used to exchange a cassette from a DNA donor template.

FIG. 2 shows in 1) the PE-mediated synthesis of a SSR target site in a human cell genome and 2) the use of that SSR target site to integrate a DNA donor template comprising a GFP expression marker. Once successfully integrated, the GFP causes the cell to fluoresce.

FIG. 3 provides an overview of TwinPE. TwinPE systems target genomic DNA sequences that contain two protospacer sequences on opposite strands of DNA. PE2·PEgRNA complexes target each protospacer, generate a single-stranded nick, and reverse transcribe the PEgRNA-encoded template containing the desired insertion sequence. After synthesis and release of the 3′ DNA flaps, a hypothetical intermediate exists possessing annealed 3′ flaps containing the edited DNA sequence and annealed 5′ flaps containing the original DNA sequence. Excision of the original DNA sequence contained in the 5′ flap, followed by ligation of the 3′ flaps to the corresponding excision site, generates the desired edited product.

FIG. 4 provides a schematic illustrating the design differences between twinPE and PrimeDel and paired PEgRNAs study in plants.

FIGS. 5A-5G show site-specific genomic integration of DNA cargo with twinPE and Bxb1 recombinase in human cells. FIG. 5A provides a schematic of twinPE and Bxb1 recombinase-mediated site-specific genomic integration of DNA cargo. FIG. 5B shows screening of twinPE pegRNA pairs for insertion of the Bxb1 attB sequence at the CCR5 locus in HEK293T cells. FIG. 5C shows screening of twinPE pegRNA pairs for installation of the Bxb1 attP sequence at the AAVS1 locus in HEK293T cells. FIG. 5D shows single transfection knock-in of 5.6-kb DNA donors using twinPE pegRNA pairs targeting CCR5 or AAVS1. The twinPE pegRNAs install attB at CCR5 or attP at AAVS1. Bxb1 integrates a donor bearing the corresponding attachment site into the genomic attachment site. The number of integration events per 100 genomes is defined as the ratio of the target amplicon spanning the donor-genome junction to a reference amplicon in ACTB, as determined by ddPCR. FIG. 5E shows optimization of single-transfection integration at CCR5 using the A531+B584 spacers for the twinPE pegRNA pair. Identity of the templated edit (attB or attP), identity of the central dinucleotide (wild-type GT or orthogonal mutant GA), and length of the overlap between flaps were varied to identify combinations that supported the highest integration efficiency. Percent knock-in quantified as in FIG. 5D. FIG. 5F shows that pairs of pegRNAs were assessed for their ability to insert Bxb1 attB into the first intron of ALB. Protospacer sequences (277 and 358) are constant across the pegRNA pairs. The pegRNAs vary in their PBS lengths (variant b or c). The 277c/358c pair that performs best in HEK293T cells can also introduce the desired edit in Huh7 cells. FIG. 5G shows a comparison of single transfection knock-in efficiencies at CCR5 and ALB in HEK293T and Huh7 cell lines. Percent knock-in quantified as in FIG. 5D. Values and error bars reflect the mean and s.d. of three independent biological replicates.

FIGS. 6A-6C show that twin prime editing mediates sequence replacements at CCR5. FIG. 6A shows replacement of endogenous sequence within CCR5 region 1 with a 108-bp fragment of FKBP12 cDNA using twinPE (FKBP12 sequence oriented in the forward direction) or PE3 (FKBP12 sequence oriented in the reverse direction). For PE3 editing, PEgRNA RT templates were designed to encode 108 base pairs of FKBP12 cDNA sequence and one of three different target-site homology sequence lengths. For PE3 edits, each PEgRNA was tested with three nicking sgRNAs. FIG. 6B shows replacement of endogenous sequence within CCR5 region 2 with a 108-bp fragment of FKBP12 cDNA sequence using twinPE (FKBP12 sequence oriented in the forward direction) or PE3 (FKBP12 sequence oriented in the reverse direction). As in FIG. 6A, PE3 edits were tested with PEgRNAs containing RT templates that were designed to encode 108 base pairs of FKBP12 cDNA sequence and one of three different target-site homology sequence lengths. For PE3 edits, each PEgRNA was tested with three nicking sgRNAs. Values and error bars reflect the mean and s.d. of three independent biological replicates. FIG. 6C shows that transfection of HEK293T cells with a pair of PEgRNAs targeting CCR5 leads to replacement of 53 base pairs of endogenous sequence with 113 base pairs (attB-[27bp spacer]-attP) or 103 base pairs (attB-[27bp spacer]-attB) of exogenous sequence. Values and error bars reflect the mean and s.d. of three independent biological replicates.

FIGS. 7A-7B show recoding of PAH exon sequences in HEK293T cells via twinPE. Spacer pairs targeting exons 2, 4, and 5 (FIG. 7A) and exon 7 (FIG. 7B) for partial sequence recoding with twin prime editing. For each spacer pair, nine PEgRNA combinations were tested using three PBS variants for each spacer in a three-by-three matrix, with RT templates encoding the recoded exonic sequence held constant. Values reflect single biological replicates.

FIGS. 8A-8C show installation of a 50-bp Bxb1 attP site at AAVS1 with twinPE. Spacer pairs targeting the AAVS1 locus were designed for twinPE-mediated insertion of the Bxb1 attP attachment site. For each spacer, three PEgRNAs were designed having three different PBS lengths and a fixed RT template that encodes a portion (43-44 bp) of the Bxb1 attP sequence. For each spacer pair, a three-by-three matrix of PEgRNA combinations was tested by plasmid DNA cotransfection with PE2 in HEK293T cells. Each PEgRNA pair is specified below the x-axis. Values reflect single biological replicates.

FIGS. 9A-9B show installation of a 38-bp Bxb1 attB site at CCR5 with twinPE. Spacer pairs targeting the CCR5 locus were designed for twinPE-mediated insertion of the Bxb1 attB attachment site. For each spacer, three PEgRNAs were designed having three different PBS lengths and a fixed RT template that encodes the full-length Bxb1 attB sequence (38 bp). For each spacer pair, a three-by-three matrix of PEgRNA combinations was tested by plasmid DNA cotransfection with PE2 in HEK293T cells. Each PEgRNA pair is specified below the x-axis. Values reflect single biological replicates.

FIGS. 10A-10B show a comparison of twinPE and PE3 for Bxb1 attB insertion at CCR5. FIG. 10A shows replacement of endogenous sequence within CCR5 region 1 with the Bxb1 attB site using twinPE or PE3. For PE3 editing systems, PEgRNA RT templates were designed to encode the Bxb1 attB sequence and one of three different target-site homology sequence lengths. For PE3 edits, each PEgRNA was tested with three nicking sgRNAs. FIG. 10B shows replacement of endogenous sequence within CCR5 region 2 with the Bxb1 attB sequence using twinPE or PE3. As in FIG. 10A, PE3 edits were tested with PEgRNAs containing RT templates that were designed to encode the Bxb1 attB sequence and one of three different target-site homology sequence lengths and tested with three nicking sgRNAs. Values and error bars reflect the mean and s.d. of three independent biological replicates.

FIGS. 11A-11E show twinPE combined with Bxb1 recombinase for targeted knock-in of donor DNA plasmids. FIG. 11A shows Bxb1-mediated DNA donor knock-in in clonal HEK293T cell lines. Transfection of a HEK293T clonal cell line containing homozygous attB site insertion at CCR5 with varying amounts of Bxb1-expressing plasmid and attP-containing donor DNA plasmid. Knock-in efficiency was quantified by ddPCR. Values and error bars reflect the mean and s.d. of two independent biological replicates. FIG. 11B shows assessment of genome-donor junction purity by high-throughput sequencing. Genomic DNA from single-transfection knock-in experiments was amplified with a forward primer that binds the genome and a reverse primer that binds within the donor plasmid. Values and error bars reflect the mean and s.d. of three independent biological replicates. FIG. 11C shows multiplexed single transfection knock-in at AAVS1 and CCR5. HEK293T cells were transfected with plasmids encoding PE2, Bxb1, a pair of PEgRNAs for the insertion of attP at AAVS1, an attB-donor, a PEgRNA pair for the insertion of one of four attachment sites (attB, attB-GA, attP, or attP-GA) at CCR5, and a corresponding donor. Knock-in was observed at both target loci under all four conditions. Insertion of attP at AAVS1 and attB at CCR5 gave the lowest knock-in efficiencies overall (0.2% at AAVS1, 0.4% at CCR5). Insertion of attP at both sites yielded the highest levels of knock-in at AAVS1 (1.8%) but low levels (0.2%) at CCR5. When an orthogonal edit (attB-GA or attP-GA) was introduced at CCR5, AAVS1 knock-in was 0.7-0.8%. Higher knock-in at CCR5 was observed with attB-GA (1.4%) than with attP-GA (0.4%), consistent with single locus knock-in results. Values and error bars reflect the mean and s.d. of three independent biological replicates. FIGS. 11D and 11E show the effects of reducing PEgRNA overlap on twinPE efficiency and donor/PEgRNA recombination. FIG. 11D shows measurement of the editing efficiencies of pairs of PEgRNAs for insertion of Bxb1 attB at CCR5 by high-throughput sequencing. The pairs differed in the amount of overlap shared between their flaps, from 38 bp (full-length attB sequence) down to 20 bp. Editing efficiency of the pairs with shorter overlaps was comparable to the pair with full-length overlap. Values and error bars reflect the mean and s.d. of three independent biological replicates. FIG. 11E shows assessment of recombination between attB-containing PEgRNA plasmids and attP-containing donor plasmids. Following transfection of HEK293T cells with the indicated samples, isolated DNA was amplified with a forward primer that binds the PEgRNA expression plasmid (TTGAAAAAGTGGCACCGAGT (SEQ ID NO: 1)) and a reverse primer that binds the donor plasmid (CTCCCACTCATGATCTA (SEQ ID NO: 2)). A positive 256-bp PCR band confirms recombination between the two plasmids. When the PEgRNA encodes full-length attB (38-bp) or a truncated version of attB with 30-bp of overlap between flaps, a band is observed; however, recombination is not observed when the PEgRNAs encode a truncated attB with only 20-bp of flap overlap. The “No PE2” control uses the 38-bp overlap PEgRNA pair. No recombination is observed in the absence of Bxb1 or if the donor and PEgRNA plasmids both bear attB (Mismatch, “M”).

FIG. 12 shows expression of human Factor IX from the ALB promoter following twinPE-recombinase knock-in. Huh7 cells were transfected with Bxb1, donor (attP-splice acceptor-cDNA of F9 exons 2-8), PE2, and PEgRNAs for installation of attB in the first intron of ALB or at CCR5. Three days post-transfection, cells were split and allowed to grow to confluence. The media was changed, and cells were left to condition the fresh media, with aliquots taken at days 4, 7, and 10. Factor IX was present at detectable levels by ELISA (dashed line represents the lower limit of detection) in two of three samples treated with ALB PEgRNAs at Day 4, and in all samples treated with ALB PEgRNAs at Day 7 and Day 10. Factor IX was never detected in the conditioned media of any samples treated with CCR5 PEgRNAs. Values and error bars reflect the mean and s.d. of two or three independent biological replicates.

FIGS. 13A-13B show twinPE and Bxb1-mediated inversion in HEK293T GFP reporter cells. FIG. 13A shows the lentiviral fluorescent reporter construct used to assess inversion efficiency with twinPE and Bxb1 recombinase. The reporter contains an EFlα promoter followed by an inverted H2B-EGFP coding sequence that is flanked by partial AAVS1 DNA sequence, an internal ribosome entry site (IRES), and a puromycin resistance gene. Successful installation of opposite-facing attB (left) and attP (right) sequences at the AAVS1 target sequences and subsequent inversion by Bxb1 corrects the orientation of GFP for functional expression. FIG. 13B shows that the fluorescent reporter construct was stably integrated into HEK293T cells via lentiviral transduction and puromycin selection. The polyclonal GFP reporter cell line was then transfected with twinPE plasmid components (PE2 and four PEgRNAs) and varying amounts of Bxb1 plasmid for single-transfection inversion. Cells were analyzed by flow cytometry and gated for live single cells. Quantification of GFP positive cells was done by flow cytometry. Values and error bars reflect the mean and s.d. of two independent biological replicates.

FIGS. 14A-14B show twinPE and Bxb1 recombinase-mediated inversion between IDS and IDS2. FIG. 14A shows assessment of the inverted IDS junction purity by high-throughput sequencing in HEK293T cells. Frequency of expected junction sequences containing attR and attL recombination products after twinPE and BxB1-mediated single-step inversion. The product purities range from 81-89%. Values and error bars reflect the mean and s.d. of three independent biological replicates. FIG. 14B shows screening of PEgRNA pairs for the insertion of Bxb1 attB and attP sequences at IDS and IDS2. TwinPE editing was tested with standard PEgRNAs and ePEgRNAs containing a 3′ evoPreQ1 motif. Values and error bars reflect the mean and s.d. of three independent biological replicates.

FIGS. 15A-15B show twin prime editing-mediated insertion in CCR5 region 2 in HEK293T cells and twin prime editing in multiple human cell lines. FIG. 15A shows twinPE-mediated endogenous sequence replacement with Bxb1 attB attachment site in CCR5 region 2 in HEK293T cells. FIG. 15B shows twinPE-mediated endogenous sequence replacement with attP, attB, or 22-nt DNA sequences in multiple human cell lines. Six different PEgRNA pairs targeting five loci were tested in HEK293T, HeLa, U2OS, and K562 cells. HEK293T and HeLa cells were transfected with PE2 and PEgRNA plasmids via Lipofectamine 2000 (Thermo Fisher) and TransIT-HeLaMonster (Mirus), respectively. U2OS and K562 cells were nucleofected using Lonza 4D-Nucleofector and SE kit. DNA loci and the specified insertion edits are shown in the x-axis. Values and error bars reflect the mean and s.d. of at least two independent biological replicates.

FIGS. 16A-16B show installation of a 38-bp Bxb1 attB site at Rosa26 (a “safe harbor” locus in the human and mouse genomes) with twinPE in mouse N2A cells. Spacer pairs targeting the Rosa26 locus were designed for twinPE-mediated replacement of endogenous sequences with insertion of the Bxb1 attB attachment site. For each spacer, three PEgRNAs were designed having three different PBS lengths, a fixed 3′ EvoPreQ1 motif, a fixed RT template, that encodes the full-length Bxb1 attB sequence (38 bp). For each spacer pair, a three-by-three matrix of PEgRNA combinations was tested by plasmid DNA cotransfection with PE2 in HEK293T cells. Each PEgRNA pair is specified below the x-axis. Values reflect single biological replicate.

FIG. 17 shows installation of a 38-bp Bxb1 attB site at Rosa26 with twinPE in mouse N2A cells. A PEgRNA pair for each spacer pair was tested (9 different combinations of PEgRNA 1 and PEgRNA 2 with different PBS lengths) targeting the Rosa26 locus are shown. 6 of 18 tested spacer pairs show twinPE mediated attB insertion efficiency above 50%.

FIGS. 18A-18B show PE and quad-flap mediated insertion of H2B-EGFP with minicircle DNA donor in HEK293T reporter cells. FIG. 18A shows the lentiviral fluorescent reporter construct used to assess inversion efficiency with PE quad-flap and DNA donor. The reporter contains an EFlα promoter followed by partial AAVS1 DNA sequence, an internal ribosome entry site (IRES), and a puromycin resistance gene. Successful integration of the promoter-less H2B-EGFP at the targeted location after EFlα in reporter cells will result in the expression of H2B-EGFP. FIG. 18B shows that the reporter construct was stably integrated into HEK293T cells via lentiviral transduction and puromycin selection. The polyclonal reporter cell line was then transfected with PE2 plasmid, quadruple PEgRNAs, and minicircle DNA donor for single-transfection insertion. Cells were analyzed by flow cytometry and gated for live single cells 21 days after transfection. Quantification of GFP positive cells was done by flow cytometry. Values reflect single biological replicate.

FIGS. 19A-19B show PE and quad-flap mediated inversion in HEK293T GFP reporter cells. FIG. 19A shows the lentiviral fluorescent reporter construct used to assess inversion efficiency with PE and quad-flap. The reporter contains an EFlα promoter followed by an inverted H2B-EGFP coding sequence that is flanked by partial AAVS1 DNA sequence, an internal ribosome entry site (IRES), and a puromycin resistance gene. Successful reversion of the inverted H2B-EGFP, mediated by the annealing between the two attB flaps in the sense DNA strand and annealing between the attP flaps in the antisense DNA strand, result in the expression of H2B-EGFP. FIG. 19B shows that the fluorescent reporter construct was stably integrated into HEK293T cells via lentiviral transduction and puromycin selection. The polyclonal GFP reporter cell line was then transfected with PE2 and quadruple PEgRNAs for single-transfection inversion. Cells were analyzed by flow cytometry and gated for live single cells. Quantification of GFP positive cells was done by flow cytometry. Values and error bars reflect the mean and s.d. of more than three independent biological replicates.

FIGS. 20A-20B show PE and quad-flap mediated inversion at CCR5 in HEK293T cells. FIG. 20A shows the workflow of the transfection, TOPO cloning, and Sanger sequencing assessment of PE quad-flap mediated inversion in endogenous CCR5 locus in HEK293T cells. Briefly, HEK293T cells were transfected with PE2 and quadruple PEgRNAs that target CCR5 sequences for inversion. Genomic DNAs (treated and untreated control) were harvested 72 hours post transfection and were amplified by locus specific primers and cloned into TOPO vector. Next, bacterial competent cells were transformed with the cloned Topo and bacterial colonies were picked for sanger sequencing of both inversion junctions. FIG. 20B shows a representative clone with the precise inversion product from among the 96 clones sequenced.

FIG. 21A-21E show various embodiments of the structures for PEgRNA that may be used in connection with the PE, twinPE, and multi-flap PE systems described herein.

FIG. 21A depicts a PEgRNA comprising a 5′ extension arm.

FIG. 21B depicts a PEgRNA comprising a 3′ extension arm.

FIG. 21C provides the structure of an exemplary PEgRNA contemplated herein. The PEgRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an DNA synthesis template (or “edit template”) (B), and an optionally a homology arm (C) (which is not required for twinPE or multi-flap PE). In addition, the PEgRNA may comprise an optional 3′ end modifier region (e1) and an optional 5′ end modifier region (e2). Still further, the PEgRNA may comprise a transcriptional termination signal at the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The PEgRNA could comprise, in certain embodiments, secondary RNA structure, such as, but not limited to, hairpins, stem/loops, toe loops, RNA-binding protein recruitment domains (e.g., the MS2 aptamer which recruits and binds to the MS2cp protein). For instance, such secondary structures could be position within the spacer, the gRNA core, or the extension arm, and in particular, within the e1 and/or e2 modifier regions. In addition to secondary RNA structures, the PEgRNAs could comprise (e.g., within the e1 and/or e2 modifier regions) a chemical linker or a poly(N) linker or tail, where “N” can be any nucleobase. In some embodiments, the chemical linker may function to prevent reverse transcription of the sgRNA scaffold or core. In addition, in certain embodiments, the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the PEgRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the PEgRNA (e.g., in the e1 or e2 region, or elsewhere and an MS2cp protein fused to the DNA polymerase, thereby co-localizing the DNA polymerase to the PEgRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

FIG. 21D provides the structure of another PEgRNA contemplated herein. The PEgRNA comprises three main component elements ordered in the 5′ to 3′ direction, namely: an extension arm, a spacer, and a gRNA core. The extension arm may further be divided into the following structural elements in the 5′ to 3′ direction, namely: a primer binding site (A), an edit template (B), and an optional homology arm (C). The homology arm is not required in the twinPE and multi-flap PE embodiments. In addition, the PEgRNA may comprise an optional 3′ end modifier region (e1) and an optional 5′ end modifier region (e2). Still further, the PEgRNA may comprise a transcriptional termination signal on the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The PEgRNA could comprise, in certain embodiments, secondary RNA structures, such as, but not limited to, hairpins, stem/loops, toe loops, RNA-binding protein recruitment domains (e.g., the MS2 aptamer which recruits and binds to the MS2cp protein). These secondary structures could be positioned anywhere in the PEgRNA molecule. For instance, such secondary structures could be position within the spacer, the gRNA core, or the extension arm, and in particular, within the e1 and/or e2 modifier regions. In addition to secondary RNA structures, the PEgRNAs could comprise (e.g., within the e1 and/or e2 modifier regions) a chemical linker or a poly(N) linker or tail, where “N” can be any nucleobase. In some embodiments, the chemical linker may function to prevent reverse transcription of the sgRNA scaffold or core. In addition, in certain embodiments, the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the PEgRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the PEgRNA (e.g., in the e1 or e2 region, or elsewhere and an MS2cp protein fused to the DNA polymerase, thereby co-localizing the DNA polymerase to the PEgRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the optional “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

FIG. 21E depicts the interaction of a typical PEgRNA with a target site of a double stranded DNA and the concomitant production of a 3′ single stranded DNA flap containing the genetic change of interest. The double strand DNA is shown with the top strand (i.e., the target strand) in the 3′ to 5′ orientation and the lower strand (i.e., the PAM strand or non-target strand) in the 5′ to 3′ direction. The top strand comprises the complement of the “protospacer” and the complement of the PAM sequence and is referred to as the “target strand” because it is the strand that is targeted by and anneals to the spacer of the PEgRNA. The complementary lower strand is referred to as the “non-target strand” or the “PAM strand” or the “protospacer strand” since it contains the PAM sequence (e.g., NGG) and the protospacer. Although not shown, the PEgRNA depicted would be complexed with a Cas9 or equivalent domain of a prime editor fusion protein. As shown in the schematic, the spacer of the PEgRNA anneals to the complementary region of the protospacer on the target strand. This interaction forms as DNA/RNA hybrid between the spacer RNA and the complement of the protospacer DNA, and induces the formation of an R loop in the protospacer. As taught elsewhere herein, the Cas9 protein (not shown) then induces a nick in the non-target strand, as shown. This then leads to the formation of the 3′ ssDNA flap region immediately upstream of the nick site which, in accordance with *z*, interacts with the 3′ end of the PEgRNA at the primer binding site. The 3′ end of the ssDNA flap (i.e., the reverse transcriptase primer sequence) anneals to the primer binding site (A) on the PEgRNA, thereby priming reverse transcriptase. Next, reverse transcriptase (e.g., provided in trans or provided cis as a fusion protein, attached to the Cas9 construct) then polymerizes a single strand of DNA which is coded for by the DNA synthesis template (including the edit template (B) and homology arm (C)). The polymerization continues towards the 5′ end of the extension arm. The polymerized strand of ssDNA forms a ssDNA 3′ end flap which, as describe elsewhere (e.g., as shown in FIG. 1G), invades the endogenous DNA, displacing the corresponding endogenous strand (which is removed as a 5′ ended DNA flap of endogenous DNA), and installing the desired nucleotide edit (single nucleotide base pair change, deletions, insertions (including whole genes) through naturally occurring DNA repair/replication rounds.

FIG. 22 shows editing of iPSC cells to insert Bxb1 recombinase recognition sites by twin prime editing. TwinPE (with PE2 mRNA and dual synthetic pegRNAs) was used to successfully insert 38 bp Bxb1 at the CCR5 safe harbor locus with up to 45% editing efficiency in bulk iPSCs without any selection.

FIGS. 23A-23E show site-specific large genomic sequence inversion with twinPE and Bxb1 recombinase in human cells. FIG. 23A provides a schematic diagram of DNA recombination hot spots in IDS and IDS2 that lead to pathogenic 39-kb inversions, and the combined twinPE-Bxb1 strategy for installing or correcting the IDS inversion. FIG. 23B shows a screen of pegRNA pairs at IDS and IDS2 for insertion of attP or attB recombination sites. Values and error bars reflect the mean and s.d. three independent biological replicates. FIG. 23C shows DNA sequencing analysis of the IDS and IDS2 loci after twinPE-mediated insertion of attP or attB sequences, with or without subsequent transfection with Bxb1 recombinase. P-values were derived from a Student's two-tailed t-test. Values and error bars reflect the mean and s.d. of three independent biological replicates. FIG. 23D shows 40,167-bp IDS inversion product purities at the anticipated inversion junctions after twinPE-mediated attachment site installation and sequential transfection with Bxb1 recombinase. Values and error bars reflect the mean and s.d. of three independent biological replicates. FIG. 23E shows analysis of inversion efficiency by amplicon sequencing at IDS and IDS2 loci after sequential transfection or single-step transfection of twinPE editing components and Bxb1 recombinase. Values and error bars for sequential transfection reflect the mean and s.d. of three independent biological replicates; values for single-transfection reflect the mean of two independent biological replicates.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S. A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 13). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 13). In some embodiments, the Cas9 variant comprises a fragment of SEQ ID NO: 13 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 13). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 13).

cDNA

The term “cDNA” refers to a strand of DNA copied from an RNA template. cDNA is complementary to the RNA template.

### Circular Permutant

As used herein, the term “circular permutant” refers to a protein or polypeptide (e.g., a Cas9) comprising a circular permutation, which is a change in the protein's structural configuration involving a change in the order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including, reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques.

### Circularly Permuted Cas9

The term “circularly permuted Cas9” refers to any Cas9 protein, or variant thereof, that has been occurs as a circular permutant, whereby its N- and C-termini have been topically rearranged. Such circularly permuted Cas9 proteins (“CP-Cas9”), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of which are incorporated herein by reference. The instant disclosure contemplates any previously known CP-Cas9 or use of a new CP-Cas9 so long as the resulting circularly permuted protein retains the ability to bind DNA when complexed with a guide RNA (gRNA).

### Cleavage Site, Nick Site, Cut Site

The terms “cleavage site,” “nick site,” and “cut site” as used interchangeably herein in the context of prime editing, refer to a specific position in between two nucleotides or two base pairs in the double-stranded target DNA sequence. In some embodiments, the position of a nick site is determined relative to the position of a specific PAM sequence. In some embodiments, the nick site is the particular position where a nick will occur when the double stranded target DNA is contacted with a napDNAbp, e.g., a nickase such as a Cas nickase, that recognizes a specific PAM sequence. For each PEgRNA described herein, a nick site is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence).

In some embodiments, a nick site is in a target strand of the double-stranded target DNA sequence. In some embodiments, a nick site is in a non-target strand of the double-stranded target DNA sequence. In some embodiments, the nick site is in a protospacer sequence. In some embodiments, the nick site is adjacent to a protospacer sequence. In some embodiments, a nick site is downstream of a region, e.g., on a non-target strand, that is complementary to a primer binding site of a PEgRNA. In some embodiments, a nick site is downstream of a region, e.g., on a non-target strand, that binds to a primer binding site of a PEgRNA. In some embodiments, a nick site is immediately downstream of a region, e.g., on a non-target strand, that is complementary to a primer binding site of a PEgRNA. In some embodiments, the nick site is upstream of a specific PAM sequence on the non-target strand of the double stranded target DNA, wherein the PAM sequence is specific for recognition by a napDNAbp that associates with the gRNA core of a PEgRNA. In some embodiments, the nick site is downstream of a specific PAM sequence on the non-target strand of the double stranded target DNA. wherein the PAM sequence is specific for recognition by a napDNAbp that associates with the gRNA core of a PEgRNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S. A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a PEgRNA that is utilized as a template strand by a polymerase of a prime editor to encode a 3′ single-strand DNA flap that contains the desired edit and which then, through the mechanism of prime editing, replaces the corresponding endogenous strand of DNA at the target site. In various embodiments, the DNA synthesis template codes for one or more site-specific recombinase recognition sequences. Equivalent terms are “RT edit template” or “edit template.”

### Downstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Effective Amount

The term “effective amount,” as used herein, refers to an amount of a biologically active agent that is sufficient to elicit a desired biological response. For example, in some embodiments, an effective amount of a prime editor (PE) may refer to the amount of the editor that is sufficient to edit a target site nucleotide sequence, e.g., a genome. In some embodiments, an effective amount of a prime editor (PE) provided herein, e.g., of a fusion protein comprising a nickase Cas9 domain and a reverse transcriptase may refer to the amount of the fusion protein that is sufficient to induce editing of a target site specifically bound and edited by the fusion protein. As will be appreciated by the skilled artisan, the effective amount of an agent, e.g., a fusion protein, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Extension Arm

The term “extension arm” refers to a nucleotide sequence component of a PEgRNA which provides several functions, including a primer binding site and a DNA synthesis template that encodes an edit, such as a sequence for a site-specific recombinase recognition sequence. In some embodiments, the extension arm is located at the 3′ end of the guide RNA. In other embodiments, the extension arm is located at the 5′ end of the guide RNA. In various embodiments, the extension arm comprises the following components in a 5′ to 3′ direction: the DNA synthesis template and the primer binding site. Since polymerization activity of the reverse transcriptase is in the 5′ to 3′ direction, the preferred arrangement of the DNA synthesis template and primer binding site is in the 5′ to 3′ direction such that the reverse transcriptase (or other polymerase), once primed by an annealed primer sequence, polymerizes a single strand of DNA using the DNA synthesis template as a complementary template strand.

The extension arm may also be described as comprising generally two regions: a primer binding site (PBS) and a DNA synthesis template. The primer binding site binds to the primer sequence that is formed from the endogenous DNA strand of the target site when it becomes nicked by the prime editor complex, thereby exposing a 3′ end on the endogenous nicked strand. The binding of the primer sequence to the primer binding site on the extension arm of the PEgRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information, e.g., a SSR recognition sequence) by the polymerase of the prime editor complex and which ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the PEgRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Flap Endonuclease (e.g., FEN1)

As used herein, the term “flap endonuclease” refers to an enzyme that catalyzes the removal of 5′ single strand DNA flaps. These are naturally occurring enzymes that process the removal of 5′ flaps formed during cellular processes, including DNA replication. The prime editing methods herein described may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519, Tsutakawa et al., “Human flap endonuclease structures, DNA double-base flipping, and a unified understanding of the FEN1 superfamily,” Cell, 2011, 145(2): 198-211, and Balakrishnan et al., “Flap Endonuclease 1,” Annu Rev Biochem, 2013, Vol 82: 119-138 (each of which are incorporated herein by reference).

### Functional Equivalent

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, mutated, or synthetic version of protein X which bears an equivalent function.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Gene

As used herein, a “gene” is the basic physical unit of inheritance. Genes are passed from parents to offspring and contain the information needed to specify traits. Genes are arranged, one after another, on structures called chromosomes. A chromosome contains a single, long DNA molecule, only a portion of which corresponds to a single gene. Genes may encode proteins or other nucleic acids, such as mRNA, tRNA, or rRNA, guide RNA, or other nucleic acid molecules (e.g., small interfering RNA (siRNA), microRNA (miRNA), small nuclear RNA (snRNA), small nucleolar RNA (snoRNA), and short hairpin RNA (shRNA)). Genes can be as short as about several tens or hundreds of base pairs, and as long as several hundred thousand base pairs.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “PEgRNAs”) which have been invented for the prime editing methods and composition disclosed herein.

Guide RNAs or PEgRNAs may comprise various structural elements that include, but are not limited to: (a) spacer sequence—the sequence in the guide RNA or PEgRNA (having about 20 nts in length) which binds to the complement of the protospacer in the target DNA. The spacer of the RNA and the protospacer of the DNA may comprise the same sequence. (b) gRNA core (or gRNA scaffold or backbone sequence)—refers to the sequence within the gRNA that is responsible for Cas9 binding, it does not include the 20 bp spacer/targeting sequence that is used to guide Cas9 to target DNA. (c) extension arm—a single strand extension at the 3′ end or the 5′ end of the PEgRNA which comprises a primer binding site and a DNA synthesis template sequence that encodes via a polymerase (e.g., a reverse transcriptase) a single stranded DNA flap containing the genetic change of interest (e.g., SSR recognition sequence), which then integrates into the endogenous DNA by replacing the corresponding endogenous strand, thereby installing the desired genetic change. (d) Transcription terminator—the guide RNA or PEgRNA may comprise a transcriptional termination sequence at the 3′ of the molecule.

### Homology Arm

The term “homology arm” refers to a portion of the extension arm that encodes a portion of the resulting reverse transcriptase-encoded single strand DNA flap that is to be integrated into the target DNA site by replacing the endogenous strand. The portion of the single strand DNA flap encoded by the homology arm is complementary to the non-edited strand of the target DNA sequence, which facilitates the displacement of the endogenous strand and annealing of the single strand DNA flap in its place, thereby installing the edit. This component is further defined elsewhere. The homology arm is part of the DNA synthesis template since it is by definition encoded by the polymerase of the prime editors described herein.

### Host Cell

The term “host cell,” as used herein, refers to a cell that can host, replicate, and express a vector described herein, e.g., a vector comprising a nucleic acid molecule encoding a fusion protein comprising a Cas9 or Cas9 equivalent and a reverse transcriptase.

### Inteins

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.” Inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. O., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Ligand-Dependent Intein

The term “ligand-dependent intein,” as used herein refers to an intein that comprises a ligand-binding domain. Typically, the ligand-binding domain is inserted into the amino acid sequence of the intein, resulting in a structure intein (N)—ligand-binding domain—intein (C). Typically, ligand-dependent inteins exhibit no or only minimal protein splicing activity in the absence of an appropriate ligand, and a marked increase of protein splicing activity in the presence of the ligand. In some embodiments, the ligand-dependent intein does not exhibit observable splicing activity in the absence of ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art, and in include those provided below and those described in published U.S. Patent Application U.S. 2014/0065711 A1; Mootz et al., “Protein splicing triggered by a small molecule.” J. Am. Chem. Soc. 2002; 124, 9044-9045; Mootz et al., “Conditional protein splicing: a new tool to control protein structure and function in vitro and in vivo.” J. Am. Chem. Soc. 2003; 125, 10561-10569; Buskirk et al., Proc. Natl. Acad. Sci. USA. 2004; 101, 10505-10510); Skretas & Wood, “Regulation of protein activity with small-molecule-controlled inteins.” Protein Sci. 2005; 14, 523-532; Schwartz, et al., “Post-translational enzyme activation in an animal via optimized conditional protein splicing.” Nat. Chem. Biol. 2007; 3, 50-54; Peck et al., Chem. Biol. 2011; 18 (5), 619-630; the entire contents of each are hereby incorporated by reference

### Linker

The term “linker,” as used herein, refers to a molecule linking two other molecules or moieties. The linker can be an amino acid sequence in the case of a linker joining two fusion proteins. For example, a Cas9 can be fused to a polymerase (e.g., a reverse transcriptase) by an amino acid linker sequence. The linker can also be a nucleotide sequence in the case of joining two nucleotide sequences together. For example, in the instant case, the traditional guide RNA is linked via a spacer or linker nucleotide sequence to the RNA extension of a prime editing guide RNA which may comprise a RT template sequence and an RT primer binding site. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Locus

As used herein, a locus (plural loci) or “genetic locus” or “chromosomal locus” is a specific, fixed position on a chromosome where a particular gene or genetic marker is located. Each chromosome carries many genes, with each gene occupying a different position or locus. In other words, a locus is the specific physical location of a gene or other DNA sequence on a chromosome, like a genetic street address.

### Isolated

“Isolated” means altered or removed from the natural state. For example, a nucleic 20 acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In some embodiments, a gene of interest is encoded by an isolated nucleic acid. As used herein, the term “isolated,” refers to the characteristic of a material as provided herein being removed from its original or native environment (e.g., the natural environment if it is naturally occurring). Therefore, a naturally-occurring polynucleotide or protein or polypeptide present in a living animal is not isolated, but the same polynucleotide or polypeptide, separated by human intervention from some or all of the coexisting materials in the natural system, is isolated. An artificial or engineered material, for example, a non-naturally occurring nucleic acid construct, such as the expression constructs and vectors described herein, are, accordingly, also referred to as isolated. A material does not have to be purified in order to be isolated. Accordingly, a material may be part of a vector and/or part of a composition, and still be isolated in that such vector or composition is not part of the environment in which the material is found in nature.

napDNAbn

As used herein, the term “nucleic acid programmable DNA binding protein” or “napDNAbp,” of which Cas9 is an example, refer to proteins that use RNA:DNA hybridization to target and bind to specific sequences in a DNA molecule. Each napDNAbp is associated with at least one guide nucleic acid (e.g., guide RNA), or in the case of prime editing or twin prime editing, at least one PEgRNA, which localizes the napDNAbp to a DNA sequence that comprises a DNA strand (i.e., a target strand) that is complementary to the guide nucleic acid, or a portion thereof. In other words, the guide nucleic-acid “programs” the napDNAbp (e.g., Cas9 or equivalent) to localize and bind to a complementary sequence.

Without being bound by theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guide RNA or PEgRNA spacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions (e.g., a nick or a double-strand break). For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

As used herein, a “nickase” refers to a nucleic acid programmable DNA binding protein (napDNAbp) (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in a HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical Cas9 sequence or to an equivalent amino acid position in other Cas9 variants or Cas9 equivalents. In some embodiments, the nickase is a Cas9 that comprises a H840A, N854A, and/or N863A mutation relative to a canonical Cas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that one DNA strand is cut preferentially to the other DNA strand.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### PEgRNA

As used herein, the terms “prime editing guide RNA” or “PEgRNA” or “extended guide RNA” refer to a specialized form of a guide RNA that has been modified to include one or more additional sequences for implementing the prime editing methods (including twinPE methods) and compositions described herein. As described herein, the prime editing guide RNA comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with a portion of the endogenous target DNA to be edited, and (b) which comprises at least one desired nucleotide change (e.g., a transition, a transversion, a deletion, or an insertion, or a functional sequence, such as a SSR recognition sequence) to be introduced or integrated into the endogenous target DNA. In the case of twinPE, the PEgRNA does not require an extension arm having homology to the endogenous DNA since in twinPE a pair of 3′ flaps are formed by two separate PEgRNA binding on either side of a target site. The pair of 3′ flaps comprise a region of complementarity to one another such they the two 3′ flaps are capable of forming a duplex, which is then integrated in the target site of the DNA, replacing the endogenous region. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein the “primer binding site” comprises a sequence that hybridizes to a single-strand DNA sequence having a 3′ end generated from the nicked DNA of the R-loop.

### PE1

As used herein, “PE1” refers to a PE complex comprising a fusion protein comprising Cas9(H840A) and a wild type MMLV RT having the following structure: [NLS]-[Cas9(H840A)]-[linker]-[MMLV_RT(wt)]+a desired PEgRNA, wherein the PE fusion has the amino acid sequence of SEQ ID NO: 3, which is shown as follows;

(SEQ ID NO: 3)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFDE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGTAGFCRLWIPGFAEMAAPLYPLTKTGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLOHNCLDILAEAHGTRPDLTDQPLPD
ADHTWYTDGSSLLQEGQRKAGAAVTTETEVIWAKALPAGTSAQRA
ELIALTQALKMAEGKKLNVYTDSRYAFATAHIHGEIYRRRGLLTS
EGKEIKNKDEILALLKALFLPKRLSIIHCPGHQKGHSAEARGNRM
ADQAARKAAITETPDTSTLLIENSSPSGGSKRTADGSEFEPKKKR
KV
KEY:
NUCLEAR LOCALIZATION SEQUENCE (NLS)
TOP: (SEQ ID NO: 4),
BOTTOM: (SEQ ID NO: 5)
CAS9(H840A) (SEQ ID NO: 6)
33-AMINO ACID LINKER (SEQ ID NO: 7)
M-MLV reverse transcriptase (SEQ ID NO: 8).

### PE2

As used herein, “PE2” refers to a PE complex comprising a fusion protein comprising Cas9(H840A) and a variant MMLV RT having the following structure:[NLS]-[Cas9(H840A)]-[linker]-[MMLV_RT(D200N)(T330P)(L603W)(T306K)(W313F)]+a desired PEgRNA, wherein the PE fusion has the amino acid sequence of SEQ ID NO: 9, which is shown as follows:

(SEQ ID NO: 9)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFNE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGKAGFCRLFIPGFAEMAAPLYPLTKPGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLQHNCLDILAEAHGTRPDLTDQPLPD
ADHTWYTDGSSLLQEGQRKAGAAVTTETEVIWAKALPAGTSAQRA
ELIALTQALKMAEGKKLNVYTDSRYAFATAHIHGEIYRRRGWLTS
EGKEIKNKDEILALLKALFLPKRLSIIHCPGHQKGHSAEARGNRM
ADQAARKAAITETPDTSTLLIENSSPSGGSKRTADGSEFEPKKKR
KV
KEY:
NUCLEAR LOCALIZATION SEQUENCE (NLS)
TOP: (SEQ ID NO: 4),
BOTTOM: (SEQ ID NO: 5)
CAS9(H840A) (SEQ ID NO: 6)
33-AMINO ACID LINKER (SEQ ID NO: 7)
M-MLV reverse transcriptase (SEQ ID NO: 10).

### PE3

As used herein, “PE3” refers to PE2 plus a second-strand nicking guide RNA that complexes with the PE2 and introduces a nick in the non-edited DNA strand in order to induce preferential replacement of the edited strand.

### PE3b

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE-Short

As used herein, “PE-short” refers to a PE construct that is fused to a C-terminally truncated reverse transcriptase, and has the following amino acid sequence:

(SEQ ID NO: 11)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTFDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLFDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYFDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFNE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGKAGFCRLFIPGFAEMAAPLYPLTKPGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLOHNCLDNSRLINSGGSKRTADGSEF
EPKKKRKV
KEY:
NUCLEAR LOCALIZATION SEQUENCE (NLS)
TOP: (SEQ ID NO: 4),
BOTTOM: (SEQ ID NO: 5)
CAS9(H840A) (SEQ ID NO: 6)
33-AMINO ACID LINKER 1 (SEQ ID NO: 7)
M-MLV TRUNCATED REVERSE TRANSCRIPTASE
(SEQ ID NO: 12)

### Polymerase

As used herein, the term “polymerase” refers to an enzyme that synthesizes a nucleotide strand and that may be used in connection with the prime editor systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a PEgRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing and Multi-Flap Prime Editing (Aka Twin Prime Editing)

As used herein, the term “prime editing” or “classical prime editing” refers to an approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Classical prime editing is described in the inventors publication of Anzalone, A. V. et al. Search-and-replace genome editing without double-strand breaks or donor DNA. Nature 576, 149-157 (2019), which is incorporated herein by reference in its entirety.

Prime editing represents a platform for genome editing that is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same (or is homologous to) sequence as the endogenous strand (immediately downstream of the nick site) of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. The inventors have herein used Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., PEgRNA) containing a spacer sequence that anneals to a complement of a protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA.

In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand.

In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). The prime editing guide RNA (PEgRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp.

This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “PE system” or “PE editing system” refers the compositions involved in the method of genome editing using target-primed reverse transcription (TPRT) describe herein, including, but not limited to the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation, and in some embodiments, a donor DNA molecule comprising one or more site-specific recombinase recognition sequences.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template (e.g., the PEgRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

A prime editor system can comprise one or more prime editing guide RNAs (PEgRNAs). In some embodiments, a prime editor system has one PEgRNA (the “single flap prime editing system”) that targets one strand of a double stranded DNA, e.g., a target genomic site. For example, a single flap prime editing system may comprise a spacer sequence that comprises complementarity to a target strand of a double stranded target DNA, a primer binding site that comprises complementarity to a non-target strand of the double stranded target DNA, and a DNA synthesis template that comprises (and encodes) a nucleotide edit compared to the double stranded target DNA sequence, e.g., an SSR recognition site. In some embodiments, a prime editor system (the “dual-flap prime editing system” or “twin prime editing” or “twinPE”) comprises at least two different PEgRNAs that can target opposite strands of a double stranded target DNA, e.g., a target genomic site. For example, a twin prime editing system may comprise two PEgRNAs, wherein each of the two PEgRNAs comprises a DNA synthesis template having a region of complementarity to each other, and direct the synthesis of two 3′ flaps having a region of complementarity to each other and contains a nucleotide edit compared to the double stranded target DNA sequence, (e.g., an SSR recognition sequence). Unlike single flap prime editing, there is no requirement for the pair of edited DNA strands (3′ flaps) to directly compete with 5′ flaps in endogenous genomic DNA (i.e., no requirement for a homology arm in the extension arm which would generate a region having complementarity to the endogenous DNA), as the complementary edited strand is available for hybridization instead. Since both strands of the duplex are synthesized as edited DNA, the dual-flap prime editing system obviates the need for the replacement of the non-edited complementary DNA strand required by classical prime editing. Instead, cellular DNA repair machinery need only excise the paired 5′ flaps (original genomic DNA) and ligate the paired 3′ flaps (edited DNA) into the locus. Therefore, there is also no need to include sequences homologous to genomic DNA in the newly synthesized DNA strands, allowing selective hybridization of the new strands and facilitating edits that contain minimal genomic homology. Nuclease-active versions of prime editors that cut both strands of DNA could also be used to accelerate the removal of the original DNA sequence.

Variants of twin prime editing include quadruple-flap prime editing whereby the two sets of twin prime editors are used to introduce a genetic change at two different genetic loci, e.g., two different SSR recognition sequences located at the 5′ end and 3′ end of a gene.

Like classical prime editing, twin prime editing (including dual-flap and quadruple-flap prime editing) is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand.

### Prime Editor

The present disclosure relates to both prime editors and twin prime editing systems. The term “prime editor” refers to the herein described fusion constructs comprising a napDNAbp (e.g., Cas9 nickase) and a polymerase (e.g., a reverse transcriptase) and is capable of carrying out prime editing on a target nucleotide sequence in the presence of a PEgRNA (or “extended guide RNA”). The term “prime editor” may refer to the fusion protein alone. In some embodiments, the “prime editor” or “prime editor system” or “prime editing system” may refer to the complex between a prime editor fusion protein when it becomes associated with a PEgRNA, or to the nucleic acid molecules encoding same (e.g., vectors encoding a prime editor fusion protein and/or a PEgRNA that may be used clinically to deliver prime editing to a subject). The prime editing system may further be complexed with a second-strand nicking sgRNA. In some embodiments, the prime editor may be provided and/or delivered as separate components of a napDNAbp and a polymerase (e.g., reverse transcriptase) which are separately delivered (e.g., on separate vectors) and thus, provided in trans.

The dual-flap or twin prime editing system described herein comprises a pair of prime editor or a pair of prime editing systems and at least two PEgRNAs and are capable of installing an edit (e.g., an insertion, inversion, deletion, substitution or the like) at a target site in the DNA. For example, a twin prime editing system can be used to install an SSR recognition sequence.

The quadruple-flap prime editing system described herein comprises four prime editors and at least four PEgRNAs, and can be used to install at least two edits at two separate DNA target sites, e.g., an insertion, inversion, deletion, substitution or the like at two different DNA target sites. For example, a quadruple prime editing system can be used to install two SSR recognition sequences at two different DNA target sites.

### Primer Binding Site

The term “primer binding site” or “the PBS” refers to the nucleotide sequence located on a PEgRNA as a component of the extension arm and serves to bind to the primer sequence that is formed after Cas9 nicking of the target sequence by the prime editor. As detailed elsewhere, when the Cas9 nickase component of a prime editor nicks one strand of the target DNA sequence, a 3′-ended ssDNA flap is formed, which serves a primer sequence that anneals to the primer binding site on the PEgRNA to prime polymerization of DNA by the polymerase of the prime editing system (e.g., wherein the polymerase can be a reverse transcriptase).

### Promoter

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

With respect to endogenous eukaryotic genes and as a general principle, the 5′-flanking region of transcribed genes contains the promoter. The promoter contains specific sequences for binding the proteins necessary for transcription by RNA polymerase. The specific sequence in the promoter that positions the pol II is called the TATA box (consensus 5′-TATAAA-3′; some variants exist). Typically, the TATA box is located 25-30 bp upstream of the transcription start site (that is, −25 to −30 bp position), and for any given gene the position of the TATA box is fixed. However, many gene promoters lack the TATA box (TATA-less promoters). Accurate positioning of pol II in TATA-less promoters is thought to be mediated by two other cis-acting sequence elements, the initiator element (Inr) and the downstream promoter element (DPE). Inr has a consensus sequence of Y-+1-N-T/A-Y-Y (where Y is a pyrimidine, +1 is the transcription initiation site, N is any nucleotide), and DPE has a consensus sequence of (A/G)+28G(A/T)(C/T)(G/A/C)+32. Therefore, Inr occurs around the transcription start site and DPE occurs between 28 and 32 bases downstream from the transcription start site. Many variants of the Inr sequence have been reported. DPE has been most extensively studied in Drosophila. Some other sequences in the promoter that are found in most genes are the CAAT-box (around −75 to −80 bp position) and the GC-box (around −90 bp position). Various regions of the promoter have been termed the core (or basal), proximal, and distal promotor depending on their distance from the transcription start site. The core promoter is about 35 bp long and extends 35 bp upstream or downstream from the transcription site (−35 to +35), the proximal promoter is around 250 bp long, whereas the distal promoter is located further upstream. Therefore, the TATA box, Inr, and DPE are all contained within the core promoter, whereas the CAAT-box and the GC-box are contained within the proximal promoter. Core, proximal, and distal promoter elements cooperate to regulate transcription.

The proximal promoter contains additional cis-acting sequences that are necessary for the regulation of gene expression in response to specific stimuli. These sequences are called response elements or regulatory elements (RE). For example, genes that are induced by glucocorticoids have a glucocorticoid response element (GRE) in their promoters. Many such response elements have been identified so far in a number of animal and plant gene promoters. These response elements bind specific transcription regulatory proteins called transcription factors that control gene expression. Regulatory elements can also be found far upstream of the TATA box, far downstream in the 3′-flanking sequence, and even within introns. These elements typically act as enhancers because they significantly upregulate the expression of genes.

### Protospacer

As used herein, the term “protospacer” refers to the sequence (−20 bp) in DNA adjacent to the PAM (protospacer adjacent motif) sequence. The protospacer shares the same sequence as the spacer sequence of the guide RNA or PEgRNA. The guide RNA or PEgRNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 13, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Recombinase

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange. Recombinases have numerous applications, including the creation of gene knockouts/knock-ins and gene therapy applications. See, e.g., Brown et al., “Serine recombinases as tools for genome engineering.” Methods. 2011; 53(4):372-9; Hirano et al., “Site-specific recombinases as tools for heterologous gene integration.” Appl. Microbiol. Biotechnol. 2011; 92(2):227-39; Chavez and Calos, “Therapeutic applications of the (ΦC31 integrase system.” Curr. Gene Ther. 2011; 11(5):375-81; Turan and Bode, “Site-specific recombinases: from tag-and-target-to tag-and-exchange-based genomic modifications.” FASEB J. 2011; 25(12):4088-107; Venken and Bellen, “Genome-wide manipulations of Drosophila melanogaster with transposons, Flp recombinase, and (ΦC31 integrase.” Methods Mol. Biol. 2012; 859:203-28; Murphy, “Phage recombinases and their applications.” Adv. Virus Res. 2012; 83:367-414; Zhang et al., “Conditional gene manipulation: Cre-ating a new biological era.” J. Zhejiang Univ. Sci. B. 2012; 13(7):511-24; Karpenshif and Bernstein, “From yeast to mammals: recent advances in genetic control of homologous recombination.” DNA Repair (Amst). 2012; 1; 11(10):781-8; the entire contents of each are hereby incorporated by reference in their entirety.

The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety).

Other examples of recombinases that are useful in the methods and compositions described herein are known to those of skill in the art, and any new recombinase that is discovered or generated is expected to be able to be used in the different embodiments of the invention. In some embodiments, the catalytic domains of a recombinase are fused to a nuclease-inactivated RNA-programmable nuclease (e.g., dCas9, or a fragment thereof), such that the recombinase domain does not comprise a nucleic acid binding domain or is unable to bind to a target nucleic acid (e.g., the recombinase domain is engineered such that it does not have specific DNA binding activity). Recombinases lacking DNA binding activity and methods for engineering such are known, and include those described by Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.” Mol Microbiol. 2009; 74: 282-298; Akopian et al., “Chimeric recombinases with designed DNA sequence recognition.” Proc Nat Acad Sci USA. 2003; 100: 8688-8691; Gordley et al., “Evolution of programmable zinc finger-recombinases with activity in human cells. J Mol Biol. 2007; 367: 802-813; Gordley et al., “Synthesis of programmable integrases.” Proc Nat Acad Sci USA. 2009; 106: 5053-5058; Arnold et al., “Mutants of Tn3 resolvase which do not require accessory binding sites for recombination activity.” EMBO J. 1999; 18: 1407-1414; Gaj et al., “Structure-guided reprogramming of serine recombinase DNA sequence specificity.” Proc Natl Acad Sci USA. 2011; 108(2):498-503; and Proudfoot et al., “Zinc finger recombinases with adaptable DNA sequence specificity.” PLoS One. 2011; 6(4):e19537; the entire contents of each are hereby incorporated by reference.

For example, serine recombinases of the resolvase-invertase group, e.g., Tn3 and γδ resolvases and the Hin and Gin invertases, have modular structures with autonomous catalytic and DNA-binding domains (See, e.g., Grindley et al., “Mechanism of site-specific recombination.” Ann Rev Biochem. 2006; 75: 567-605, the entire contents of which are incorporated by reference). The catalytic domains of these recombinases are thus amenable to being recombined with nuclease-inactivated RNA-programmable nucleases (e.g., dCas9, or a fragment thereof) as described herein, e.g., following the isolation of ‘activated’ recombinase mutants which do not require any accessory factors (e.g., DNA binding activities) (See, e.g., Klippel et al., “Isolation and characterisation of unusual gin mutants.” EMBO J. 1988; 7: 3983-3989: Burke et al., “Activating mutations of Tn3 resolvase marking interfaces important in recombination catalysis and its regulation. Mol Microbiol. 2004; 51: 937-948; Olorunniji et al., “Synapsis and catalysis by activated Tn3 resolvase mutants.” Nucleic Acids Res. 2008; 36: 7181-7191; Rowland et al., “Regulatory mutations in Sin recombinase support a structure-based model of the synaptosome.” Mol Microbiol. 2009; 74: 282-298; Akopian et al., “Chimeric recombinases with designed DNA sequence recognition.” Proc Natl Acad Sci USA. 2003; 100: 8688-8691).

Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein (See, e.g., Smith et al., “Diversity in the serine recombinases.” Mol Microbiol. 2002; 44: 299-307, the entire contents of which are incorporated by reference).

Similarly, the core catalytic domains of tyrosine recombinases (e.g., Cre, λ integrase) are known, and can be similarly co-opted to engineer programmable site-specific recombinases as described herein (See, e.g., Guo et al., “Structure of Cre recombinase complexed with DNA in a site-specific recombination synapse.” Nature. 1997; 389:40-46; Hartung et al., “Cre mutants with altered DNA binding properties.” J Biol Chem 1998; 273:22884-22891; Shaikh et al., “Chimeras of the Flp and Cre recombinases: Tests of the mode of cleavage by Flp and Cre. J Mol Biol. 2000; 302:27-48; Rongrong et al., “Effect of deletion mutation on the recombination activity of Cre recombinase.” Acta Biochim Pol. 2005; 52:541-544; Kilbride et al., “Determinants of product topology in a hybrid Cre-Tn3 resolvase site-specific recombination system.” J Mol Biol. 2006; 355:185-195; Warren et al., “A chimeric cre recombinase with regulated directionality.” Proc Natl Acad Sci USA. 2008 105:18278-18283; Van Duyne, “Teaching Cre to follow directions.” Proc Natl Acad Sci USA. 2009 Jan. 6; 106(1):4-5; Numrych et al., “A comparison of the effects of single-base and triple-base changes in the integrase arm-type binding sites on the site-specific recombination of bacteriophage λ.” Nucleic Acids Res. 1990; 18:3953-3959; Tirumalai et al., “The recognition of core-type DNA sites by λ integrase.” J Mol Biol. 1998; 279:513-527; Aihara et al., “A conformational switch controls the DNA cleavage activity of λ integrase.” Mol Cell. 2003; 12:187-198; Biswas et al., “A structural basis for allosteric control of DNA recombination by λ integrase.” Nature. 2005; 435:1059-1066; and Warren et al., “Mutations in the amino-terminal domain of λ-integrase have differential effects on integrative and excisive recombination.” Mol Microbiol. 2005; 55:1104-1112; the entire contents of each are incorporated by reference).

### Recombinase Recognition Sequence

The term “recombinase recognition sequence”, or equivalently as “RRS” or “recombinase target sequence” or “recombinase site,” as used herein, refers to a nucleotide sequence target recognized by a recombinase and which undergoes strand exchange with another DNA molecule having a the RRS that results in excision, integration, inversion, or exchange of DNA fragments between the recombinase recognition sequences. In various embodiments, the multi-strand prime editors may install one or more recombinase sites in a target sequence, or in more than one target sequence. When more than one recombinase site is installed by a multi-strand prime editor, the recombinase sites can be installed at adjacent target sites or non-adjacent target sites (e.g., separate chromosomes). In various embodiments, single installed recombinase sites can be used as “landing sites” for a recombinase-mediated reaction between the genomic recombinase site and a second recombinase site within an exogenously supplied nucleic acid molecule, e.g., a plasmid. This enables the targeted integration of a desired nucleic acid molecule. In other embodiments, where two recombinase sites are inserted in adjacent regions of DNA (e.g., separated by 25-50 bp, 50-100 bp, 100-200 bp, 200-300 bp, 300-400 bp, 400-500 bp, 500-600 bp, 600-700 bp, 700-800 bp, 800-900 bp, 900-1000 bp, 1000-2000 bp, 2000-3000 bp, 3000-4000 bp, 4000-5000 bp, or more), the recombinase sites can be used for recombinase-mediated excision or inversion of the intervening sequence, or for recombinase-mediated cassette exchange with exogenous DNA having the same recombinase sites. When the two or more recombinase sites are installed by multi-flap prime editors on two different chromosomes, translocation of the intervening sequence can occur from a first chromosomal location to the second.

### Recombine or Recombination

The term “recombine,” or “recombination,” in the context of a nucleic acid modification (e.g., a genomic modification), is used to refer to the process by which two or more nucleic acid molecules, or two or more regions of a single nucleic acid molecule, are modified by the action of a recombinase protein (e.g., an inventive recombinase fusion protein provided herein). Recombination can result in, inter alia, the insertion, inversion, excision, or translocation of nucleic acids, e.g., in or between one or more nucleic acid molecules.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes which are error-prone in their DNA polymerization activity.

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### Protein Splicing

The term “protein splicing,” as used herein, refers to a process in which a sequence, an intein (or split inteins, as the case may be), is excised from within an amino acid sequence, and the remaining fragments of the amino acid sequence, the exteins, are ligated via an amide bond to form a continuous amino acid sequence. The term “trans” protein splicing refers to the specific case where the inteins are split inteins and they are located on different proteins.

### Second-Strand Nicking

The resolution of heteroduplex DNA (i.e., containing one edited and one non-edited strand) formed as a result of prime editing determines long-term editing outcomes. In words, a goal of prime editing is to resolve the heteroduplex DNA (the edited strand paired with the endogenous non-edited strand) formed as an intermediate of PE by permanently integrating the edited strand into the complement, endogenous strand. The approach of “second-strand nicking” can be used herein to help drive the resolution of heteroduplex DNA in favor of permanent integration of the edited strand into the DNA molecule. As used herein, the concept of “second-strand nicking” refers to the introduction of a second nick at a location downstream of the first nick (i.e., the initial nick site that provides the free 3′ end for use in priming of the reverse transcriptase on the extended portion of the guide RNA), preferably on the unedited strand. In certain embodiments, the first nick and the second nick are on opposite strands. In other embodiments, the first nick and the second nick are on opposite strands. In yet another embodiment, the first nick is on the non-target strand (i.e., the strand that forms the single strand portion of the R-loop), and the second nick is on the target strand. In still other embodiments, the first nick is on the edited strand, and the second nick is on the unedited strand. The second nick can be positioned at least 5 nucleotides downstream of the first nick, or at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream of the first nick. The second nick, in certain embodiments, can be introduced between about 5-150 nucleotides on the unedited strand away from the site of the PEgRNA-induced nick, or between about 5-140, or between about 5-130, or between about 5-120, or between about 5-110, or between about 5-100, or between about 5-90, or between about 5-80, or between about 5-70, or between about 5-60, or between about 5-50, or between about 5-40, or between about 5-30, or between about 5-20, or between about 5-10. In one embodiment, the second nick is introduced between 14-116 nucleotides away from the PEgRNA-induced nick. Without being bound by theory, the second nick induces the cell's endogenous DNA repair and replication processes towards replacement or editing of the unedited strand, thereby permanently installing the edited sequence on both strands and resolving the heteroduplex that is formed as a result of PE. In some embodiments, the edited strand is the non-target strand and the unedited strand is the target strand. In other embodiments, the edited strand is the target strand, and the unedited strand is the non-target strand.

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

In the context of a PEgRNA, the first step is the synthesis of a single-strand complementary DNA (i.e., the 3′ ssDNA flap, which becomes incorporated) oriented in the 5′ to 3′ direction which is templated off of the PEgRNA extension arm. Whether the 3′ ssDNA flap should be regarded as a sense or antisense strand depends on the direction of transcription since it well accepted that both strands of DNA may serve as a template for transcription (but not at the same time). Thus, in some embodiments, the 3′ ssDNA flap (which overall runs in the 5′ to 3′ direction) will serve as the sense strand because it is the coding strand. In other embodiments, the 3′ ssDNA flap (which overall runs in the 5′ to 3′ direction) will serve as the antisense strand and thus, the template for transcription.

### Sequence Homology

A “homologous sequence” or a sequence exhibiting “homology” to another sequence means a sequence of a nucleic acid molecule exhibiting at least about 65%, 70%, 75%, 80%, 85%, or 90% sequence identity to another nucleic acid molecule. In other embodiments, a “homologous sequence” of nucleic acids may exhibit 93%, 95% or 98% sequence identity to the reference nucleic acid.

When a percentage of sequence homology or identity is specified, in the context of two nucleic acid sequences or two polypeptide sequences, the percentage of homology or identity generally refers to the alignment of two or more sequences across a portion of their length when, compared and aligned for maximum correspondence. Unless stated otherwise, sequence homology or identity is assessed over the specified length of the nucleic acid, polypeptide, or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available internet interface for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at ebi.ac.uk/Tools/psa/emboss_needle/) which is part of the EMBOSS package (Rice P et al., Trends Genet., 2000; 16: 276-277), and the GGSEARCH program fasta.bioch. Virginia. edu/fasta_www2/fasta_www.cgi?rm=compare&pgm=gnw), which is part of the FASTA package (Pearson W and Lipman D, 1988, Proc. Natl. Acad. Sci. US A, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

### Spacer Sequence

As used herein, the term “spacer sequence” in connection with a guide RNA or a PEgRNA refers to the portion of the guide RNA or PEgRNA of about 20 nucleotides which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds. The term “target site” may equivalently be referred to as “target locus.”

### Temporal Second-Strand Nicking

As used herein, the term “temporal second-strand nicking” refers to a variant of second strand nicking whereby the installation of the second nick in the unedited strand occurs only after the desired edit is installed in the edited strand. This avoids concurrent nicks on both strands that could lead to double-stranded DNA breaks. The second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, mismatches between the protospacer and the unedited allele should disfavor nicking by the sgRNA until after the editing event on the PAM strand takes place.

### Transitions

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transversions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversions

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature as distinguished from mutant or variant forms.

### 5′ Endogenous DNA Flap

As used herein, the term “5′ endogenous DNA flap” refers to the strand of DNA situated immediately downstream of the PE-induced nick site in the target DNA. The nicking of the target DNA strand by PE exposes a 3′ hydroxyl group on the upstream side of the nick site and a 5′ hydroxyl group on the downstream side of the nick site. The endogenous strand ending in the 3′ hydroxyl group is used to prime the DNA polymerase of the prime editor (e.g., wherein the DNA polymerase is a reverse transcriptase). The endogenous strand on the downstream side of the nick site and which begins with the exposed 5′ hydroxyl group is referred to as the “5′ endogenous DNA flap” and is ultimately removed and replaced by the newly synthesized replacement strand (i.e., “3′ replacement DNA flap”) the encoded by the extension of the PEgRNA.

### 5′ Endogenous DNA Flap Removal

As used herein, the term “5′ endogenous DNA flap removal” or “5′ flap removal” refers to the removal of the 5′ endogenous DNA flap that forms when the RT-synthesized single-strand DNA flap competitively invades and hybridizes to the endogenous DNA, displacing the endogenous strand in the process. Removing this endogenous displaced strand can drive the reaction towards the formation of the desired product comprising the desired nucleotide change. The cell's own DNA repair enzymes may catalyze the removal or excision of the 5′ endogenous flap (e.g., a flap endonuclease, such as EXO1 or FEN1). Also, host cells may be transformed to express one or more enzymes that catalyze the removal of said 5′ endogenous flaps, thereby driving the process toward product formation (e.g., a flap endonuclease). Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference).

### 3′ Replacement DNA Flap

As used herein, the term “3′ replacement DNA flap” or simply, “replacement DNA flap,” refers to the strand of DNA that is synthesized by the prime editor and which is encoded by the extension arm of the prime editor PEgRNA. More in particular, the 3′ replacement DNA flap is encoded by the polymerase template of the PEgRNA. The 3′ replacement DNA flap comprises the same sequence as the 5′ endogenous DNA flap except that it also contains the edited sequence (e.g., single nucleotide change). The 3′ replacement DNA flap anneals to the target DNA, displacing or replacing the 5′ endogenous DNA flap (which can be excised, for example, by a 5′ flap endonuclease, such as FEN1 or EXO1) and then is ligated to join the 3′ end of the 3′ replacement DNA flap to the exposed 5′ hydoxyl end of endogenous DNA (exposed after excision of the 5′ endogenous DNA flap, thereby reforming a phosophodiester bond and installing the 3′ replacement DNA flap to form a heteroduplex DNA containing one edited strand and one unedited strand. DNA repair processes resolve the heteroduplex by copying the information in the edited strand to the complementary strand permanently installs the edit in to the DNA. This resolution process can be driven further to completion by nicking the unedited strand, i.e., by way of “second-strand nicking,” as described herein.

### DETAILED DESCRIPTION OF CERTAIN EMBODIMENTS

The instant disclosure provides constructs, systems, and methodologies that leverage the power of prime editing (PE), e.g., single-flap or “classical” PE or twinPE or multi-flap PE, to carry out site-specific and large-scale genetic modification, such as, but not limited to, insertions, deletions, inversions, replacements, and chromosomal translocations of whole or partial genes (e.g., whole gene, gene exons and/or introns, and gene regulatory regions). In certain embodiments, the disclosure provides constructs, systems, and methods using prime editing (PE), e.g., single-flap or “classical” PE or twinPE or multi-flap PE, to install a target site for site-specific recombination in a target genomic locus (e.g., a specific gene, exon, intron, or regulatory sequence). In certain other embodiments, the disclosure provides constructs, systems, and methods using prime editing (PE), e.g., single-flap or “classical” PE or twinPE or multi-flap PE, to install one or more target sites for site-specific recombination in a target genomic locus (e.g., a specific gene, exon, intron, or regulatory sequence). In still other embodiments, the disclosure provides constructs, systems, and methods using prime editing (PE), e.g., single-flap or “classical” PE or twinPE or multi-flap PE, to install one or more target sites for site-specific recombination in one or more target genomic loci (e.g., a specific gene, exon, intron, or regulatory sequence).

Once installed in the genome, a cognate SSR which recognizes the installed SSR recognition sequence may be used to catalyze the precise cleavage, strand exchange, and rejoining of DNA fragments at defined recombination targets without relying on endogenous repair mechanisms in a cell for repairing double-strand breaks which can induce indels and other undesirable DNA rearrangements. The reactions catalyzed by SSRs result in large-scale genomic changes, such as, insertions, deletions, inversions, replacements, and chromosomal translocations of whole or partial genes (e.g., whole gene, gene exons and/or introns, and gene regulatory regions).

In certain embodiments, the one or more SSR recognition sites can be inserted or introduced anywhere within genome. In some organism, a genome is organized as a single chromosome (e.g., bacteria). In other organisms, e.g., humans, the genome is organized into more than one chromosome. For instance, in humans, the genome comprises 23 pairs of chromosomes. In addition, the genome also may comprise mitochondrial DNA.

This U.S. Provisional Application incorporates by reference in its entirety International PCT Application No. PCT/US2021/031439, filed May 7, 2021. In addition, this U.S. Provisional Application also incorporates by reference in their entireties U.S. Provisional Application No. 63/022,397, filed May 8, 2020, and U.S. Provisional Application No. 63/116,785, filed Nov. 20, 2020, to which priority is claimed by the aforementioned PCT application.

This U.S. Provisional Application also refers to and incorporates by reference the following applications, U.S. Provisional Application No. 62/820,813, filed Mar. 19, 2019, U.S. Provisional Application No. 62/858,958, filed Jun. 7, 2019, U.S. Provisional Application No. 62/889,996, filed Aug. 21, 2019, U.S. Provisional Application No. 62/922,654, filed Aug. 21, 2019, U.S. Provisional Application No. 62/913,553, filed Oct. 10, 2019, U.S. Provisional Application No. 62/973,558, filed Oct. 10, 2019, U.S. Provisional Application No. 62/931,195, filed Nov. 5, 2019, U.S. Provisional Application No. 62/944,231, filed Dec. 5, 2019, U.S. Provisional Application No. 62/974,537, U.S. Provisional Application No. 62/991,069, filed Mar. 17, 2020, and U.S. Provisional Application No. (63/100,548), filed Mar. 17, 2020. In addition, this U.S. Provisional Application refers to and incorporates by reference in their entireties International PCT Application Nos.: PCT/US20/23721; PCT/US20/23730; PCT/US20/23713; PCT/US20/23712; PCT/US20/23727; PCT/US20/23724; PCT/US20/23725; PCT/US20/23728; PCT/US20/23732; PCT/US20/23723; PCT/US20/23553; and PCT/US20/23583, each filed on Mar. 19, 2020.

PE napDNAbps

The prime editors (PE) and/or twin prime editors (twinPE) and/or multi-flap prime editors (e.g., quadruple flap prime editor) utilized in the methods and compositions described herein may comprise a nucleic acid programmable DNA binding protein (napDNAbp). In one aspect, a napDNAbp can be associated with or complexed with at least one guide nucleic acid (e.g., guide RNA or a PEgRNA), which localizes the napDNAbp to a DNA sequence that comprises a DNA strand (i.e., a target strand) that is complementary to the guide nucleic acid, or a portion thereof (e.g., the spacer of a guide RNA that anneals to the protospacer of the DNA target). In other words, the guide nucleic-acid “programs” the napDNAbp (e.g., Cas9 or equivalent) to localize and bind to a complementary sequence of the protospacer in the DNA.

Any suitable napDNAbp may be used in the prime editors described herein. In various embodiments, the napDNAbp may be any Class 2 CRISPR-Cas system, including any type II, type V, or type VI CRISPR-Cas enzyme.

The below description of various napDNAbps which can be used in connection with the presently disclosed methods and compositions is not meant to be limiting in any way. The prime editors used in the methods and compositions described herein may comprise the canonical SpCas9, or any ortholog Cas9 protein, or any variant Cas9 protein—including any naturally occurring variant, mutant, or otherwise engineered version of Cas9—that is known or that can be made or evolved through a directed evolutionary or otherwise mutagenic process. In various embodiments, the Cas9 or Cas9 variants have a nickase activity, i.e., they only cleave one strand of the target DNA sequence. In other embodiments, the Cas9 or Cas9 variants have inactive nucleases, i.e., they are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

The prime editors utilized herein may also comprise Cas9 equivalents, including Cas12a (Cpf1) and Cas12b1 proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence or a reference Cas9 equivalent (e.g., Cas12a (Cpf1)).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12b1 (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S. A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The prime editors used in the methods and compositions of the present disclosure may use any suitable napDNAbp, including any suitable Cas9 or Cas9 equivalent.

In one embodiment, the prime editor constructs used in the methods and compositions described herein may comprise the “canonical SpCas9” nuclease from S. pyogenes, which has been widely used as a tool for genome engineering and is categorized as the type II subgroup of enzymes of the Class 2 CRISPR-Cas systems. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9, or a variant thereof (e.g., nCas9) can target that protein to virtually any DNA sequence simply by co-expression with an appropriate sgRNA. As used herein, the canonical SpCas9 protein refers to the wild-type protein from Streptococcus pyogenes having the following amino acid sequence:

| Description | Sequence | SEQ ID NO: |
|---|---|---|
| SpCas9 | MDKKYSIGLDIGTNSVGWAVI | 13 |
| Streptococcus | TDEYKVPSKKFKVLGNTDRH | |
| pyogenes | SIKKNLIGALLFDSGETAEA | |
| M1 | TRLKRTARRRYTRRKNRICY | |
| SwissProt | LQEIFSNEMAKVDDSFFHRL | |
| Accession | EESFLVEEDKKHERHPIFGN | |
| No. | IVDEVAYHEKYPTIYHLRKK | |
| Q99ZW2 | LVDSTDKADLRLIYLALAHM | |
| Wild type | IKFRGHFLIEGDLNPDNSDV | |
| | DKLFIQLVQTYNQLFEENPI | |
| | NASGVDAKAILSARLSKSRR | |
| | LENLIAQLPGEKKNGLFGNL | |
| | IALSLGLTPNFKSNFDLAED | |
| | AKLQLSKDTYDDDLDNLLAQ | |
| | IGDQYADLFLAAKNLSDAIL | |
| | LSDILRVNTEITKAPLSASM | |
| | IKRYDEHHQDLTLLKALVRQ | |
| | QLPEKYKEIFFDQSKNGYAG | |
| | YIDGGASQEEFYKFIKPILE | |
| | KMDGTEELLVKLNREDLLRK | |
| | QRTFDNGSIPHQIHLGELHA | |
| | ILRRQEDFYPFLKDNREKIE | |
| | KILTFRIPYYVGPLARGNSR | |
| | FAWMTRKSEETITPWNFEEV | |
| | VDKGASAQSFIERMTNFDKN | |
| | LPNEKVLPKHSLLYEYFTVY | |
| | NELTKVKYVTEGMRKPAFLS | |
| | GEQKKAIVDLLFKTNRKVTV | |
| | KQLKEDYFKKIECFDSVEIS | |
| | GVEDRFNASLGTYHDLLKII | |
| | KDKDFLDNEENEDILEDIVL | |
| | TLTLFEDREMIEERLKTYAH | |
| | LFDDKVMKQLKRRRYTGWGR | |
| | LSRKLINGIRDKQSGKTILD | |
| | FLKSDGFANRNFMQLIHDDS | |
| | LTFKEDIQKAQVSGQGDSLH | |
| | EHIANLAGSPAIKKGILQTV | |
| | KVVDELVKVMGRHKPENIVI | |
| | EMARENQTTQKGQKNSRERM | |
| | KRIEEGIKELGSQILKEHPV | |
| | ENTQLQNEKLYLYYLQNGRD | |
| | MYVDQELDINRLSDYDVDHI | |
| | VPQSFLKDDSIDNKVLTRSD | |
| | KNRGKSDNVPSEEVVKKMKN | |
| | YWRQLLNAKLITQRKFDNLT | |
| | KAERGGLSELDKAGFIKRQL | |
| | VETRQITKHVAQILDSRMNT | |
| | KYDENDKLIREVKVITLKSK | |
| | LVSDFRKDFQFYKVREINNY | |
| | HHAHDAYLNAVVGTALIKKY | |
| | PKLESEFVYGDYKVYDVRKM | |
| | IAKSEQEIGKATAKYFFYSN | |
| | IMNFFKTEITLANGEIRKRP | |
| | LIETNGETGEIVWDKGRDFA | |
| | TVRKVLSMPQVNIVKKTEVQ | |
| | TGGFSKESILPKRNSDKLIA | |
| | RKKDWDPKKYGGFDSPTVAY | |
| | SVLVVAKVEKGKSKKLKSVK | |
| | ELLGITIMERSSFEKNPIDF | |
| | LEAKGYKEVKKDLIIKLPKY | |
| | SLFELENGRKRMLASAGELQ | |
| | KGNELALPSKYVNFLYLASH | |
| | YEKLKGSPEDNEQKQLFVEQ | |
| | HKHYLDEIIEQISEFSKRVI | |
| | LADANLDKVLSAYNKHRDKP | |
| | IREQAENIIHLFTLTNLGAP | |
| | AAFKYFDTTIDRKRYTSTKE | |
| | VLDATLIHQSITGLYETRID | |
| | LSQLGGD | |

The prime editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 (SEQ ID NO: 13) entry, which include:

| SpCas9 mutation | Function/Characteristic |
|---|---|
| (relative to the amino | (as reported) (see UniProtKB- |
| acid sequence | Q99ZW2 (CAS9_STRPT1) |
| of the canonical SpCas9 | entry-incorporated |
| sequence, SEQ ID NO: 13) | herein by reference) |
| D10A | Nickase mutant which |
| | cleaves the protospacer |
| | strand (but no cleavage |
| | of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased |
| | DNA cleavage |
| 312-409 deletion | No nuclease activity |
| E762A | Nickase |
| H840A | Nickase mutant which |
| | cleaves the non-protospacer |
| | strand but does not |
| | cleave the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

In some embodiments, the Cas9 protein is any wild-type Cas9 protein. In some embodiments, the Cas9 protein can be a wild type Cas9 ortholog from another bacterial species different from the canonical Cas9 from S. pyogenes. For example, the following Cas9 orthologs can be used in connection with the prime editor constructs described in this specification: LfCas9 Lactobacillus fermentum wild type (GenBank: SNX31424.1); SaCas9 Staphylococcus aureus wild type (GenBank: AYD60528.1); SaCas9 Staphylococcus aureus; StCas9 Streptococcus thermophilus (UniProtKB/Swiss-Prot: G3ECR1.2) wild type; LcCas9 Lactobacillus crispatus (NCBI Reference Sequence: WP_133478044.1) wild type; PdCas9 Pediococcus damnosus (NCBI Reference Sequence: WP_062913273.1) wild type; FnCas9 Fusobacterium nucleatum (NCBI Reference Sequence: WP_060798984.1) wild type; EcCas9 Enterococcus cecorum (NCBI Reference Sequence: WP_047338501.1); AhCas9 Anaerostipes hadrus (NCBI Reference Sequence: WP_044924278.1) wild type; KvCas9 Kandleria vitulina (NCBI Reference Sequence: WP_031589969.1) wild type; EfCas9 Enterococcus faecalis (NCBI Reference Sequence: WP_016631044.1 wild type); Staphylococcus aureus Cas9; Geobacillus thermodenitrificans Cas9; and ScCas9 S. canis. In addition, any variant Cas9 orthologs having at least 80%, at least 85%, at least 90%, at least 95%, or at least 99% sequence identity to any of these orthologs may also be used with the prime editors utilized in the methods and compositions of the present disclosure.

In certain embodiments, the prime editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In one embodiment, the dead Cas9 may be based on the canonical SpCas9 sequence of Q99ZW2 and may have the following sequence, which comprises a D10X and an H810X, wherein X may be any amino acid, substitutions (underlined and bolded), or a variant be variant of SEQ ID NO: 13 having at least 80%, at least 85%, at least 90%, at least 95%, or at least 99% sequence identity thereto.

In one embodiment, the prime editors used in the methods and compositions described herein comprise a Cas9 nickase. The term “Cas9 nickase” or “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, H983A, D986A, or E762A, or a combination thereof.

In some embodiments, the prime editors utilized in the methods and compositions provided herein comprise other Cas9 variants, small-sized Cas9 variants, Cas9 equivalents (e.g., Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof), Cas9 circular permutants, Cas9 variants with modified PAM specificities, or divided napDNAbp domains for split PE delivery. Each of these classes of napDNAbp are described in detail in International Patent Application Nos. PCT/US2020/023721 and PCT/US2021/031439, each of which is incorporated by reference herein in its entirety.

### PE Polymerases

In various embodiments, the multi-flap prime editor system disclosed herein includes a polymerase (e.g., DNA-dependent DNA polymerase or RNA-dependent DNA polymerase, such as, reverse transcriptase), or a variant thereof, which can be provided as a fusion protein with a napDNAbp or other programmable nuclease, or provide in trans.

Any polymerase may be used in the multi-flap prime editors disclosed herein. The polymerases may be wild type polymerases, functional fragments, mutants, variants, or truncated variants, and the like. The polymerases may include wild type polymerases from eukaryotic, prokaryotic, archaeal, or viral organisms, and/or the polymerases may be modified by genetic engineering, mutagenesis, directed evolution-based processes. The polymerases may include T7 DNA polymerase, T5 DNA polymerase, T4 DNA polymerase, Klenow fragment DNA polymerase, DNA polymerase III and the like. The polymerases may also be thermostable, and may include Taq, Tne, Tma, Pfu, Tfl, Tth, Stoffel fragment, VENT® and DEEPVENT® DNA polymerases, KOD, Tgo, JDF3, and mutants, variants and derivatives thereof (see U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22(15):3259-3260 (1994), each of which are incorporated by reference). For synthesis of longer nucleic acid molecules (e.g, nucleic acid molecules longer than about 3-5 Kb in length), at least two DNA polymerases can be employed. In certain embodiments, one of the polymerases can be substantially lacking a 3′ exonuclease activity and the other may have a 3′ exonuclease activity. Such pairings may include polymerases that are the same or different. Examples of DNA polymerases substantially lacking in 3′ exonuclease activity include, but are not limited to, Taq, Tne(exo-), Tma(exo-), Pfu(exo-), Pwo(exo-), exo-KOD and Tth DNA polymerases, and mutants, variants and derivatives thereof.

Preferably, the polymerase usable in the multi-flap prime editors disclosed herein are “template-dependent” polymerase (since the polymerases are intended to rely on the DNA synthesis template to specify the sequence of the DNA strand under synthesis during prime editing. As used herein, the term “template DNA molecule” refers to that strand of a nucleic acid from which a complementary nucleic acid strand is synthesized by a DNA polymerase, for example, in a primer extension reaction of the DNA synthesis template of a PEgRNA.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)). The term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide. As such, in the case of prime editing, it can be said that the single strand of DNA synthesized by the polymerase of the prime editor against the DNA synthesis template is said to be “complementary” to the sequence of the DNA synthesis template.

### Exemplary Polymerases

In various embodiments, the multi-flap prime editors described herein comprise a polymerase. The disclosure contemplates any wild type polymerase obtained from any naturally-occurring organim or virus, or obtained from a commercial or non-commercial source. In addition, the polymerases usable in the multi-flap prime editors of the disclosure can include any naturally-occurring mutant polymerase, engineered mutant polymerase, or other variant polymerase, including truncated variants that retain function. The polymerases usable herein may also be engineered to contain specific amino acid substitutions, such as those specifically disclosed herein. In certain preferred embodiments, the polymerases usable in the multi-flap prime editors of the disclosure are template-based polymerases, i.e., they synthesize nucleotide sequences in a template-dependent manner.

A polymerase is an enzyme that synthesizes a nucleotide strand and which may be used in connection with the multi-flap prime editor systems described herein. The polymerases are preferrably “template-dependent” polymerases (i.e., a polymerase which synthesizes a nucleotide strand based on the order of nucleotide bases of a template strand). In certain configurations, the polymerases can also be a “template-independent” (i.e., a polymerase which synthesizes a nucleotide strand without the requirement of a template strand). A polymerase may also be further categorized as a “DNA polymerase” or an “RNA polymerase.” In various embodiments, the multi-flap prime editor systems comprise a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a PEgRNA), and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

In some embodiments, the polymerases can be from bacteriophage. Bacteriophage DNA polymerases are generally devoid of 5′ to 3′ exonuclease activity, as this activity is encoded by a separate polypeptide. Examples of suitable DNA polymerases are T4, T7, and phi29 DNA polymerase. The enzymes available commercially are: T4 (available from many sources e.g., Epicentre) and T7 (available from many sources, e.g. Epicentre for unmodified and USB for 3′ to 5′ exo T7 “Sequenase” DNA polymerase).

The other embodiments, the polymerases are archaeal polymerases. There are 2 different classes of DNA polymerases which have been identified in archaea: 1. Family B/pol I type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol I or pol II) can be derived from archaea with optimal growth temperatures that are similar to the desired assay temperatures.

Thermostable archaeal DNA polymerases are isolated from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to 3′ exonuclease activity, but in some cases 3′ to 5′ exonuclease activity is located in the same polypeptide.

There are a variety of commercially available Pol I DNA polymerases, some of which have been modified to reduce or abolish 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

Additional eubacteria related to those listed above are described in Thermophilic Bacteria (Kristjansson, J. K., ed.) CRC Press, Inc., Boca Raton, Fla., 1992.

The invention further provides for chimeric or non-chimeric DNA polymerases that are chemically modified according to methods disclosed in U.S. Pat. Nos. 5,677,152, 6,479,264 and 6,183,998, the contents of which are hereby incorporated by reference in their entirety.

Additional archaea DNA polymerases related to those listed above are described in the following references: Archaea: A Laboratory Manual (Robb, F. T. and Place, A. R., eds.), Cold Spring Harbor Laboratory Press, Cold Spring Harbor, N.Y., 1995 and Thermophilic Bacteria (Kristjansson, J. K., ed.) CRC Press, Inc., Boca Raton, Fla., 1992.

### Exemplary Reverse Transcriptases

In various embodiments, the multi-flap prime editors described herein comprise a reverse transcriptase as the polymerase. The disclosure contemplates any wild type reverse transcriptase obtained from any naturally-occurring organism or virus, or obtained from a commercial or non-commercial source. In addition, the reverse transcriptases usable in the multi-flap prime editors of the disclosure can include any naturally-occurring mutant RT, engineered mutant RT, or other variant RT, including truncated variants that retain function. The RTs may also be engineered to contain specific amino acid substitutions, such as those specifically disclosed herein.

Reverse transcriptases are multi-functional enzymes typically with three enzymatic activities including RNA- and DNA-dependent DNA polymerization activity, and an RNaseH activity that catalyzes the cleavage of RNA in RNA-DNA hybrids. Some mutants of reverse transcriptases have disabled the RNaseH moiety to prevent unintended damage to the mRNA. These enzymes that synthesize complementary DNA (cDNA) using mRNA as a template were first identified in RNA viruses. Subsequently, reverse transcriptases were isolated and purified directly from virus particles, cells or tissues. (e.g., see Kacian et al., 1971, Biochim. Biophys. Acta 46: 365-83; Yang et al., 1972, Biochem. Biophys. Res. Comm. 47: 505-11; Gerard et al., 1975, J. Virol. 15: 785-97; Liu et al., 1977, Arch. Virol. 55 187-200; Kato et al., 1984, J. Virol. Methods 9: 325-39; Luke et al., 1990, Biochem. 29: 1764-69 and Le Grice et al., 1991, J. Virol. 65: 7004-07, each of which are incorporated by reference). More recently, mutants and fusion proteins have been created in the quest for improved properties such as thermostability, fidelity and activity. Any of the wild type, variant, and/or mutant forms of reverse transcriptase which are known in the art or which can be made using methods known in the art are contemplated herein.

The reverse transcriptase (RT) gene (or the genetic information contained therein) can be obtained from a number of different sources. For instance, the gene may be obtained from eukaryotic cells which are infected with retrovirus, or from a number of plasmids which contain either a portion of or the entire retrovirus genome. In addition, messenger RNA-like RNA which contains the RT gene can be obtained from retroviruses. Examples of sources for RT include, but are not limited to, Moloney murine leukemia virus (M-MLV or MLVRT); human T-cell leukemia virus type 1 (HTLV-1); bovine leukemia virus (BLV); Rous Sarcoma Virus (RSV); human immunodeficiency virus (HIV); yeast, including Saccharomyces, Neurospora, Drosophila; primates; and rodents. See, for example, Weiss, et al., U.S. Pat. No. 4,663,290 (1987); Gerard, G. R., DNA:271-79 (1986); Kotewicz, M. L., et al., Gene 35:249-58 (1985); Tanese, N., et al., Proc. Natl. Acad. Sci. (USA):4944-48 (1985); Roth, M. J., at al., J. Biol. Chem. 260:9326-35 (1985); Michel, F., et al., Nature 316:641-43 (1985); Akins, R. A., et al., Cell 47:505-16 (1986), EMBO J. 4:1267-75 (1985); and Fawcett, D. F., Cell 47:1007-15 (1986) (each of which are incorporated herein by reference in their entireties).

### Wild Type RTs

Exemplary enzymes for use with the herein disclosed multi-flap prime editors can include, but are not limited to, M-MLV reverse transcriptase and RSV reverse transcriptase. Enzymes having reverse transcriptase activity are commercially available. In certain embodiments, the reverse transcriptase provided in trans to the other components of the multi-flap prime editor (PE) system. That is, the reverse transcriptase is expressed or otherwise provided as an individual component, i.e., not as a fusion protein with a napDNAbp.

A person of ordinary skill in the art will recognize that wild type reverse transcriptases, including but not limited to, Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomnatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

Exemplary wild type RT enzymes are as follows:

| DESCRIPTION | SEQUENCE |
|---|---|
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) WILD | VPCQSPWNTPLLPVKKPGTNDYRPVQDLREVNKRVEDIHPTV |
| TYPE | PNPYNLLSGLPPSHQWYTVLDLKDAFFCLRLHPTSQPLFAFEW |
| MOLONEY MURINE | RDPEMGISGQLTWTRLPQGFKNSPTLFDEALHRDLADFRIQHP |
| LEUKEMIA VIRUS | DLILLQYVDDLLLAATSELDCQQGTRALLQTLGNLGYRASAK |
| USED IN PE1 | KAQICQKQVKYLGYLLKEGQRWLTEARKETVMGQPTPKTPR |
| (PRIME EDITOR 1 | QLREFLGTAGFCRLWIPGFAEMAAPLYPLTKTGTLFNWGPDQ |
| FUSION PROTEIN | QKAYQEIKQALLTAPALGLPDLTKPFELFVDEKQGYAKGVLTQ |
| DISCLOSED | KLGPWRRPVAYLSKKLDPVAAGWPPCLRMVAAIAVLTKDAGK |
| HEREIN) | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 8) |
| REVERSE | AFPLERPDWDYTTQAGRNHLVHYRQLLLAGLQNAGRSPTNL |
| TRANSCRIPTASE | AKVKGITQGPNESPSAFLERLKEAYRRYTPYDPEDPGQETNVS |
| MOLONEY MURINE | MSFIWQSAPDIGRKLGRLEDLKSKTLGDLVREAEKIFNKRETP |
| LEUKEMIA VIRUS | EEREERIRRETEEKEERRRTVDEQKEKERDRRRHREMSKLLAT |
| REF SEQ. | VVIGQEQDRQEGERKRPQLDKDQCAYCKEKGHWAKDCPKKP |
| AAA66622.1 | RGPRGPRPQTSLLTLGDXGGQGQDPPPEPRITLKVGGQPVTFL |
| | VDTGAQHSVLTQNPGPLSDKSAWVQGATGGKRYRWTTDRKV |
| | HLATGKVTHSFLHVPDCPYPLLGRDLLTKLKAQIHFEGSGAQV |
| | VGPMGQPLQVLTLNIEDEYRLHETSKEPDVSLGFTWLSDFPQA |
| | WAESGGMGLAVRQAPLIIPLKATSTPVSIKQYPMSQEARLGIKP |
| | HIQRLLDQGILVPCQSPWNTPLLPVKKPGTNDYRPVQDLREV |
| | NKRVEDIHPTVPNPYNLLSGLPPSHQWYTVLDLKDAFFCLRL |
| | HPTSQPLFAFEWRDPEMGISGQLTWTRLPQGFKNSPTLFDEAL |
| | HRDLADFR (SEQ ID NO: 14) |
| REVERSE | TLQLEEEYRLFEPESTQKQEMDIWLKNFPQAWAETGGMGTAH |
| TRANSCRIPTASE | CQAPVLIQLKATATPISIRQYPMPHEAYQGIKPHIRRMLDQGIL |
| FELINE LEUKEMIA | KPCQSPWNTPLLPVKKPGTEDYRPVQDLREVNKRVEDIHPTV |
| VIRUS | PNPYNLLSTLPPSHPWYTVLDLKDAFFCLRLHSESQLLFAFEW |
| REF SEQ. NP955579.1 | RDPEIGLSGQLTWTRLPQGFKNSPTLFDEALHSDLADFRVRYP |
| | ALVLLQYVDDLLLAAATRTECLEGTKALLETLGNKGYRASAK |
| | KAQICLQEVTYLGYSLKDGQRWLTKARKEAILSIPVPKNSRQV |
| | REFLGTAGYCRLWIPGFAELAAPLYPLTRPGTLFQWGTEQQLA |
| | FEDIKKALLSSPALGLPDITKPFELFIDENSGFAKGVLVQKLGP |
| | WKRPVAYLSKKLDTVASGWPPCLRMVAAIAILVKDAGKLTLG |
| | QPLTILTSHPVEALVRQPPNKWLSNARMTHYQAMLLDAERVH |
| | FGPTVSLNPATLLPLPSGGNHHDCLQILAETHGTRPDLTDQPLP |
| | DADLTWYTDGSSFIRNGEREAGAAVTTESEVIWAAPLPPGTSA |
| | QRAELIALTQALKMAEGKKLTVYTDSRYAFATTHVHGEIYRRR |
| | GLLTSEGKEIKNKNEILALLEALFLPKRLSIIHCPGHQKGDSPQ |
| | AKGNRLADDTAKKAATETHSSLTVL |
| | SEQ ID NO: 15) |
| REVERSE | PISPIETVPVKLKPGMDGPKVKQWPLTEEKIKALVEICTEMEKE |
| TRANSCRIPTASE | GKISKIGPENPYNTPVFAIKKKDSTKWRKLVDFRELNKRTQDF |
| HIV-1 RT, CHAIN A | WEVQLGIPHPAGLKKKKSVTVLDVGDAYFSVPLDEDFRKYTA |
| REF SEQ. ITL3-A | FTIPSINNETPGIRYQYNVLPQGWKGSPAIFQSSMTKILEPFRKQ |
| | NPDIVIYQYMDDLYVGSDLEIGQHRTKIEELRQHLLRWGLTTP |
| | DKKHQKEPPFLWMGYELHPDKWTVQPIVLPEKDSWTVNDIQ |
| | KLVGKLNWASQIYPGIKVRQLXKLLRGTKALTEVIPLTEEAEL |
| | ELAENREILKEPVHGVYYDPSKDLIAEIQKQGQGQWTYQIYQ |
| | EPFKNLKTGKYARMRGAHTNDVKQLTEAVQKITTESIVIWGK |
| | TPKFKLPIQKETWETWWTEYWQATWIPEWEFVNTPPLVKLW |
| | YQLEKEPIVGAETFYVDGAANRETKLGKAGYVTNRGRQKVV |
| | TLTDTTNQKTELQAIYLALQDSGLEVNIVTDSQYALGIIQAQPD |
| | QSESELVNQIIEQLIKKEKVYLAWVPAHKGIGGNEQVDKLVSA |
| | GIRKV (SEQ ID NO: 16) |
| | SEE MARTINELLI ET AL., VIROLOGY, 1990, 174 (1): |
| | 135-144, WHICH IS INCORPORATED BY REFERENCE |
| REVERSE | PISPIETVPVKLKPGMDGPKVKQWPLTEEKIKALVEICTEMEKE |
| TRANSCRIPTASE | GKISKIGPENPYNTPVFAIKKKDSTKWRKLVDFRELNKRTQDF |
| HIV-1 RT, CHAIN B | WEVQLGIPHPAGLKKKKSVTVLDVGDAYFSVPLDEDFRKYTA |
| REF SEQ. ITL3-B | FTIPSINNETPGIRYQYNVLPQGWKGSPAIFQSSMTKILEPFRKQ |
| | NPDIVIYQYMDDLYVGSDLEIGQHRTKIEELRQHLLRWGLTTP |
| | DKKHQKEPPFLWMGYELHPDKWTVQPIVLPEKDSWTVNDIQ |
| | KLVGKLNWASQIYPGIKVRQLCKLLRGTKALTEVIPLTEEAELE |
| | LAENREILKEPVHGVYYDPSKDLIAEIQKQGQGQWTYQIYQEP |
| | FKNLKTGKYARMRGAHTNDVKQLTEAVQKITTESIVIWGKTP |
| | KFKLPIQKETWETWWTEYWQATWIPEWEFVNTPPLVKLWYQ |
| | LEKEPIVGAETF (SEQ ID NO: 17) |
| | SEE STAMMERS ET AL., J. MOL. BIOL., 1994, 242 (4): |
| | 586-588, WHICH IS INCORPORATED BY REFERENCE |
| REVERSE | TVALHLAIPLKWKPNHTPVWIDQWPLPEGKLVALTQLVEKEL |
| TRANSCRIPTASE | QLGHIEPSLSCWNTPVFVIRKASGSYRLLHDLRAVNAKLVPFG |
| ROUS SARCOMA | AVQQGAPVLSALPRGWPLMVLDLKDCFFSIPLAEQDREAFAF |
| VIRUS RT | TLPSVNNQAPARRFQWKVLPQGMTCSPTICQLIVGQILEPLRL |
| REF SEQ. ACL14945 | KHPSLRMLHYMDDLLLAASSHDGLEAAGEEVISTLERAGFTIS |
| | PDKVQKEPGVQYLGYKLGSTYAAPVGLVAEPRIATLWDVQKL |
| | VGSLQWLRPALGIPPRLRGPFYEQLRGSDPNEAREWNLDMK |
| | MAWREIVQLSTTAALERWDPALPLEGAVARCEQGAIGVLGQG |
| | LSTHPRPCLWLFSTQPTKAFTAWLEVLTLLITKLRASAVRTFGK |
| | EVDILLLPACFRDELPLPEGILLALRGFAGKIRSSDTPSIFDIARP |
| | LHVSLKVRVTDHPVPGPTVFTDASSSTHKGVVVWREGPRWEI |
| | KEIADLGASVQQLEARAVAMALLLWPTTPTNVVTDSAFVAKM |
| | LLKMGQEGVPSTAAAFILEDALSQRSAMAAVLHVRSHSEVPG |
| | FFTEGNDVADSQATFQAYPLREAKDLHTALHIGPRALSKACNI |
| | SMQQAREVVQTCPHCNSAPALEAGVNPRGLGPLQIWQTDFTL |
| | EPRMAPRSWLAVTVDTASSAIVVTQHGRVTSVAAQHHWATVI |
| | AVLGRPKAIKTDNGSCFTSKSTREWLARWGIAHTTGIPGNSQG |
| | QAMVERANRLLKDKIRVLAEGDGFMKRIPTSKQGELLAKAM |
| | YALNHFERGENTKTPIQKHWRPTVLTEGPPVKIRIETGEWEKG |
| | WNVLVWGRGYAAVKNRDTDKVIWVPSRKVKPDIAQKDEVT |
| | KKDEASPLFA (SEQ ID NO: 18) |
| | SEE YASUKAWA ET AL., J. BIOCHEM. 2009, 145 (3): |
| | 315-324, WHICH IS INCORPORATED BY REFERENCE |
| REVERSE | MMDHLLQKTQIQNQTEQVMNITNPNSIYIKGRLYFKGYKKIEL |
| TRANSCRIPTASE | HCFVDTGASLCIASKFVIPEEHWINAERPIMVKIADGSSITINKV |
| CAULIFLOWER | CRDIDLIIAGEIFHIPTVYQQESGIDFIIGNNFCQLYEPFIQFTDRV |
| MOSAIC VIRUS RT | IFTKDRTYPVHIAKLTRAVRVGTEGFLESMKKRSKTQQPEPVNI |
| REF SEQ. AGT42196 | STNKIAILSEGRRLSEEKLFITQQRMQKIEELLEKVCSENPLDP |
| | NKTKQWMKASIKLSDPSKAIKVKPMKYSPMDREEFDKQIKEL |
| | LDLKVIKPSKSPHMAPAFLVNNEAEKRRGKKRMVVNYKAMN |
| | KATVGDAYNLPNKDELLTLIRGKKIFSSFDCKSGFWQVLLDQD |
| | SRPLTAFTCPQGHYEWNVVPFGLKQAPSIFQRHMDEAFRVFR |
| | KFCCVYVDDILVFSNNEEDHLLHVAMILQKCNQHGIILSKKKA |
| | QLFKKKINFLGLEIDEGTHKPQGHILEHINKFPDTLEDKKQLQR |
| | FLGILTYASDYIPKLAQIRKPLQAKLKENVPWKWTKEDTLYM |
| | QKVKKNLQGFPPLHHPLPEEKLIIETDASDDYWGGMLKAIKIN |
| | EGTNTELICRYASGSFKAAEKNYHSNDKETLAVINTIKKFSIYL |
| | TPVHFLIRTDNTHFKSFVNLNYKGDSKLGRNIRWQAWLSHYS |
| | FDVEHIKGTDNHFADFLSREFNRVNS (SEQ ID NO: 19) |
| | SEE FARZADFAR ET AL., VIRUS GENES, 2013, 47 (2): |
| | 347-356, WHICH IS INCORPORATED BY REFERENCE |
| REVERSE | MKEKISKIDKNFYTDIFIKTSFQNEFEAGGVIPPIAKNQVSTISN |
| TRANSCRIPTASE | KNKTFYSLAHSSPHYSIQTRIEKFLLKNIPLSASSFAFRKERSYL |
| KLEBSIELLA | HYLEPHTQNVKYCHLDIVSFFHSIDVNIVRDTFSVYFSDEFLVK |
| PNEUMONIA | EKQSLLDAFMASVTLTAELDGVEKTFIPMGFKSSPSISNIIFRKI |
| REF SEQ. RFF81513.1 | DILIQKFCDKNKITYTRYADDLLFSTKKENNILSSTFFINEISSIL |
| | SINKFKLNKSKYLYKEGTISLGGYVIENILKDNSSGNIRLSSSKL |
| | NPLYKALYEIKKGSSSKHICIKVFNLKLKRFIYKKNKEKFEAKF |
| | YSSQLKNKLLGYRSYLLSFVIFHKKYKCINPIFLEKCVFLISEIE |
| | SIMNRKF (SEQ ID NO: 20) |
| REVERSE | MKITSNNVTAVINGKGWHSINWKKCHQHVKTIQTRIAKAACN |
| TRANSCRIPTASE | QQWRTVGRLQRLLVRSFSARALAVKRVTENSGRKTPGVDGQI |
| ESCHERICHIA COLI | WSTPESKWEAIFKLRRKGYKPLPLKRVFIPKSNGKKRPLGIPV |
| RT | MLDRAMQALHLLGLEPVSETNADHNSYGFRPARCTADAIQQ |
| REF SEQ. TGH57013 | VCNMYSSRNASKWVLEGDIKGCFEHISHEWLLENIPMDKQIL |
| | RNWLKAGIIEKSIFSKTLSGTPQGGIISPVLANMALDGLERLLQ |
| | NRFGRNRLI (SEQ ID NO: 21) |
| REVERSE | MSKIKINYEKYHIKPFPHFDQRIKVNKKVKENLQNPFYIAAHS |
| TRANSCRIPTASE | FYPFIHYKKISYKFKNGTLSSPKERDIFYSGHMDGYIYKHYGEI |
| BACILLUS SUBTILIS | LNHKYNNTCIGKGIDHVSLAYRNNKMGKSNIHFAAEVINFISE |
| RT | QQQAFIFVSDFSSYFDSLDHAILKEKLIEVLEEQDKLSKDWWN |
| REF SEQ. QBJ66766 | VFKHITRYNWVEKEEVISDLECTKEKIARDKKSRERYYTPAEF |
| | REFRKRVNIKSNDTGVGIPQGTAISAVLANVYAIDLDQKLNQY |
| | ALKYGGIYRRYSDDIIMVLPMTSDGQDPSNDHVSFIKSVVKRN |
| | KVTMGDSKTSVLYYANNNIYEDYQRKRESKMDYLGFSFDGM |
| | TVKIREKSLFKYYHRTYKKINSINWASVKKEKKVGRKKLYLL |
| | YSHLGRNYKGHGNFISYCKKAHAVFEGNKKIESLINQQIKRH |
| | WKKIQKRLVDV (SEQ ID NO: 22) |
| EUBACTERIUM | DTSNLMEQILSSDNLNRAYLQVVRNKGAEGVDGMKYTELKE |
| RECTALE GROUP II | HLAKNGETIKGQLRTRKYKPQPARRVEIPKPDGGVRNLGVPT |
| INTRON RT | VTDRFIQQAIAQVLTPIYEEQFHDHSYGFRPNRCAQQAILTALN |
| | IMNDGNDWIVDIDLEKFFDTVNHDKLMTLIGRTIKDGDVISIV |
| | RKYLVSGIMIDDEYEDSIVGTPQGGNLSPLLANIMLNELDKEM |
| | EKRGLNFVRYADDCIIMVGSEMSANRVMRNISRFIEEKLGLKV |
| | NMTKSKVDRPSGLKYLGFGFYFDPRAHQFKAKPHAKSVAKF |
| | KKRMKELTCRSWGVSNSYKVEKLNQLIRGWINYFKIGSMKTL |
| | CKELDSRIRYRLRMCIWKQWKTPQNQEKNLVKLGIDRNTARR |
| | VAYTGKRIAYVCNKGAVNVAISNKRLASFGLISMLDYYIEKCV |
| | TC (SEQ ID NO: 23) |
| GEOBACILLUS | ALLERILARDNLITALKRVEANQGAPGIDGVSTDQLRDYIRAH |
| STEAROTHERMOPHILUS | WSTIHAQLLAGTYRPAPVRRVEIPKPGGGTRQLGIPTVVDRLIQ |
| GROUP II | QAILQELTPIFDPDFSSSSFGFRPGRNAHDAVRQAQGYIQEGYR |
| INTRON RT | YVVDMDLEKFFDRVNHDILMSRVARKVKDKRVLKLIRAYLQA |
| | GVMIEGVKVQTEEGTPQGGPLSPLLANILLDDLDKELEKRGL |
| | KFCRYADDCNIYVKSLRAGQRVKQSIQRFLEKTLKLKVNEEKS |
| | AVDRPWKRAFLGFSFTPERKARIRLAPRSIQRLKQRIRQLTNPN |
| | WSISMPERIHRVNQYVMGWIGYFRLVETPSVLQTIEGWIRRRL |
| | RLCQWLQWKRVRTRIRELRALGLKETAVMEIANTRKGAWRTT |
| | KTPQLHQALGKTYWTAQGLKSLTQR (SEQ ID NO: 24) |

### Variant and Error-Prone RTs

Reverse transcriptases are essential for synthesizing complementary DNA (cDNA) strands from RNA templates. Reverse transcriptases are enzymes composed of distinct domains that exhibit different biochemical activities. The enzymes catalyze the synthesis of DNA from an RNA template, as follows: In the presence of an annealed primer, reverse transcriptase binds to an RNA template and initiates the polymerization reaction. RNA-dependent DNA polymerase activity synthesizes the complementary DNA (cDNA) strand, incorporating dNTPs. RNase H activity degrades the RNA template of the DNA:RNA complex. Thus, reverse transcriptases comprise (a) a binding activity that recognizes and binds to a RNA/DNA hybrid, (b) an RNA-dependent DNA polymerase activity, and (c) an RNase H activity. In addition, reverse transcriptases generally are regarded as having various attributes, including their thermostability, processivity (rate of dNTP incorporation), and fidelity (or error-rate). The reverse transcriptase variants contemplated herein may include any mutations to reverse transcriptase that impacts or changes any one or more of these enzymatic activities (e.g., RNA-dependent DNA polymerase activity, RNase H activity, or DNA/RNA hybrid-binding activity) or enzyme properties (e.g., thermostability, processivity, or fidelity). Such variants may be available in the art in the public domain, available commercially, or may be made using known methods of mutagenesis, including directed evolutionary processes (e.g., PACE or PANCE).

In various embodiments, the reverse transcriptase may be a variant reverse transcriptase. As used herein, a “variant reverse transcriptase” includes any naturally occurring or genetically engineered variant comprising one or more mutations (including singular mutations, inversions, deletions, insertions, and rearrangements) relative to a reference sequences (e.g., a reference wild type sequence). RT naturally have several activities, including an RNA-dependent DNA polymerase activity, ribonuclease H activity, and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. Variant RT's may comprise a mutation which impacts one or more of these activities (either which reduces or increases these activities, or which eliminates these activities all together). In addition, variant RTs may comprise one or more mutations which render the RT more or less stable, less prone to aggregation, and facilitates purification and/or detection, and/or other the modification of properties or characteristics.

A person of ordinary skill in the art will recognize that variant reverse transcriptases derived from other reverse transcriptases, including but not limited to Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase may be suitably used in the subject methods and composition described herein.

One method of preparing variant RTs is by genetic modification (e.g., by modifying the DNA sequence of a wild-type reverse transcriptase). A number of methods are known in the art that permit the random as well as targeted mutation of DNA sequences (see for example, Ausubel et. al. Short Protocols in Molecular Biology (1995) 3.sup.rd Ed. John Wiley & Sons, Inc.). In addition, there are a number of commercially available kits for site-directed mutagenesis, including both conventional and PCR-based methods. Examples include the QuikChange Site-Directed Mutagenesis Kits (AGILENT®), the Q5© Site-Directed Mutagenesis Kit (NEW ENGLAND BIOLABS®), and GeneArt™ Site-Directed Mutagenesis System (THERMOFISHER SCIENTIFIC®).

In addition, mutant reverse transcriptases may be generated by insertional mutation or truncation (N-terminal, internal, or C-terminal insertions or truncations) according to methodologies known to one skilled in the art. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation.

More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Methods of random mutagenesis, which will result in a panel of mutants bearing one or more randomly situated mutations, exist in the art. Such a panel of mutants may then be screened for those exhibiting the desired properties, for example, increased stability, relative to a wild-type reverse transcriptase.

An example of a method for random mutagenesis is the so-called “error-prone PCR method.” As the name implies, the method amplifies a given sequence under conditions in which the DNA polymerase does not support high fidelity incorporation. Although the conditions encouraging error-prone incorporation for different DNA polymerases vary, one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

In various aspects, the RT of the multi-flap prime editors may be an “error-prone” reverse transcriptase variant. Error-prone reverse transcriptases that are known and/or available in the art may be used. It will be appreciated that reverse transcriptases naturally do not have any proofreading function; thus the error rate of reverse transcriptase is generally higher than DNA polymerases comprising a proofreading activity. The error-rate of any particular reverse transcriptase is a property of the enzyme's “fidelity,” which represents the accuracy of template-directed polymerization of DNA against its RNA template. An RT with high fidelity has a low-error rate. Conversely, an RT with low fidelity has a high-error rate. The fidelity of M-MLV-based reverse transcriptases are reported to have an error rate in the range of one error in 15,000 to 27,000 nucleotides synthesized. See Boutabout et al., “DNA synthesis fidelity by the reverse transcriptase of the yeast retrotransposon Ty1,” Nucleic Acids Res, 2001, 29: 2217-2222, which is incorporated by reference. Thus, for purposes of this application, those reverse transcriptases considered to be “error-prone” or which are considered to have an “error-prone fidelity” are those having an error rate that is less than one error in 15,000 nucleotides synthesized.

Error-prone reverse transcriptase also may be created through mutagenesis of a starting RT enzyme (e.g., a wild type M-MLV RT). The method of mutagenesis is not limited and may include directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

Error-prone reverse transcriptases may also be obtain by phage-assisted non-continuous evolution (PANCE),” which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

Other error-prone reverse transcriptases have been described in the literature, each of which are contemplated for use in the herein methods and compositions. For example, error-prone reverse transcriptases have been described in Bebenek et al., “Error-prone Polymerization by HIV-1 Reverse Transcriptase,” J Biol Chem, 1993, Vol. 268: 10324-10334 and Sebastian-Martin et al., “Transcriptional inaccuracy threshold attenuates differences in RNA-dependent DNA synthesis fidelity between retroviral reverse transcriptases,” Scientific Reports, 2018, Vol. 8: 627, each of which are incorporated by reference. Still further, reverse transcriptases, including error-prone reverse transcriptases can be obtained from a commercial supplier, including ProtoScript® (II) Reverse Transcriptase, AMV Reverse Transcriptase, WarmStart® Reverse Transcriptase, and M-MuLV Reverse Transcriptase, all from NEW ENGLAND BIOLABS®, or AMV Reverse Transcriptase XL, SMARTScribe Reverse Transcriptase, GPR ultra-pure MMLV Reverse Transcriptase, all from TAKARA BIO US A, INC. (formerly CLONTECH).

The herein disclosure also contemplates reverse transcriptases having mutations in RNaseH domain. As mentioned above, one of the intrinsic properties of reverse transcriptases is the RNase H activity, which cleaves the RNA template of the RNA:cDNA hybrid concurrently with polymerization. The RNase H activity can be undesirable for synthesis of long cDNAs because the RNA template may be degraded before completion of full-length reverse transcription. The RNase H activity may also lower reverse transcription efficiency, presumably due to its competition with the polymerase activity of the enzyme. Thus, the present disclosure contemplates any reverse transcriptase variants that comprise a modified RNaseH activity.

The herein disclosure also contemplates reverse transcriptases having mutations in the RNA-dependent DNA polymerase domain. As mentioned above, one of the intrinsic properties of reverse transcriptases is the RNA-dependent DNA polymerase activity, which incorporates the nucleobases into the nascent cDNA strand as coded by the template RNA strand of the RNA:cDNA hybrid. The RNA-dependent DNA polymerase activity can be increased or decreased (i.e., in terms of its rate of incorporation) to either increase or decrease the processivity of the enzyme. Thus, the present disclosure contemplates any reverse transcriptase variants that comprise a modified RNA-dependent DNA polymerase activity such that the processivity of the enzyme of either increased or decreased relative to an unmodified version.

Also contemplated herein are reverse transcriptase variants that have altered thermostability characteristics. The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields, which can lead to an improved generation of the 3′ flap ssDNA as a result of the multi-flap prime editing process. Wild type M-MLV reverse transcriptase typically has an optimal temperature in the range of 37-48° C.; however, mutations may be introduced that allow for the reverse transcription activity at higher temperatures of over 48° C., including 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., and higher.

The variant reverse transcriptases contemplated herein, including error-prone RTs, thermostable RTs, increase-processivity RTs, can be engineered by various routine strategies, including mutagenesis or evolutionary processes. In some cases, the variants can be produced by introducing a single mutation. In other cases, the variants may require more than one mutation. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

Variant RT enzymes used herein may also include other “RT variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference RT protein, including any wild type RT, or mutant RT, or fragment RT, or other variant of RT disclosed or contemplated herein or known in the art.

In some embodiments, an RT variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT. In some embodiments, the RT variant comprises a fragment of a reference RT, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference RT. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type RT (M-MLV reverse transcriptase) (e.g., SEQ ID NO: 8) or to any of the reverse transcriptases of SEQ ID NOs: 14-24.

In some embodiments, the disclosure also may utilize RT fragments which retain their functionality and which are fragments of any herein disclosed RT proteins. In some embodiments, the RT fragment is at least 100 amino acids in length. In some embodiments, the fragment is at least 100, 150, 200, 250, 300, 350, 400, 450, 500, 550, or up to 600 or more amino acids in length.

In still other embodiments, the disclosure also may utilize RT variants which are truncated at the N-terminus or the C-terminus, or both, by a certain number of amino acids which results in a truncated variant which still retains sufficient polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end of the protein. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end of the protein. In still other embodiments, the RT truncated variant has a truncation at the N-terminal and the C-terminal end which are the same or different lengths.

For example, the multi-flap prime editors disclosed herein may include a truncated version of M-MLV reverse transcriptase. In this embodiment, the reverse transcriptase contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e., adeno-associated virus and lentivirus delivery). This embodiment is referred to as MMLV-RT(trunc) and has the following amino acid sequence:

| | |
|---|---|
| MMLV-RT | TLNIEDEYRLHETSKEPDVSLGSTWLSDFPQAWAETGGMGLAVRQ |
| (TRUNC) | APLIIPLKATSTPVSIKQYPMSQEARLGIKPHIQRLLDQGILVPCQSP |
| | WNTPLLPVKKPGTNDYRPVQDLREVNKRVEDIHPTVPNPYNLLSGL |
| | PPSHQWYTVLDLKDAFFCLRLHPTSQPLFAFEWRDPEMGISGQLT |
| | WTRLPQGFKNSPTLFNEALHRDLADFRIQHPDLILLQYVDDLLLAA |
| | TSELDCQQGTRALLQTLGNLGYRASAKKAQICQKQVKYLGYLLKEG |
| | QRWLTEARKETVMGQPTPKTPRQLREFLGKAGFCRLFIPGFAEMA |
| | APLYPLTKPGTLFNWGPDQQKAYQEIKQALLTAPALGLPDLTKPFE |
| | LFVDEKQGYAKGVLTQKLGPWRRPVAYLSKKLDPVAAGWPPCLR |
| | MVAAIAVLTKDAGKLTMGQPLVILAPHAVEALVKQPPDRWLSNAR |
| | MTHYQALLLDTDRVQFGPVVALNPATLLPLPEEGLQHNCLDNSRLI |
| | N (SEQ ID NO: 12) |

In various embodiments, the multi-flap prime editors disclosed herein may comprise one of the RT variants described herein, or a RT variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

In still other embodiments, the present methods and compositions may utilize a DNA polymerase that has been evolved into a reverse transcriptase, as described in Effefson et al., “Synthetic evolutionary origin of a proofreading reverse transcriptase,” Science, Jun. 24, 2016, Vol. 352: 1590-1593, the contents of which are incorporated herein by reference.

In certain other embodiments, the reverse transcriptase is provided as a component of a fusion protein also comprising a napDNAbp. In other words, in some embodiments, the reverse transcriptase is fused to a napDNAbp as a fusion protein.

In various embodiments, variant reverse transcriptases can be engineered from wild type M-MLV reverse transcriptase as represented by SEQ ID NO: 8.

In various embodiments, the multi-flap prime editors described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 8 or at a corresponding amino acid position in another wild type RT polypeptide sequence.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes:

| Description | Sequence (variant substitutions relative to wild type) |
|---|---|
| Reverse | TLNIEDEYRLHETSKEPDVSLGSTWLSDFPQAWAETGGMGLAVRQAP |
| transcriptase | LIIPLKATSTPVSIKQYPMSQEARLGIKPHIQRLLDQGILVPCQSPWNTPL |
| (M-MLV RT) | LPVKKPGTNDYRPVQDLREVNKRVEDIHPTVPNPYNLLSGLPPSHQWY |
| wild type | TVLDLKDAFFCLRLHPTSQPLFAFEWRDPEMGISGQLTWTRLPQGFKN |
| moloney | SPTLFDEALHRDLADFRIQHPDLILLQYVDDLLLAATSELDCQQGTRAL |
| murine | LQTLGNLGYRASAKKAQICQKQVKYLGYLLKEGQRWLTEARKETVM |
| leukemia | GQPTPKTPRQLREFLGTAGFCRLWIPGFAEMAAPLYPLTKTGTLFNWG |
| virus | PDQQKAYQEIKQALLTAPALGLPDLTKPFELFVDEKQGYAKGVLTQKL |
| Used in PE1 | GPWRRPVAYLSKKLDPVAAGWPPCLRMVAAIAVLTKDAGKLTMGQP |
| (prime editor | LVILAPHAVEALVKQPPDRWLSNARMTHYQALLLDTDRVQFGPVVAL |
| 1 fusion | NPATLLPLPEEGLQHNCLDILAEAHGTRPDLTDQPLPDADHTWYTDGS |
| protein | SLLQEGQRKAGAAVTTETEVIWAKALPAGTSAQRAELIALTQALKMA |
| disclosed | EGKKLNVYTDSRYAFATAHIHGEIYRRRGLLTSEGKEIKNKDEILALLK |
| herein) | ALFLPKRLSIIHCPGHQKGHSAEARGNRMADQAARKAAITETPDTSTLL |
| | IENSSP (SEQ ID NO: 8) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKTGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 25) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 26) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 27) |
| M-MLV RT | TLNIEDEYRLHETSKEPDVSLGSTWLSDFPQAWAETGGMGLAVRQAP |
| D200N | LIIPLKATSTPVSIKQYPMSQKARLGIKPHIQRLLDQGILVPCQSPWNTP |
| T330P | LLPVKKPGTNDYRPVQDLREVNKRVEDIHPTVPNPYNLLSGLPPSHQW |
| L603W | YTVLDLKDAFFCLRLHPTSQPLFAFEWRDPEMGISGQLTWTRLPQGFK |
| E69K | NSPTLFNEALHRDLADFRIQHPDLILLQYVDDLLLAATSELDCQQGTRA |
| | LLQTLGNLGYRASAKKAQICQKQVKYLGYLLKEGQRWLTEARKETV |
| | MGQPTPKTPRQLREFLGTAGFCRLWIPGFAEMAAPLYPLTKPGTLFNW |
| | GPDQQKAYQEIKQALLTAPALGLPDLTKPFELFVDEKQGYAKGVLTQ |
| | KLGPWRRPVAYLSKKLDPVAAGWPPCLRMVAAIAVLTKDAGKLTMG |
| | QPLVILAPHAVEALVKQPPDRWLSNARMTHYQALLLDTDRVQFGPVV |
| | ALNPATLLPLPEEGLQHNCLDILAEAHGTRPDLTDQPLPDADHTWYTD |
| | GSSLLQEGQRKAGAAVTTETEVIWAKALPAGTSAQRAELIALTQALK |
| | MAEGKKLNVYTDSRYAFATAHIHGEIYRRRGWLTSEGKEIKNKDEILA |
| | LLKALFLPKRLSIIHCPGHQKGHSAEARGNRMADQAARKAAITETPDT |
| | STLLIENSSP (SEQ ID NO: 28) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLRRFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP(SEQ ID NO: 29) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 30) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| L139P | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 31) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| L435G | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVIGAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 32) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| N454K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSKARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 33) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGKAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 34) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| W313F | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLFIPGFAEMAAPLYPLTKPGTLFNWGP |
| | DQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 35) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| D524G | SPTLFNEALHRDLADFRIQHPDLILLQYVDDLLLAATSELDCQQGTRAL |
| E562Q | LQTLGNLGYRASAKKAQICQKQVKYLGYLLKEGQRWLTEARKETVM |
| D583N | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTGGS |
| | SLLQEGQRKAGAAVTTETEVIWAKALPAGTSAQRAQLIALTQALKMA |
| | EGKKLNVYTNSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 36) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLRRFLGTAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 37) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| L139P | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 38) |
| M-MLV RT | TLNIEDEYRLHETSKEPDVSLGSTWLSDFPQAWAETGGMGLAVRQAP |
| P51L S67K | LIILLKATSTPVSIKQYPMKQEARLGIKPHIQRLLDQGILVPCQSPWNTP |
| T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFDEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P | WGPDQQKAYQEIKQALLTAPALGLPDLTKPFELFVDEKQGYAKGVLT |
| L435G | QKLGPWRRPVAYLSKKLDPVAAGWPPCLRMVAAIAVLTKDAGKLTM |
| N454K | GQPLVIGAPHAVEALVKQPPDRWLSKARMTHYQALLLDTDRVQFGPV |
| D524G | VALNPATLLPLPEEGLQHNCLDILAEAHGTRPDLTDQPLPDADHTWYT |
| D583N | GGSSLLQEGQRKAGAAVTTETEVIWAKALPAGTSAQRAELIALTQALK |
| H594Q | MAEGKKLNVYTNSRYAFATAHIQGEIYRRRGLLTSEGKEIKNKDEILA |
| D653N | LLKALFLPKRLSIIHCPGHQKGHSAEARGNRMANQAARKAAITETPDT |
| | STLLIENSSP (SEQ ID NO: 39) |
| M-MLV RT | TLNIEDEYRLHETSKEPDVSLGSTWLSDFPQAWAETGGMGLAVRQAP |
| D200N P51L | LIILLKATSTPVSIKQYPMKQEARLGIKPHIQRLLDQGILVPCQSPWNTP |
| S67K T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFNEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P L345G | WGPDQQKAYQEIKQALLTAPALGLPDLTKPFELFVDEKQGYAKGVLT |
| N454K | QKLGPWRRPVAYLSKKLDPVAAGWPPCLRMVAAIAVLTKDAGKLTM |
| D524G | GQPLVIGAPHAVEALVKQPPDRWLSKARMTHYQALLLDTDRVQFGPV |
| D583N | VALNPATLLPLPEEGLQHNCLDILAEAHGTRPDLTDQPLPDADHTWYT |
| H594Q | GGSSLLQEGQRKAGAAVTTETEVIWAKALPAGTSAQRAELIALTQALK |
| D653N | MAEGKKLNVYTNSRYAFATAHIQGEIYRRRGLLTSEGKEIKNKDEILA |
| | LLKALFLPKRLSIIHCPGHQKGHSAEARGNRMANQAARKAAITETPDT |
| | STLLIENSSP (SEQ ID NO: 40) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| in PE2 | GQPTPKTPRQLREFLGKAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 10) |

In various other embodiments, the multi-flap prime editors described herein (with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 8 or at a corresponding amino acid position in another wild type RT polypeptide sequence, wherein “X” can be any amino acid.

In various other embodiments, the multi-flap prime editors described herein (with RT provided as either a fusion partner or in trans) can include a variant RT comprising a P51X mutation in the wild type M-MLV RT of SEQ ID NO: 8 or at a corresponding amino acid position in another wild type RT polypeptide sequence, wherein “X” can be any amino acid. In certain embodiments, X is L.

In various other embodiments, the multi-flap prime editors described herein (with RT provided as either a fusion partner or in trans) can include a variant RT comprising a S67X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a E69X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a L139X mutation in the wild type M-MLV RT of SEQ ID NO: 8 or at a corresponding amino acid position in another wild type RT polypeptide sequence, wherein “X” can be any amino acid. In certain embodiments, X is P.

In various other embodiments, the multi-flap prime editors described herein (with RT provided as either a fusion partner or in trans) can include a variant RT comprising a T197X mutation in the wild type M-MLV RT of SEQ ID NO: 8 or at a corresponding amino acid position in another wild type RT polypeptide sequence, wherein “X” can be any amino acid. In certain embodiments, X is A.

In various other embodiments, the multi-flap prime editors described herein (with RT provided as either a fusion partner or in trans) can include a variant RT comprising a D200X mutation in the wild type M-MLV RT of SEQ ID NO: 8 or at a corresponding amino acid position in another wild type RT polypeptide sequence, wherein “X” can be any amino acid. In certain embodiments, X is N.

In various other embodiments, the multi-flap prime editors described herein (with RT provided as either a fusion partner or in trans) can include a variant RT comprising a H204X mutation in the wild type M-MLV RT of SEQ ID NO: 8 or at a corresponding amino acid position in another wild type RT polypeptide sequence, wherein “X” can be any amino acid. In certain embodiments, X is R.

In various other embodiments, the multi-flap prime editors described herein (with RT provided as either a fusion partner or in trans) can include a variant RT comprising a F209X mutation in the wild type M-MLV RT of SEQ ID NO: 8 or at a corresponding amino acid position in another wild type RT polypeptide sequence, wherein “X” can be any amino acid. In certain embodiments, X is N.

In various other embodiments, the multi-flap prime editors described herein (with RT provided as either a fusion partner or in trans) can include a variant RT comprising a E302X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a E302X mutation in the wild type M-MLV RT of SEQ ID NO: 89 or at a corresponding amino acid position in another wild type RT polypeptide sequence, wherein “X” can be any amino acid. In certain embodiments, X is R.

In various other embodiments, the multi-flap prime editors described herein (with RT provided as either a fusion partner or in trans) can include a variant RT comprising a T306X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a F309X mutation in the wild type M-MLV RT of SEQ ID NO: 8 or at a corresponding amino acid position in another wild type RT polypeptide sequence, wherein “X” can be any amino acid. In certain embodiments, X is N.

In various other embodiments, the multi-flap prime editors described herein (with RT provided as either a fusion partner or in trans) can include a variant RT comprising a W313X mutation in the wild type M-MLV RT of SEQ ID NO: 8 or at a corresponding amino acid position in another wild type RT polypeptide sequence, wherein “X” can be any amino acid. In certain embodiments, X is F.

In various other embodiments, the multi-flap prime editors described herein (with RT provided as either a fusion partner or in trans) can include a variant RT comprising a T330X mutation in the wild type M-MLV RT of SEQ ID NO: 8 or at a corresponding amino acid position in another wild type RT polypeptide sequence, wherein “X” can be any amino acid. In certain embodiments, X is P.

In various other embodiments, the multi-flap prime editors described herein (with RT provided as either a fusion partner or in trans) can include a variant RT comprising a L345X mutation in the wild type M-MLV RT of SEQ ID NO: 8 or at a corresponding amino acid position in another wild type RT polypeptide sequence, wherein “X” can be any amino acid. In certain embodiments, X is G.

In various other embodiments, the multi-flap prime editors described herein (with RT provided as either a fusion partner or in trans) can include a variant RT comprising a L435X mutation in the wild type M-MLV RT of SEQ ID NO: 8 or at a corresponding amino acid position in another wild type RT polypeptide sequence, wherein “X” can be any amino acid. In certain embodiments, X is G.

In various other embodiments, the multi-flap prime editors described herein (with RT provided as either a fusion partner or in trans) can include a variant RT comprising a N454X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a D524X mutation in the wild type M-MLV RT of SEQ ID NO: 8 or at a corresponding amino acid position in another wild type RT polypeptide sequence, wherein “X” can be any amino acid. In certain embodiments, X is G.

In various other embodiments, the multi-flap prime editors described herein (with RT provided as either a fusion partner or in trans) can include a variant RT comprising a E562X mutation in the wild type M-MLV RT of SEQ ID NO: 8 or at a corresponding amino acid position in another wild type RT polypeptide sequence, wherein “X” can be any amino acid. In certain embodiments, X is Q.

In various other embodiments, the multi-flap prime editors described herein (with RT provided as either a fusion partner or in trans) can include a variant RT comprising a D583X mutation in the wild type M-MLV RT of SEQ ID NO: 8 or at a corresponding amino acid position in another wild type RT polypeptide sequence, wherein “X” can be any amino acid. In certain embodiments, X is N.

In various other embodiments, the multi-flap prime editors described herein (with RT provided as either a fusion partner or in trans) can include a variant RT comprising a H594X mutation in the wild type M-MLV RT of SEQ ID NO: 8 or at a corresponding amino acid position in another wild type RT polypeptide sequence, wherein “X” can be any amino acid. In certain embodiments, X is Q.

In various other embodiments, the multi-flap prime editors described herein (with RT provided as either a fusion partner or in trans) can include a variant RT comprising a L603X mutation in the wild type M-MLV RT of SEQ ID NO: 8 or at a corresponding amino acid position in another wild type RT polypeptide sequence, wherein “X” can be any amino acid. In certain embodiments, X is W.

In various other embodiments, the multi-flap prime editors described herein (with RT provided as either a fusion partner or in trans) can include a variant RT comprising a E607X mutation in the wild type M-MLV RT of SEQ ID NO: 8 or at a corresponding amino acid position in another wild type RT polypeptide sequence, wherein “X” can be any amino acid. In certain embodiments, X is K.

In various other embodiments, the multi-flap prime editors described herein (with RT provided as either a fusion partner or in trans) can include a variant RT comprising a D653X mutation in the wild type M-MLV RT of SEQ ID NO: 8 or at a corresponding amino acid position in another wild type RT polypeptide sequence, wherein “X” can be any amino acid. In certain embodiments, X is N.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95% or at least 99% sequence identity to the wild-type enzymes or partial enzymes represented by SEQ ID NOs: 8, 10, 12, and 14-40.

The multi-flap prime editor system described here contemplates any publicly-available reverse transcriptase described or disclosed in any of the following U.S. patents (each of which are incorporated by reference in their entireties): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references describe reverse transcriptases in art. Each of their disclosures are incorporated herein by reference in their entireties.

Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).

Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).

Zhao, C., Liu, F. & Pyle, A. M. An ultraprocessive, accurate reverse transcriptase encoded by a metazoan group II intron. RNA 24, 183-195 (2018).

Zimmerly, S. & Wu, L. An Unexplored Diversity of Reverse Transcriptases in Bacteria. Microbiol Spectr 3, MDNA3-0058-2014 (2015).

Ostertag, E. M. & Kazazian Jr, H. H. Biology of Mammalian L1 Retrotransposons. Annual Review of Genetics 35, 501-538 (2001).

Perach, M. & Hizi, A. Catalytic Features of the Recombinant Reverse Transcriptase of Bovine Leukemia Virus Expressed in Bacteria. Virology 259, 176-189 (1999).

Lim, D. et al. Crystal structure of the moloney murine leukemia virus RNase H domain. J. Virol. 80, 8379-8389 (2006).

Zhao, C. & Pyle, A. M. Crystal structures of a group II intron maturase reveal a missing link in spliceosome evolution. Nature Structural & Molecular Biology 23, 558-565 (2016).

Griffiths, D. J. Endogenous retroviruses in the human genome sequence. Genome Biol. 2, REVIEWS1017 (2001).

Baranauskas, A. et al. Generation and characterization of new highly thermostable and processive M-MuLV reverse transcriptase variants. Protein Eng Des Sel 25, 657-668 (2012).

Zimmerly, S., Guo, H., Perlman, P. S. & Lambowltz, A. M. Group II intron mobility occurs by target DNA-primed reverse transcription. Cell 82, 545-554 (1995).

Feng, Q., Moran, J. V., Kazazian, H. H. & Boeke, J. D. Human L1 retrotransposon encodes a conserved endonuclease required for retrotransposition. Cell 87, 905-916 (1996).

Berkhout, B., Jebbink, M. & Zsíros, J. Identification of an Active Reverse Transcriptase Enzyme Encoded by a Human Endogenous HERV-K Retrovirus. Journal of Virology 73, 2365-2375 (1999).

Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).

Arezi, B. & Hogrefe, H. Novel mutations in Moloney Murine Leukemia Virus reverse transcriptase increase thermostability through tighter binding to template-primer. Nucleic Acids Res 37, 473-481 (2009).

Blain, S. W. & Goff, S. P. Nuclease activities of Moloney murine leukemia virus reverse transcriptase. Mutants with altered substrate specificities. J. Biol. Chem. 268, 23585-23592 (1993).

Xiong, Y. & Eickbush, T. H. Origin and evolution of retroelements based upon their reverse transcriptase sequences. EMBO J 9, 3353-3362 (1990).

Herschhorn, A. & Hizi, A. Retroviral reverse transcriptases. Cell. Mol. Life Sci. 67, 2717-2747 (2010).

Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).

Liu, M. et al. Reverse Transcriptase-Mediated Tropism Switching in Bordetella Bacteriophage. Science 295, 2091-2094 (2002).

Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).

Nottingham, R. M. et al. RNA-seq of human reference RNA samples using a thermostable group II intron reverse transcriptase. RNA 22, 597-613 (2016).

Telesnitsky, A. & Goff, S. P. RNase H domain mutations affect the interaction between Moloney murine leukemia virus reverse transcriptase and its primer-template. Proc. Natl. Acad. Sci. U.S. A. 90, 1276-1280 (1993).

Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).

Nowak, E. et al. Structural analysis of monomeric retroviral reverse transcriptase in complex with an RNA/DNA hybrid. Nucleic Acids Res 41, 3874-3887 (2013).

Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).

Das, D. & Georgiadis, M. M. The Crystal Structure of the Monomeric Reverse Transcriptase from Moloney Murine Leukemia Virus. Structure 12, 819-829 (2004).

Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).

Gerard, G. F. et al. The role of template-primer in protection of reverse transcriptase from thermal inactivation. Nucleic Acids Res 30, 3118-3129 (2002).

Monot, C. et al. The Specificity and Flexibility of L1 Reverse Transcription Priming at Imperfect T-Tracts. PLOS Genetics 9, e1003499 (2013).

Mohr, S. et al. Thermostable group II intron reverse transcriptase fusion proteins and their use in cDNA synthesis and next-generation RNA sequencing. RNA 19, 958-970 (2013).

Any of the references noted above which relate to reverse transcriptases are hereby incorporated by reference in their entireties, if not already stated so.

### Prime Editors

The disclosure provides systems comprising prime editors.

In one aspect, a prime editing system comprises (i) a napDNAbp and a DNA polymerase, or a polynucleotide encoding the napDNAbp and/or the DNA polymerase; and (ii) a prime editing guide RNA (PEgRNA) comprising a spacer, a gRNA core, and a DNA synthesis template, wherein the DNA synthesis template comprises one or more recombinase recognition site as compared to the target DNA.

In one aspect, a prime editing system can be used for simultaneously editing both strands of a double-stranded DNA sequence at a target site to be edited comprising a first prime editor complex and a second prime editor complex, wherein each of the first and second prime editor complexes comprises (1) a prime editor comprising (i) a nucleic acid programmable DNA binding protein (napDNAbp), and (ii) a polypeptide having an RNA-dependent DNA polymerase activity; and (2) a PEgRNA comprising a spacer sequence, gRNA core, a DNA synthesis template, and a primer binding site, wherein the DNA synthesis template of the PEgRNA of the first prime editor complex encodes a first single-stranded DNA sequence and the DNA synthesis template of the PEgRNA of the second prime editor complex encodes a second single-stranded DNA sequence, wherein the first single-stranded DNA sequence and the second single-stranded DNA sequence each comprises a region of complementarity to the other, and wherein the first single-stranded DNA sequence and the second single-stranded DNA sequence form a duplex comprising an edited portion as compared to the DNA sequence at the target site to be edited, which integrates into the target site to be edited.

In some aspect, a prime editor system comprises (i) a napDNAbp, a DNA polymerase or a polynucleotide encoding the napDNAbp and/or the DNA polymerase; (ii) a first prime editing guide RNA (PEgRNA) comprising a first spacer, a first gRNA core, and a first DNA synthesis template, or a polynucleotide encoding the first PEgRNA; and (iii) a second prime editing guide RNA (PEgRNA) comprising a second spacer, a second gRNA core, and a second DNA synthesis template, or a polynucleotide encoding the second PEgRNA, wherein the first DNA synthesis template and the second DNA synthesis template comprises a region of complementarity to each other, and wherein the sequence (first DNA synthesis template+second DNA synthesis template—region of complementarity between the first DNA synthesis template and second DNA synthesis template) comprises a recombinase recognition site as compared to the target DNA. In some embodiments, a prime editing system comprises a prime editor comprising (i) a napDNAbp and a DNA polymerase or a polynucleotide encoding the napDNAbp and/or the DNA polymerase; (ii) a first prime editing guide RNA (PEgRNA) comprising a first spacer, a first gRNA core, and a first DNA synthesis template, or a polynucleotide encoding the first PEgRNA; (iii) a second prime editing guide RNA (PEgRNA) comprising a second spacer, a second gRNA core, and a second DNA synthesis template, or a polynucleotide encoding the second PEgRNA; (iv) a third prime editing guide RNA (PEgRNA) comprising a third spacer, a third gRNA core, and a third DNA synthesis template, or a polynucleotide encoding the third PEgRNA; and (v) a fourth prime editing guide RNA (PEgRNA) comprising a fourth spacer, a fourth gRNA core, and a fourth DNA synthesis template, or a polynucleotide encoding the fourth PEgRNA; wherein the first DNA synthesis template and the second DNA synthesis template comprises a region of complementarity to each other, wherein the sequence (first DNA synthesis template+second DNA synthesis template—region of complementarity between the first DNA synthesis template and second DNA synthesis template) comprises a first recombinase recognition site as compared to the target DNA, wherein the third DNA synthesis template and the fourth DNA synthesis template comprises a region of complementarity to each other, and wherein the sequence (third DNA synthesis template+fourth DNA synthesis template—region of complementarity between the third DNA synthesis template and fourth DNA synthesis template) comprises a second recombinase recognition site as compared to the target DNA.

In another aspect, the present disclosure provides systems for editing one or more double-stranded DNA sequences, the system comprising:
a) a first prime editor complex comprising:
i.a first prime editor comprising a first nucleic acid programmable DNA binding protein (first napDNAbp) and a first polypeptide comprising an RNA-dependent DNA polymerase activity; and
ii.a first prime editing guide RNA (first PEgRNA) that binds to a first binding site on a first strand of a first double-stranded DNA sequence at a first target site to be edited;
b) a second prime editor complex comprising:
i.a second prime editor comprising a second nucleic acid programmable DNA binding protein (second napDNAbp) and a second polypeptide comprising an RNA-dependent DNA polymerase activity; and
ii.a second prime editing guide RNA (second PEgRNA) that binds to a second binding site on a second strand of the first double-stranded DNA sequence at the first target site to be edited;
c) a third prime editor complex comprising:
i.a third prime editor comprising a third nucleic acid programmable DNA binding protein (third napDNAbp) and a third polypeptide comprising an RNA-dependent DNA polymerase activity; and
ii.a third prime editing guide RNA (third PEgRNA) that binds to a first binding site on a first strand of a second double-stranded DNA sequence at a second target site to be edited;
d) a fourth prime editor complex comprising:
i.a fourth prime editor comprising a fourth nucleic acid programmable DNA binding protein (fourth napDNAbp) and a fourth polypeptide comprising an RNA-dependent DNA polymerase activity; and
ii.a fourth prime editing guide RNA (fourth PEgRNA) that binds to a second binding site on a second strand of the second double-stranded DNA sequence at the second target site to be edited;
   wherein the first PEgRNA comprises a first DNA synthesis template encoding a first single-stranded DNA sequence, the second PEgRNA comprises a second DNA synthesis template encoding a second single-stranded DNA sequence, the third PEgRNA comprises a third DNA synthesis template encoding a third single-stranded DNA sequence, and the fourth PEgRNA comprises a fourth DNA synthesis template encoding a fourth single-stranded DNA sequence;
   wherein the first and the third single-stranded DNA sequence each comprise a region of complementarity to the other; and wherein, wherein the second and the fourth single-stranded DNA sequence each comprise a region of complementarity to the other.

In some embodiments, the prime editing system further comprises a site specific recombinase, or a polynucleotide encoding the site specific recombinase.

In some embodiments, the prime editors used in the systems described herein (e.g., the first prime editor, second prime editor, third prime editor, and/or fourth prime editor in the systems described above) are provided as fusion proteins. In some embodiments, the prime editor fusion proteins comprise a napDNAbp and a polymerase (e.g., DNA-dependent DNA polymerase or RNA-dependent DNA polymerase, such as, reverse transcriptase). In some embodiments, the napDNAbp and the polymerase are optionally joined by linker to form the fusion protein. Various configurations of the prime editor fusion proteins and additional domains of the fusion proteins are described further herein. In various embodiments, the systems and methods provided by the present disclosure contemplate the use of any of the prime editor fusion proteins described herein.

In some embodiments, the prime editor complexes used in the systems described herein comprise a prime editor (e.g., the first prime editor, second prime editor, third prime editor, and/or fourth prime editor in the systems described above) where the components of one or more of the prime editors are provided in trans, as is described in additional detail throughout the present specification. In some embodiments, the prime editor comprises a napDNAbp and a polymerase expressed in trans. In some embodiments, the napDNAbp and the polymerase are expressed from one or more vectors (e.g., both components are expressed from the same vector, or each component is expressed from a different vector). In certain embodiments, the prime editors comprise additional components as described herein expressed in trans. In some embodiments, the prime editors used in the systems described herein may comprise both one or more prime editors provided as fusion proteins and one or more prime editors whose components are provided in trans.

In some embodiments, the prime editors and/or multi-flap prime editors (e.g., twinPE or quadruple flap) described herein contemplate fusion proteins comprising a napDNAbp and a polymerase (e.g., DNA-dependent DNA polymerase or RNA-dependent DNA polymerase, such as, reverse transcriptase), and optionally joined by a linker. The application contemplates any suitable napDNAbp and polymerase (e.g., DNA-dependent DNA polymerase or RNA-dependent DNA polymerase, such as, reverse transcriptase) to be combined in a single fusion protein. Examples of napDNAbps and polymerases (e.g., DNA-dependent DNA polymerase or RNA-dependent DNA polymerase, such as, reverse transcriptase) are each defined herein. Since polymerases are well-known in the art, and the amino acid sequences are readily available, this disclosure is not meant in any way to be limited to those specific polymerases identified herein.

In various embodiments, the fusion proteins may comprise any suitable structural configuration. For example, the fusion protein may comprise from the N-terminus to the C-terminus direction, a napDNAbp fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[27bp spacer]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂-[RT]-[napDNAbp]-COOH, wherein each instance of “]-[” indicates the presence of an optional linker sequence.

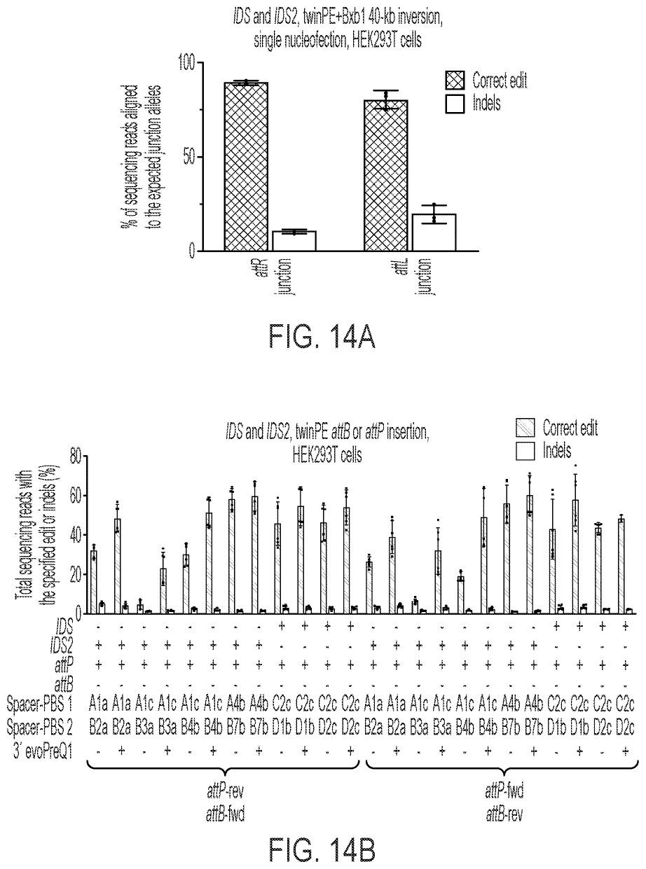

An exemplary fusion protein is depicted in FIG. 14, which shows a fusion protein comprising an MLV reverse transcriptase (“MLV-RT”) fused to a nickase Cas9 (“Cas9(H840A)”) via a linker sequence. This example is not intended to limit scope of fusion proteins that may be utilized for the prime editor (PE) system described herein.

In various embodiments, the prime editors and/or multi-flap prime editors (e.g., twinPE or quadruple flap) may have the following amino acid sequence (referred to herein as “PE1”), which includes a Cas9 variant comprising an H840A mutation (i.e., a Cas9 nickase) and an M-MLV RT wild type, as well as an N-terminal NLS sequence (19 amino acids) and an amino acid linker (32 amino acids) that joins the C-terminus of the Cas9 nickase domain to the N-terminus of the RT domain. The PE1 fusion protein has the following structure: [NLS]-[Cas9(H840A)]-[linker]-[MMLV_RT(wt)]. The amino acid sequence of PE1 and its individual components are as follows:

| DESCRIPTION | SEQUENCE |
|---|---|
| PE1 FUSION | MKRTADGSEFESPKKKRKVDKKYSIGLDIGTNSVGWAVITDEY |
| PROTEIN | KVPSKKFKVLGNTDRHSIKKNLIGALLFDSGETAEATRLKRTA |
| CAS9(H840A)- | RRRYTRRKNRICYLQEIFSNEMAKVDDSFFHRLEESFLVEEDK |
| MMLV_RT(WT) | KHERHPIFGNIVDEVAYHEKYPTIYHLRKKLVDSTDKADLRLI |
| | YLALAHMIKFRGHFLIEGDLNPDNSDVDKLFIQLVQTYNQLFE |
| | ENPINASGVDAKAILSARLSKSRRLENLIAQLPGEKKNGLFGN |
| | LIALSLGLTPNFKSNFDLAEDAKLQLSKDTYDDDLDNLLAQIG |
| | DQYADLFLAAKNLSDAILLSDILRVNTEITKAPLSASMIKRYDE |
| | HHQDLTLLKALVRQQLPEKYKEIFFDQSKNGYAGYIDGGASQ |
| | EEFYKFIKPILEKMDGTEELLVKLNREDLLRKQRTFDNGSIPH |
| | QIHLGELHAILRRQEDFYPFLKDNREKIEKILTFRIPYYVGPLA |
| | RGNSRFAWMTRKSEETITPWNFEEVVDKGASAQSFIERMTNF |
| | DKNLPNEKVLPKHSLLYEYFTVYNELTKVKYVTEGMRKPAFL |
| | SGEQKKAIVDLLFKTNRKVTVKQLKEDYFKKIECFDSVEISGV |
| | EDRFNASLGTYHDLLKIIKDKDFLDNEENEDILEDIVLTLTLFE |
| | DREMIEERLKTYAHLFDDKVMKQLKRRRYTGWGRLSRKLIN |
| | GIRDKQSGKTILDFLKSDGFANRNFMQLIHDDSLTFKEDIQKA |
| | QVSGQGDSLHEHIANLAGSPAIKKGILQTVKVVDELVKVMGR |
| | HKPENIVIEMARENQTTQKGQKNSRERMKRIEEGIKELGSQIL |
| | KEHPVENTQLQNEKLYLYYLQNGRDMYVDQELDINRLSDYD |
| | VDAIVPQSFLKDDSIDNKVLTRSDKNRGKSDNVPSEEVVKKM |
| | KNYWRQLLNAKLITQRKFDNLTKAERGGLSELDKAGFIKRQL |
| | VETRQITKHVAQILDSRMNTKYDENDKLIREVKVITLKSKLVS |
| | DFRKDFQFYKVREINNYHHAHDAYLNAVVGTALIKKYPKLES |
| | EFVYGDYKVYDVRKMIAKSEQEIGKATAKYFFYSNIMNFFKT |
| | EITLANGEIRKRPLIETNGETGEIVWDKGRDFATVRKVLSMPQ |
| | VNIVKKTEVQTGGFSKESILPKRNSDKLIARKKDWDPKKYGG |
| | FDSPTVAYSVLVVAKVEKGKSKKLKSVKELLGITIMERSSFEK |
| | NPIDFLEAKGYKEVKKDLIIKLPKYSLFELENGRKRMLASAGE |
| | LQKGNELALPSKYVNFLYLASHYEKLKGSPEDNEQKQLFVEQ |
| | HKHYLDEIIEQISEFSKRVILADANLDKVLSAYNKHRDKPIREQ |
| | AENIIHLFTLTNLGAPAAFKYFDTTIDRKRYTSTKEVLDATLIH |
| | QSITGLYETRIDLSQLGGDSGGSSGGSSGSETPGTSESATPESSG |
| | GSSGGSSTLNIEDEYRLHETSKEPDVSLGSTWLSDFPQAWAETGGMG |
| | LAVRQAPLIIPLKATSTPVSIKQYPMSQEARLGIKPHIQRLLDQGILVPC |
| | QSPWNTPLLPVKKPGTNDYRPVQDLREVNKRVEDIHPTVPNPYNLLS |
| | GLPPSHQWYTVLDLKDAFFCLRLHPTSQPLFAFEWRDPEMGISGQLT |
| | WTRLPQGFKNSPTLFDEALHRDLADFRIQHPDLILLQYVDDLLLAATS |
| | ELDCQQGTRALLQTLGNLGYRASAKKAQICQKQVKYLGYLLKEGQR |
| | WLTEARKETVMGQPTPKTPRQLREFLGTAGFCRLWIPGFAEMAAPLY |
| | PLTKTGTLFNWGPDQQKAYQEIKQALLTAPALGLPDLTKPFELFVDE |
| | KQGYAKGVLTQKLGPWRRPVAYLSKKLDPVAAGWPPCLRMVAAIAVL |
| | TKDAGKLTMGQPLVILAPHAVEALVKQPPDRWLSNARMTHYQALLLD |
| | TDRVQFGPVVALNPATLLPLPEEGLQHNCLDILAEAHGTRPDLTDQP |
| | LPDADHTWYTDGSSLLQEGQRKAGAAVTTETEVIWAKALPAGTSAQR |
| | AELIALTQALKMAEGKKLNVYTDSRYAFATAHIHGEIYRRRGLLTSEGK |
| | EIKNKDEILALLKALFLPKRLSIIHCPGHQKGHSAEARGNRMADQAAR |
| | KAAITETPDTSTLLIENSSPSGGSKRTADGSEFEPKKKRKV (SEQ ID |
| | NO: 3) |
| | KEY: |
| | NUCLEAR LOCALIZATION SEQUENCE (NLS) TOP: (SEQ ID NO: 4), |
| | BOTTOM: (SEQ ID NO: 5) |
| | CAS9(H840A) (SEQ ID NO: 6) |
| | 33-AMINO ACID LINKER (SEQ ID NO: 7) |
| | M-MLV REVERSE TRANSCRIPTASE (SEQ ID NO: 8) |
| PE1-N- | MKRTADGSEFESPKKKRKV (SEQ ID NO: 4) |
| TERMINAL NLS | |
| PE1-CAS9 | DKKYSIGLDIGTNSVGWAVITDEYKVPSKKFKVLGNTDRHSIKKN |
| (H840A) (MET | LIGALLFDSGETAEATRLKRTARRRYTRRKNRICYLQEIFSNEMAK |
| MINUS) | VDDSFFHRLEESFLVEEDKKHERHPIFGNIVDEVAYHEKYPTIYHL |
| | RKKLVDSTDKADLRLIYLALAHMIKFRGHFLIEGDLNPDNSDVDK |
| | LFIQLVQTYNQLFEENPINASGVDAKAILSARLSKSRRLENLIAQLP |
| | GEKKNGLFGNLIALSLGLTPNFKSNFDLAEDAKLQLSKDTYDDDL |
| | DNLLAQIGDQYADLFLAAKNLSDAILLSDILRVNTEITKAPLSASM |
| | IKRYDEHHQDLTLLKALVRQQLPEKYKEIFFDQSKNGYAGYIDGG |
| | ASQEEFYKFIKPILEKMDGTEELLVKLNREDLLRKQRTFDNGSIPH |
| | QIHLGELHAILRRQEDFYPFLKDNREKIEKILTFRIPYYVGPLARGN |
| | SRFAWMTRKSEETITPWNFEEVVDKGASAQSFIERMTNFDKNLPN |
| | EKVLPKHSLLYEYFTVYNELTKVKYVTEGMRKPAFLSGEQKKAIV |
| | DLLFKTNRKVTVKQLKEDYFKKIECFDSVEISGVEDRFNASLGTY |
| | HDLLKIIKDKDFLDNEENEDILEDIVLTLTLFEDREMIEERLKTYAH |
| | LFDDKVMKQLKRRRYTGWGRLSRKLINGIRDKQSGKTILDFLKS |
| | DGFANRNFMQLIHDDSLTFKEDIQKAQVSGQGDSLHEHIANLAGS |
| | PAIKKGILQTVKVVDELVKVMGRHKPENIVIEMARENQTTQKGQ |
| | KNSRERMKRIEEGIKELGSQILKEHPVENTQLQNEKLYLYYLQNG |
| | RDMYVDQELDINRLSDYDVDAIVPQSFLKDDSIDNKVLTRSDKN |
| | RGKSDNVPSEEVVKKMKNYWRQLLNAKLITQRKFDNLTKAERG |
| | GLSELDKAGFIKRQLVETRQITKHVAQILDSRMNTKYDENDKLIR |
| | EVKVITLKSKLVSDFRKDFQFYKVREINNYHHAHDAYLNAVVGT |
| | ALIKKYPKLESEFVYGDYKVYDVRKMIAKSEQEIGKATAKYFFYS |
| | NIMNFFKTEITLANGEIRKRPLIETNGETGEIVWDKGRDFATVRKV |
| | LSMPQVNIVKKTEVQTGGFSKESILPKRNSDKLIARKKDWDPKKY |
| | GGFDSPTVAYSVLVVAKVEKGKSKKLKSVKELLGITIMERSSFEKN |
| | PIDFLEAKGYKEVKKDLIIKLPKYSLFELENGRKRMLASAGELQK |
| | GNELALPSKYVNFLYLASHYEKLKGSPEDNEQKQLFVEQHKHYL |
| | DEIIEQISEFSKRVILADANLDKVLSAYNKHRDKPIREQAENIIHLFT |
| | LTNLGAPAAFKYFDTTIDRKRYTSTKEVLDATLIHQSITGLYETRID |
| | LSQLGGD (SEQ ID NO: 6) |
| PE1-LINKER | SGGSSGGSSGSETPGTSESATPESSGGSSGGSS (SEQ ID NO: 7) |
| BETWEEN CAS9 | |
| DOMAIN AND RT | |
| DOMAIN (33 | |
| AMINO ACIDS) | |
| PE1-M-MLV RT | TLNIEDEYRLHETSKEPDVSLGSTWLSDFPQAWAETGGMGLAVR |
| | QAPLIIPLKATSTPVSIKQYPMSQEARLGIKPHIQRLLDQGILVPCQS |
| | PWNTPLLPVKKPGTNDYRPVQDLREVNKRVEDIHPTVPNPYNLLS |
| | GLPPSHQWYTVLDLKDAFFCLRLHPTSQPLFAFEWRDPEMGISGQ |
| | LTWTRLPQGFKNSPTLFDEALHRDLADFRIQHPDLILLQYVDDLL |
| | LAATSELDCQQGTRALLQTLGNLGYRASAKKAQICQKQVKYLGY |
| | LLKEGQRWLTEARKETVMGQPTPKTPRQLREFLGTAGFCRLWIPG |
| | FAEMAAPLYPLTKTGTLFNWGPDQQKAYQEIKQALLTAPALGLPD |
| | LTKPFELFVDEKQGYAKGVLTQKLGPWRRPVAYLSKKLDPVAAG |
| | WPPCLRMVAAIAVLTKDAGKLTMGQPLVILAPHAVEALVKQPPDR |
| | WLSNARMTHYQALLLDTDRVQFGPVVALNPATLLPLPEEGLQHN |
| | CLDILAEAHGTRPDLTDQPLPDADHTWYTDGSSLLQEGQRKAGA |
| | AVTTETEVIWAKALPAGTSAQRAELIALTQALKMAEGKKLNVYT |
| | DSRYAFATAHIHGEIYRRRGLLTSEGKEIKNKDEILALLKALFLPKR |
| | LSIIHCPGHQKGHSAEARGNRMADQAARKAAITETPDTSTLLIENS |
| | SP (SEQ ID NO: 8) |
| PE1-C- | SGGSKRTADGSEFEPKKKRKV (SEQ ID NO: 5) |
| TERMINAL NLS | |

In another embodiment, the prime editors and/or multi-flap prime editors (e.g., twinPE or quadruple flap) may have the following amino acid sequence (referred to herein as “PE2”), which includes a Cas9 variant comprising an H840A mutation (i.e., a Cas9 nickase) and an M-MLV RT comprising mutations D200N, T330P, L603W, T306K, and W313, as well as an N-terminal NLS sequence (19 amino acids) and an amino acid linker (33 amino acids) that joins the C-terminus of the Cas9 nickase domain to the N-terminus of the RT domain. The PE2 fusion protein has the following structure:[NLS]-[Cas9(H840A)]-[linker]-[MMLV_RT(D200N)(T330P)(L603)(T306K)(W313F)]. The amino acid sequence of PE2 is as follows:

| | |
|---|---|
| PE2 FUSION | MKRTADGSEFESPKKKRKVDKKYSIGLDIGTNSVGWAVITDEYK |
| PROTEIN | VPSKKFKVLGNTDRHSIKKNLIGALLFDSGETAEATRLKRTAR |
| CAS9(H840A)- | RRYTRRKNRICYLQEIFSNEMAKVDDSFFHRLEESFLVEEDKK |
| MMLV_RT | HERHPIFGNIVDEVAYHEKYPTIYHLRKKLVDSTDKADLRLIYL |
| D200N | ALAHMIKFRGHFLIEGDLNPDNSDVDKLFIQLVQTYNQLFEEN |
| T330P | PINASGVDAKAILSARLSKSRRLENLIAQLPGEKKNGLFGNLIA |
| L603W | LSLGLTPNFKSNFDLAEDAKLQLSKDTYDDDLDNLLAQIGDQY |
| T306K | ADLFLAAKNLSDAILLSDILRVNTEITKAPLSASMIKRYDEHHQ |
| W313F | DLTLLKALVRQQLPEKYKEIFFDQSKNGYAGYIDGGASQEEFY |
| | KFIKPILEKMDGTEELLVKLNREDLLRKQRTFDNGSIPHQIHLG |
| | ELHAILRRQEDFYPFLKDNREKIEKILTFRIPYYVGPLARGNSR |
| | FAWMTRKSEETITPWNFEEVVDKGASAQSFIERMTNFDKNLPN |
| | EKVLPKHSLLYEYFTVYNELTKVKYVTEGMRKPAFLSGEQKK |
| | AIVDLLFKTNRKVTVKQLKEDYFKKIECFDSVEISGVEDRFNAS |
| | LGTYHDLLKIIKDKDFLDNEENEDILEDIVLTLTLFEDREMIEER |
| | LKTYAHLFDDKVMKQLKRRRYTGWGRLSRKLINGIRDKQSG |
| | KTILDFLKSDGFANRNFMQLIHDDSLTFKEDIQKAQVSGQGDS |
| | LHEHIANLAGSPAIKKGILQTVKVVDELVKVMGRHKPENIVIE |
| | MARENQTTQKGQKNSRERMKRIEEGIKELGSQILKEHPVENT |
| | QLQNEKLYLYYLQNGRDMYVDQELDINRLSDYDVDAIVPQSFL |
| | KDDSIDNKVLTRSDKNRGKSDNVPSEEVVKKMKNYWRQLLNA |
| | KLITQRKFDNLTKAERGGLSELDKAGFIKRQLVETRQITKHVA |
| | QILDSRMNTKYDENDKLIREVKVITLKSKLVSDFRKDFQFYKV |
| | REINNYHHAHDAYLNAVVGTALIKKYPKLESEFVYGDYKVYDV |
| | RKMIAKSEQEIGKATAKYFFYSNIMNFFKTEITLANGEIRKRPL |
| | IETNGETGEIVWDKGRDFATVRKVLSMPQVNIVKKTEVQTGG |
| | FSKESILPKRNSDKLIARKKDWDPKKYGGFDSPTVAYSVLVVAK |
| | VEKGKSKKLKSVKELLGITIMERSSFEKNPIDFLEAKGYKEVK |
| | KDLIIKLPKYSLFELENGRKRMLASAGELQKGNELALPSKYVN |
| | FLYLASHYEKLKGSPEDNEQKQLFVEQHKHYLDEIIEQISEFSK |
| | RVILADANLDKVLSAYNKHRDKPIREQAENIIHLFTLTNLGAPA |
| | AFKYFDTTIDRKRYTSTKEVLDATLIHQSITGLYETRIDLSQLG |
| | GDSGGSSGGSSGSETPGTSESATPESSGGSSGGSSTLNIEDEYRLH |
| | ETSKEPDVSLGSTWLSDFPQAWAETGGMGLAVRQAPLIIPLKATSTPVS |
| | IKQYPMSQEARLGIKPHIQRLLDQGILVPCQSPWNTPLLPVKKPGTND |
| | YRPVQDLREVNKRVEDIHPTVPNPYNLLSGLPPSHQWYTVLDLKDAFF |
| | CLRLHPTSQPLFAFEWRDPEMGISGQLTWTRLPQGFKNSPTLFNEALH |
| | RDLADFRIQHPDLILLQYVDDLLLAATSELDCQQGTRALLQTLGNLGY |
| | RASAKKAQICQKQVKYLGYLLKEGQRWLTEARKETVMGQPTPKTPRQ |
| | LREFLGKAGFCRLFIPGFAEMAAPLYPLTKPGTLFNWGPDQQKAYQEI |
| | KQALLTAPALGLPDLTKPFELFVDEKQGYAKGVLTQKLGPWRRPVAYL |
| | SKKLDPVAAGWPPCLRMVAAIAVLTKDAGKLTMGQPLVILAPHAVEAL |
| | VKQPPDRWLSNARMTHYQALLLDTDRVQFGPVVALNPATLLPLPEEGL |
| | QHNCLDILAEAHGTRPDLTDQPLPDADHTWYTDGSSLLQEGQRKAGA |
| | AVTTETEVIWAKALPAGTSAQRAELIALTQALKMAEGKKLNVYTDSRYAF |
| | ATAHIHGEIYRRRGWLTSEGKEIKNKDEILALLKALFLPKRLSIIHCPGH |
| | QKGHSAEARGNRMADQAARKAAITETPDTSTLLIENSSPSGGSKRTAD |
| | GSEFEPKKKRKV (SEQ ID NO: 9) |
| | KEY: |
| | NUCLEAR LOCALIZATION SEQUENCE (NLS) TOP: (SEQ ID NO: 4), |
| | BOTTOM: (SEQ ID NO: 5) |
| | CAS9(H840A) (SEQ ID NO: 6) |
| | 33-AMINO ACID LINKER (SEQ ID NO: 7) |
| | M-MLV REVERSE TRANSCRIPTASE (SEQ ID NO: 10) |
| PE2-N- | MKRTADGSEFESPKKKRKV (SEQ ID NO: 4) |
| TERMINAL NLS | |
| PE2-CAS9 | DKKYSIGLDIGTNSVGWAVITDEYKVPSKKFKVLGNTDRHSIKKNL |
| (H840A) (MET | IGALLFDSGETAEATRLKRTARRRYTRRKNRICYLQEIFSNEMAKV |
| MINUS) | DDSFFHRLEESFLVEEDKKHERHPIFGNIVDEVAYHEKYPTIYHLRK |
| | KLVDSTDKADLRLIYLALAHMIKFRGHFLIEGDLNPDNSDVDKLFI |
| | QLVQTYNQLFEENPINASGVDAKAILSARLSKSRRLENLIAQLPGE |
| | KKNGLFGNLIALSLGLTPNFKSNFDLAEDAKLQLSKDTYDDDLDN |
| | LLAQIGDQYADLFLAAKNLSDAILLSDILRVNTEITKAPLSASMIKR |
| | YDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHL |
| | GELHAILRRQEDFYPFLKDNREKIEKILTFRIPYYVGPLARGNSRFA |
| | WMTRKSEETITPWNFEEVVDKGASAQSFIERMTNFDKNLPNEKVL |
| | PKHSLLYEYFTVYNELTKVKYVTEGMRKPAFLSGEQKKAIVDLLF |
| | KTNRKVTVKQLKEDYFKKIECFDSVEISGVEDRFNASLGTYHDLL |
| | KIIKDKDFLDNEENEDILEDIVLTLTLFEDREMIEERLKTYAHLFDD |
| | KVMKQLKRRRYTGWGRLSRKLINGIRDKQSGKTILDFLKSDGFAN |
| | RNFMQLIHDDSLTFKEDIQKAQVSGQGDSLHEHIANLAGSPAIKKG |
| | ILQTVKVVDELVKVMGRHKPENIVIEMARENQTTQKGQKNSRER |
| | MKRIEEGIKELGSQILKEHPVENTQLQNEKLYLYYLQNGRDMYVD |
| | QELDINRLSDYDVDAIVPQSFLKDDSIDNKVLTRSDKNRGKSDNVP |
| | SEEVVKKMKNYWRQLLNAKLITQRKFDNLTKAERGGLSELDKAG |
| | FIKRQLVETRQITKHVAQILDSRMNTKYDENDKLIREVKVITLKSKL |
| | VSDFRKDFQFYKVREINNYHHAHDAYLNAVVGTALIKKYPKLESE |
| | FVYGDYKVYDVRKMIAKSEQEIGKATAKYFFYSNIMNFFKTEITLA |
| | NGEIRKRPLIETNGETGEIVWDKGRDFATVRKVLSMPQVNIVKKTE |
| | VQTGGFSKESILPKRNSDKLIARKKDWDPKKYGGFDSPTVAYSVL |
| | VVAKVEKGKSKKLKSVKELLGITIMERSSFEKNPIDFLEAKGYKEV |
| | KKDLIIKLPKYSLFELENGRKRMLASAGELQKGNELALPSKYVNFL |
| | YLASHYEKLKGSPEDNEQKQLFVEQHKHYLDEIIEQISEFSKRVILA |
| | DANLDKVLSAYNKHRDKPIREQAENIIHLFTLTNLGAPAAFKYFDT |
| | TIDRKRYTSTKEVLDATLIHQSITGLYETRIDLSQLGGD (SEQ ID |
| | NO: 6) |
| PE2-LINKER | SGGSSGGSSGSETPGTSESATPESSGGSSGGSS (SEQ ID NO: 7) |
| BETWEEN CAS9 | |
| DOMAIN AND | |
| RT DOMAIN (33 | |
| AMINO ACIDS) | |
| PE2-MMLV_RT | TLNIEDEYRLHETSKEPDVSLGSTWLSDFPQAWAETGGMGLAVRQ |
| D200N | APLIIPLKATSTPVSIKQYPMSQEARLGIKPHIQRLLDQGILVPCQSP |
| T330P | WNTPLLPVKKPGTNDYRPVQDLREVNKRVEDIHPTVPNPYNLLSG |
| L603W | LPPSHQWYTVLDLKDAFFCLRLHPTSQPLFAFEWRDPEMGISGQLT |
| T306K | WTRLPQGFKNSPTLFNEALHRDLADFRIQHPDLILLQYVDDLLLAA |
| W313F | TSELDCQQGTRALLQTLGNLGYRASAKKAQICQKQVKYLGYLLK |
| | EGQRWLTEARKETVMGQPTPKTPRQLREFLGKAGFCRLFIPGFAE |
| | MAAPLYPLTKPGTLFNWGPDQQKAYQEIKQALLTAPALGLPDLTKP |
| | FELFVDEKQGYAKGVLTQKLGPWRRPVAYLSKKLDPVAAGWPPC |
| | LRMVAAIAVLTKDAGKLTMGQPLVILAPHAVEALVKQPPDRWLSN |
| | ARMTHYQALLLDTDRVQFGPVVALNPATLLPLPEEGLQHNCLDIL |
| | AEAHGTRPDLTDQPLPDADHTWYTDGSSLLQEGQRKAGAAVTTE |
| | TEVIWAKALPAGTSAQRAELIALTQALKMAEGKKLNVYTDSRYAF |
| | ATAHIHGEIYRRRGWLTSEGKEIKNKDEILALLKALFLPKRLSIIHCP |
| | GHQKGHSAEARGNRMADQAARKAAITETPDTSTLLIENSSP (SEQ |
| | ID NO: 10) |
| PE2-C- | SGGSKRTADGSEFEPKKKRKV (SEQ ID NO: 5) |
| TERMINAL NLS | |

In still other embodiments, the prime editor fusion protein may have the following amino acid sequences:

| | |
|---|---|
| PE FUSION | MKRTADGSEFESPKKKRKVTLNIEDEYRLHETSKEPDVSLGSTWLSD |
| PROTEIN | FPQAWAETGGMGLAVRQAPLIIPLKATSTPVSIKQYPMSQEARLGIKPH |
| MMLV_RT | IQRLLDQGILVPCQSPWNTPLLPVKKPGTNDYRPVQDLREVNKRVEDI |
| (WT)-32 AA- | HPTVPNPYNLLSGLPPSHQWYTVLDLKDAFFCLRLHPTSQPLFAFEWR |
| CAS9(H840A) | DPEMGISGQLTWTRLPQGFKNSPTLFDEALHRDLADFRIQHPDLILLQ |
| | YVDDLLLAATSELDCQQGTRALLQTLGNLGYRASAKKAQICQKQVKYL |
| | GYLLKEGQRWLTEARKETVMGQPTPKTPRQLREFLGTAGFCRLWIPG |
| | FAEMAAPLYPLTKTGTLFNWGPDQQKAYQEIKQALLTAPALGLPDLTK |
| | PFELFVDEKQGYAKGVLTQKLGPWRRPVAYLSKKLDPVAAGWPPCLR |
| | MVAAIAVLTKDAGKLTMGQPLVILAPHAVEALVKQPPDRWLSNARMTH |
| | YQALLLDTDRVQFGPVVALNPATLLPLPEEGLQHNCLDILAEAHGTRP |
| | DLTDQPLPDADHTWYTDGSSLLQEGQRKAGAAVTTETEVIWAKALPA |
| | GTSAQRAELIALTQALKMAEGKKLNVYTDSRYAFATAHIHGEIYRRRGLL |
| | TSEGKEIKNKDEILALLKALFLPKRLSIIHCPGHQKGHSAEARGNRMA |
| | DQAARKAAITETPDTSTLLIENSSPSGGSSGGSSGSETPGTSESATPE |
| | SSGGSSGGSSDKKYSIGLDIGTNSVGWAVITDEYKVPSKKFKVL |
| | GNTDRHSIKKNLIGALLFDSGETAEATRLKRTARRRYTRRKNRI |
| | CYLQEIFSNEMAKVDDSFFHRLEESFLVEEDKKHERHPIFGNIV |
| | DEVAYHEKYPTIYHLRKKLVDSTDKADLRLIYLALAHMIKFRG |
| | HFLIEGDLNPDNSDVDKLFIQLVQTYNQLFEENPINASGVDAKA |
| | ILSARLSKSRRLENLIAQLPGEKKNGLFGNLIALSLGLTPNFKS |
| | NFDLAEDAKLQLSKDTYDDDLDNLLAQIGDQYADLFLAAKNLS |
| | DAILLSDILRVNTEITKAPLSASMIKRYDEHHQDLTLLKALVRQ |
| | QLPEKYKEIFFDQSKNGYAGYIDGGASQEEFYKFIKPILEKMD |
| | GTEELLVKLNREDLLRKQRTFDNGSIPHQIHLGELHAILRRQE |
| | DFYPFLKDNREKIEKILTFRIPYYVGPLARGNSRFAWMTRKSEE |
| | TITPWNFEEVVDKGASAQSFIERMTNFDKNLPNEKVLPKHSLL |
| | YEYFTVYNELTKVKYVTEGMRKPAFLSGEQKKAIVDLLFKTN |
| | RKVTVKQLKEDYFKKIECFDSVEISGVEDRFNASLGTYHDLLK |
| | IIKDKDFLDNEENEDILEDIVLTLTLFEDREMIEERLKTYAHLFD |
| | DKVMKQLKRRRYTGWGRLSRKLINGIRDKQSGKTILDFLKSD |
| | GFANRNFMQLIHDDSLTFKEDIQKAQVSGQGDSLHEHIANLAG |
| | SPAIKKGILQTVKVVDELVKVMGRHKPENIVIEMARENQTTQK |
| | GQKNSRERMKRIEEGIKELGSQILKEHPVENTQLQNEKLYLYY |
| | LQNGRDMYVDQELDINRLSDYDVDAIVPQSFLKDDSIDNKVLT |
| | RSDKNRGKSDNVPSEEVVKKMKNYWRQLLNAKLITQRKFDNL |
| | TKAERGGLSELDKAGFIKRQLVETRQITKHVAQILDSRMNTKY |
| | DENDKLIREVKVITLKSKLVSDFRKDFQFYKVREINNYHHAHD |
| | AYLNAVVGTALIKKYPKLESEFVYGDYKVYDVRKMIAKSEQEI |
| | GKATAKYFFYSNIMNFFKTEITLANGEIRKRPLIETNGETGEIV |
| | WDKGRDFATVRKVLSMPQVNIVKKTEVQTGGFSKESILPKRNS |
| | DKLIARKKDWDPKKYGGFDSPTVAYSVLVVAKVEKGKSKKLK |
| | SVKELLGITIMERSSFEKNPIDFLEAKGYKEVKKDLIIKLPKYSL |
| | FELENGRKRMLASAGELQKGNELALPSKYVNFLYLASHYEKL |
| | KGSPEDNEQKQLFVEQHKHYLDEIIEQISEFSKRVILADANLDK |
| | VLSAYNKHRDKPIREQAENIIHLFTLTNLGAPAAFKYFDTTIDR |
| | KRYTSTKEVLDATLIHQSITGLYETRIDLSQLGGDSGGSKRTAD |
| | GSEFEPKKKRKV (SEQ ID NO: 41) |
| | KEY: |
| | NUCLEAR LOCALIZATION SEQUENCE (NLS) TOP: (SEQ ID NO: 4), |
| | BOTTOM: (SEQ ID NO: 5) |
| | CAS9(H840A) (SEQ ID NO: 6) |
| | 33-AMINO ACID LINKER (SEQ ID NO: 7) |
| | M-MLV REVERSE TRANSCRIPTASE (SEQ ID NO: 8) |
| PE FUSION | MKRTADGSEFESPKKKRKVTLNIEDEYRLHETSKEPDVSLGSTWLSD |
| PROTEIN | FPQAWAETGGMGLAVRQAPLIIPLKATSTPVSIKQYPMSQEARLGIKPH |
| MMLV_RT(WT)- | IQRLLDQGILVPCQSPWNTPLLPVKKPGTNDYRPVQDLREVNKRVEDI |
| 60 AA- | HPTVPNPYNLLSGLPPSHQWYTVLDLKDAFFCLRLHPTSQPLFAFEWR |
| CAS9(H840A) | DPEMGISGQLTWTRLPQGFKNSPTLFDEALHRDLADFRIQHPDLILLQ |
| | YVDDLLLAATSELDCQQGTRALLQTLGNLGYRASAKKAQICQKQVKYL |
| | GYLLKEGQRWLTEARKETVMGQPTPKTPRQLREFLGTAGFCRLWIPG |
| | FAEMAAPLYPLTKTGTLFNWGPDQQKAYQEIKQALLTAPALGLPDLTK |
| | PFELFVDEKQGYAKGVLTQKLGPWRRPVAYLSKKLDPVAAGWPPCLR |
| | MVAAIAVLTKDAGKLTMGQPLVILAPHAVEALVKQPPDRWLSNARMTH |
| | YQALLLDTDRVQFGPVVALNPATLLPLPEEGLQHNCLDILAEAHGTRP |
| | DLTDQPLPDADHTWYTDGSSLLQEGQRKAGAAVTTETEVIWAKALPA |
| | GTSAQRAELIALTQALKMAEGKKLNVYTDSRYAFATAHIHGEIYRRRGLL |
| | TSEGKEIKNKDEILALLKALFLPKRLSIIHCPGHQKGHSAEARGNRMA |
| | DQAARKAAITETPDTSTLLIENSSPSGGSSGGSSGSETPGTSESATPE |
| | SAGSYPYDVPDYAGSAAPAAKKKKLDGSGSGGSSGGSDKKYSI |
| | GLDIGTNSVGWAVITDEYKVPSKKFKVLGNTDRHSIKKNLIGA |
| | LLFDSGETAEATRLKRTARRRYTRRKNRICYLQEIFSNEMAKV |
| | DDSFFHRLEESFLVEEDKKHERHPIFGNIVDEVAYHEKYPTIYH |
| | LRKKLVDSTDKADLRLIYLALAHMIKFRGHFLIEGDLNPDNSD |
| | VDKLFIQLVQTYNQLFEENPINASGVDAKAILSARLSKSRRLEN |
| | LIAQLPGEKKNGLFGNLIALSLGLTPNFKSNFDLAEDAKLQLSK |
| | DTYDDDLDNLLAQIGDQYADLFLAAKNLSDAILLSDILRVNTEI |
| | TKAPLSASMIKRYDEHHQDLTLLKALVRQQLPEKYKEIFFDQS |
| | KNGYAGYIDGGASQEEFYKFIKPILEKMDGTEELLVKLNREDL |
| | LRKQRTFDNGSIPHQIHLGELHAILRRQEDFYPFLKDNREKIEK |
| | ILTFRIPYYVGPLARGNSRFAWMTRKSEETITPWNFEEVVDKG |
| | ASAQSFIERMTNFDKNLPNEKVLPKHSLLYEYFTVYNELTKVK |
| | YVTEGMRKPAFLSGEQKKAIVDLLFKTNRKVTVKQLKEDYFK |
| | KIECFDSVEISGVEDRFNASLGTYHDLLKIIKDKDFLDNEENEDI |
| | LEDIVLTLTLFEDREMIEERLKTYAHLFDDKVMKQLKRRRYTG |
| | WGRLSRKLINGIRDKQSGKTILDFLKSDGFANRNFMQLIHDDS |
| | LTFKEDIQKAQVSGQGDSLHEHIANLAGSPAIKKGILQTVKVV |
| | DELVKVMGRHKPENIVIEMARENQTTQKGQKNSRERMKRIEE |
| | GIKELGSQILKEHPVENTQLQNEKLYLYYLQNGRDMYVDQEL |
| | DINRLSDYDVDAIVPQSFLKDDSIDNKVLTRSDKNRGKSDNVPS |
| | EEVVKKMKNYWRQLLNAKLITQRKFDNLTKAERGGLSELDK |
| | AGFIKRQLVETRQITKHVAQILDSRMNTKYDENDKLIREVKVIT |
| | LKSKLVSDFRKDFQFYKVREINNYHHAHDAYLNAVVGTALIKK |
| | YPKLESEFVYGDYKVYDVRKMIAKSEQEIGKATAKYFFYSNIM |
| | NFFKTEITLANGEIRKRPLIETNGETGEIVWDKGRDFATVRKVL |
| | SMPQVNIVKKTEVQTGGFSKESILPKRNSDKLIARKKDWDPKK |
| | YGGFDSPTVAYSVLVVAKVEKGKSKKLKSVKELLGITIMERSSF |
| | EKNPIDFLEAKGYKEVKKDLIIKLPKYSLFELENGRKRMLASA |
| | GELQKGNELALPSKYVNFLYLASHYEKLKGSPEDNEQKQLFV |
| | EQHKHYLDEIIEQISEFSKRVILADANLDKVLSAYNKHRDKPIR |
| | EQAENIIHLFTLTNLGAPAAFKYFDTTIDRKRYTSTKEVLDATLI |
| | HQSITGLYETRIDLSQLGGDSGGSKRTADGSEFEPKKKRKV (SEQ |
| | ID NO: 42) |
| | KEY: |
| | NUCLEAR LOCALIZATION SEQUENCE (NLS) TOP: (SEQ ID NO: 4), |
| | BOTTOM: (SEQ ID NO: 5) |
| | CAS9(H840A) (SEQ ID NO: 6) |
| | AMINO ACID LINKER (SEQ ID NO: 43) |
| | M-MLV REVERSE TRANSCRIPTASE (SEQ ID NO: 8) |
| PE FUSION | MKRTADGSEFESPKKKRKVDKKYSIGLDIGTNSVGWAVITDEYK |
| PROTEIN | VPSKKFKVLGNTDRHSIKKNLIGALLFDSGETAEATRLKRTAR |
| CAS9(H840A)- | RRYTRRKNRICYLQEIFSNEMAKVDDSFFHRLEESFLVEEDKK |
| FEN1-MMLV_RT | HERHPIFGNIVDEVAYHEKYPTIYHLRKKLVDSTDKADLRLIYL |
| D200N | ALAHMIKFRGHFLIEGDLNPDNSDVDKLFIQLVQTYNQLFEEN |
| T330P | PINASGVDAKAILSARLSKSRRLENLIAQLPGEKKNGLFGNLIA |
| L603W | LSLGLTPNFKSNFDLAEDAKLQLSKDTYDDDLDNLLAQIGDQY |
| T306K | ADLFLAAKNLSDAILLSDILRVNTEITKAPLSASMIKRYDEHHQ |
| W313F | DLTLLKALVRQQLPEKYKEIFFDQSKNGYAGYIDGGASQEEFY |
| | KFIKPILEKMDGTEELLVKLNREDLLRKQRTFDNGSIPHQIHLG |
| | ELHAILRRQEDFYPFLKDNREKIEKILTFRIPYYVGPLARGNSR |
| | FAWMTRKSEETITPWNFEEVVDKGASAQSFIERMTNFDKNLPN |
| | EKVLPKHSLLYEYFTVYNELTKVKYVTEGMRKPAFLSGEQKK |
| | AIVDLLFKTNRKVTVKQLKEDYFKKIECFDSVEISGVEDRFNAS |
| | LGTYHDLLKIIKDKDFLDNEENEDILEDIVLTLTLFEDREMIEER |
| | LKTYAHLFDDKVMKQLKRRRYTGWGRLSRKLINGIRDKQSG |
| | KTILDFLKSDGFANRNFMQLIHDDSLTFKEDIQKAQVSGQGDS |
| | LHEHIANLAGSPAIKKGILQTVKVVDELVKVMGRHKPENIVIE |
| | MARENQTTQKGQKNSRERMKRIEEGIKELGSQILKEHPVENT |
| | QLQNEKLYLYYLQNGRDMYVDQELDINRLSDYDVDAIVPQSFL |
| | KDDSIDNKVLTRSDKNRGKSDNVPSEEVVKKMKNYWRQLLNA |
| | KLITQRKFDNLTKAERGGLSELDKAGFIKRQLVETRQITKHVA |
| | QILDSRMNTKYDENDKLIREVKVITLKSKLVSDFRKDFQFYKV |
| | REINNYHHAHDAYLNAVVGTALIKKYPKLESEFVYGDYKVYDV |
| | RKMIAKSEQEIGKATAKYFFYSNIMNFFKTEITLANGEIRKRPL |
| | IETNGETGEIVWDKGRDFATVRKVLSMPQVNIVKKTEVQTGG |
| | FSKESILPKRNSDKLIARKKDWDPKKYGGFDSPTVAYSVLVVAK |
| | VEKGKSKKLKSVKELLGITIMERSSFEKNPIDFLEAKGYKEVK |
| | KDLIIKLPKYSLFELENGRKRMLASAGELQKGNELALPSKYVN |
| | FLYLASHYEKLKGSPEDNEQKQLFVEQHKHYLDEIIEQISEFSK |
| | RVILADANLDKVLSAYNKHRDKPIREQAENIIHLFTLTNLGAPA |
| | AFKYFDTTIDRKRYTSTKEVLDATLIHQSITGLYETRIDLSQLG |
| | GDSGGSSGGSSGSETPGTSESATPESSGGSSGGSSGIQGLAKLIAD |
| | VAPSAIRENDIKSYFGRKVAIDASMSIYQFLIAVRQGGDVLQNEEGE |
| | TTSHLMGMFYRTIRMMENGIKPVYVFDGKPPQLKSGELAKRSERR |
| | AEAEKQLQQAQAAGAEQEVEKFTKRLVKVTKQHNDECKHLLSL |
| | MGIPYLDAPSEAEASCAALVKAGKVYAAATEDMDCLTFGSPVLMR |
| | HLTASEAKKLPIQEFHLSRILQELGLNQEQFVDLCILLGSDYCESIRG |
| | IGPKRAVDLIQKHKSIEEIVRRLDPNKYPVPENWLHKEAHQLFLEP |
| | EVLDPESVELKWSEPNEEELIKFMCGEKQFSEERIRSGVKRLSKSR |
| | QGSTQGRLDDFFKVTGSLSSAKRKEPEPKGSTKKKAKTGAAGKFK |
| | RGKSGGSSGGSSGSETPGTSESATPESSGGSSGGSSTLNIEDEYRL |
| | HETSKEPDVSLGSTWLSDFPQAWAETGGMGLAVRQAPLIIPLKATSTPV |
| | SIKQYPMSQEARLGIKPHIQRLLDQGILVPCQSPWNTPLLPVKKPGTN |
| | DYRPVQDLREVNKRVEDIHPTVPNPYNLLSGLPPSHQWYTVLDLKDAF |
| | FCLRLHPTSQPLFAFEWRDPEMGISGQLTWTRLPQGFKNSPTLFNEAL |
| | HRDLADFRIQHPDLILLQYVDDLLLAATSELDCQQGTRALLQTLGNLG |
| | YRASAKKAQICQKQVKYLGYLLKEGQRWLTEARKETVMGQPTPKTPR |
| | QLREFLGKAGFCRLFIPGFAEMAAPLYPLTKPGTLFNWGPDQQKAYQ |
| | EIKQALLTAPALGLPDLTKPFELFVDEKQGYAKGVLTQKLGPWRRPVA |
| | YLSKKLDPVAAGWPPCLRMVAAIAVLTKDAGKLTMGQPLVILAPHAVE |
| | ALVKQPPDRWLSNARMTHYQALLLDTDRVQFGPVVALNPATLLPLPEE |
| | GLQHNCLDILAEAHGTRPDLTDQPLPDADHTWYTDGSSLLQEGQRKA |
| | GAAVTTETEVIWAKALPAGTSAQRAELIALTQALKMAEGKKLNVYTDSR |
| | YAFATAHIHGEIYRRRGWLTSEGKEIKNKDEILALLKALFLPKRLSIIHCP |
| | GHQKGHSAEARGNRMADQAARKAAITETPDTSTLLIENSSPSGGSKRT |
| | ADGSEFEPKKKRKV (SEQ ID NO: 44) |
| | KEY: |
| | NUCLEAR LOCALIZATION SEQUENCE (NLS) TOP: (SEQ ID NO: 4), |
| | BOTTOM: (SEQ ID NO: 5) |
| | CAS9(H840A) (SEQ ID NO: 6) |
| | 33-AMINO ACID LINKER 1 (SEQ ID NO: 7) |
| | M-MLV REVERSE TRANSCRIPTASE (SEQ ID NO: 10) |
| | 33-AMINO ACID LINKER 2 (SEQ ID NO: 7) |
| | FEN1 (SEQ ID NO: 45) |

In other embodiments, the prime editors and/or multi-flap prime editors (e.g., twinPE or quadruple flap) can be based on SaCas9 or on SpCas9 nickases with altered PAM specificities, such as the following exemplary sequences:

| | |
|---|---|
| SACAS9-M-MLV | MKRTADGSEFESPKKKRKVGKRNYILGLDIGITSVGYGIIDYETR |
| RT PRIME EDITOR | DVIDAGVRLFKEANVENNEGRRSKRGARRLKRRRRHRIQRVKK |
| | LLFDYNLLTDHSELSGINPYEARVKGLSQKLSEEEFSAALLHLA |
| | KRRGVHNVNEVEEDTGNELSTKEQISRNSKALEEKYVAELQLE |
| | RLKKDGEVRGSINRFKTSDYVKEAKQLLKVQKAYHQLDQSFID |
| | TYIDLLETRRTYYEGPGEGSPFGWKDIKEWYEMLMGHCTYFPE |
| | ELRSVKYAYNADLYNALNDLNNLVITRDENEKLEYYEKFQIIEN |
| | VFKQKKKPTLKQIAKEILVNEEDIKGYRVTSTGKPEFTNLKVYH |
| | DIKDITARKEIIENAELLDQIAKILTIYQSSEDIQEELTNLNSELTQE |
| | EIEQISNLKGYTGTHNLSLKAINLILDELWHTNDNQIAIFNRLKL |
| | VPKKVDLSQQKEIPTTLVDDFILSPVVKRSFIQSIKVINAIIKKYG |
| | LPNDIIIELAREKNSKDAQKMINEMQKRNRQTNERIEEIIRTTGK |
| | ENAKYLIEKIKLHDMQEGKCLYSLEAIPLEDLLNNPFNYEVDHII |
| | PRSVSFDNSFNNKVLVKQEEASKKGNRTPFQYLSSSDSKISYETF |
| | KKHILNLAKGKGRISKTKKEYLLEERDINRFSVQKDFINRNLVD |
| | TRYATRGLMNLLRSYFRVNNLDVKVKSINGGFTSFLRRKWKFK |
| | KERNKGYKHHAEDALIIANADFIFKEWKKLDKAKKVMENQMF |
| | EEKQAESMPEIETEQEYKEIFITPHQIKHIKDFKDYKYSHRVDKK |
| | PNRELINDTLYSTRKDDKGNTLIVNNLNGLYDKDNDKLKKLIN |
| | KSPEKLLMYHHDPQTYQKLKLIMEQYGDEKNPLYKYYEETGN |
| | YLTKYSKKDNGPVIKKIKYYGNKLNAHLDITDDYPNSRNKVVK |
| | LSLKPYRFDVYLDNGVYKFVTVKNLDVIKKENYYEVNSKCYE |
| | EAKKLKKISNQAEFIASFYNNDLIKINGELYRVIGVNNDLLNRIE |
| | VNMIDITYREYLENMNDKRPPRIIKTIASKTQSIKKYSTDILGNL |
| | YEVKSKKHPQIIKKGSGGSSGGSSGSETPGTSESATPESSGGSSG |
| | GSSTLNIEDEYRLHETSKEPDVSLGSTWLSDFPQAWAETGGMGL |
| | AVRQAPLIIPLKATSTPVSIKQYPMSQEARLGIKPHIQRLLDQGIL |
| | VPCQSPWNTPLLPVKKPGTNDYRPVQDLREVNKRVEDIHPTVP |
| | NPYNLLSGLPPSHQWYTVLDLKDAFFCLRLHPTSQPLFAFEWR |
| | DPEMGISGQLTWTRLPQGFKNSPTLFDEALHRDLADFRIQHPDL |
| | ILLQYVDDLLLAATSELDCQQGTRALLQTLGNLGYRASAKKAQ |
| | ICQKQVKYLGYLLKEGQRWLTEARKETVMGQPTPKTPRQLREF |
| | LGTAGFCRLWIPGFAEMAAPLYPLTKTGTLFNWGPDQQKAYQEI |
| | KQALLTAPALGLPDLTKPFELFVDEKQGYAKGVLTQKLGPWRR |
| | PVAYLSKKLDPVAAGWPPCLRMVAAIAVLTKDAGKLTMGQPLVI |
| | LAPHAVEALVKQPPDRWLSNARMTHYQALLLDTDRVQFGPVV |
| | ALNPATLLPLPEEGLQHNCLDILAEAHGTRPDLTDQPLPDADHT |
| | WYTDGSSLLQEGQRKAGAAVTTETEVIWAKALPAGTSAQRAEL |
| | IALTQALKMAEGKKLNVYTDSRYAFATAHIHGEIYRRRGLLTSE |
| | GKEIKNKDEILALLKALFLPKRLSIIHCPGHQKGHSAEARGNRM |
| | ADQAARKAAITETPDTSTLLIENSSPSGGSKRTADGSEFEPKKKR |
| | KV (SEQ ID NO: 46) |
| SPCAS9(H840A)- | MKRTADGSEFESPKKKRKVDKKYSIGLDIGTNSVGWAVITDEY |
| VRQR-MALONEY | KVPSKKFKVLGNTDRHSIKKNLIGALLFDSGETAEATRLKRTAR |
| MURINE | RRYTRRKNRICYLQEIFSNEMAKVDDSFFHRLEESFLVEEDKKH |
| LEUKEMIA VIRUS | ERHPIFGNIVDEVAYHEKYPTIYHLRKKLVDSTDKADLRLIYLAL |
| REVERSE | AHMIKFRGHFLIEGDLNPDNSDVDKLFIQLVQTYNQLFEENPIN |
| TRANSCRIPTASE | ASGVDAKAILSARLSKSRRLENLIAQLPGEKKNGLFGNLIALSL |
| PRIME EDITOR | GLTPNFKSNFDLAEDAKLQLSKDTYDDDLDNLLAQIGDQYADL |
| | FLAAKNLSDAILLSDILRVNTEITKAPLSASMIKRYDEHHQDLTL |
| | LKALVRQQLPEKYKEIFFDQSKNGYAGYIDGGASQEEFYKFIKPI |
| | LEKMDGTEELLVKLNREDLLRKQRTFDNGSIPHQIHLGELHAIL |
| | RRQEDFYPFLKDNREKIEKILTFRIPYYVGPLARGNSRFAWMTR |
| | KSEETITPWNFEEVVDKGASAQSFIERMTNFDKNLPNEKVLPKH |
| | SLLYEYFTVYNELTKVKYVTEGMRKPAFLSGEQKKAIVDLLFKT |
| | NRKVTVKQLKEDYFKKIECFDSVEISGVEDRFNASLGTYHDLL |
| | KIIKDKDFLDNEENEDILEDIVLTLTLFEDREMIEERLKTYAHLFD |
| | DKVMKQLKRRRYTGWGRLSRKLINGIRDKQSGKTILDFLKSDG |
| | FANRNFMQLIHDDSLTFKEDIQKAQVSGQGDSLHEHIANLAGSP |
| | AIKKGILQTVKVVDELVKVMGRHKPENIVIEMARENQTTQKGQ |
| | KNSRERMKRIEEGIKELGSQILKEHPVENTQLQNEKLYLYYLQN |
| | GRDMYVDQELDINRLSDYDVDAIVPQSFLKDDSIDNKVLTRSD |
| | KNRGKSDNVPSEEVVKKMKNYWRQLLNAKLITQRKFDNLTKA |
| | ERGGLSELDKAGFIKRQLVETRQITKHVAQILDSRMNTKYDEND |
| | KLIREVKVITLKSKLVSDFRKDFQFYKVREINNYHHAHDAYLNA |
| | VVGTALIKKYPKLESEFVYGDYKVYDVRKMIAKSEQEIGKATA |
| | KYFFYSNIMNFFKTEITLANGEIRKRPLIETNGETGEIVWDKGRD |
| | FATVRKVLSMPQVNIVKKTEVQTGGFSKESILPKRNSDKLIARK |
| | KDWDPKKYGGFVSPTVAYSVLVVAKVEKGKSKKLKSVKELLGI |
| | TIMERSSFEKNPIDFLEAKGYKEVKKDLIIKLPKYSLFELENGRK |
| | RMLASARELQKGNELALPSKYVNFLYLASHYEKLKGSPEDNEQ |
| | KQLFVEQHKHYLDEIIEQISEFSKRVILADANLDKVLSAYNKHR |
| | DKPIREQAENIIHLFTLTNLGAPAAFKYFDTTIDRKQYRSTKEVL |
| | DATLIHQSITGLYETRIDLSQLGGDSGGSSGGSSGSETPGTSESAT |
| | PESSGGSSGGSSTLNIEDEYRLHETSKEPDVSLGSTWLSDFPQAW |
| | AETGGMGLAVRQAPLIIPLKATSTPVSIKQYPMSQEARLGIKPHI |
| | QRLLDQGILVPCQSPWNTPLLPVKKPGTNDYRPVQDLREVNKR |
| | VEDIHPTVPNPYNLLSGLPPSHQWYTVLDLKDAFFCLRLHPTSQ |
| | PLFAFEWRDPEMGISGQLTWTRLPQGFKNSPTLFNEALHRDLAD |
| | FRIQHPDLILLQYVDDLLLAATSELDCQQGTRALLQTLGNLGYR |
| | ASAKKAQICQKQVKYLGYLLKEGQRWLTEARKETVMGQPTPK |
| | TPRQLREFLGKAGFCRLFIPGFAEMAAPLYPLTKPGTLFNWGPD |
| | QQKAYQEIKQALLTAPALGLPDLTKPFELFVDEKQGYAKGVLTQ |
| | KLGPWRRPVAYLSKKLDPVAAGWPPCLRMVAAIAVLTKDAGKL |
| | TMGQPLVILAPHAVEALVKQPPDRWLSNARMTHYQALLLDTDR |
| | VQFGPVVALNPATLLPLPEEGLQHNCLDILAEAHGTRPDLTDQP |
| | LPDADHTWYTDGSSLLQEGQRKAGAAVTTETEVIWAKALPAGT |
| | SAQRAELIALTQALKMAEGKKLNVYTDSRYAFATAHIHGEIYRR |
| | RGWLTSEGKEIKNKDEILALLKALFLPKRLSIIHCPGHQKGHSAE |
| | ARGNRMADQAARKAAITETPDTSTLLIENSSPSGGSKRTADGSE |
| | FEPKKKRKV (SEQ ID NO: 47) |
| SPCAS9(H840A)- | MKRTADGSEFESPKKKRKVDKKYSIGLDIGTNSVGWAVITDEY |
| VRER-MALONEY | KVPSKKFKVLGNTDRHSIKKNLIGALLFDSGETAEATRLKRTAR |
| MURINE | RRYTRRKNRICYLQEIFSNEMAKVDDSFFHRLEESFLVEEDKKH |
| LEUKEMIA VIRUS | ERHPIFGNIVDEVAYHEKYPTIYHLRKKLVDSTDKADLRLIYLAL |
| REVERSE | AHMIKFRGHFLIEGDLNPDNSDVDKLFIQLVQTYNQLFEENPIN |
| TRANSCRIPTASE | ASGVDAKAILSARLSKSRRLENLIAQLPGEKKNGLFGNLIALSL |
| PRIME EDITOR | GLTPNFKSNFDLAEDAKLQLSKDTYDDDLDNLLAQIGDQYADL |
| | FLAAKNLSDAILLSDILRVNTEITKAPLSASMIKRYDEHHQDLTL |
| | LKALVRQQLPEKYKEIFFDQSKNGYAGYIDGGASQEEFYKFIKPI |
| | LEKMDGTEELLVKLNREDLLRKQRTFDNGSIPHQIHLGELHAIL |
| | RRQEDFYPFLKDNREKIEKILTFRIPYYVGPLARGNSRFAWMTR |
| | KSEETITPWNFEEVVDKGASAQSFIERMTNFDKNLPNEKVLPKH |
| | SLLYEYFTVYNELTKVKYVTEGMRKPAFLSGEQKKAIVDLLFKT |
| | NRKVTVKQLKEDYFKKIECFDSVEISGVEDRFNASLGTYHDLL |
| | KIIKDKDFLDNEENEDILEDIVLTLTLFEDREMIEERLKTYAHLFD |
| | DKVMKQLKRRRYTGWGRLSRKLINGIRDKQSGKTILDFLKSDG |
| | FANRNFMQLIHDDSLTFKEDIQKAQVSGQGDSLHEHIANLAGSP |
| | AIKKGILQTVKVVDELVKVMGRHKPENIVIEMARENQTTQKGQ |
| | KNSRERMKRIEEGIKELGSQILKEHPVENTQLQNEKLYLYYLQN |
| | GRDMYVDQELDINRLSDYDVDAIVPQSFLKDDSIDNKVLTRSD |
| | KNRGKSDNVPSEEVVKKMKNYWRQLLNAKLITQRKFDNLTKA |
| | ERGGLSELDKAGFIKRQLVETRQITKHVAQILDSRMNTKYDEND |
| | KLIREVKVITLKSKLVSDFRKDFQFYKVREINNYHHAHDAYLNA |
| | VVGTALIKKYPKLESEFVYGDYKVYDVRKMIAKSEQEIGKATA |
| | KYFFYSNIMNFFKTEITLANGEIRKRPLIETNGETGEIVWDKGRD |
| | FATVRKVLSMPQVNIVKKTEVQTGGFSKESILPKRNSDKLIARK |
| | KDWDPKKYGGFVSPTVAYSVLVVAKVEKGKSKKLKSVKELLGI |
| | TIMERSSFEKNPIDFLEAKGYKEVKKDLIIKLPKYSLFELENGRK |
| | RMLASARELQKGNELALPSKYVNFLYLASHYEKLKGSPEDNEQ |
| | KQLFVEQHKHYLDEIIEQISEFSKRVILADANLDKVLSAYNKHR |
| | DKPIREQAENIIHLFTLTNLGAPAAFKYFDTTIDRKEYRSTKEVL |
| | DATLIHQSITGLYETRIDLSQLGGDSGGSSGGSSGSETPGTSESAT |
| | PESSGGSSGGSSTLNIEDEYRLHETSKEPDVSLGSTWLSDFPQAW |
| | AETGGMGLAVRQAPLIIPLKATSTPVSIKQYPMSQEARLGIKPHI |
| | QRLLDQGILVPCQSPWNTPLLPVKKPGTNDYRPVQDLREVNKR |
| | VEDIHPTVPNPYNLLSGLPPSHQWYTVLDLKDAFFCLRLHPTSQ |
| | PLFAFEWRDPEMGISGQLTWTRLPQGFKNSPTLFNEALHRDLAD |
| | FRIQHPDLILLQYVDDLLLAATSELDCQQGTRALLQTLGNLGYR |
| | ASAKKAQICQKQVKYLGYLLKEGQRWLTEARKETVMGQPTPK |
| | TPRQLREFLGKAGFCRLFIPGFAEMAAPLYPLTKPGTLFNWGPD |
| | QQKAYQEIKQALLTAPALGLPDLTKPFELFVDEKQGYAKGVLTQ |
| | KLGPWRRPVAYLSKKLDPVAAGWPPCLRMVAAIAVLTKDAGKL |
| | TMGQPLVILAPHAVEALVKQPPDRWLSNARMTHYQALLLDTDR |
| | VQFGPVVALNPATLLPLPEEGLQHNCLDILAEAHGTRPDLTDQP |
| | LPDADHTWYTDGSSLLQEGQRKAGAAVTTETEVIWAKALPAGT |
| | SAQRAELIALTQALKMAEGKKLNVYTDSRYAFATAHIHGEIYRR |
| | RGWLTSEGKEIKNKDEILALLKALFLPKRLSIIHCPGHQKGHSAE |
| | ARGNRMADQAARKAAITETPDTSTLLIENSSPSGGSKRTADGSE |
| | FEPKKKRKV (SEQ ID NO: 48) |

In yet other embodiments, the prime editors and/or multi-flap prime editors (e.g., twinPE or quadruple flap) contemplated herein may include a Cas9 nickase (e.g., Cas9 (H840A)) fused to a truncated version of M-MLV reverse transcriptase. In this embodiment, the reverse transcriptase also contains 4 mutations (D200N, T306K, W313F, T330P; noting that the L603W mutation present in PE2 is no longer present due to the truncation). The DNA sequence encoding this truncated editor is 522 bp smaller than PE2, and therefore makes its potentially useful for applications where delivery of the DNA sequence is challenging due to its size (i.e. adeno-associated virus and lentivirus delivery). This embodiment is referred to as Cas9(H840A)-MMLV-RT(trunc) or “PE2-short” or “PE2-trunc” and has the following amino acid sequence:

| | |
|---|---|
| CAS9(H840A)- | MKRTADGSEFESPKKKRKVDKKYSIGLDIGTNSVGWAVITDE |
| MMLV- | YKVPSKKFKVLGNTDRHSIKKNLIGALLFDSGETAEATRLK |
| RT(TRUNC) OR | RTARRRYTRRKNRICYLQEIFSNEMAKVDDSFFHRLEESFLV |
| PE2-SHORT | EEDKKHERHPIFGNIVDEVAYHEKYPTIYHLRKKLVDSTDK |
| | ADLRLIYLALAHMIKFRGHFLIEGDLNPDNSDVDKLFIQLVQ |
| | TYNQLFEENPINASGVDAKAILSARLSKSRRLENLIAQLPGE |
| | KKNGLFGNLIALSLGLTPNFKSNFDLAEDAKLQLSKDTYDD |
| | DLDNLLAQIGDQYADLFLAAKNLSDAILLSDILRVNTEITKA |
| | PLSASMIKRYDEHHQDLTLLKALVRQQLPEKYKEIFFDQSK |
| | NGYAGYIDGGASQEEFYKFIKPILEKMDGTEELLVKLNRED |
| | LLRKQRTFDNGSIPHQIHLGELHAILRRQEDFYPFLKDNREK |
| | IEKILTFRIPYYVGPLARGNSRFAWMTRKSEETITPWNFEEV |
| | VDKGASAQSFIERMTNFDKNLPNEKVLPKHSLLYEYFTVYN |
| | ELTKVKYVTEGMRKPAFLSGEQKKAIVDLLFKTNRKVTVK |
| | QLKEDYFKKIECFDSVEISGVEDRFNASLGTYHDLLKIIKDK |
| | DFLDNEENEDILEDIVLTLTLFEDREMIEERLKTYAHLFDDK |
| | VMKQLKRRRYTGWGRLSRKLINGIRDKQSGKTILDFLKSD |
| | GFANRNFMQLIHDDSLTFKEDIQKAQVSGQGDSLHEHIANL |
| | AGSPAIKKGILQTVKVVDELVKVMGRHKPENIVIEMARENQ |
| | TTQKGQKNSRERMKRIEEGIKELGSQILKEHPVENTQLQNE |
| | KLYLYYLQNGRDMYVDQELDINRLSDYDVDAIVPQSFLKDD |
| | SIDNKVLTRSDKNRGKSDNVPSEEVVKKMKNYWRQLLNAK |
| | LITQRKFDNLTKAERGGLSELDKAGFIKRQLVETRQITKHV |
| | AQILDSRMNTKYDENDKLIREVKVITLKSKLVSDFRKDFQF |
| | YKVREINNYHHAHDAYLNAVVGTALIKKYPKLESEFVYGDY |
| | KVYDVRKMIAKSEQEIGKATAKYFFYSNIMNFFKTEITLAN |
| | GEIRKRPLIETNGETGEIVWDKGRDFATVRKVLSMPQVNIV |
| | KKTEVQTGGFSKESILPKRNSDKLIARKKDWDPKKYGGFDS |
| | PTVAYSVLVVAKVEKGKSKKLKSVKELLGITIMERSSFEKN |
| | PIDFLEAKGYKEVKKDLIIKLPKYSLFELENGRKRMLASAG |
| | ELQKGNELALPSKYVNFLYLASHYEKLKGSPEDNEQKQLFV |
| | EQHKHYLDEIIEQISEFSKRVILADANLDKVLSAYNKHRDKP |
| | IREQAENIIHLFTLTNLGAPAAFKYFDTTIDRKRYTSTKEVL |
| | DATLIHQSITGLYETRIDLSQLGGDSGGSSGGSSGSETPGTSE |
| | SATPESSGGSSGGSSTLNIEDEYRLHETSKEPDVSLGSTWLSDFPQ |
| | AWAETGGMGLAVRQAPLIIPLKATSTPVSIKQYPMSQEARLGIKPHI |
| | QRLLDQGILVPCQSPWNTPLLPVKKPGTNDYRPVQDLREVNKRVE |
| | DIHPTVPNPYNLLSGLPPSHQWYTVLDLKDAFFCLRLHPTSQPLFA |
| | FEWRDPEMGISGQLTWTRLPQGFKNSPTLFNEALHRDLADFRIQH |
| | PDLILLQYVDDLLLAATSELDCQQGTRALLQTLGNLGYRASAKKAQI |
| | CQKQVKYLGYLLKEGQRWLTEARKETVMGQPTPKTPRQLREFLGK |
| | AGFCRLFIPGFAEMAAPLYPLTKPGTLFNWGPDQQKAYQEIKQAL |
| | LTAPALGLPDLTKPFELFVDEKQGYAKGVLTQKLGPWRRPVAYLSK |
| | KLDPVAAGWPPCLRMVAAIAVLTKDAGKLTMGQPLVILAPHAVEA |
| | LVKQPPDRWLSNARMTHYQALLLDTDRVQFGPVVALNPATLLPLP |
| | EEGLQHNCLDNSRLINSGGSKRTADGSEFEPKKKRKV (SEQ ID |
| | NO: 11) |

KEY:
NUCLEAR LOCALIZATION SEQUENCE (NLS) TOP:(SEQ ID NO: 4), BOTTOM: (SEQ ID NO: 5)
CAS9(H840A) (SEQ ID NO: 6)
33-AMINO ACID LINKER 1 (SEQ ID NO: 7)
M-MLV TRUNCATED REVERSE TRANSCRIPTASE (SEQ ID NO: 12)

In various embodiments, the prime editors and/or multi-flap prime editors (e.g., twinPE or quadruple flap) contemplated herein may also include any variants of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the above indicated prime editor fusion sequences.

In certain embodiments, linkers may be used to link any of the peptides or peptide domains or moieties of the invention (e.g., a napDNAbp linked or fused to a reverse transcriptase).

### Linkers and Other Domains

The PE, twinPE, and multi-flap PE embodiments may comprise various other domains besides the napDNAbp (e.g., Cas9 domain) and the polymerase domain (e.g., RT domain). For example, in the case where the napDNAbp is a Cas9 and the polymerase is a RT, the PE fusion proteins may comprise one or more linkers that join the Cas9 domain with the RT domain. The linkers may also join other functional domains, such as nuclear localization sequences (NLS) or a FEN1 (or other flap endonuclease) to the PE fusion proteins or a domain thereof.

### Linkers

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a polymerase (e.g., a reverse transcriptase). In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 49), (G)ₙ(SEQ ID NO: 50), (EAAAK)ₙ (SEQ ID NO: 51), (GGS)ₙ (SEQ ID NO: 52), (SGGS)ₙ(SEQ ID NO: 53), (XP)ₙ (SEQ ID NO: 54), or any combination thereof, wherein n is independently an integer between 1 and 30, and wherein X is any amino acid. In some embodiments, the linker comprises the amino acid sequence (GGS)ₙ(SEQ ID NO: 52), wherein n is 1, 3, or 7. In some embodiments, the linker comprises the amino acid sequence SGSETPGTSESATPES (SEQ ID NO: 55). In some embodiments, the linker comprises the amino acid sequence SGGSSGGSSGSETPGTSESATPESSGGSSGGS (SEQ ID NO: 56). In some embodiments, the linker comprises the amino acid sequence SGGSGGSGGS (SEQ ID NO: 57). In some embodiments, the linker comprises the amino acid sequence SGGS (SEQ ID NO: 58). In other embodiments, the linker comprises the amino acid sequence

(SEQ ID NO: 43, 60AA)
SGGSSGGSSGSETPGTSESATPESAGSYPYDVPDYAGSAAPAAKKKKLDG
SGSGGSSGGS.

In certain embodiments, linkers may be used to link any of the peptides or peptide domains or moieties of the invention (e.g., a napDNAbp linked or fused to a reverse transcriptase).

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 49), (G)ₙ (SEQ ID NO: 50), (EAAAK)ₙ (SEQ ID NO: 51), (GGS)ₙ (SEQ ID NO: 52), (SGGS)ₙ (SEQ ID NO: 53), (XP)ₙ (SEQ ID NO: 54), or any combination thereof, wherein n is independently an integer between 1 and 30, and wherein X is any amino acid. In some embodiments, the linker comprises the amino acid sequence (GGS)ₙ (SEQ ID NO: 52), wherein n is 1, 3, or 7. In some embodiments, the linker comprises the amino acid sequence SGSETPGTSESATPES (SEQ ID NO: 55). In some embodiments, the linker comprises the amino acid sequence SGGSSGGSSGSETPGTSESATPESSGGSSGGS (SEQ ID NO: 56). In some embodiments, the linker comprises the amino acid sequence SGGSGGSGGS (SEQ ID NO: 57). In some embodiments, the linker comprises the amino acid sequence SGGS (SEQ ID NO: 58).

In particular, the following linkers can be used in various embodiments to join prime editor domains with one another:

(SEQ ID NO: 59)
GGS;
(SEQ ID NO: 60)
GGSGGS;
(SEQ ID NO: 61)
GGSGGSGGS;
(SEQ ID NO: 7)
SGGSSGGSSGSETPGTSESATPESSGGSSGGSS;
(SEQ ID NO: 55)
SGSETPGTSESATPES;
(SEQ ID NO: 43)
SGGSSGGSSGSETPGTSESATPESAGSYPYDVPDYAGSAAPAAKKKKLDG
SGSGGSSGGS.

### Nuclear Localization Sequence (NLS)

In various embodiments, the the PE, twinPE, and/or multi-flap PE embodiments may comprise one or more nuclear localization sequences (NLS), which help promote translocation of a protein into the cell nucleus. Such sequences are well-known in the art and can include the following examples:

| DESCRIPTION | SEQUENCE | SEQ ID NO: |
|---|---|---|
| NLS OF SV40 | PKKKRKV | (SEQ ID NO: 62) |
| LARGE T-AG | | |
| NLS | MKRTADGSEFESPKKKRKV | (SEQ ID NO: 4) |
| NLS | MDSLLMNRRKFLYQFKNVRW | (SEQ ID NO: 63) |
| | AKGRRETYLC | |
| NLS OF | AVKRPAATKKAGQAKKKKLD | (SEQ ID NO: 64) |
| NUCLEOPLAS | | |
| MIN | | |
| NLS OF EGL-13 | MSRRRKANPTKLSENAKKLA | (SEQ ID NO: 65) |
| | KEVEN | |
| NLS OF C-MYC | PAAKRVKLD | (SEQ ID NO: 66) |
| NLS OF TUS- | KLKIKRPVK | (SEQ ID NO: 67) |
| PROTEIN | | |
| NLS OF | VSRKRPRP | (SEQ ID NO: 68) |
| POLYOMA | | |
| LARGE T-AG | | |
| NLS OF | EGAPPAKRAR | (SEQ ID NO: 69) |
| HEPATITIS D | | |
| VIRUS | | |
| ANTIGEN | | |
| NLS OF | PPQPKKKPLDGE | (SEQ ID NO: 70) |
| MURINE P53 | | |
| NLS OF PE1 | SGGSKRTADGSEFEPKKKRKV | (SEQ ID NO: 5) |
| AND PE2 | | |

The NLS examples above are non-limiting. The the PE, twinPE, and/or multi-flap PE embodiments may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the multi-flap prime editors and constructs encoding the prime editors disclosed herein further comprise one or more, preferably, at least two nuclear localization signals. In certain embodiments, the multi-flap prime editors comprise at least two NLSs. In embodiments with at least two NLSs, the NLSs can be the same NLSs or they can be different NLSs. In addition, the NLSs may be expressed as part of a fusion protein with the remaining portions of the multi-flap prime editors. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 62), MDSLLMNRRKFLYQFKNVRWAKGRRETYLC (SEQ ID NO: 63), KRTADGSEFESPKKKRKV (SEQ ID NO: 71), or KRTADGSEFEPKKKRKV (SEQ ID NO: 72). In other embodiments, NLS comprises the amino acid sequences

(SEQ ID NO: 73)
NLSKRPAAIKKAGQAKKKK,
(SEQ ID NO: 66)
PAAKRVKLD,
(SEQ ID NO: 74)
RQRRNELKRSF,
(SEQ ID NO: 75)
NQSSNFGPMKGGNFGGRSSGPYGGGGQYFAKPRNQGGY.

In one aspect of the disclosure, a multi-flap prime editor may be modified with one or more nuclear localization signals (NLS), preferably at least two NLSs. In certain embodiments, the multi-flap prime editors are modified with two or more NLSs. The disclosure contemplates the use of any nuclear localization signal known in the art at the time of the disclosure, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S. A. 89:7442-46; Moede et al., (1999) FEBS Lett. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 62)); (ii) a bipartite motif consisting of two basic domains separated by a variable number of spacer amino acids and exemplified by the Xenopus nucleoplasmin NLS (KRXXXXXXXXXXKKKL (SEQ ID NO: 76))); and (iii) noncanonical sequences such as M9 of the hnRNP A1 protein, the influenza virus nucleoprotein NLS, and the yeast Gal4 protein NLS (Dingwall and Laskey 1991).

Nuclear localization signals appear at various points in the amino acid sequences of proteins. NLS's have been identified at the N-terminus, the C-terminus, and in the central region of proteins. Thus, the disclosure provides multi-flap prime editors that may be modified with one or more NLSs at the C-terminus, the N-terminus, as well as at an internal region of the multi-flap prime editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a multi-flap prime editor to include one or more NLSs. In one aspect, the multi-flap prime editors may be engineered to express a prime editor protein that is translationally fused at its N-terminus or its C-terminus (or both) to one or more NLSs, i.e., to form a prime editor-NLS fusion construct. In other embodiments, the prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g, and in the central region of proteins. Thus, the present disclosure also provides for nucleotide constructs, vectors, and host cells for expressing fusion proteins that comprise a prime editor and one or more NLSs.

The multi-flap prime editors described herein may also comprise nuclear localization signals which are linked to a prime editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the prime editor by any suitable strategy that effectuates forming a bond (e.g., covalent linkage, hydrogen bonding) between the prime editor and the one or more NLSs.

### Flap Endonucleases (e.g., FEN1)

In various embodiments, the prime editing embodiments may comprise one or more flap endonucleases (e.g., FEN1), which refers to an enzyme that catalyzes the removal of 5′ single strand DNA flaps. These are naturally occurring enzymes that process the removal of 5′ flaps formed during cellular processes, including DNA replication. The multi-flap prime editing methods herein described may utilize endogenously supplied flap endonucleases or those provided in trans to remove the 5′ flap of endogenous DNA formed at the target site during multi-flap prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference). An exemplary flap endonuclease is FEN1, which can be represented by the following amino acid sequence:

| Description | Sequence | SEQ ID NO: |
|---|---|---|
| FEN1 | MGIQGLAKLIADVAPSAIRENDIKSYFGRKVAIDASMSI | SEQ ID NO: 77 |
| Wild type | YQFLIAVRQGGDVLQNEEGETTSHLMGMFYRTIRMME | |
| (wt) | NGIKPVYVFDGKPPQLKSGELAKRSERRAEAEKQLQQ | |
| | AQAAGAEQEVEKFTKRLVKVTKQHNDECKHLLSLMGI | |
| | PYLDAPSEAEASCAALVKAGKVYAAATEDMDCLTFGS | |
| | PVLMRHLTASEAKKLPIQEFHLSRILQELGLNQEQFVD | |
| | LCILLGSDYCESIRGIGPKRAVDLIQKHKSIEEIVRRLDP | |
| | NKYPVPENWLHKEAHQLFLEPEVLDPESVELKWSEPN | |
| | EEELIKFMCGEKQFSEERIRSGVKRLSKSRQGSTQGRLD | |
| | DFFKVTGSLSSAKRKEPEPKGSTKKKAKTGAAGKFKR | |
| | GK | |

The flap endonucleases may also include any FEN1 variant, mutant, or other flap endonuclease ortholog, homolog, or variant. Non-limiting FEN1 variant examples are as follows:

| Description | Sequence | SEQ ID NO: |
|---|---|---|
| FEN1 | MGIQGLAKLIADVAPSAIRENDIKSYFGRKVAIDASMSI | SEQ ID NO: 78 |
| K168R | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVRAGKVYAAATEDMDCLTFGS | |
| | PVLMRHLTASEAKKLPIQEFHLSRILQELGLNQEQFVD | |
| | LCILLGSDYCESIRGIGPKRAVDLIQKHKSIEEIVRRLDP | |
| | NKYPVPENWLHKEAHQLFLEPEVLDPESVELKWSEPN | |
| | EEELIKFMCGEKQFSEERIRSGVKRLSKSRQGSTQGRLD | |
| | DFFKVTGSLSSAKRKEPEPKGSTKKKAKTGAAGKFKR | |
| | GK | |
| FEN1 | MGIQGLAKLIADVAPSAIRENDIKSYFGRKVAIDASMSI | SEQ ID NO: 79 |
| S187A | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVKAGKVYAAATEDMDCLTFG | |
| | APVLMRHLTASEAKKLPIQEFHLSRILQELGLNQEQFV | |
| | DLCILLGSDYCESIRGIGPKRAVDLIQKHKSIEEIVRRLD | |
| | PNKYPVPENWLHKEAHQLFLEPEVLDPESVELKWSEP | |
| | NEEELIKFMCGEKQFSEERIRSGVKRLSKSRQGSTQGRL | |
| | DDFFKVTGSLSSAKRKEPEPKGSTKKKAKTGAAGKFK | |
| | RGK | |
| FEN1 | MGIQGLAKLIADVAPSAIRENDIKSYFGRKVAIDASMSI | SEQ ID NO: 80 |
| K354R | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVKAGKVYAAATEDMDCLTFGS | |
| | PVLMRHLTASEAKKLPIQEFHLSRILQELGLNQEQFVD | |
| | LCILLGSDYCESIRGIGPKRAVDLIQKHKSIEEIVRRLDP | |
| | NKYPVPENWLHKEAHQLFLEPEVLDPESVELKWSEPN | |
| | EEELIKFMCGEKQFSEERIRSGVKRLSKSRQGSTQGRLD | |
| | DFFKVTGSLSSARRKEPEPKGSTKKKAKTGAAGKFKR | |
| | GK | |
| GEN1 | MGVNDLWQILEPVKQHIPLRNLGGKTIAVDLSLWVCE | SEQ ID NO: 81 |
| | AQTVKKMMGSVMKPHLRNLFFRISYLTQMDVKLVFV | |
| | MEGEPPKLKADVISKRNQSRYGSSGKSWSQKTGRSHF | |
| | KSVLRECLHMLECLGIPWVQAAGEAEAMCAYLNAGG | |
| | HVDGCLTNDGDTFLYGAQTVYRNFTMNTKDPHVDCY | |
| | TMSSIKSKLGLDRDALVGLAILLGCDYLPKGVPGVGKE | |
| | QALKLIQILKGQSLLQRFNRWNETSCNSSPQLLVTKKL | |
| | AHCSVCSHPGSPKDHERNGCRLCKSDKYCEPHDYEYC | |
| | CPCEWHRTEHDRQLSEVENNIKKKACCCEGFPFHEVIQ | |
| | EFLLNKDKLVKVIRYQRPDLLLFQRFTLEKMEWPNHY | |
| | ACEKLLVLLTHYDMIERKLGSRNSNQLQPIRIVKTRIRN | |
| | GVHCFEIEWEKPEHYAMEDKQHGEFALLTIEEESLFEA | |
| | AYPEIVAVYQKQKLEIKGKKQKRIKPKENNLPEPDEVM | |
| | SFQSHMTLKPTCEIFHKQNSKLNSGISPDPTLPQESISAS | |
| | LNSLLLPKNTPCLNAQEQFMSSLRPLAIQQIKAVSKSLI | |
| | SESSQPNTSSHNISVIADLHLSTIDWEGTSFSNSPAIQRN | |
| | TFSHDLKSEVESELSAIPDGFENIPEQLSCESERYTANIK | |
| | KVLDEDSDGISPEEHLLSGITDLCLQDLPLKERIFTKLSY | |
| | PQDNLQPDVNLKTLSILSVKESCIANSGSDCTSHLSKDL | |
| | PGIPLQNESRDSKILKGDQLLQEDYKVNTSVPYSVSNT | |
| | VVKTCNVRPPNTALDHSRKVDMQTTRKILMKKSVCLD | |
| | RHSSDEQSAPVFGKAKYTTQRMKHSSQKHNSSHFKES | |
| | GHNKLSSPKIHIKETEQCVRSYETAENEESCFPDSTKSS | |
| | LSSLQCHKKENNSGTCLDSPLPLRQRLKLRFQST | |
| ERCC5 | MGVQGLWKLLECSGRQVSPEALEGKILAVDISIWLNQ | SEQ ID NO: 82 |
| | ALKGVRDRHGNSIENPHLLTLFHRLCKLLFFRIRPIFVF | |
| | DGDAPLLKKQTLVKRRQRKDLASSDSRKTTEKLLKTF | |
| | LKRQAIKTAFRSKRDEALPSLTQVRRENDLYVLPPLQE | |
| | EEKHSSEEEDEKEWQERMNQKQALQEEFFHNPQAIDIE | |
| | SEDFSSLPPEVKHEILTDMKEFTKRRRTLFEAMPEESDD | |
| | FSQYQLKGLLKKNYLNQHIEHVQKEMNQQHSGHIRRQ | |
| | YEDEGGFLKEVESRRVVSEDTSHYILIKGIQAKTVAEV | |
| | DSESLPSSSKMHGMSFDVKSSPCEKLKTEKEPDATPPSP | |
| | RTLLAMQAALLGSSSEEELESENRRQARGRNAPAAVD | |
| | EGSISPRTLSAIKRALDDDEDVKVCAGDDVQTGGPGAE | |
| | EMRINSSTENSDEGLKVRDGKGIPFTATLASSSVNSAEE | |
| | HVASTNEGREPTDSVPKEQMSLVHVGTEAFPISDESMI | |
| | KDRKDRLPLESAVVRHSDAPGLPNGRELTPASPTCTNS | |
| | VSKNETHAEVLEQQNELCPYESKFDSSLLSSDDETKCK | |
| | PNSASEVIGPVSLQETSSIVSVPSEAVDNVENVVSFNAK | |
| | EHENFLETIQEQQTTESAGQDLISIPKAVEPMEIDSEESE | |
| | SDGSFIEVQSVISDEELQAEFPETSKPPSEQGEEELVGTR | |
| | EGEAPAESESLLRDNSERDDVDGEPQEAEKDAEDSLHE | |
| | WQDINLEELETLESNLLAQQNSLKAQKQQQERIAATVT | |
| | GQMFLESQELLRLFGIPYIQAPMEAEAQCAILDLTDQTS | |
| | GTITDDSDIWLFGARHVYRNFFNKNKFVEYYQYVDFH | |
| | NQLGLDRNKLINLAYLLGSDYTEGIPTVGCVTAMEILN | |
| | EFPGHGLEPLLKFSEWWHEAQKNPKIRPNPHDTKVKK | |
| | KLRTLQLTPGFPNPAVAEAYLKPVVDDSKGSFLWGKP | |
| | DLDKIREFCQRYFGWNRTKTDESLFPVLKQLDAQQTQ | |
| | LRIDSFFRLAQQEKEDAKRIKSQRLNRAVTCMLRKEKE | |
| | AAASEIEAVSVAMEKEFELLDKAKRKTQKRGITNTLEE | |
| | SSSLKRKRLSDSKRKNTCGGFLGETCLSESSDGSSSEDA | |
| | ESSSLMNVQRRTAAKEPKTSASDSQNSVKEAPVKNGG | |
| | ATTSSSSDSDDDGGKEKMVLVTARSVFGKKRRKLRRA | |
| | RGRKRKT | |

In various embodiments, the multi-flap prime editor fusion proteins contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences.

Other endonucleases that may be utilized by the instant methods to facilitate removal of the 5′ end single strand DNA flap include, but are not limited to (1) trex 2, (2) exol endonuclease (e.g., Keijzers et al., Biosci Rep. 2015, 35(3): e00206)

### Trex 2

3′ three prime repair exonuclease 2 (TREX2)-human
Accession No. NM_080701
(SEQ ID NO: 83)
MSEAPRAETFVFLDLEATGLPSVEPEIAELSLFAVHRSSLENPEHDESGA
LVLPRVLDKLTLCMCPERPFTAKASEITGLSSEGLARCRKAGFDGAVVRT
LQAFLSRQAGPICLVAHNGFDYDFPLLCAELRRLGARLPRDTVCLDTLPA
LRGLDRAHSHGTRARGRQGYSLGSLFHRYFRAEPSAAHSAEGDVHTLLLI
FLHRAAELLAWADEQARGWAHIEPMYLPPDDPSLEA.
3′ three prime repair exonuclease 2 (TREX2)-mouse
Accession No. NM_011907
(SEQ ID NO: 84)
MSEPPRAETFVFLDLEATGLPNMDPEIAEISLFAVHRSSLENPERDDSGS
LVLPRVLDKLTLCMCPERPFTAKASEITGLSSESLMHCGKAGFNGAVVRT
LQGFLSRQEGPICLVAHNGFDYDFPLLCTELQRLGAHLPQDTVCLDTLPA
LRGLDRAHSHGTRAQGRKSYSLASLFHRYFQAEPSAAHSAEGDVHTLLLI
FLHRAPELLAWADEQARSWAHIEPMYVPPDGPSLEA.
3′ three prime repair exonuclease 2 (TREX2)-rat
Accession No. NM_001107580
(SEQ ID NO: 85)
MSEPLRAETFVFLDLEATGLPNMDPEIAEISLFAVHRSSLENPERDDSGS
LVLPRVLDKLTLCMCPERPFTAKASEITGLSSEGLMNCRKAAFNDAVVRT
LQGFLSRQEGPICLVAHNGFDYDFPLLCTELQRLGAHLPRDTVCLDTLPA
LRGLDRVHSHGTRAQGRKSYSLASLFHRYFQAEPSAAHSAEGDVNTLLLI
FLHRAPELLAWADEQARSWAHIEPMYVPPDGPSLEA.

### ExoI

Human exonuclease 1 (EXO1) has been implicated in many different DNA metabolic processes, including DNA mismatch repair (MMR), micro-mediated end-joining, homologous recombination (HR), and replication. Human EXO1 belongs to a family of eukaryotic nucleases, Rad2/XPG, which also include FEN1 and GEN1. The Rad2/XPG family is conserved in the nuclease domain through species from phage to human. The EXO1 gene product exhibits both 5′ exonuclease and 5′ flap activity. Additionally, EXO1 contains an intrinsic 5′ RNase H activity. Human EXO1 has a high affinity for processing double stranded DNA (dsDNA), nicks, gaps, pseudo Y structures and can resolve Holliday junctions using its inherit flap activity. Human EXO1 is implicated in MMR and contain conserved binding domains interacting directly with MLH1 and MSH2. EXO1 nucleolytic activity is positively stimulated by PCNA, MutSα (MSH2/MSH6 complex), 14-3-3, MRN and 9-1-1 complex.

exonuclease 1 (EXO1) Accession No. NM_003686
(Homo sapiens exonuclease 1 (EXO1), transcript
variant 3)-isoform A
(SEQ ID NO: 86)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSAELSEDDLLSQYSLSFTKKTKKNSSEGNKSLSFSE
VFVPDLVNGPTNKKSVSTPPRTRNKFATFLQRKNEESGAVVVPGTRSRFFCSSDSTDC
VSNKVSIQPLDETAVTDKENNLHESEYGDQEGKRLVDTDVARNSSDDIPNNHIPGDH
IPDKATVFTDEESYSFESSKFTRTISPPTLGTLRSCFSWSGGLGDFSRTPSPSPSTALQQ
FRRKSDSPTSLPENNMSDVSQLKSEESSDDESHPLREEACSSQSQESGEFSLQSSNASK
LSQCSSKDSDSEESDCNIKLLDSQSDQTSKLRLSHFSKKDTPLRNKVPGLYKSSSADS
LSTTKIKPLGPARASGLSKKPASIQKRKHHNAENKPGLQIKLNELWKNFGFKKF.
exonuclease 1 (EXO1) Accession No. NM_006027
(Homo sapiens exonuclease 1 (EXO1), transcript
variant 3)-isoform B
(SEQ ID NO: 87)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSAELSEDDLLSQYSLSFTKKTKKNSSEGNKSLSFSE
VFVPDLVNGPTNKKSVSTPPRTRNKFATFLQRKNEESGAVVVPGTRSRFFCSSDSTDC
VSNKVSIQPLDETAVTDKENNLHESEYGDQEGKRLVDTDVARNSSDDIPNNHIPGDH
IPDKATVFTDEESYSFESSKFTRTISPPTLGTLRSCFSWSGGLGDFSRTPSPSPSTALQQ
FRRKSDSPTSLPENNMSDVSQLKSEESSDDESHPLREEACSSQSQESGEFSLQSSNASK
LSQCSSKDSDSEESDCNIKLLDSQSDQTSKLRLSHFSKKDTPLRNKVPGLYKSSSADS
LSTTKIKPLGPARASGLSKKPASIQKRKHHNAENKPGLQIKLNELWKNFGFKKDSEK
LPPCKKPLSPVRDNIQLTPEAEEDIFNKPECGRVQRAIFQ.
exonuclease 1 (EXO1) Accession No. NM_001319224
(Homo sapiens exonuclease 1 (EXO1), transcript
variant 4)-isoform C
(SEQ ID NO: 88)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSELSEDDLLSQYSLSFTKKTKKNSSEGNKSLSFSEV
FVPDLVNGPTNKKSVSTPPRTRNKFATFLQRKNEESGAVVVPGTRSRFFCSSDSTDCV
SNKVSIQPLDETAVTDKENNLHESEYGDQEGKRLVDTDVARNSSDDIPNNHIPGDHIP
DKATVFTDEESYSFESSKFTRTISPPTLGTLRSCFSWSGGLGDFSRTPSPSPSTALQQFR
RKSDSPTSLPENNMSDVSQLKSEESSDDESHPLREEACSSQSQESGEFSLQSSNASKLS
QCSSKDSDSEESDCNIKLLDSQSDQTSKLRLSHFSKKDTPLRNKVPGLYKSSSADSLS
TTKIKPLGPARASGLSKKPASIQKRKHHNAENKPGLQIKLNELWKNFGFKKDSEKLP
PCKKPLSPVRDNIQLTPEAEEDIFNKPECGRVQRAIFQ.

### Inteins and Split-Inteins

It will be understood that in some embodiments (e.g., delivery of a multi-flap prime editor in vivo using AAV particles), it may be advantageous to split a polypeptide (e.g., a deaminase or a napDNAbp) or a fusion protein (e.g., a multi-flap prime editor) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketone, aldehyde, Cys residues and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the −12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g. micromolar) concentrations of proteins and can be carried out under physiological conditions.

Exemplary sequences are as follows:

| NAME | SEQUENCE OF LIGAND-DEPENDENT INTEIN |
|---|---|
| 2-4 INTEIN: | CLAEGTRIFDPVTGTTHRIEDVVDGRKPIHVVAAAKDGTLLARPVV |
| | SWFDQGTRDVIGLRIAGGAIVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLECAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEELRYSVIREVLPTRRARTFDLEVEELHTLVAEGVVVH |
| | NC (SEQ ID NO: 89) |
| 3-2 INTEIN | CLAEGTRIFDPVTGTTHRIEDVVDGRKPIHVVAVAKDGTLLARPVVS |
| | WFDQGTRDVIGLRIAGGAIVWATPDHKVLTEYGWRAAGELRKGDR |
| | VAGPGGSGNSLALSLTADQMVSALLDAEPPILYSEYDPTSPFSEASM |
| | MGLLTNLADRELVHMINWAKRVPGFVDLTLHDQAHLLECAWLEIL |
| | MIGLVWRSMEHPGKLLFAPNLLLDRNQGKCVEGMVEIFDMLLATS |
| | SRFRMMNLQGEEFVCLKSIILLNSGVYTFLSSTLKSLEEKDHIHRAL |
| | DKITDTLIHLMAKAGLTLQQQHQRLAQLLLILSHIRHMSNKGMEHL |
| | YSMKYTNVVPLYDLLLEMLDAHRLHAGGSGASRVQAFADALDDK |
| | FLHDMLAEELRYSVIREVLPTRRARTFDLEVEELHTLVAEGVVVHN |
| | C (SEQ ID NO: 90) |
| 30R3-1 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPIPYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEGLRYSVIREVLPTRRARTFDLEVEELHTLVAEGVV |
| | VHNC (SEQ ID NO: 91) |
| 30R3-2 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPILYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEELRYSVIREVLPTRRARTFDLEVEELHTLVAEGVV |
| | VHNC (SEQ ID NO: 92) |
| 30R3-3 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPIPYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEELRYSVIREVLPTRRARTFDLEVEELHTLVAEGVV |
| | VHNC (SEQ ID NO: 93) |
| 37R3-1 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPILYSEYNPTSPFSEA |
| | SMMGLLTNLADRELVHMINWAKRVPGFVDLTLHDQAHLLERAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEGLRYSVIREVLPTRRARTFDLEVEELHTLVAEGVV |
| | VHNC (SEQ ID NO: 94) |
| 37R3-2 INTEIN | CLAEGTRIFDPVTGTTHRIEDVVDGRKPIHVVAAAKDGTLLARPVV |
| | SWFDQGTRDVIGLRIAGGAIVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLERAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEGLRYSVIREVLPTRRARTFDLEVEELHTLVAEGVVVH |
| | NC (SEQ ID NO: 95) |
| 37R3-3 INTEIN | CLAEGTRIFDPVTGTTHRIEDVVDGRKPIHVVAVAKDGTLLARPVVS |
| | WFDQGTRDVIGLRIAGGATVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLERAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEELRYSVIREVLPTRRARTFDLEVEELHTLVAEGVVVH |
| | NC (SEQ ID NO: 96) |

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-C, which encode the DnaE-N and DnaE-C subunits, respectively. DnaE is a naturally occurring split intein in Synechocystis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol. 114: 8538-8543; Iwai et al., “Highly efficient protein trans-splicing by a naturally split DnaE intein from Nostc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. US A, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product with regard to the formation of a complete PE fusion protein from two separately-expressed halves.

### RNA-Protein Interaction Domain

In various embodiments, two separate protein domains (e.g., a Cas9 domain and a polymerase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a multi-flap prime editor, as well as to recruitment additional functionalities to a multi-flap prime editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 97).

The amino acid sequence of the MCP or MS2cp is:

(SEQ ID NO: 98)
GSASNFTQFVLVDNGGTGDVTVAPSNFANGVAEWISSNSRSQAYKVTCSV
RQSSAQNRKYTIKVEVPKVATQTVGGEELPVAGWRSYLNMELTIPIFATN
SDCELIVKAMQGLLKDGNPIPSAIAANSGIY.

### Additional PE Elements

In certain embodiments, the PEs, twinPEs, and multi-flap PEs described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, MBD4 inhibitor or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 100 (human TDG).

Some exemplary glycosylases are provided below. The catalytically inactivated variants of any of these glycosylase domains are iBERs that may be fused to the napDNAbp or polymerase domain of the multi-flap prime editors provided in this disclosure.

OGG (human)
(SEQ ID NO: 100)
MPARALLPRRMGHRTLASTPALWASIPCPRSELRLDLVLPSGQSFRWREQSPA
HWSGVLADQVWTLTQTEEQLHCTVYRGDKSQASRPTPDELEAVRKYFQLDVTLAQ
LYHHWGSVDSHFQEVAQKFQGVRLLRQDPIECLFSFICSSNNNIARITGMVERLCQAF
GPRLIQLDDVTYHGFPSLQALAGPEVEAHLRKLGLGYRARYVSASARAILEEQGGLA
WLQQLRESSYEEAHKALCILPGVGTKVADCICLMALDKPQAVPVDVHMWHIAQRD
YSWHPTTSQAKGPSPQTNKELGNFFRSLWGPYAGWAQAVLFSADLRQSRHAQEPPA
KRRKGSKGPEG
MPG (human)
(SEQ ID NO: 101)
MVTPALQMKKPKQFCRRMGQKKQRPARAGQPHSSSDAAQAPAEQPHSSSDA
AQAPCPRERCLGPPTTPGPYRSIYFSSPKGHLTRLGLEFFDQPAVPLARAFLGQVLVR
RLPNGTELRGRIVETEAYLGPEDEAAHSRGGRQTPRNRGMFMKPGTLYVYIIYGMYF
CMNISSQGDGACVLLRALEPLEGLETMRQLRSTLRKGTASRVLKDRELCSGPSKLCQ
ALAINKSFDQRDLAQDEAVWLERGPLEPSEPAVVAAARVGVGHAGEWARKPLRFY
VRGSPWVSVVDRVAEQDTQA
MBD4 (human)
(SEQ ID NO: 102)
MGTTGLESLSLGDRGAAPTVTSSERLVPDPPNDLRKEDVAMELERVGEDEEQ
MMIKRSSECNPLLQEPIASAQFGATAGTECRKSVPCGWERVVKQRLFGKTAGRFDV
YFISPQGLKFRSKSSLANYLHKNGETSLKPEDFDFTVLSKRGIKSRYKDCSMAALTSH
LQNQSNNSNWNLRTRSKCKKDVFMPPSSSSELQESRGLSNFTSTHLLLKEDEGVDDV
NFRKVRKPKGKVTILKGIPIKKTKKGCRKSCSGFVQSDSKRESVCNKADAESEPVAQ
KSQLDRTVCISDAGACGETLSVTSEENSLVKKKERSLSSGSNFCSEQKTSGIINKFCSA
KDSEHNEKYEDTFLESEEIGTKVEVVERKEHLHTDILKRGSEMDNNCSPTRKDFTGE
KIFQEDTIPRTQIERRKTSLYFSSKYNKEALSPPRRKAFKKWTPPRSPFNLVQETLFHD
PWKLLIATIFLNRTSGKMAIPVLWKFLEKYPSAEVARTADWRDVSELLKPLGLYDLR
AKTIVKFSDEYLTKQWKYPIELHGIGKYGNDSYRIFCVNEWKQVHPEDHKLNKYHD
WLWENHEKLSLS
TDG (human)
(SEQ ID NO: 103)
MEAENAGSYSLQQAQAFYTFPFQQLMAEAPNMAVVNEQQMPEEVPAPAPA
QEPVQEAPKGRKRKPRTTEPKQPVEPKKPVESKKSGKSAKSKEKQEKITDTFKVKRK
VDRFNGVSEAELLTKTLPDILTFNLDIVIIGINPGLMAAYKGHHYPGPGNHFWKCLFM
SGLSEVQLNHMDDHTLPGKYGIGFTNMVERTTPGSKDLSSKEFREGGRILVQKLQKY
QPRIAVFNGKCIYEIFSKEVFGVKVKNLEFGLQPHKIPDTETLCYVMPSSSARCAQFPR
AQDKVHYYIKLKDLRDQLKGIERNMDVQEVQYTFDLQLAQEDAKKMAVKEEKYDP
GYEAAYGGAYGENPCSSEPCGESSNGLIESVELRGESAFSGIPNGQWMTQSFTDQIPS
FSNHCGTQEQEEESHA

In some embodiments, the PEs, twinPEs, and multi-flap PEs described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a PE, twinPE, and multi-flap PE or component thereof (e.g., the napDNAbp domain, the polymerase domain, or the NLS domain) include, without limitation, epitope tags, and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A multi-flap prime editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011 and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments of the present disclosure, the activity of the multi-flap prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the PEgRNA may deliver the guide prior to the vector encoding the PE system. In some embodiments, the vectors encoding the PE system and PEgRNA are delivered simultaneously. In certain embodiments, the simultaneously delivered vectors temporally deliver, e.g., the PE, PEgRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the PEgRNA may be self-destroyed via cleavage of a target sequence present on the vector by the PE system. The cleavage may prevent continued transcription of a PE or a PEgRNA from the vector. Although transcription may occur on the linearized vector for some amount of time, the expressed transcripts or proteins subject to intracellular degradation will have less time to produce off-target effects without continued supply from expression of the encoding vectors.

### PEgRNAs

The prime editing systems described herein (e.g., PE, twinPE, and multi-flap PE) contemplates the use of any suitable PEgRNAs. The mechanism of target-primed reverse transcription (TPRT) can be leveraged or adapted for conducting precision and versatile CRISPR/Cas-based genome editing through the use of a specially configured guide RNA comprising a reverse transcription (RT) template sequence that codes for the desired nucleotide change. The application refers to this specially configured guide RNA as an “extended guide RNA” or a “PEgRNA” since the RT template sequence can be provided as an extension of a standard or traditional guide RNA molecule. The application contemplates any suitable configuration or arrangement for the extended guide RNAs for use in dual-flap and quadruple-flap prime editing.

PEgRNAs used for twinPE and multi-flap PE have a similar design to those used for classic prime editing, however it is not necessary for the RT template region to encode any homology to the target locus. Instead, the two PEgRNAs can in various embodiments contain RT templates that encode the synthesis of 3′ flaps whose 3′ ends are reverse complement sequences of one another. This complementarity between the 3′ flaps promotes their annealing and replacement of the endogenous DNA sequence with the intended new DNA sequence. This necessitates that the 5′ regions of the RT templates in the two PEgRNAs are reverse complement sequences to one another, and this amount of complementarity can vary.

### PEgRNA Architecture

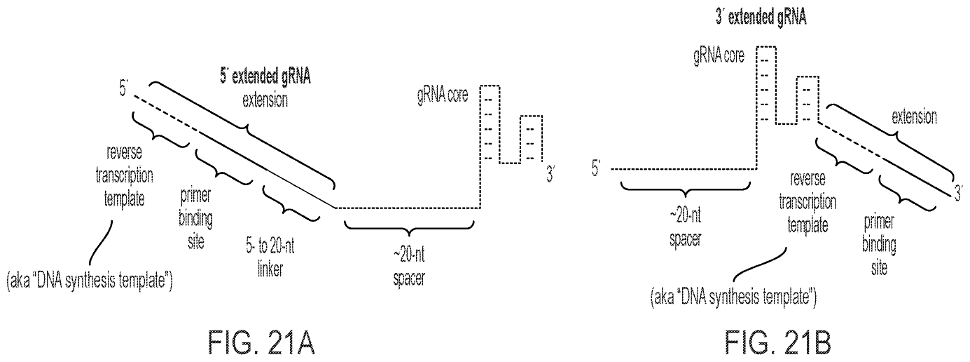

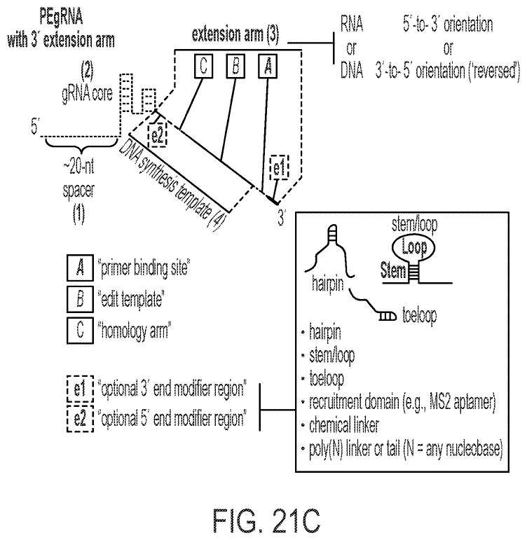

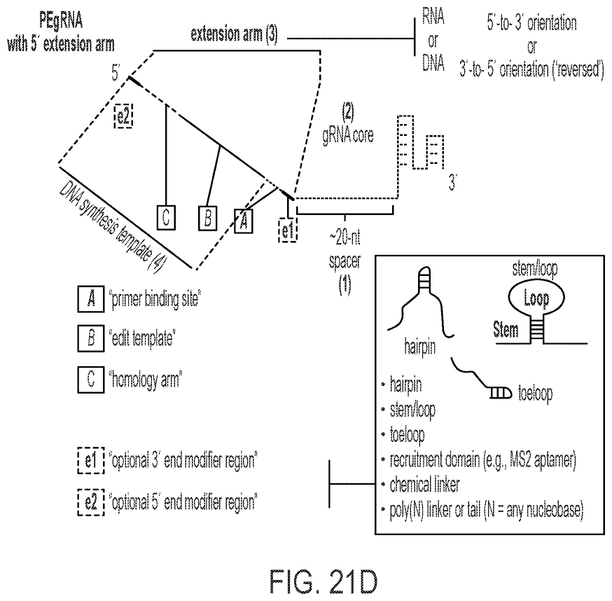

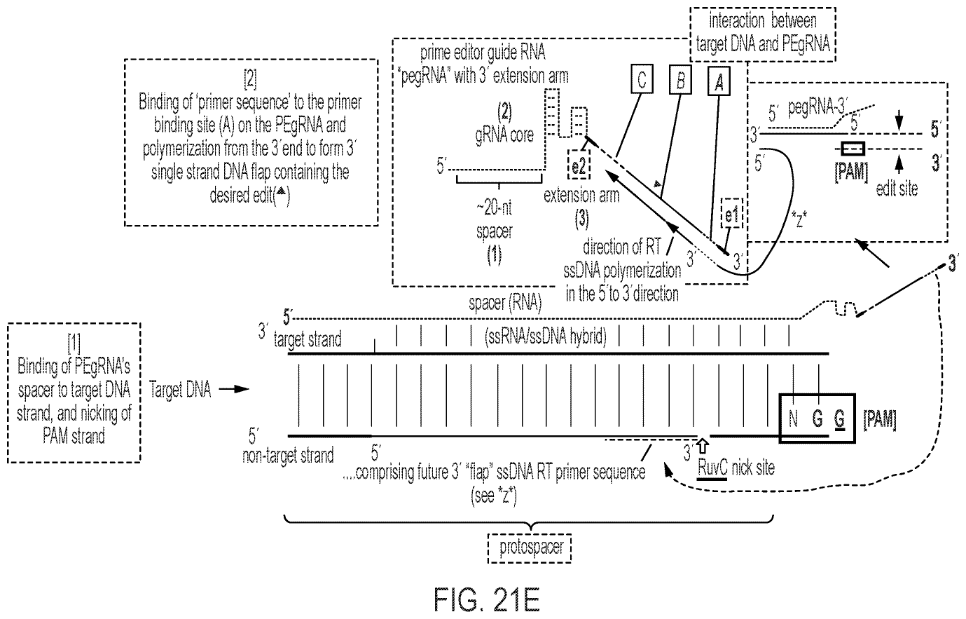

FIG. 21A shows one embodiment of an PEgRNA usable in the prime editing systems disclosed herein (e.g., PE, twinPE, and multi-flap PE) whereby a traditional guide RNA includes a ˜20 nt protospacer sequence and a gRNA core region, which binds with the napDNAbp. In this embodiment, the guide RNA includes an extended RNA segment at the 5′ end, i.e., a 5′ extension. In this embodiment, the 5′ extension includes a reverse transcription template sequence, a reverse transcription primer binding site, and an optional 5-20 nucleotide linker sequence. The primer binding site hybridizes to the free 3′ end that is formed after a nick is formed in the non-target strand of the R-loop, thereby priming reverse transcriptase for DNA polymerization in the 5′ to 3′ direction.

FIG. 21B shows another embodiment of an extended guide RNA usable in the prime editing systems disclosed herein (e.g., PE, twinPE, and multi-flap PE) whereby a traditional guide RNA includes a ˜20 nt protospacer sequence and a gRNA core, which binds with the napDNAbp. In this embodiment, the guide RNA includes an extension arm at the 3′ end, i.e., a 3′ extension. In this embodiment, the 3′ extension includes a DNA synthesis template, and a primer binding site. The primer binding site hybridizes to the free 3′ end that is formed after a nick is formed in the non-target strand of the R-loop, thereby priming reverse transcriptase for DNA polymerization in the 5′ to 3′ direction.

The length of the extension arm can be any useful length. In various embodiments, the RNA extension arm is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The DNA synthesis template sequence can also be any suitable length. For example, the template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, wherein the primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The DNA synthesis template sequence, in certain embodiments, encodes a single-stranded DNA molecule which is homologous to the non-target strand (and thus, complementary to the corresponding site of the target strand) but includes one or more nucleotide changes. The least one nucleotide change may include one or more single-base nucleotide changes, one or more deletions, and one or more insertions.

In various embodiments of the extended guide RNAs, the template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site. In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs may be naturally occurring, expressed from an encoding nucleic acid, or synthesized chemically. Methods are well known in the art for obtaining or otherwise synthesizing guide RNAs and for determining the appropriate sequence of the guide RNA, including the protospacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in the multi-flap prime editing systems described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a multi-flap prime editor to a target sequence may be assessed by any suitable assay. For example, the components of a multi-flap prime editor, including the guide sequence to be tested, may be provided to a host cell having the corresponding target sequence, such as by transfection with vectors encoding the components of a multi-flap prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a multi-flap prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything). A unique target sequence in a genome may include an S. pyogenes Cas9 target site of the form MMMMMMMMMNNNNNNNNNNNXGG where NNNNNNNNNNNXGG (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW where NNNNNNNNNNNNXXAGAAW (N is A, G, T, or C; X can be anything; and W is A or T). A unique target sequence in a genome may include an S. thermophilus CRISPR1 Cas9 target site of the form MMMMMMMMMNNNNNNNNNNNXXAGAAW where NNNNNNNNNNNXXAGAAW (N is A, G, T, or C; X can be anything; and W is A or T). For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything). A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything). In each of these sequences “M” may be A, G, T, or C, and need not be considered in identifying a sequence as unique.

As used herein in a PEgRNA or guide RNA sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(SEQ ID NO: 104)
(1) NNNNNNNNgtttttgtactctcaagatttaGAAAtaaatcttgcag
aagctacaaagataaggcttcatgccgaaatcaacaccctgtcattttat
ggcagggtgttttcgttatttaaTTTTTT;
(SEQ ID NO: 105)
(2) NNNNNNNNNNNNNNNNNNgtttttgtactctcaGAAAtgcagaagc
tacaaagataaggcttcatgccgaaatcaacaccctgtcattttatggca
gggtgttttcgttatttaaTTTTTT;
(SEQ ID NO: 106)
(3) NNNNNNNNNNNNNNNNNNNNgtttttgtactctcaGAAAtgcagaa
gctacaaagataaggcttcatgccgaaatcaacaccctgtcattttatgg
cagggtgtTTTTT;
(SEQ ID NO: 107)
(4) NNNNNNNNNNNNNNNNNNNNgttttagagctaGAAAtagcaagtta
aaataaggctagtccgttatcaacttgaaaaagtggcaccgagtcggtgc
TTTTTT;
(SEQ ID NO: 108)
(5) NNNNNNNNNNNNNNNNNNNNgttttagagctaGAAATAGcaagtta
aaataaggctagtccgttatcaacttgaaaaagtgTTTTTTT;
and
(SEQ ID NO: 109)
(6) NNNNNNNNNNNNNNNNNNNNgttttagagctagAAATAGcaagtta
aaataaggctagtccgttatcaTTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[27bp spacer]-guuuuagagcuagaaauagcaaguuaaaauaaaggcuaguccguuaucaacuugaaaaaguggcaccgagucggugcuu uuu-3′ (SEQ ID NO: 110), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the multi-flap prime editor described herein.

In other embodiments, the PEgRNAs include those depicted in FIG. 21A-21D.

FIG. 21C provides the structure of an exemplary PEgRNA contemplated herein. The PEgRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an DNA synthesis template (or “edit template”) (B), and an optionally a homology arm (C) (which is not required for twinPE or multi-flap PE). In addition, the PEgRNA may comprise an optional 3′ end modifier region (e1) and an optional 5′ end modifier region (e2). Still further, the PEgRNA may comprise a transcriptional termination signal at the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The PEgRNA could comprise, in certain embodiments, secondary RNA structure, such as, but not limited to, hairpins, stem/loops, toe loops, RNA-binding protein recruitment domains (e.g., the MS2 aptamer which recruits and binds to the MS2cp protein). For instance, such secondary structures could be position within the spacer, the gRNA core, or the extension arm, and in particular, within the e1 and/or e2 modifier regions. In addition to secondary RNA structures, the PEgRNAs could comprise (e.g., within the e1 and/or e2 modifier regions) a chemical linker or a poly(N) linker or tail, where “N” can be any nucleobase. In some embodiments, the chemical linker may function to prevent reverse transcription of the sgRNA scaffold or core. In addition, in certain embodiments, the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the PEgRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the PEgRNA (e.g., in the e1 or e2 region, or elsewhere and an MS2cp protein fused to the DNA polymerase, thereby co-localizing the DNA polymerase to the PEgRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

The embodiment of FIG. 21D provides the structure of another PEgRNA contemplated herein. The PEgRNA comprises three main component elements ordered in the 5′ to 3′ direction, namely: an extension arm, a spacer, and a gRNA core. The extension arm may further be divided into the following structural elements in the 5′ to 3′ direction, namely: a primer binding site (A), an edit template (B), and an optional homology arm (C). The homology arm is not required in the twinPE and multi-flap PE embodiments. In addition, the PEgRNA may comprise an optional 3′ end modifier region (e1) and an optional 5′ end modifier region (e2). Still further, the PEgRNA may comprise a transcriptional termination signal on the 3′ end of the PEgRNA (not depicted). These structural elements are further defined herein. The depiction of the structure of the PEgRNA is not meant to be limiting and embraces variations in the arrangement of the elements. For example, the optional sequence modifiers (e1) and (e2) could be positioned within or between any of the other regions shown, and not limited to being located at the 3′ and 5′ ends. The PEgRNA could comprise, in certain embodiments, secondary RNA structures, such as, but not limited to, hairpins, stem/loops, toe loops, RNA-binding protein recruitment domains (e.g., the MS2 aptamer which recruits and binds to the MS2cp protein). These secondary structures could be positioned anywhere in the PEgRNA molecule. For instance, such secondary structures could be position within the spacer, the gRNA core, or the extension arm, and in particular, within the e1 and/or e2 modifier regions. In addition to secondary RNA structures, the PEgRNAs could comprise (e.g., within the e1 and/or e2 modifier regions) a chemical linker or a poly(N) linker or tail, where “N” can be any nucleobase. In some embodiments, the chemical linker may function to prevent reverse transcription of the sgRNA scaffold or core. In addition, in certain embodiments, the extension arm (3) could be comprised of RNA or DNA, and/or could include one or more nucleobase analogs (e.g., which might add functionality, such as temperature resilience). Still further, the orientation of the extension arm (3) can be in the natural 5′-to-3′ direction, or synthesized in the opposite orientation in the 3′-to-5′ direction (relative to the orientation of the PEgRNA molecule overall). It is also noted that one of ordinary skill in the art will be able to select an appropriate DNA polymerase, depending on the nature of the nucleic acid materials of the extension arm (i.e., DNA or RNA), for use in prime editing that may be implemented either as a fusion with the napDNAbp or as provided in trans as a separate moiety to synthesize the desired template-encoded 3′ single-strand DNA flap that includes the desired edit. For example, if the extension arm is RNA, then the DNA polymerase could be a reverse transcriptase or any other suitable RNA-dependent DNA polymerase. However, if the extension arm is DNA, then the DNA polymerase could be a DNA-dependent DNA polymerase. In various embodiments, provision of the DNA polymerase could be in trans, e.g., through the use of an RNA-protein recruitment domain (e.g., an MS2 hairpin installed on the PEgRNA (e.g., in the e1 or e2 region, or elsewhere and an MS2cp protein fused to the DNA polymerase, thereby co-localizing the DNA polymerase to the PEgRNA). It is also noted that the primer binding site does not generally form a part of the template that is used by the DNA polymerase (e.g., reverse transcriptase) to encode the resulting 3′ single-strand DNA flap that includes the desired edit. Thus, the designation of the “DNA synthesis template” refers to the region or portion of the extension arm (3) that is used as a template by the DNA polymerase to encode the desired 3′ single-strand DNA flap containing the edit and regions of homology to the 5′ endogenous single strand DNA flap that is replaced by the 3′ single strand DNA strand product of prime editing DNA synthesis. In some embodiments, the DNA synthesis template includes the “edit template” and the optional “homology arm”, or one or more homology arms, e.g., before and after the edit template. The edit template can be as small as a single nucleotide substitution, or it may be an insertion, or an inversion of DNA. In addition, the edit template may also include a deletion, which can be engineered by encoding homology arm that contains a desired deletion. In other embodiments, the DNA synthesis template may also include the e2 region or a portion thereof. For instance, if the e2 region comprises a secondary structure that causes termination of DNA polymerase activity, then it is possible that DNA polymerase function will be terminated before any portion of the e2 region is actual encoded into DNA. It is also possible that some or even all of the e2 region will be encoded into DNA. How much of e2 is actually used as a template will depend on its constitution and whether that constitution interrupts DNA polymerase function.

### PEgRNA Improvements

The PEgRNAs may also include additional design improvements that may modify the properties and/or characteristics of PEgRNAs thereby improving the efficacy of PE, twinPE, or multi-flap prime editing. In various embodiments, these improvements may belong to one or more of a number of different categories, including but not limited to: (1) designs to enable efficient expression of functional PEgRNAs from non-polymerase III (pol III) promoters, which would enable the expression of longer PEgRNAs without burdensome sequence requirements; (2) improvements to the core, Cas9-binding PEgRNA scaffold, which could improve efficacy; (3) modifications to the PEgRNA to improve RT processivity, enabling the insertion of longer sequences at targeted genomic loci; and (4) addition of RNA motifs to the 5′ or 3′ termini of the PEgRNA that improve PEgRNA stability, enhance RT processivity, prevent misfolding of the PEgRNA, or recruit additional factors important for genome editing.

In one embodiment, PEgRNA could be designed with polIII promoters to improve the expression of longer-length PEgRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a PEgRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed PEgRNA, which has been shown to result in markedly reduced Cas9:sgRNA activity in a site-dependent manner. Additionally, while pol III-transcribed PEgRNAs can simply terminate in a run of 6-7 U's, PEgRNAs transcribed from pol II or pol I would require a different termination signal. Often such signals also result in polyadenylation, which would result in undesired transport of the PEgRNA from the nucleus. Similarly, RNAs expressed from pol II promoters such as pCMV are typically 5′-capped, also resulting in their nuclear export.

In various embodiments, the PEgRNA may include various elements, as exemplified by the following sequence.

Non-limiting example 1-PEgRNA expression platform
consisting of pCMV, Csy4 hairpin, the PEgRNA, and
MALATI ENE:
(SEQ ID NO: 111)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTAGGGTCATGAAGGTT
TTTCTTTTCCTGAGAAAACAACACGTATTGTTTTCTCAGGTTTTGCTTTT
TGGCCTTTTTCTAGCTTAAAAAAAAAAAAAGCAAAAGATGCTGGTGGTTG
GCACTCCTGGTTTCCAGGACGGGGTTCAAATCCCTGCGGCGTCTTTGCTT
TGACT
Non-limiting example 2-PEgRNA expression platform
consisting of pCMV, Csy4 hairpin, the PEgRNA, and
PAN ENE:
(SEQ ID NO: 112)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAA
Non-limiting example 3-PEgRNA expression platform
consisting of pCMV, Csy4 hairpin, the PEgRNA, and
3xPAN ENE
(SEQ ID NO: 113)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAAACACACTG
TTTTGGCTGGGTTTTTCCTTGTTCGCACCGGACACCTCCAGTGACCAGAC
GGCAAGGTTTTTATCCCAGTGTATATTGGAAAAACATGTTATACTTTTGA
CAATTTAACGTGCCTAGAGCTCAAATTAAACTAATACCATAACGTAATGC
AACTTACAACATAAATAAAGGTCAATGTTTAATCCATAAAAAAAAAAAAA
AAAAAATCTCTCTGTTTTGGCTGGGTTTTTCCTTGTTCGCACCGGACACC
TCCAGTGACCAGACGGCAAGGTTTTTATCCCAGTGTATATTGGAAAAACA
TGTTATACTTTTGACAATTTAACGTGCCTAGAGCTCAAATTAAACTAATA
CCATAACGTAATGCAACTTACAACATAAATAAAGGTCAATGTTTAATCCA
TAAAAAAAAAAAAAAAAAAA
Non-limiting example 4-PEgRNA expression platform
consisting of pCMV, Csy4 hairing, the PEgRNA, and
3′ box
(SEQ ID NO: 114)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTGTTTCAAAAGTAGACT
GTACGCTAAGGGTCATATCTTTTTTTGTTTGGTTTGTGTCTTGGTTGGCG
TCTTAAA
Non-limiting example 5-PEgRNA expression platform
consisting of pU1, Csy4 hairping, the PERNA, and
3′ box
(SEQ ID NO: 115)
CTAAGGACCAGCTTCTTTGGGAGAGAACAGACGCAGGGGGGGGAGGGAAA
AAGGGAGAGGCAGACGTCACTTCCCCTTGGCGGCTCTGGCAGCAGATTGG
TCGGTTGAGTGGCAGAAAGGCAGACGGGGACTGGGCAAGGCACTGTCGGT
GACATCACGGACAGGGCGACTTCTATGTAGATGAGGCAGCGCAGAGGCTG
CTGCTTCGCCACTTGCTGCTTCACCACGAAGGAGTTCCCGTGCCCTGGGA
GCGGGTTCAGGACCGCTGATCGGAAGTGAGAATCCCAGCTGTGTGTCAGG
GCTGGAAAGGGCTCGGGAGTGCGCGGGGCAAGTGACCGTGTGTGTAAAGA
GTGAGGCGTATGAGGCTGTGTCGGGGCAGAGGCCCAAGATCTCAGTTCAC
TGCCGTATAGGCAGGGCCCAGACTGAGCACGTGAGTTTTAGAGCTAGAAA
TAGCAAGTTAAAATAAGGCTAGTCCGTTATCAACTTGAAAAAGTGGGACC
GAGTCGGTCCTCTGCCATCAAAGCGTGCTCAGTCTGTTTCAGCAAGTTCA
GAGAAATCTGAACTTGCTGGATTTTTGGAGCAGGGAGATGGAATAGGAGC
TTGCTCCGTCCACTCCACGCATCGACCTGGTATTGCAGTACCTCCAGGAA
CGGTGCACCCACTTTCTGGAGTTTCAAAAGTAGACTGTACGCTAAGGGTC
ATATCTTTTTTTGTTTGGTTTGTGTCTTGGTTGGCGTCTTAAA.

In various other embodiments, the PEgRNA may be improved by introducing improvements to the scaffold or core sequences. This can be done by introducing known

The core, Cas9-binding PEgRNA scaffold can likely be improved to enhance PE activity. Several such approaches have already been demonstrated. For instance, the first pairing element of the scaffold (P1) contains a GTTTT-AAAAC (SEQ ID NO: 116) pairing element. Such runs of Ts have been shown to result in pol III pausing and premature termination of the RNA transcript. Rational mutation of one of the T-A pairs to a G-C pair in this portion of P1 has been shown to enhance sgRNA activity, suggesting this approach would also be feasible for PEgRNAs¹⁹⁵. Additionally, increasing the length of P1 has also been shown to enhance sgRNA folding and lead to improved activity, suggesting it as another avenue for the improvement of PEgRNA activity. Example improvements to the core can include:

PEgRNA containing a 6 nt extension to P1
(SEQ ID NO: 117)
GGCCCAGACTGAGCACGTGAGTTTTAGAGCTAGCTCATGAAAATGAGCTA
GCAAGTTAAAATAAGGCTAGTCCGTTATCAACTTGAAAAAGTGGGACCGA
GTCGGTCCTCTGCCATCAAAGCGTGCTCAGTCTGTTTTTTT
PEgRNA containing a T-A to G-C mutation within P1
(SEQ ID NO: 118)
GGCCCAGACTGAGCACGTGAGTTTGAGAGCTAGAAATAGCAAGTTTAAAT
AAGGCTAGTCCGTTATCAACTTGAAAAAGTGGGACCGAGTCGGTCCTCTG
CCATCAAAGCGTGCTCAGTCTGTTTTTTT

In various other embodiments, the PEgRNA may be improved by introducing modifications to the edit template region. As the size of the insertion templated by the PEgRNA increases, it is more likely to be degraded by endonucleases, undergo spontaneous hydrolysis, or fold into secondary structures unable to be reverse-transcribed by the RT or that disrupt folding of the PEgRNA scaffold and subsequent Cas9-RT binding. Accordingly, it is likely that modification to the template of the PEgRNA might be necessary to affect large insertions, such as the insertion of whole genes. Some strategies to do so include the incorporation of modified nucleotides within a synthetic or semi-synthetic PEgRNA that render the RNA more resistant to degradation or hydrolysis or less likely to adopt inhibitory secondary structures¹⁹⁶. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the PEgRNA to enhance stability and activity. Alternatively or additionally, the template of the PEgRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate PEgRNAs. In such a design, a PE would be used to initiate transcription and also recruit a separate template RNA to the targeted site via an RNA-binding protein fused to Cas9 or an RNA recognition element on the PEgRNA itself such as the MS2 aptamer. The RT could either directly bind to this separate template RNA, or initiate reverse transcription on the original PEgRNA before swapping to the second template. Such an approach could enable long insertions by both preventing misfolding of the PEgRNA upon addition of the long template and also by not requiring dissociation of Cas9 from the genome for long insertions to occur, which could possibly be inhibiting PE-based long insertions.

In still other embodiments, the PEgRNA may be improved by introducing additional RNA motifs at the 5′ and 3′ termini of the PEgRNAs, or even at positions therein between (e.g., in the gRNA core region, or the the spacer). Several such motifs—such as the PAN ENE from KSHV and the ENE from MALAT1 were discussed above as possible means to terminate expression of longer PEgRNAs from non-pol III promoters. These elements form RNA triple helices that engulf the polyA tail, resulting in their being retained within the nucleus^{184,187} However, by forming complex structures at the 3′ terminus of the PEgRNA that occlude the terminal nucleotide, these structures would also likely help prevent exonuclease-mediated degradation of PEgRNAs.

Other structural elements inserted at the 3′ terminus could also enhance RNA stability, albeit without enabling termination from non-pol III promoters. Such motifs could include hairpins or RNA quadruplexes that would occlude the 3′ terminus, or self-cleaving ribozymes such as HDV that would result in the formation of a 2′-3′-cyclic phosphate at the 3′ terminus and also potentially render the PEgRNA less likely to be degraded by exonucleases. Inducing the PEgRNA to cyclize via incomplete splicing—to form a ciRNA—could also increase PEgRNA stability and result in the PEgRNA being retained within the nucleus.

Additional RNA motifs could also improve RT processivity or enhance PEgRNA activity by enhancing RT binding to the DNA-RNA duplex. Addition of the native sequence bound by the RT in its cognate retroviral genome could enhance RT activity. This could include the native primer binding site (PBS), polypurine tract (PPT), or kissing loops involved in retroviral genome dimerization and initiation of transcription.

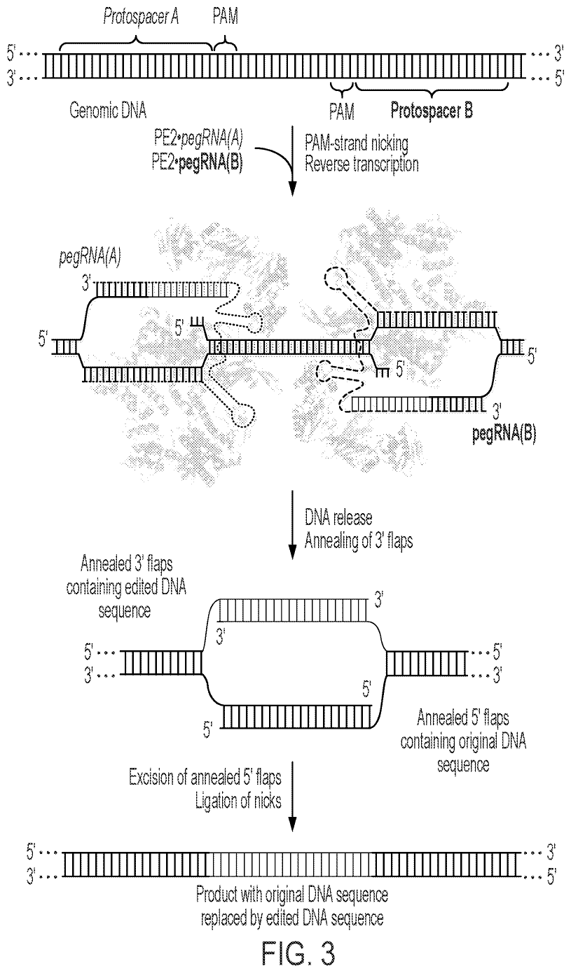

Addition of dimerization motifs—such as kissing loops or a GNRA tetraloop/tetraloop receptor pair—at the 5′ and 3′ termini of the PEgRNA could also result in effective circularization of the PEgRNA, improving stability. Additionally, it is envisioned that addition of these motifs could enable the physical separation of the PEgRNA spacer and primer, prevention occlusion of the spacer which would hinder PE activity. Short 5′ extensions or 3′ extensions to the PEgRNA that form a small toehold hairpin in the spacer region or along the primer binding site could also compete favorably against the annealing of intracomplementary regions along the length of the PEgRNA, e.g., the interaction between the spacer and the primer binding site that can occur. Finally, kissing loops could also be used to recruit other template RNAs to the genomic site and enable swapping of RT activity from one RNA to the other. As exemplary embodiments of various secondary structures, the PEgRNA depicted in FIG. 3D and FIG. 3E list a number secondary RNA structures that may be engineered into any region of the PEgRNA, including in the terminal portions of the extension arm (i.e., eland e2), as shown.

Example improvements include, but are not limited to:

PEgRNA-HDV fusion
(SEQ ID NO: 119)
GGCCCAGACTGAGCACGTGAGTTTTAGAGCTAGAAATAGCAAGTTAAAAT
AAGGCTAGTCCGTTATCAACTTGAAAAAGTGGGACCGAGTCGGTCCTCTG
CCATCAAAGCGTGCTCAGTCTGGGCCGGCATGGTCCCAGCCTCCTCGCTG
GCGCCGGCTGGGCAACATGCTTCGGCATGGCGAATGGGACTTTTTTT
PEgRNA-MMLV kissing loop
(SEQ ID NO: 120)
GGTGGGAGACGTCCCACCGGCCCAGACTGAGCACGTGAGTTTTAGAGCTA
GAAATAGCAAGTTAAAATAAGGCTAGTCCGTTATCAACTTGAAAAAGTGG
GACCGAGTCGGTCCTCTGCCATCAAAGCTTCGACCGTGCTCAGTCTGGTG
GGAGACGTCCCACCTTTTTTT
PEgRNA-VS ribozyme kissing loop
(SEQ ID NO: 121)
GAGCAGCATGGCGTCGCTGCTCACGGCCCAGACTGAGCACGTGAGTTTTA
GAGCTAGAAATAGCAAGTTAAAATAAGGCTAGTCCGTTATCAACTTGAAA
AAGTGGGACCGAGTCGGTCCTCTGCCATCAAAGCTTCGACCGTGCTCAGT
CTCCATCAGTTGACACCCTGAGGTTTTTTT
PEgRNA-GNRA tetraloop/tetraloop receptor
(SEQ ID NO: 122)
GCAGACCTAAGTGGUGACATATGGTCTGGGCCCAGACTGAGCACGTGAGT
TTTAGAGCTAUACGTAGCAAGTTAAAATAAGGCTAGTCCGTTATCAACTT
UACGAAGTGGGACCGAGTCGGTCCTCTGCCATCAAAGCTTCGACCGTGCT
CAGTCTGCATGCGATTAGAAATAATCGCATGTTTTTTT
PEgRNA template switching secondary RNA-HDV
fusion
(SEQ ID NO: 123)
TCTGCCATCAAAGCTGCGACCGTGCTCAGTCTGGTGGGAGACGTCCCACC
GGCCGGCATGGTCCCAGCCTCCTCGCTGGCGCCGGCTGGGCAACATGCTT
CGGCATGGCGAATGGGACTTTTTTT

PEgRNA scaffolds could be further improved via directed evolution, in an analogous fashion to how SpCas9 and prime editors (PE) have been improved. Directed evolution could enhance PEgRNA recognition by Cas9 or evolved Cas9 variants. Additionally, it is likely that different PEgRNA scaffold sequences would be optimal at different genomic loci, either enhancing PE activity at the site in question, reducing off-target activities, or both. Finally, evolution of PEgRNA scaffolds to which other RNA motifs have been added would almost certainly improve the activity of the fused PEgRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-PEgRNA fusions as well. In addition, while Cas9 currently does not generally tolerate 5′ extension of the sgRNA, directed evolution will likely generate enabling mutations that mitigate this intolerance, allowing additional RNA motifs to be utilized.

The present disclosure contemplates any such ways to further improve the efficacy of the multi-flap prime editing systems disclosed here.

In various embodiments, it may be advantageous to limit the appearance of consecutive sequence of Ts from the extension arm as consecutive series of T's may limit the capacity of the PEgRNA to be transcribed. For example, strings of at least consecutive three T's, at least consecutive four T's, at least consecutive five T's, at least consecutive six T's, at least consecutive seven T's, at least consecutive eight T's, at least consecutive nine T's, at least consecutive ten T's, at least consecutive eleven T's, at least consecutive twelve T's, at least consecutive thirteen T's, at least consecutive fourteen T's, or at least consecutive fifteen T's should be avoided when designing the PEgRNA, or should be at least removed from the final designed sequence. In one embodiment, one can avoid the includes of unwanted strings of consecutive T's in PEgRNA extension arms but avoiding target sites that are rich in consecutive A:T nucleobase pairs.

### PEgRNA Design Method

The present disclosure also relates to methods for designing PEgRNAs for use in the single flap prime editing, twinPE, and multi-flap PE embodiments.

In one aspect of design, the design approach can take into account the particular application for which prime editing is being used. For instance, and as exampled and discussed herein, prime editing can be used, without limitation, to (a) install mutation-correcting changes to a nucleotide sequence, (b) install protein and RNA tags, (c) install immunoepitopes on proteins of interest, (d) install inducible dimerization domains in proteins, (e) install or remove sequences to alter that activity of a biomolecule, (f) install recombinase target sites to direct specific genetic changes, and (g) mutagenesis of a target sequence by using an error-prone RT. In addition to these methods which, in general, insert, change, or delete nucleotide sequences at target sites of interest, prime editors can also be used to construct highly programmable libraries, as well as to conduct cell data recording and lineage tracing studies. In these various uses, there may be as described herein particular design aspects pertaining to the preparation of a PEgRNA that is particularly useful for any given of these applications.

When designing a PEgRNA for any particular application or use of prime editing, a number of considerations may be taken into account, which include, but are not limited to:
(a) the target sequence, i.e., the nucleotide sequence in which one or more nucleobase edits are desired to be installed by the prime editor;
(b) the location of the nicking site within the target sequence, i.e., the specific nucleobase position at which the prime editor will induce a single-stand nick to create a 3′ end RT primer sequence on one side of the nick and the 5′ end endogenous flap on the other side of the nick (which ultimately is removed by FEN1 or equivalent thereto and replaced by the 3′ ssDNA flap.

(c) the available PAM sequences (including the canonical SpCas9 PAM sites, as well as non-canonical PAM sites recognized by Cas9 variants and equivalents with expanded or differing PAM specificities);
(d) the spacing between the available PAM sequences and the location of the cut site in the target sequence;
(e) the particular Cas9, Cas9 variant, or Cas9 equivalent of the prime editor being used;
(f) the sequence and length of the primer binding site;
(g) the sequence and length of the edit template;
(h) the sequence and length of the homology arm;
(i) the spacer sequence and length; and
(j) the core sequence.

The instant disclosure discusses these aspects above.

In one embodiment, an approach to designing a suitable PEgRNA, and optionally a nicking-sgRNA design guide for second-site nicking, is hereby provided. This embodiment provides a step-by-step set of instructions for designing PEgRNAs and nicking-sgRNAs for prime editing which takes into account one or more of the above considerations.
1. Define the target sequence and the edit. Retrieve the sequence of the target DNA region (˜200 bp) centered around the location of the desired edit (point mutation, insertion, deletion, or combination thereof).
2. Locate target PAMs. Identify PAMs in the proximity to the desired edit location. PAMs can be identified on either strand of DNA proximal to the desired edit location. While PAMs close to the edit position are preferred (i.e., wherein the nick site is less than 30 nt from the edit position, or less than 29 nt, 28 nt, 27 nt, 26 nt, 25 nt, 24 nt, 23 nt, 22 nt, 21 nt, 20 nt, 19 nt, 18 nt, 17 nt, 16 nt, 15 nt, 14 nt, 13 nt, 12 nt, 11 nt, 10 nt, 9 nt, 8 nt, 7 nt, 6 nt, 5 nt, 4 nt, 3 nt, or 2 nt from the edit position to the nick site), it is possible to install edits using protospacers and PAMs that place the nick ≥30 nt from the edit position.
3. Locate the nick sites. For each PAM being considered, identify the corresponding nick site and on which strand. For Sp Cas9 H840A nickase, cleavage occurs in the PAM-containing strand between the 3^{rd} and 4^{th} bases 5′ to the NGG PAM. All edited nucleotides must exist 3′ of the nick site, so appropriate PAMs must place the nick 5′ to the target edit on the PAM-containing strand. In the example shown below, there are two possible PAMs. For simplicity, the remaining steps will demonstrate the design of a PEgRNA using PAM 1 only.
4. Design the spacer sequence. The protospacer of Sp Cas9 corresponds to the 20 nucleotides 5′ to the NGG PAM on the PAM-containing strand. Efficient Pol III transcription initiation requires a G to be the first transcribed nucleotide. If the first nucleotide of the protospacer is a G, the spacer sequence for the PEgRNA is simply the protospacer sequence. If the first nucleotide of the protospacer is not a G, the spacer sequence of the PEgRNA is G followed by the protospacer sequence.
5. Design a primer binding site (PBS). Using the starting allele sequence, identify the DNA primer on the PAM-containing strand. The 3′ end of the DNA primer is the nucleotide just upstream of the nick site (i.e. the 4^{th} base 5′ to the NGG PAM for Sp Cas9). As a general design principle for use with PE2 and PE3, a PEgRNA primer binding site (PBS) containing 12 to 13 nucleotides of complementarity to the DNA primer can be used for sequences that contain ˜40-60% GC content. For sequences with low GC content, longer (14- to 15-nt) PBSs should be tested. For sequences with higher GC content, shorter (8- to 11-nt) PBSs should be tested. Optimal PBS sequences should be determined empirically, regardless of GC content. To design a length-p PBS sequence, take the reverse complement of the first p nucleotides 5′ of the nick site in the PAM-containing strand using the starting allele sequence.
6. Design an RT template (or DNA synthesis template). The RT template (or DNA synthesis template where the polymerase is not reverse transcriptase) encodes the designed edit and homology to the sequence adjacent to the edit. In one embodiment, these regions correspond to the DNA synthesis template of FIG. 3D and FIG. 3E, wherein the DNA synthesis template comprises the “edit template” and the “homology arm.” Optimal RT template lengths vary based on the target site. For short-range edits (positions +1 to +6), it is recommended to test a short (9 to 12 nt), a medium (13 to 16 nt), and a long (17 to 20 nt) RT template. For long-range edits (positions +7 and beyond), it is recommended to use RT templates that extend at least 5 nt (preferably 10 or more nt) past the position of the edit to allow for sufficient 3′ DNA flap homology. For long-range edits, several RT templates should be screened to identify functional designs. For larger insertions and deletions (≥5 nt), incorporation of greater 3′ homology (˜20 nt or more) into the RT template is recommended. Editing efficiency is typically impaired when the RT template encodes the synthesis of a G as the last nucleotide in the reverse transcribed DNA product (corresponding to a C in the RT template of the PEgRNA). As many RT templates support efficient prime editing, avoidance of G as the final synthesized nucleotide is recommended when designing RT templates. To design a length-r RT template sequence, use the desired allele sequence and take the reverse complement of the first r nucleotides 3′ of the nick site in the strand that originally contained the PAM. Note that compared to SNP edits, insertion or deletion edits using RT templates of the same length will not contain identical homology.
7. Assemble the full PEgRNA sequence. Concatenate the PEgRNA components in the following order (5′ to 3′): spacer, scaffold, RT template and PBS.
8. Designing nicking-sgRNAs for PE3. Identify PAMs on the non-edited strand upstream and downstream of the edit. Optimal nicking positions are highly locus-dependent and should be determined empirically. In general, nicks placed 40 to 90 nucleotides 5′ to the position across from the PEgRNA-induced nick lead to higher editing yields and fewer indels. A nicking sgRNA has a spacer sequence that matches the 20-nt protospacer in the starting allele, with the addition of a 5′-G if the protospacer does not begin with a G.
9. Designing PE3b nicking-sgRNAs. If a PAM exists in the complementary strand and its corresponding protospacer overlaps with the sequence targeted for editing, this edit could be a candidate for the PE3b system. In the PE3b system, the spacer sequence of the nicking-sgRNA matches the sequence of the desired edited allele, but not the starting allele. The PE3b system operates efficiently when the edited nucleotide(s) falls within the seed region (˜10 nt adjacent to the PAM) of the nicking-sgRNA protospacer. This prevents nicking of the complementary strand until after installation of the edited strand, preventing competition between the PEgRNA and the sgRNA for binding the target DNA. PE3b also avoids the generation of simultaneous nicks on both strands, thus reducing indel formation significantly while maintaining high editing efficiency. PE3b sgRNAs should have a spacer sequence that matches the 20-nt protospacer in the desired allele, with the addition of a 5′ G if needed.

The above step-by-step process for designing a suitable PEgRNA and a second-site nicking sgRNA is not meant to be limiting in any way. The disclosure contemplates variations of the above-described step-by-step process which would be derivable therefrom by a person of ordinary skill in the art.

Methods of Editing with PE

In various embodiments, the disclosure provides compositions and methods for installing site-specific recombinase recognition sequences using prime editing (or “prime editing”) or classical prime editing (PE).

PE operates by contacting a target DNA molecule (for which a change in the nucleotide sequence is desired to be introduced) with a nucleic acid programmable DNA binding protein (napDNAbp) complexed with an extended guide RNA. The extended guide RNA comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer sequence of the extended gRNA) or the “non-target strand” (i.e, the strand forming the single-stranded portion of the R-loop and which is complementary to the target strand). In step (c), the 3′ end of the DNA strand (formed by the nick) interacts with the extended portion of the guide RNA in order to prime reverse transcription (i.e, “target-primed RT”). In certain embodiments, the 3′ end DNA strand hybridizes to a specific RT priming sequence on the extended portion of the guide RNA, i.e, the “reverse transcriptase priming sequence.” In step (d), a reverse transcriptase is introduced (as a fusion protein with the napDNAbp or in trans) which synthesizes a single strand of DNA from the 3′ end of the primed site towards the 5′ end of the extended guide RNA. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap (e.g., by FEN1 or similar enzyme that is provide in trans, as a fusion with the prime editor, or endogenously provided) that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking” or “temporal second strand nicking,” as discussed herein.

The process of prime editing may introduce at least one or more of the following genetic changes: transversions, transitions, deletions, and insertions, and in particular, may be used to insert one or more SSR recognition sequences. In addition, prime editing may be implemented for specific applications. For example, and as exemplified and discussed herein, prime editing can be used to install SSR recognition sequences to direct specific genetic changes using recombinases. The inventors have also contemplated additional design features of PEgRNAs that are aimed to improve the efficacy of prime editing. Still further, the inventors have conceived of methods for successfully delivering prime editors using vector delivery systems and which involve splitting the napDNAbp using intein domains.

The term “prime editing system” or “prime editor system” refers the compositions involved in the method of genome editing using target-primed reverse transcription (TPRT) describe herein, including, but not limited to the napDNAbps, reverse transcriptases, fusion proteins (e.g., comprising napDNAbps and reverse transcriptases), extended guide RNAs, and complexes comprising fusion proteins and extended guide RNAs, as well as accessory elements, such as second strand nicking components and 5′ endogenous DNA flap removal endonucleases (e.g., FEN1) for helping to drive the prime editing process towards the edited product formation.

In another embodiment, the schematic of FIG. 21E depicts the interaction of a typical PEgRNA with a target site of a double stranded DNA and the concomitant production of a 3′ single stranded DNA flap containing the genetic change of interest. The double strand DNA is shown with the top strand in the 3′ to 5′ orientation and the lower strand in the 5′ to 3′ direction. The top strand comprises the “protospacer” and the PAM sequence and is referred to as the “target strand.” The complementary lower strand is referred to as the “non-target strand.” Although not shown, the PEgRNA depicted would be complexed with a Cas9 or equivalent. As shown in the schematic, the spacer of the PEgRNA anneals to a complementary region on the target strand, which is referred to as the protospacer, which is located just downstream of the PAM sequence is approximately 20 nucleotides in length. This interaction forms as DNA/RNA hybrid between the spacer RNA and the protospacer DNA, and induces the formation of an R loop in the region opposite the protospacer. As taught elsewhere herein, the Cas9 protein (not shown) then induces a nick in the non-target strand, as shown. This then leads to the formation of the 3′ ssDNA flap region which, in accordance with *z*, interacts with the 3′ end of the PEgRNA at the primer binding site. The 3′ end of the ssDNA flap (i.e., the reverse transcriptase primer sequence) anneals to the primer binding site (A) on the PEgRNA, thereby priming reverse transcriptase. Next, reverse transcriptase (e.g., provided in trans or provided cis as a fusion protein, attached to the Cas9 construct) then polymerizes a single strand of DNA which is coded for by the edit template (B) and homology arm (C). The polymerization continues towards the 5′ end of the extension arm. The polymerized strand of ssDNA forms a ssDNA 3′ end flap which, as describe herein invades the endogenous DNA, displacing the corresponding endogenous strand (which is removed as a 5′ DNA flap of endogenous DNA), and installing the desired nucleotide edit (single nucleotide base pair change, deletions, insertions (including whole genes) through naturally occurring DNA repair/replication rounds.

Methods of Editing with twinPE and/or Multi-Flap PE

In various embodiments, the disclosure provides compositions and methods for installing one or more site-specific recombinase recognition sequences using twin prime editing (or twinPE) or multi-flap PE.

This Specification describes twinPE and multi-flap PE systems. In twinPE, two PEgRNAs are used to target opposite strands of a genomic site and direct the synthesis of two complementary 3′ flaps containing edited DNA sequence (FIG. 3). In some embodiments, there is no requirement for the pair of edited DNA strands (3′ flaps) to directly compete with 5′ flaps in endogenous genomic DNA, as the complementary edited strand is available for hybridization instead. Since both strands of the duplex are synthesized as edited DNA, the dual-flap prime editing system obviates the need for the replacement of the non-edited complementary DNA strand required by classical prime editing. Instead, cellular DNA repair machinery need only excise the paired 5′ flaps (original genomic DNA) and ligate the paired 3′ flaps (edited DNA) into the locus. Therefore, in some embodiments, a twinPE system comprises a pair of PEgRNAs, wherein each of the PEgRNAs comprises a DNA synthesis template comprising a region of complementarity to each other, and each does not have substantial complementarity or substantial homology to the endogenous sequence of the target DNA.

Accordingly, in certain embodiments, twinPE involves a pair of newly synthesized DNA strands (e.g. 3′ flaps) that may or may not share homology with the endogenous DNA sequence at a target site to be edited. In some embodiments, twinPE involves a pair of newly synthesized DNA strands, where at least one of the newly synthesized DNA strands does not share homology with the endogeneous DNA sequence at the target site to be edited. In some embodiments, twinPE involves a pair of newly synthesized DNA strands, where neither of the newly synthesized DNA strands share homology with the endogenous DNA sequence at the target site to be edited. Rather, the two newly synthesized DNA strands (e.g. 3′ flaps) each comprises a region of complementarity to each other and may form a duplex by the complementarity. A desired edited portion as compared to the endogenous DNA sequence target site to be edited in the duplex may then be incorporated at the target site.

In some embodiments, a twinPE system comprises a pair of PEgRNAs, wherein each of the PEgRNAs comprises a DNA synthesis template comprising a region of complementarity to each other, and one or both of the DNA synthesis comprises a region of complementarity to the endogenous sequence of the target DNA.

In some embodiments, a twinPE system comprises a pair of PEgRNAs, wherein at least one of the pair of PEgRNAs comprises a DNA synthesis template comprising a region of complementarity to the endogenous sequence of the target DNA, and does not have complementarity to the DNA synthesis template of the other PEgRNA of the pair. In some embodiments, one of the newly synthesized DNA strands comprises homology with the endogenous DNA sequence at the target site and not with the other newly synthesized DNA strand. In some embodiments, each of the newly synthesized DNA strands comprises homology with the endogenous DNA sequence at the target site and not with the other newly synthesized DNA strand. For example, in some embodiments, a newly synthesized 3′ flap encoded by one of the dual-flap PEgRNAs may comprise a region of complementarity to a protospacer sequence of the other dual-flap PEgRNA. Accordingly, in some embodiments, a pair of dual-flap PEgRNAs each having complementarity to a spacer sequence of the other PEgRNA may result in deletion of the endogenous DNA sequence positioned between protospacer sequences of the pair of dual-flap PEgRNAs.

Single flap prime editing systems, dual flap editing systems, and multi flap editing systems can comprise any one of the prime editor protein components as described herein. In some embodiments, a napDNAbp a dual flap editing system comprises a nuclease inactive napDNAbp, e.g., dCas9, or a napDNAbp nickase, e.g., Cas9 nickase. In some embodiments, a napDNAbp a dual flap editing system comprises a nuclease active napDNAbp, e.g., a nuclease active Cas9 that cut both strands, for example, to accelerate the removal of the original DNA sequence.

Like classical prime editing, dual prime editing is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand.

Installing SSR Recognition Sequences with PE, twinPE and Multi-Flap

In various embodiments, the disclosure provides compositions and methods for installing one or more site-specific recombinase recognition sequences using single flap prime editing (“classical PE”), twin prime editing (or twinPE) or multi-flap PE.

In one aspect, classical PE may be used to insert one or more or two or more SSR recognition sequences into a desired genomic site.

In another aspect, twinPE may be used to insert one or more or two or more SSR recognition sequences into a desired genomic site.

In still another aspect, multi-flap PE may be used to insert one or more or two or more SSR recognition sequences into one more desired genomic sites.

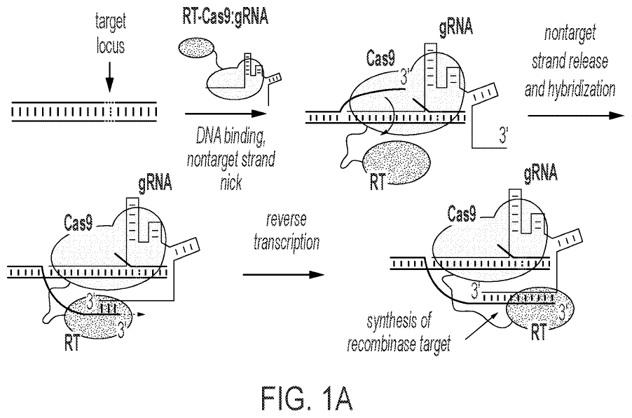

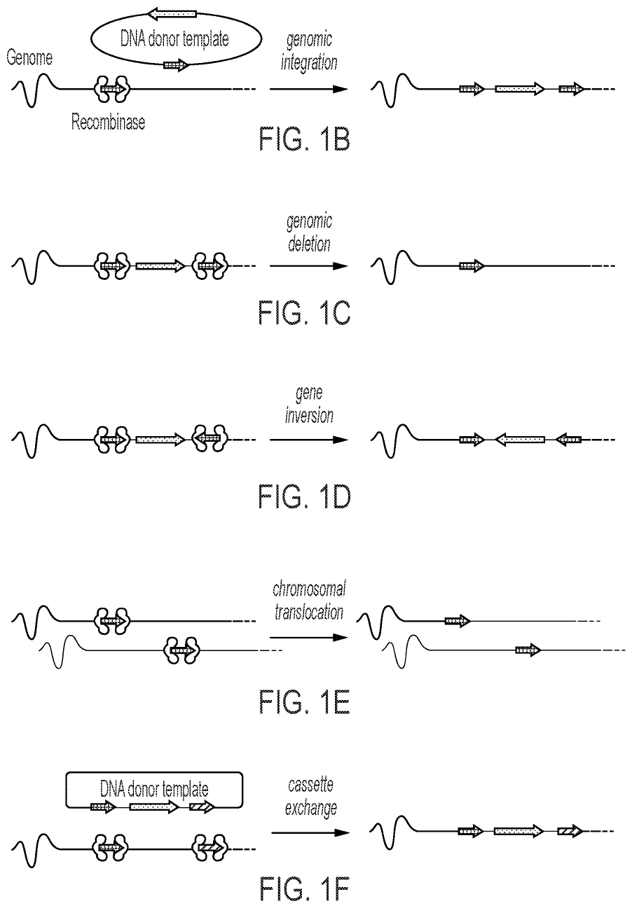

Insertion of recombinase sites provides a programmed location for effecting one or more site-specific intended edit in a target DNA, e.g., genetic changes in a target gene or a genome. Non-limiting examples of intended edit via SSR mediated recombination include insertion of an exogenous sequence into a target DNA, deletion (excision) of an endogenous sequence in a target DNA, inversion of an endogenous sequence in a target DNA, replacement of an endogenous sequence in a target DNA by an exogenous sequence, and any combination thereof. Accordingly, when the target DNA is a target gene or target genome, genetic changes via SSR mediated recombination can include, for example, genomic integration of an exogenous DNA sequence, e.g., sequence of a plasmid or a part thereof, genomic deletion or insertion, chromosomal translocations, and replacement of an endogenous genomic sequence in a target genome by an exogenous sequence (“cassette exchanges”), among other genetic changes. These exemplary types of genetic changes are illustrated in FIG. 1. The installed recombinase recognition sequences may be used to conduct site-specific recombination at recombinase recognition site(s) to effectuate a variety of recombination outcomes, such as, excision, integration, inversion, or exchange of DNA fragments, or for example, insertion of an SSR recognition sequence. For example, FIG. 65 illustrates the installation of a recombinase site that can then be used to integrate a DNA donor template comprising a GFP expression marker. Cells containing the integrated GFP expression system into the recombinase site will fluoresce.

The mechanism of installing a recombinase site into the genome is analogous to installing other sequences, such as peptide/protein and RNA tags, into the genome. Recombinase sites can be installed in a target DNA, e.g., a target genome, with single flap prime editing, twin prime editing, or multi-flap prime editing systems described herein.

A schematic exemplifying the installation of one or more recombinase target sequence with a single flap prime editing system is shown in FIG. 1a. The process begins with selecting a desired target locus into which the recombinase target sequence will be introduced. Next, a prime editor fusion is provided (“RT-Cas9:gRNA”). Here, the “gRNA” refers to a PEgRNA, which can be designed using the principles described herein. The PEgRNA in various embodiments will comprise an architecture corresponding to FIG. 21C (5′-[˜20-nt spacer]-[gRNA core]-[extension arm]-3′, wherein the extension arm comprises in the 3′ to 5′ direction, a primer binding site (“A”), an edit template of DNA synthesis template (“B”), and optionally a homology arm (“C”) The edit template (“B”) will comprise a sequence corresponding to one or more recombinase site, i.e., a single strand RNA of the PEgRNA that codes for a complementary single strand DNA that is either the sense or the antisense strand of the recombinase site and which is incorporated into the genomic DNA target locus through the prime editing process. In some embodiments, the edit template comprises a sequence corresponding to a single recombinase site. In some embodiments, the edit template comprises a sequence corresponding to two recombinase sites. In some embodiments, the edit template comprises a sequence corresponding to two or more recombinase sites.

In some embodiments, a prime editing system comprises multiple PEgRNAs for installation of two or more recombinase sites in a target DNA (multiplexed single flap prime editing), wherein each PEgRNA independently comprises a spacer targeting a target site in the target DNA, and wherein each PEgRNA independent comprises a DNA synthesis template that comprises (and encodes) a recombinase site for integration in the target DNA. in some embodiments, the recombinase sites in each of the DNA synthesis templates are different from each other. For example, in some embodiments, a prime editing system comprises a first and a second PEgRNA, wherein the first PEgRNA comprises a spacer a first spacer and a first DNA synthesis template, wherein the second PEgRNA comprises a second spacer and a second DNA synthesis template, wherein the first spacer has complementarity to a first target site in the target DNA and wherein the second spacer has complementarity to a second target site in the target DNA, wherein the first target site and the second target site are different from each other, wherein the first DNA synthesis template comprises (and encodes) a first recombinase site and wherein the second DNA synthesis template comprises (and encodes) a second recombinase site. In some embodiments, the first target site and the second target site are 10, 20, 30, 50, 100, 150, 200, 250, 300, 400, 500, 600, 700, 800, 900, 1000 or more base pairs apart from each other. In some embodiments, the first recombinase site and the second recombinase site are the same. In some embodiments, the first recombinase site and the second recombinase site are recognized by the same recombinase. In some embodiments, the first recombinase site and the second recombinase site are different. In some embodiments, the first recombinase site and the second recombinase site are recognized by different recombinases.

In some embodiments, the prime editing system used for installation of one or more recombinase sites in a target DNA is a twinPE system. A schematic exemplifying the installation of a recombinase target sequence with a twin flap prime editing system is shown in FIG. 3. The twin prime editing system comprises a pair of PEgRNAs and a prime editor fusion protein, e.g., RT-Cas9 nickase. The pair of PEgRNAs each comprises an architecture (5′-[˜20-nt spacer]-[gRNA core]-[extension arm]-3′), wherein the extension arm comprises in the 3′ to 5′ direction, a primer binding site and a DNA synthesis template, wherein the DNA synthesis template of a first PEgRNA of the pair comprises a region of complementarity to the DNA synthesis template of the second PEgRNA of the pair. In some embodiments, the region of complementarity between the first DNA synthesis template and the second DNA synthesis template comprises a recombinase site, and each strand of the region of complementarity codes for a single stranded DNA that is either the sense or the antisense strand of the recombinase site and which is incorporated into the genomic DNA target locus through the twin prime editing process. In some embodiments, the DNA sequence that corresponds to the regions (the first DNA synthesis template not complement to the second DNA synthesis template+the region of complementarity between the first DNA synthesis template+the second DNA synthesis template not complement to the first DNA synthesis template) comprises one or more recombinase site, and each strand of the region of complementarity codes for a single stranded DNA that is either the sense or the antisense strand of the recombinase site and which is incorporated into the genomic DNA target locus through the twin prime editing process. The DNA sequence corresponding to (the first DNA synthesis template not complementary to the second DNA synthesis template+the region of complementarity between the first DNA synthesis template+the second DNA synthesis template not complementary to the first DNA synthesis template), as used in the context of twin PE, represents a duplex of double stranded DNA sequence having sequences of the first DNA synthesis template and the second DNA synthesis template, and may be referred to as a “replacement duplex”. This replacement duplex can also be understood as the first DNA synthesis template+the second DNA synthesis template—the region of complementarity between the first DNA synthesis template and the second DNA synthesis template.

In some embodiments, the replacement duplex between the first DNA synthesis template and the second DNA synthesis template comprises a sequence corresponding to a single recombinase site. In some embodiments, the replacement duplex between the first DNA synthesis template and the second DNA synthesis template comprises a sequence corresponding to two recombinase sites. In some embodiments, the replacement duplex between the first DNA synthesis template and the second DNA synthesis template comprises a sequence corresponding to two or more recombinase sites.

In some embodiments, the prime editing system used to install recombinase sites in a target DNA is a twinPE system comprising two or more pairs of PEgRNAs (multiplexed twin prime editing). For example, in some embodiments, a prime editing system comprises (i) a first PEgRNA that comprises a first spacer and a first DNA synthesis template, (ii) a second PEgRNA that comprises a second spacer and a second DNA synthesis template (iii) a third PEgRNA that comprises a third spacer and a third DNA synthesis template, (iv) a fourth PEgRNA that comprises a fourth spacer and a fourth DNA synthesis template, wherein the first spacer has complementarity to a first target site in the target DNA, wherein the second spacer has complementarity to a second target site in the target DNA, wherein the third spacer has complementarity to a third target site in the target DNA, wherein the fourth spacer has complementarity to a fourth target site in the target DNA, wherein the first DNA synthesis template and the second DNA synthesis template comprises a region of complementarity to each other, wherein the replacement duplex between the first DNA synthesis template and the second DNA synthesis template comprises a first recombinase recognition site as compared to the target DNA, wherein the third DNA synthesis template and the fourth DNA synthesis template comprises a region of complementarity to each other, and wherein the replacement duplex between the third DNA synthesis template and the fourth DNA synthesis template comprises a second recombinase recognition site as compared to the target DNA. In some embodiments, the region between the first target site and the second target site is 10, 20, 30, 50, 100, 150, 200, 250, 300, 400, 500, 600, 700, 800, 900, 1000 or more base pairs apart from the region between the third target site and the fourth target site. In some embodiments, the first recombinase site and the second recombinase site are the same. In some embodiments, the first recombinase site and the second recombinase site are recognized by the same recombinase. In some embodiments, the first recombinase site and the second recombinase site are different. In some embodiments, the first recombinase site and the second recombinase site are recognized by different recombinases.

In various aspects, the present disclosure provides for the use of a prime editing system, including a single flap prime editing system, a twin prime editing system, PE or a multi-flap prime editing system to introduce recombinase recognition sequences at high-value loci in human or other genomes, which, after exposure to site-specific recombinase(s), will direct precise and efficient genomic modifications (e.g., those of FIG. 1). In various embodiments show in FIG. 1, PE may be used to insert a single SSR target for use as a site for genomic integration of a DNA donor template, e.g., see FIGS. 1a and 1b. FIG. 1c shows how inserted tandem SSR target sites can be used to delete a portion of the genome. FIG. 1d shows how a tandem insertion of SSR target sites can be used to invert a portion of the genome. FIG. 1e shows how the insertion of two SSR target sites at two distal chromosomal regions can result in chromosomal translocation. FIG. if shows how the insertion of two different SSR target sites in the genome can be used to exchange a cassette from a DNA donor template. Each of the types of genome modifications are envisioned by using PE, twinPE, or multi-flap PE to insert SSR targets, but this list also is not meant to be limiting.

PE-mediated introduction of recombinase recognition sequences could be particularly useful for the treatment of genetic diseases which are caused by large-scale genomic defects, such as gene loss, inversion, or duplication, or chromosomal translocation (Table A). For example, Williams-Beuren syndrome is a developmental disorder caused by a chromosomal deletion. No technology exists currently for the efficient and targeted insertion of multiple entire genes in living cells; however, recombinase-mediated integration at a target inserted by PE offers one approach towards a permanent cure for this and other diseases. In addition, targeted introduction of recombinase recognition sequences could be highly enabling for applications including generation of transgenic plants, animal research models, bioproduction cell lines, or other custom eukaryotic cell lines. For example, recombinase-mediated genomic rearrangement in transgenic plants at PE-specific targets could overcome one of the bottlenecks to generating agricultural crops with improved properties.

TABLE A
Examples of genetic diseases linked to large-scale genomic
modifications that could be repaired through PE-based
installation of recombinase recognition sequences.

| DISEASE | CAUSE |
|---|---|
| TRISOMY 17P | GENE DUPLICATION |
| CHARCOT-MARIE- | GENE DUPLICATION |
| TOOTH DISEASE TYPE I | |
| SMITH-MAGENIS | GENE DELETION |
| SYNDROME | |
| WILLIAMS-BEUREN | GENE DELETION |
| SYNDROME | |
| DE LA CHAPELLE | CHROMOSOMAL |
| SYNDROME | TRANSLOCATION |
| DOWN SYNDROME | CHROMOSOMAL |
| (SOME FORMS) | TRANSLOCATION |
| HEMOPHILIA A | GENE INVERSION |
| HUNTER SYNDROME | GENE INVERSION |

A number of SSR family members have been characterized and their recombinase recognition sequences described, including natural and engineered tyrosine recombinases (Table B), large serine integrases (Table C), serine resolvases (Table D), and tyrosine integrases (Table E). Modified target sequences that demonstrate enhanced rates of genomic integration have also been described for several SSRs. In addition to natural recombinases, programmable recombinases with distinct specificities have been developed. Using PE, one or more of these recognition sequences could be introduced into the genomic at a specified location, such as a safe harbor locus, depending on the desired application.

For example, introduction of a single recombinase recognition sequence in the genome by prime editing would result in integrative recombination with a DNA donor template (e.g., FIG. 1b). Serine integrases, which operate robustly in human cells, may be especially well-suited for gene integration.

Additionally, introduction of two recombinase recognition sequences could result in deletion of the intervening sequence, inversion of the intervening sequence, chromosomal translocation, or cassette exchange, depending on the identity and orientation of the targets (e.g., see FIG. 1c-f). By choosing endogenous sequences that already closely resemble recombinase targets, the scope of editing required to introduce the complete recombinase target would be reduced.

Finally, several recombinases have been demonstrated to integrate into human or eukaryotic genomes at natively occurring pseudosites. PE, twinPE, and multi-flap prime editing could be used to modify these loci to enhance rates of integration at these natural pseudosites, or alternatively, to eliminate pseudosites that may serve as unwanted off-target sequences.

This disclosure describes a general methodology for introducing recombinase target sequences in eukaryotic genomes using PE, twinPE, or multi-flap PE, the applications of which are nearly limitless. The genome editing reactions are intended for use with “prime editors,” a chimeric fusion of a CRISPR/Cas9 protein and a reverse-transcriptase domain, which utilizes a custom prime editing guide RNA (PEgRNA). By extension, Cas9 tools and homology-directed repair (HDR) pathways may also be exploited to introduce recombinase recognition sequences through DNA templates by lowering the rates of indels using several techniques.

The following several tables are cited in the above description relating to PE-directed installation of recombinase recognition sequences and provide a listing of exemplary recombinases that may be used, and their cognate recombinase recognition sequences that may be installed by PE, twinPE, and/or multi-flap PE.

+0 TABLE B
Tyrosine recombinases and SSR target sequences.

| Recombinase | Recombinase recognition sequence | Name |
|---|---|---|
| Cre | ATAACTTCGTATAGCATACATTATACGAAGTTAT (SEQ ID | loxP |
| | NO: 124) | |
| Dre | TAACTTTAAATAATGCCAATTATTTAAAGTTA (SEQ ID NO: | rox |
| | 125) | |
| VCre | TCAATTTCTGAGAACTGTCATTCTCGGAAATTGA (SEQ ID | loxV |
| | NO: 126) | |
| SCre | CTCGTGTCCGATAACTGTAATTATCGGACATGAT (SEQ ID | loxS |
| | NO: 127) | |
| Flp | GAAGTTCCTATTCTCTAGAAAGTATAGGAACTTC (SEQ ID | FRT |
| | NO: 128) | |
| B2 | GAGTTTCATTAAGGAATAACTAATTCCCTAATGAAACTC | loxB |
| | (SEQ ID NO: 129) | |
| B3 | GGTTGCTTAAGAATAAGTAATTCTTAAGCAACC (SEQ ID NO: | loxB3 |
| | 130) | |
| Kw | ACGAAAAATGGTAAGGAATAGACCATTCCTTACCATTTTTG | |
| | GT (SEQ ID NO: 131) | |
| R | TTGATGAAAGAATAACGTATTCTTTCATCAA (SEQ ID NO: | RS |
| | 132) | |
| TD1-40 | GTGCGTCAAATAATAACGTATTATTTGACACTT (SEQ ID NO: | TDRS |
| | 133) | |
| Vika | AATAGGTCTGAGAACGCCCATTCTCAGACGTATT (SEQ ID | VOX |
| | NO: 134) | |
| Nigri | TGAATGTCCTATAATTACACTTATAGGACATTCA (SEQ ID | nox |
| | NO: 135) | |
| Panto | GAAACTTTAAATAATAAGTCTTATTTAAAGTTTC (SEQ ID NO: | pox |
| | 136) | |
| Kd | AAACGATATCAGACATTTGTCTGATAATGCTTCATTATCAG | loxK |
| | ACAAATGTCTGATATCGTTT (SEQ ID NO: 137) | |
| Fre | ATATATACGTATATAGACATATATACGTATATAT (SEQ ID | loxH |
| | NO: 138) | |
| CreALSHG | ATAACTCTATATAATGTATGCTATATAGAGTTAT (SEQ ID NO: | loxM7 |
| | 139) | |
| Tre | ACAACATCCTATTACACCCTATATGCCAACATGG (SEQ ID | loxLTR |
| | NO: 140) | |
| Brec1 | AACCCACTGCTTAAGCCTCAATAAAGCTTGCCTT (SEQ ID | loxBTR |
| | NO: 141) | |
| Cre-R3M3 | GATACAACGTATATACCTTTCTATACGTTGTTTA (SEQ ID NO: | loxK2 |
| | 142) | |

+0 TABLE C
Large serine integrases and
SSR target sequences.

| | Recombinase | Recombinase |
|---|---|---|
| | ecognition | recognition |
| | sequence | sequence |
| Recombinase | Left | Right |
| Bxb1 | GGTTTGTCTGGTCAA | GGCTTGTCGACGACG |
| | CCACCGCGGTCTCAG | GCGGTCTCCGTCGTC |
| | TGGTGTACGGTACAA | AGGATCAT |
| | ACC | (SEQ ID NO: 175) |
| | (SEQ ID NO: 143) | |
| phiC31 | TGTGCCCCAACTGGG | TGCGGGTGCCAGGGC |
| | GTAACCTTGAGTTCT | GTGCCCTTGGGCTCC |
| | CTCAGTTGGGGG | CCGGGCGCGTACTCC |
| | (SEQ ID NO: 144) | (SEQ ID NO: 176) |
| R4 | TGTTCCCCAAAGCGA | GCATGTTCCCCAAAG |
| | TACCACTTGAAGCAG | CGATACCACTTGAAG |
| | TGGTACTGCTTGTGG | CAGTGGTACTGCTTG |
| | GTACA | TGGGTACACTCTGCG |
| | (SEQ ID NO: 145) | GGTG |
| | | (SEQ ID NO: 177) |
| phiBT1 | GGTGCTGGGTTGTTG | CAGGTTTTTGACGAA |
| | TCTCTGGACAGTGAT | AGTGATCCAGATGAT |
| | CCATGGGAAACTACT | CCAG |
| | CAGCACC | (SEQ ID NO: 178) |
| | (SEQ ID NO: 146) | |
| MJ1 | ATTTTAGGTATATGA | CAAAGGATCACTGAA |
| (phiFC1) | TTTTGTTTATTAGTG | TCAAAAGTATTGCTC |
| | TAAATAACACTATGT | ATCCACGCGAAA |
| | ACCTAAAAT | (SEQ ID NO: 179) |
| | (SEQ ID NO: 147) | |
| MR11 | TTTGTGCGGAACTAC | CGAAAATGTATGGAG |
| | GAACAGTTCATTAAT | GCACTTGTATCAATA |
| | ACGAAGTGTACAAAC | TAGGATGTATACCTT |
| | TTCCATACAA | CGAAGACACTT |
| | (SEQ ID NO: 148) | (SEQ ID NO: 180) |
| TP901-1 | GAGTTTTTATTTCGT | ATGCCAACACAATTA |
| | TTATTTCAATTAAGG | ACATCTCAATCAAGG |
| | TAACTAAAAAACTCC | TAAATGCTTTTTGCT |
| | TTTTAAGG | TTTTTTGC |
| | (SEQ ID NO: 149) | (SEQ ID NO: 181) |
| A118 | TTCCTCGTTTTCTCT | TTTCGGATCAAGCTA |
| | CGTTGGAAGAAGAAG | TGAAGGACGCAAAGA |
| | AAACGAGAAA | GGGAACTAAA |
| | (SEQ ID NO: 150) | (SEQ ID NO: 182) |
| U153 | TTCCTCGTTTTCTCT | TTTCGGATCAAGCTA |
| | CGTTGGACGGAAACG | TGAAGGACGCAAAGA |
| | AATCGAGAAA | GGGAACTAAA |
| | (SEQ ID NO: 151) | (SEQ ID NO: 182) |
| phiRV1 | GTAGTGTATCTCACA | GAAGGTGTTGGTGCG |
| | GGTCCACGGTTGGCC | GGGTTGGCCGTGGTC |
| | GTGGACTGCTGAAGA | GAGGTGGGGT |
| | ACATTCC | (SEQ ID NO: 183) |
| | (SEQ ID NO: 152) | |
| phi370.1 | AAAAAAATACAGCGT | TTGTAAAGGAGACTG |
| | TTTTCATGTACAACT | ATAATGGCATGTACA |
| | ATACTAGTTGTAGTG | ACTATACTCGTCGGT |
| | CCTAAAA | AAAAAGGCA |
| | (SEQ ID NO: 153) | (SEQ ID NO: 184) |
| TG1 | TCCAGCCCAACAGTG | GATCAGCTCCGCGGG |
| | TTAGTCTTTGCTCTT | CAAGACCTTTCTCCT |
| | ACCCAGTTGGGCGGG | TCACGGGGTGGAAGG |
| | A | TC |
| | (SEQ ID NO: 154) | (SEQ ID NO: 185) |
| WB | CTAGTTTTAAAGTTG | CGGAAGGTAGCGTCA |
| | GTTATTAGTTACTGT | ACGATAGGTGTAACT |
| | GATATTTATCACGGT | GTCGTGTTTGTAACG |
| | ACCCAATAACCAATG | GTACTTCCAACAGCT |
| | AAT | GGCGCCGCCAC |
| | (SEQ ID NO: 155) | (SEQ ID NO: 186) |
| BL3 | CAATGAAAAACTAGG | TTTCCACAGACAACT |
| | CATGTAGAAGTTGTT | CACGTGGAGGTAGTC |
| | TGT | AC |
| | (SEQ ID NO: 156) | (SEQ ID NO: 187) |
| SprA | TGTAGTAAGTATCTT | CACCCATTGTGTTCA |
| | AATATACAGCTTTAT | CAGGAGATACAGCTT |
| | CTGTTTTTTAAGATA | TATCTGTACTGATAT |
| | CTTACTACTTT | TAATGACATGCTG |
| | (SEQ ID NO: 157) | (SEQ ID NO: 188) |
| phiJoe | AGTTGTGGCCATGTG | GGCAGACGCCGCAGT |
| | TCCATCTGGGGGCAG | CGAAGCACGATCTGG |
| | ATGGAGACGGGGTCA | ATGTGGGTGTCCATC |
| | CA | TGCGG |
| | (SEQ ID NO: 158) | (SEQ ID NO: 189) |
| — | — | ACCTTGATCTCGGTG |
| | | TCCATCGCCGGGCAG |
| | | ACGCCGCAGTCGAAG |
| | | CACGG |
| | | (SEQ ID NO: 190) |
| phiK38 | CCCTAATACGCAAGT | GAGCGCCGGATCAGG |
| | CGATAACTCTCCTGG | GAGTGGACGGCCTGG |
| | GAGCGTTGACAACTT | GAGCGCTACACGCTG |
| | GCGCACCCTGATCTG | TGGCTGCGGTCGGTG |
| | (SEQ ID NO: 160) | C |
| | | (SEQ ID NO: 191) |
| Int2 | GCTCATGTATGTGTC | GGACGGCGCAGAAGG |
| | TACGCGAGATTCTCG | GGAGTAGCTCTTCGC |
| | CCCGAGAACTTCTGC | CGGACCGTCGACATA |
| | AAGGCACTGCTCTTG | CTGCTCAGCTCGTC |
| | GCT | (SEQ ID NO: 192) |
| | (SEQ ID NO: 161) | |
| Int3 | ATGGATAAAAAAATA | GTTTGTAAAGGAGAC |
| | CAGCGTTTTTCATGT | TGATAATGGCATGTA |
| | ACAACTATACTAGTT | CAACTATACTCGTCG |
| | GTAGTGCCTAAATAA | GTAAAAAGGCATCTT |
| | TGCTT | AT |
| | (SEQ ID NO: 162) | (SEQ ID NO: 193) |
| Int4 | AAAAATTACAAAGTT | TTCCAAAGAGCGCCC |
| | TTCAACCCTTGATTT | AACGCGACCTGAAAT |
| | GAATTAGCGGTCAAA | TTGAATAAGACTGCT |
| | TAATTTGTAATTCGT | GCTTGTGTAAAGGCG |
| | TT | ATGATT |
| | (SEQ ID NO: 163) | (SEQ ID NO: 194) |
| Int7 | GTGTTATAAACCTGT | AGACGAGAAACGTTC |
| | GTGAGAGTTAAGTTT | CGTCCGTCTGGGTCA |
| | ACATGCCTAACCTTA | GTTGGGCAAAGTTGA |
| | ACTTTTACGCAGGTT | TGACCGGGTCGTCCG |
| | CAGCTT | TT |
| | (SEQ ID NO: 164) | (SEQ ID NO: 195) |
| Int8 | TTAATAAACTATGGA | CAATCATCAGATAAC |
| | AGTATGTACAGTCTT | TATGGCGGCACGTGC |
| | GCAATGTTGAGTGAA | ATTAACCACGGTTGT |
| | CAAACTTCCATAATA | ATCCCGTCTAAAGTA |
| | AAAT | CTCGT |
| | (SEQ ID NO: 165) | (SEQ ID NO: 196) |
| Int9 | GTGGTTGTTTTTGTT | TTTATATTGCGAAAA |
| | GGAAGTGTGTATCAG | ATAATTGGCGAACGA |
| | GTATCTGCATAGTTA | GGTAACTGGATACCT |
| | TTCCGAACTTCCAAT | CATCCGCCAATTAAA |
| | TA | ATTTG |
| | (SEQ ID NO: 166) | (SEQ ID NO: 197) |
| Int10 | GGAAAATATAAATAA | AGCACGCTGATAATC |
| | TTTTAGTAACCTACA | AGCAAGACCACCAAC |
| | TCTCAATCAAGGATA | ATTTCCACCAATGTA |
| | GTAAAACTCTCACTC | AAAGCTTTAACCTTA |
| | TT | GC |
| | (SEQ ID NO: 167) | (SEQ ID NO: 198) |
| Int11 | GTTTATATGTTTACT | ATGGATTTTGCAGAT |
| | AATAAGACGCTCTCA | TCCCAGATGCCCCTA |
| | ACCCATAAAGTCTTA | CAGAAAGAGGTACAA |
| | TTAGTAAACATATTT | AACATTTATTGGAAT |
| | CAACT | TAATT |
| | (SEQ ID NO: 168) | (SEQ ID NO: 199) |
| Int12 | TTTTTGTATGTTAGT | GTTCGTGGTAACTAT |
| | TGTGTCACTGGGTAG | GGGTGGTACAGGTGC |
| | ACCTAAATAGTGACA | CACATTAGTTGTACC |
| | CAACTGCTATTAAAA | ATTTATGTTTATGTG |
| | TTTAA | GTTAAC |
| | (SEQ ID NO: 169) | (SEQ ID NO: 200) |
| Int13 | CAATAACGGTTGTAT | GCATACATTGTTGTT |
| | TTGTAGAACTTGACC | GTTTTTCCAGATCCA |
| | AGTTGTTTTAGTAAC | GTTGGTCCTGTAAAT |
| | ATAAATACAACTCCG | ATAAGCAATCCATGT |
| | AATA | GAGT |
| | (SEQ ID NO: 170) | (SEQ ID NO: 201) |
| LI | GTTTAGTATCTCGTT | TAACTTTTTCGGATC |
| | ATCTCTCGTTGGAGG | GAGTTATGATGGACG |
| | GAGAAGAAACGGGAT | TAAAGAGGGAACAAA |
| | ACCAAAA | GCATCTA |
| | (SEQ ID NO: 171) | (SEQ ID NO: 202) |
| Peaches | TAGTTTCCAATGTTA | CGGTCTCCATCGGGA |
| | CAGGAACTGCTGGCA | TCTGCTGATCGAGCA |
| | GAATCCAACACATTG | GCATGCCGACCA |
| | GAAGTCG | (SEQ ID NO: 203) |
| | (SEQ ID NO: 172) | |
| Bxz2 | TAACCGCAAGTGTAC | CGGTCTCCATCGGGA |
| | ATCCCTCGGCTGGCC | TCTGCTGATCGAGCA |
| | GAGACAAGTACAGTT | GCATGCCGACCA |
| | GCGACAG | (SEQ ID NO: 203) |
| | (SEQ ID NO: 173) | |
| SV1 | ATGTGGTCCTTTAGA | CATCAGGGCGGTCAG |
| | TCCACTGACGTGGGT | GCCGTAGATGTGGAA |
| | CAGTGTCTCTAAAGG | GAAACGGCAGCACGG |
| | ACTCGCG | CGAGGACG |
| | (SEQ ID NO: 174) | (SEQ ID NO: 204) |

+0 TABLE E
Serine resolvases and SSR target sequences.

| | Recombinase recognition | Recombinase recognition |
|---|---|---|
| | sequence | sequence |
| Resolvase | Left | Right |
| Gin | CGTTTCCTGTAAACCGAGGTTTTG | CGTTTCCTGTAAACCGAGGTTTTG |
| | GATAAACA (SEQ ID NO: 205) | GATAATGG (SEQ ID NO: 210) |
| Cin | GAGTTCTCTTAAACCAAGGTTTAG | GAGTTCTCTTAAACCAAGGTATTG |
| | GATTGAAA (SEQ ID NO: 206) | GATAACAG (SEQ ID NO: 211) |
| Hin | TGGTTCTTGAAAACCAAGGTTTTT | AAATTTTCCTTTTTGGAAGGTTTTT |
| | GATAAAGC (SEQ ID NO: 207) | GATAACCA (SEQ ID NO: 212) |
| Min | GCCTTCCCCTAAACCAACGTTTTT | GCCTTCCCCCAAACCAAGGTAATC |
| | ATGCCGCC (SEQ ID NO: 208) | AAGAACGC (SEQ ID NO: 213) |
| Sin | TTGTGAAATTTGGGTACACCCTAA | CGTATGATTAGGGTGTATATTAAT |
| | TCATACAA (SEQ ID NO: 209) | TT (SEQ ID NO: 214) |

+0 TABLE F
Tyrosine integrases and target sequences.

| Integrase | attP | attB |
|---|---|---|
| HK022 | CAAATGATTTTATTTTGACTAATAAT | GCACTTTAG |
| | GACCTACTTACATTAATTTACTGATA | GTGAAAAAG |
| | ATTAAAGAGATTTTAAATATACAACT | GTT |
| | TATTCACCTAAAGGATGACAAAA | (SEQ ID |
| | (SEQ ID NO: 215) | NO: 219) |
| | TAACATTAATCACTTAAAAATCATC | |
| | GCATTACACTAATCTGTGGTTAAATG | |
| | ATAGACTACATAATGCGACAAAACG | |
| | CAACATATCCAGTCACTATGAATCA | |
| | ACTACTTAGATAGTATTAGTGACCT | |
| | (SEQ ID NO: 216) | |
| P22 | CTAAGTGGTTTGGGACAAAAATGGG | GCAGCGCATT |
| | ACATACAAATCTTTGCATCGGTTTGC | CGTAATGCGA |
| | AAGGCTTTGCATGTCTTTCGAAGATG | AGGTCGT |
| | GGACGTGTGAGCGCAGGTATGACGT | (SEQ ID |
| | GGTATGTGTTGACTTAAAAGGTAGTT | NO: 220) |
| | CTTATAATTCGTAATGCGAAGGTCGT | |
| | AGGTTCGACTCCTATTATCGGCACCA | |
| | GTTAAATCAAATACTTACGTATTATT | |
| | CGTGCCTTCCTTATTTTTACTGTGGG | |
| | ACATATTTGGGACAGAAGTACCAAA | |
| | AA (SEQ ID NO: 217) | |
| L5 | GCGATCCCCATCCGCGACGTGCCAA | GAGCGGGCGA |
| | CTAGGTCTCCTCTCGTCGTGAACAAG | CGGGAATCGA |
| | GCTACCGGGTTGCAACTCCTGTGCA | ACCCGCGTAG |
| | ACTCTCAGGCTTCAACGCGCTTCTAC | CTAGTTTGGA |
| | GACCTGCAATTTCTTTCCACTTAGAG | AGA |
| | GATGCAGCCGAGAGGGGTAAAAACC | (SEQ ID |
| | TATCTTGACCGGCCCATATGTGGTCG | NO: 221) |
| | GCAGACACCCATTCTTCCAAACTAG | |
| | CTACGCGGGTTCGATTCCCGTCGCCC | |
| | GCTCCGCTGGTCAGAGGGTGTTTTCG | |
| | CCCTCTGGCCATTTTTCTTTCCAGGG | |
| | GTCTGCAACTCTTGTGCGACTCTTCT | |
| | GACCTGGGCATACGCGGTTGCAACG | |
| | CATCCCTGATCTGGCTACTTTCGATG | |
| | CTGACAAACGAATAGAGCCCCCCGC | |
| | CTGCGCGAACAGACGAGGGGCATTC | |
| | ACA (SEQ ID NO: 218) | |

In various other aspects, the present disclosure relates to methods of using single flap PE, twinPE, or multi-flap PE to install one or more recombinase recognition sequences and their use in site-specific recombination.

In some embodiments, the site-specific recombination may effectuate a variety of recombination outcomes, such as, excision, integration, inversion, or exchange of DNA fragments.

In some embodiments, the methods are useful for inducing recombination of or between two or more regions of two or more nucleic acid (e.g., DNA) molecules. In other embodiments, the methods are useful for inducing recombination of or between two or more regions in a single nucleic acid molecule (e.g., DNA).

In some embodiments, the disclosure provides a method for integrating a donor DNA template by site-specific recombination, comprising: (a) installing a recombinase recognition sequence at a genomic locus by single flap PE, twinPE, or multi-flap PE prime editing; (b) contacting the genomic locus with a DNA donor template that also comprises the recombinase recognition sequence in the presence of a recombinase.

In other embodiments, the disclosure provides a method for deleting a genomic region by site-specific recombination, comprising: (a) installing a pair of recombinase recognition sequences at a genomic locus by single flap PE, twinPE, or multi-flap PE prime editing; (b) contacting the genomic locus with a recombinase, thereby catalyzing the deletion of the genomic region between the pair of recombinase recognition sequences.

In yet other embodiments, the disclosure provides a method for inverting a genomic region by site-specific recombination, comprising: (a) installing a pair of recombinase recognition sequences at a genomic locus by single flap PE, twinPE, or multi-flap PE prime editing; (b) contacting the genomic locus with a recombinase, thereby catalyzing the inversion of the genomic region between the pair of recombinase recognition sequences.

In still other embodiments, the disclosure provides a method for inducing chromosomal translocation between a first genomic site and a second genomic site, comprising: (a) installing a first recombinase recognition sequence at a first genomic locus by single flap PE, twinPE, or multi-flap PE; (b) installing a second recombinase recognition sequence at a second genomic locus by single flap PE, twinPE, or multi-flap PE (c) contacting the first and the second genomic loci with a recombinase, thereby catalyzing the chromosomal translocation of the first and second genomic loci.

In other embodiments, the disclosure provides a method for inducing cassette exchange between a genomic locus and a donor DNA comprising a cassette, comprising: (a) installing a first recombinase recognition sequence at a first genomic locus by single flap PE, twinPE, or multi-flap PE; (b) installing a second recombinase recognition sequence at a second genomic locus by single flap PE, twinPE, or multi-flap PE; (c) contacting the first and the second genomic loci with a donor DNA comprising a cassette that is flanked by the first and second recombinase recognition sequences and a recombinase, thereby catalyzing the exchange of the flanked genomic locus and the cassette in the DNA donor.

In various embodiments involving the insertion of more than one recombinase recognition sequences in the genome, the recombinase recognition sequences can be the same or different. In some embodiments, the recombinase recognition sequences are the same. In other embodiments, that recombinase recognition sequences are different.

In various embodiments, the recombinase can be a tyrosine recombinase, such as Cre, Dre, Vcre, Scre, Flp, B2, B3, Kw, R, TD1-40, Vika, Nigri, Panto, Kd, Fre, Cre(ALSHG), Tre, Brec1, or Cre-R3M3, as shown above. In such embodiments, the recombinase recognition sequence may be an RRS of the above tables that corresponds to the recombinase under use.

In various other embodiments, the recombinase can be a large serine recombinase, such as Bxb1, PhiC31, R4, phiBT1, MJ1, MR11, TP901-1, A118, V153, phiRV1, phi370.1, TG1, WB, BL3, SprA, phiJoe, phiK38, Int2, Int3, Int4, Int7, Int8, Int9, Int10, Int11, Int12, Int13, L1, peaches, Bxz2, or SV1, as shown in the above tables. In such embodiments, the recombinase recognition sequence may be an RRS that corresponds to the recombinase under use.

In still other embodiments, the recombinase can be a serine recombinase, such as Bxb1, PhiC31, R4, phiBT1, MJ1, MR11, TP901-1, A118, V153, phiRV1, phi370.1, TG1, WB, BL3, SprA, phiJoe, phiK38, Int2, Int3, Int4, Int7, Int8, Int9, Int10, Int11, Int12, Int13, L1, peaches, Bxz2, or SV1, as shown in the above tables. In such embodiments, the recombinase recognition sequence may be an RRS that corresponds to the recombinase under use.

In other embodiments, the recombinase can be a serine resolvase, such as Gin, Cin, Hin, Min, or Sin, as shown in the above tables. In such embodiments, the recombinase recognition sequence may be an RRS that corresponds to the recombinase under use.

In various other embodiments, the recombinase can be a tyrosine integrase, such as HK022, P22, or L5, as shown in the above tables. In such embodiments, the recombinase recognition sequence may be an RRS that corresponds to the recombinase under use.

In some embodiments, any of the methods for site-specific recombination with a prime editing system, including single flap, twin flap, and multi-flap prime editing system can be performed in vivo or in vitro. In some embodiments, any of the methods for site-specific recombination are performed in a cell (e.g., recombine genomic DNA in a cell). The cell can be prokaryotic or eukaryotic. The cell, such as a eukaryotic cell, can be in an individual, such as a subject, as described herein (e.g., a human subject). The methods described herein are useful for the genetic modification of cells in vitro and in vivo, for example, in the context of the generation of transgenic cells, cell lines, or animals, or in the alteration of genomic sequence, e.g., the correction of a genetic defect, in a cell in a subject.

In other embodiments, fragments of a recombination site may be installed by single flap PE, twinPE, or multi-flap PE so long as the recombination site fragment retains the biological activity of the full recombination site and hence facilitate a recombination event in the presence of the appropriate recombinase. Thus, fragments of a recombination site may range from at least about 5, 10, 15, 20, 25, 30, 35, 40 nucleotides, and up to the full-length of a recombination site. Active variants can comprise at least 65%, 70%, 75%, 80%, 85%, 90%, 91%, 92%, 93%, 94%, 95%, 96%, 97%, 98%, 99% or more sequence identity to the native recombination site, wherein the active variants retain biological activity and hence facilitate a recombination event in the presence of the appropriate recombinase. Assays to measure the biological activity of recombination sites are known in the art. See, for example, Senecoll et al. (1988) J. Mol. Biol. 201:406-421; Voziyanov et al. (2002) Nucleic Acid Research 30:7, U.S. Pat. No. 6,187,994, WO/01/00158, and Albert et al. (1995) The Plant Journal 7:649-659.

Recombinases are also employed in the methods and compositions provided herein. By “recombinase” is intended a native polypeptide that catalyzes site-specific recombination between compatible recombination sites. For reviews of site-specific recombinases, see Sauer (1994) Current Opinion in Biotechnology 5:521-527; and Sadowski (1993) FASEB 7:760-767; the contents of which are incorporated herein by reference. The recombinases used in the methods disclosed herein can be a naturally occurring recombinase or a biologically active fragment or variant of the recombinase. Recombinases useful in the methods and compositions include recombinases from the Integrase and Resolvase families, biologically active variants and fragments thereof, and any other naturally occurring or recombinantly produced enzyme or variant thereof that catalyzes conservative site-specific recombination between specified DNA recombination sites. Thus, recombinase can include site-specific enzymes that may be referred to in the art as integrases, resolvases, and invertases. In some embodiments, a recombinase is a serine recombinase. In some embodiments, a recombinase is a tyrosine recombinase. A recombinase can result in various edits of the DNA sequences between recombinase recognition sequences, including deletion (excision), integration (insertion), inversion, exchange between two DNA fragments, and translocation. In some embodiments, a recombinase can result in a unidirectional edit in a target DNA. In some embodiments, a recombinase can result in a bidirectional edit in a target DNA.

The Integrase family of recombinases has over one hundred members and includes, for example, FLP, Cre, Int, and R. For other members of the Integrase family, see for example, Esposito et al. (1997) Nucleic Acid Research 25:3605-3614 and Abremski et al. (1992) Protein Engineering 5:87-91, both of which are herein incorporated by reference. Other recombination systems include, for example, the streptomyces bacteriophage phi C31 (Kuhstoss et al. (1991) J. Mol. Biol. 20:897-908); the SSV1 site-specific recombination system from Sulfolobus shibatae(Maskhelishvili et al. (1993) Mol. Gen. Genet. 237:334-342); and a retroviralintegrase-based integration system (Tanaka et al. (1998) Gene 17:67-76). In other embodiments, the recombinase is one that does not require cofactors or a supercoiled substrate. Such recombinases include Cre, FLP, or active variants or fragments thereof.

The FLP recombinase is a protein that catalyzes a site-specific reaction that is involved in amplifying the copy number of the two-micron plasmid of S. cerevisiae during DNA replication. As used herein, FLP recombinase refers to a recombinase that catalyzes site-specific recombination between two FRT sites. The FLP protein has been cloned and expressed. See, for example, Cox (1993) Proc. Natl. Acad. Sci. U.S. A. 80:4223-4227. The FLP recombinase for use in the methods and with the compositions may be derived from the genus Saccharomyces. One can also synthesize a polynucleotide comprising the recombinase using plant-preferred codons for optimal expression in a plant of interest. A recombinant FLP enzyme encoded by a nucleotide sequence comprising maize preferred codons (FLPm) that catalyzes site-specific recombination events is known. See, for example, U.S. Pat. No. 5,929,301, herein incorporated by reference. Additional functional variants and fragments of FLP are known. See, for example, Buchholz et al. (1998) Nat. Biotechnol. 76:617-618, Hartung et al. (1998) J. Biol. Chem. 273:22884-22891, Saxena et al. (1997) Biochim Biophys Acta 1340{2): 187-204, and Hartley et al. (1980) Nature 286:860-864, all of which are herein incorporated by reference.

The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; Shaikh et al. (1977) J. Biol. Chem. 272:5695-5702; and, Buchholz et al. (1998) Nat. Biotechnol. 76:617-618, all of which are herein incorporated by reference. The Cre polynucleotide sequences may also be synthesized using plant-preferred codons. Such sequences (moCre) are described in WO 99/25840, herein incorporated by reference.

It is further recognized that a chimeric recombinases can be used in the methods described herein. By “chimeric recombinase” is intended a recombinant fusion protein which is capable of catalyzing site-specific recombination between recombination sites that originate from different recombination systems. That is, if a set of functional recombination sites, characterized as being dissimilar and non-recombinogenic with respect to one another, is utilized in the methods and compositions and comprises a FRT site and a LoxP site, a chimeric FLP/Cre recombinase or active variant or fragment thereof will be needed or, alternatively, both recombinases may be separately provided. Methods for the production and use of such chimeric recombinases or active variants or fragments thereof are described in WO 99/25840, herein incorporated by reference.

By utilizing various combinations of recombination sites in the transgenic SSR target sites and the transfer cassettes provided herein, the methods provide a mechanism for the site-specific integration of polynucleotides of interest into a specific site in a genome. The methods also allow for the subsequent insertion of additional polynucleotides of interest into the specific genomic site.

The following is a list of suitable tyrosine recombinase SSR target sequences that may be used, along with their cognate recombinase:

Tyrosine recombinases and SSR target sequences.

| | | SEQ ID | |
|---|---|---|---|
| Recombinase | Target | NO: | Name |
| Cre | ATAACTTCGTATAGCATACATTATACGAAGTTAT | 124 | loxP |
| Cre | ATAACTTCGTATAAAGTATCCTATACGAAGTTAT | 222 | lox2272 |
| Cre | ATAACTTCGTATAGTATACCTTATACGAAGTTAT | 223 | loxN |
| Cre | ATAACTTCGTATAATGTATACTATACGAAGTTAT | 224 | lox511 |
| Cre | ATAACTTCGTATAATGTGTACTATACGAAGTTAT | 225 | lox5171 |
| Cre | ATAACTTCGTATAAGAAACCATATACGAAGTTAT | 226 | M2 |
| Cre | ATAACTTCGTATATAATACCATATACGAAGTTAT | 227 | M3 |
| Cre | ATAACTTCGTATAAGATAGAATATACGAAGTTAT | 228 | M7 |
| Cre | ATAACTTCGTATACGATACCATATACGAAGTTAT | 229 | M11 |
| Cre | TACCGTTCGTATAGCATACATTATACGAAGTTAT | 230 | lox71 |
| Cre | ATAACTTCGTATAGCATACATTATACGAACGGTA | 231 | lox66 |
| Dre | TAACTTTAAATAATGCCAATTATTTAA AGTTA | 125 | rox |
| VCre | TCAATTTCTGAGAACTGTCATTCTCGG AAATTGA | 126 | loxV |
| SCre | CTCGTGTCCGATAACTGTAATTATCGG ACATGAT | 127 | loxS |
| Flp | GAAGTTCCTATTCTCTAGAAAGTATAG GAACTTC | 128 | FRT |
| B2 | GAGTTTCATTAAGGAATAACTAATTCC | 129 | loxB |
| | CTAATGAAACTC | | |
| B3 | GGTTGCTTAAGAATAAGTAATTCTTAA GCAACC | 130 | loxB3 |
| Kw | ACGAAAAATGGTAAGGAATAGACCAT | 131 | |
| | TCCTTACCATTTTTGGT | | |
| R | TTGATGAAAGAATAACGTATTCTTTCA TCAA | 132 | RS |
| TD1-40 | GTGCGTCAAATAATAACGTATTATTTG ACACTT | 133 | TDRS |
| Vika | AATAGGTCTGAGAACGCCCATTCTCAG ACGTATT | 134 | VOX |
| Nigri | TGAATGTCCTATAATTACACTTATAGG ACATTCA | 135 | nox |
| Panto | GAAACTTTAAATAATAAGTCTTATTTA AAGTTTC | 136 | Pox |
| Kd | AAACGATATCAGACATTTGTCTGATA | 137 | loxK |
| | ATGCTTCATTATCAGACAAATGTCTG ATATCGTTT | | |
| Fre | ATATATACGTATATAGACATATATACG TATATAT | 138 | loxH |
| CreALSHG | ATAACTCTATATAATGTATGCTATATA GAGTTAT | 139 | loxM7 |
| Tre | ACAACATCCTATTACACCCTATATGCC AACATGG | 140 | loxLTR |
| Brec 1 | AACCCACTGCTTAAGCCTCAATAAAGC TTGCCTT | 141 | loxBTR |
| Cre-R3M3 | GATACAACGTATATACCTTTCTATACG TTGTTTA | 142 | loxK2 |

The following is a list of suitable serine recombinase SSR target sequences that may be used, along with their cognate serine recombinase:

| Integrase | Left Target | Right Target |
|---|---|---|
| Bxb1 | GGTTTGTCTGGTCAA | GGCTTGTCGACGACG |
| | CCACCGCGGTCTCAG | GCGGTCTCCGTCGTC |
| | TGGTGTACGGTACAA | AGGATCAT |
| | ACC | (SEQ ID NO: 175) |
| | (SEQ ID NO: 143) | |
| Bxb1 | GGTTTGTCTGGTCAA | GGCTTGTCGACGACG |
| | CCACCGCGGACTCAG | GCGGACTCCGTCGTC |
| | TGGTGTACGGTACAA | AGGATCAT |
| | ACC | (SEQ ID NO: 248) |
| | (SEQ ID NO: 232) | |
| Bxb1 | GGTTTGTCTGGTCAA | GGCTTGTCGACGACG |
| | CCACCGCGGCCTCAG | GCGGCCTCCGTCGTC |
| | TGGTGTACGGTACAA | AGGATCAT |
| | ACC | (SEQ ID NO: 249) |
| | (SEQ ID NO: 233) | |
| Bxb1 | GGTTTGTCTGGTCAA | GGCTTGTCGACGACG |
| | CCACCGCGGGCTCAG | GCGGGCTCCGTCGTC |
| | TGGTGTACGGTACAA | AGGATCAT |
| | ACC | (SEQ ID NO: 250) |
| | (SEQ ID NO: 234) | |
| Bxb1 | GGTTTGTCTGGTCAA | GGCTTGTCGACGACG |
| | CCACCGCGAACTCAG | GCGAACTCCGTCGTC |
| | TGGTGTACGGTACAA | AGGATCAT |
| | ACC | (SEQ ID NO: 251) |
| | (SEQ ID NO: 235) | |
| Bxb1 | GGTTTGTCTGGTCAA | GGCTTGTCGACGACG |
| | CCACCGCGACCTCAG | GCGACCTCCGTCGTC |
| | TGGTGTACGGTACAA | AGGATCAT |
| | ACC | (SEQ ID NO: 252) |
| | (SEQ ID NO: 236) | |
| Bxb1 | GGTTTGTCTGGTCAA | GGCTTGTCGACGACG |
| | CCACCGCGAGCTCAG | GCGAGCTCCGTCGTC |
| | TGGTGTACGGTACAA | AGGATCAT |
| | ACC | (SEQ ID NO: 253) |
| | (SEQ ID NO: 237) | |
| Bxb1 | GGTTTGTCTGGTCAA | GGCTTGTCGACGACG |
| | CCACCGCGATCTCAG | GCGATCTCCGTCGTC |
| | TGGTGTACGGTACAA | AGGATCAT |
| | ACC | (SEQ ID NO: 254) |
| | (SEQ ID NO: 238) | |
| Bxb1 | GGTTTGTCTGGTCAA | GGCTTGTCGACGACG |
| | CCACCGCGCACTCAG | GCGCACTCCGTCGTC |
| | TGGTGTACGGTACAA | AGGATCAT |
| | ACC | (SEQ ID NO: 255) |
| | (SEQ ID NO: 239) | |
| Bxb1 | GGTTTGTCTGGTCAA | GGCTTGTCGACGACG |
| | CCACCGCGCCCTCAG | GCGCCCTCCGTCGTC |
| | TGGTGTACGGTACAA | AGGATCAT |
| | ACC | (SEQ ID NO: 256) |
| | (SEQ ID NO: 240) | |
| Bxb1 | GGTTTGTCTGGTCAA | GGCTTGTCGACGACG |
| | CCACCGCGCGCTCAG | GCGCGCTCCGTCGTC |
| | TGGTGTACGGTACAA | AGGATCAT |
| | ACC | (SEQ ID NO: 257) |
| | (SEQ ID NO: 241) | |
| Bxb1 | GGTTTGTCTGGTCAA | GGCTTGTCGACGACG |
| | CCACCGCGCTCTCAG | GCGCTCTCCGTCGTC |
| | TGGTGTACGGTACAA | AGGATCAT |
| | ACC | (SEQ ID NO: 258) |
| | (SEQ ID NO: 242) | |
| Bxb1 | GGTTTGTCTGGTCAA | GGCTTGTCGACGACG |
| | CCACCGCGTACTCAG | GCGTACTCCGTCGTC |
| | TGGTGTACGGTACAA | AGGATCAT |
| | ACC | (SEQ ID NO: 259) |
| | (SEQ ID NO: 243) | |
| Bxb1 | GGTTTGTCTGGTCAA | GGCTTGTCGACGACG |
| | CCACCGCGTCCTCAG | GCGTCCTCCGTCGTC |
| | TGGTGTACGGTACAA | AGGATCAT |
| | ACC | (SEQ ID NO: 260) |
| | (SEQ ID NO: 244) | |
| Bxb1 | GGTTTGTCTGGTCAA | GGCTTGTCGACGACG |
| | CCACCGCGTGCTCAG | GCGTGCTCCGTCGTC |
| | TGGTGTACGGTACAA | AGGATCAT |
| | ACC | (SEQ ID NO: 261) |
| | (SEQ ID NO: 245) | |
| Bxb1 | GGTTTGTCTGGTCAA | GGCTTGTCGACGACG |
| | CCACCGCGTTCTCAG | GCGTTCTCCGTCGTC |
| | TGGTGTACGGTACAA | AGGATCAT |
| | ACC | (SEQ ID NO: 262) |
| | (SEQ ID NO: 246) | |
| phiC31 | GTGCCCCAACTGGGG | TGCGGGTGCCAGGGC |
| | TAACCTTTGAGTTCT | GTGCCCTTGGGCTCC |
| | CTCAGTTGGGGG | CCGGGCGCGTACTCC |
| | (SEQ ID NO: 144) | (SEQ ID NO: 176) |
| R4 | TGTTCCCCAAAGCGA | GCATGTTCCCCAAAG |
| | TACCACTTGAAGCAG | CGATACCACTTGAAG |
| | TGGTACTGCTTGTGG | CAGTGGTACTGCTTG |
| | GTACA | TGGGTACACTCTGCG |
| | (SEQ ID NO: 145) | GGTG |
| | | (SEQ ID NO: 177) |
| phiBT1 | GGTGCTGGGTTGTTG | CAGGTTTTTGACGAA |
| | TCTCTGGACAGTGAT | AGTGATCCAGATGAT |
| | CCATGGGAAACTACT | CCAG |
| | CAGCACC | (SEQ ID NO: 178) |
| | (SEQ ID NO: 146) | |
| MJ1 | ATTTTAGGTATATGA | CAAAGGATCACTGAA |
| (phiFC1) | TTTTGTTTATTAGTG | TCAAAAGTATTGCTC |
| | TAAATAACACTATGT | ATCCACGCGAAA |
| | ACCTAAAAT | (SEQ ID NO: 179) |
| | (SEQ ID NO: 147) | |
| MR11 | TTTGTGCGGAACTAC | CGAAAATGTATGGAG |
| | GAACAGTTCATTAAT | GCACTTGTATCAATA |
| | ACGAAGTGTACAAAC | TAGGATGTATACCTT |
| | TTCCATACAA | CGAAGACACTT |
| | (SEQ ID NO: 148) | (SEQ ID NO: 180) |
| TP901-1 | GAGTTTTTATTTCGT | ATGCCAACACAATTA |
| | TTATTTCAATTAAGG | ACATCTCAATCAAGG |
| | TAACTAAAAAACTCC | TAAATGCTTTTTGCT |
| | TTTTAAGG | TTTTTTGC |
| | (SEQ ID NO: 149) | (SEQ ID NO: 181) |
| A118 | TTCCTCGTTTTCTCT | TTTCGGATCAAGCTA |
| | CGTTGGAAGAAGAAG | TGAAGGACGCAAAGA |
| | AAACGAGAAA | GGGAACTAAA |
| | (SEQ ID NO: 150) | (SEQ ID NO: 182) |
| U153 | TTCCTCGTTTTCTCT | TTTCGGATCAAGCTA |
| | CGTTGGACGGAAACG | TGAAGGACGCAAAGA |
| | AATCGAGAAA | GGGAACTAAA |
| | (SEQ ID NO: 151) | (SEQ ID NO: 182) |
| phiRV1 | GTAGTGTATCTCACA | GAAGGTGTTGGTGCG |
| | GGTCCACGGTTGGCC | GGGTTGGCCGTGGTC |
| | GTGGACTGCTGAAGA | GAGGTGGGGT |
| | ACATTCC | (SEQ ID NO: 183) |
| | (SEQ ID NO: 152) | |
| phi370.1 | AAAAAAATACAGCGT | ACAACTATACTCGTC |
| | TTTTCATGTACAACT | GGTAAAAAGGCATTG |
| | ATACTAGTTGTAGTG | TAAAGGAGACTGATA |
| | CCTAAAA | ATGGCATGT |
| | (SEQ ID NO: 153) | (SEQ ID NO: 184) |
| TG1 | TCCAGCCCAACAGTG | GATCAGCTCCGCGGG |
| | TTAGTCTTTGCTCTT | CAAGACCTTTCTCCT |
| | ACCCAGTTGGGCGGG | TCACGGGGTGGAAGG |
| | A | TC |
| | (SEQ ID NO: 154) | (SEQ ID NO: 185) |
| WB | CTAGTTTTAAAGTTG | ACTGTCGTGTTTGTA |
| | GTTATTAGTTACTGT | ACGGTACTTCCAACG |
| | GATATTTATCACGGT | GAAGGTAGCGTCAAC |
| | ACCCAATAACCAATG | GATAGGTGTACAGCT |
| | AAT | GGCGCCGCCAC |
| | (SEQ ID NO: 155) | (SEQ ID NO: 186) |
| BL3 | CAATGAAAAACTAGG | TTTCCACAGACAACT |
| | CATGTAGAAGTTGTT | CACGTGGAGGTAGTC |
| | TGT | AC |
| | (SEQ ID NO: 156) | (SEQ ID NO: 187) |
| SprA | TGTAGTAAGTATCTT | CACCCATTGTGTTCA |
| | AATATACAGCTTTAT | CAGGAGATACAGCTT |
| | CTGTTTTTTAAGATA | TATCTGTACTGATAT |
| | CTTACTACTTT | TAATGACATGCTG |
| | (SEQ ID NO: 157) | (SEQ ID NO: 188) |
| phiJoe | AGTTGTGGCCATGTG | ATCTGGATGTGGGTG |
| | TCCATCTGGGGGCAG | TCCATCTGCGGGCAG |
| | ATGGAGACGGGGTCA | ACGCCGCAGTCGAAG |
| | CA | CACGG |
| | (SEQ ID NO: 158) | (SEQ ID NO: 189) |
| phiK38 | CCCTAATACGCAAGT | GAGCGCCGGATCAGG |
| | CGATAACTCTCCTGG | GAGTGGACGGCCTGG |
| | GAGCGTTGACAACTT | GAGCGCTACACGCTG |
| | GCGCACCCTGATCTG | TGGCTGCGGTCGGTG |
| | (SEQ ID NO: 160) | C |
| | | (SEQ ID NO: 191) |
| Int2 | GCTCATGTATGTGTC | GGACGGCGCAGAAGG |
| | TACGCGAGATTCTCG | GGAGTAGCTCTTCGC |
| | CCCGAGAACTTCTGC | CGGACCGTCGACATA |
| | AAGGCACTGCTCTTG | CTGCTCAGCTCGTC |
| | GCT | (SEQ ID NO: 192) |
| | (SEQ ID NO: 161) | |
| Int3 | ATGGATAAAAAAATA | GTACAACTATACTCG |
| | CAGCGTTTTTCATGT | TCGGTAAAAAGGGTT |
| | ACAACTATACTAGTT | TGTAAAGGAGACTGA |
| | GTAGTGCCTAAATAA | TAATGGCATCATCTT |
| | TGCTT | AT |
| | (SEQ ID NO: 162) | (SEQ ID NO: 193) |
| Int4 | AAAAATTACAAAGTT | TTCCAAAGAGCGCCC |
| | TTCAACCCTTGATTT | AACGCGACCTGAAAT |
| | GAATTAGCGGTCAAA | TTGAATAAGACTGCT |
| | TAATTTGTAATTCGT | GCTTGTGTAAAGGCG |
| | TT | ATGATT |
| | (SEQ ID NO: 163) | (SEQ ID NO: 194) |
| Int7 | GTGTTATAAACCTGT | AGACGAGAAACGTTC |
| | GTGAGAGTTAAGTTT | CGTCCGTCTGGGTCA |
| | ACATGCCTAACCTTA | GTTGGGCAAAGTTGA |
| | ACTTTTACGCAGGTT | TGACCGGGTCGTCCG |
| | CAGCTT | TT |
| | (SEQ ID NO: 164) | (SEQ ID NO: 195) |
| Int8 | TTAATAAACTATGGA | CAATCATCAGATAAC |
| | AGTATGTACAGTCTT | TATGGCGGCACGTGC |
| | GCAATGTTGAGTGAA | ATTAACCACGGTTGT |
| | CAAACTTCCATAATA | ATCCCGTCTAAAGTA |
| | AAAT | CTCGT |
| | (SEQ ID NO: 165) | (SEQ ID NO: 196) |
| Int9 | GTGGTTGTTTTTGTT | TTTATATTGCGAAAA |
| | GGAAGTGTGTATCAG | ATAATTGGCGAACGA |
| | GTATCTGCATAGTTA | GGTAACTGGATACCT |
| | TTCCGAACTTCCAAT | CATCCGCCAATTAAA |
| | TA | ATTTG |
| | (SEQ ID NO: 166) | (SEQ ID NO: 197) |
| Int10 | GGAAAATATAAATAA | AGCACGCTGATAATC |
| | TTTTAGTAACCTACA | AGCAAGACCACCAAC |
| | TCTCAATCAAGGATA | ATTTCCACCAATGTA |
| | GTAAAACTCTCACTC | AAAGCTTTAACCTTA |
| | TT | GC |
| | (SEQ ID NO: 167) | (SEQ ID NO: 198) |
| Int11 | GTTTATATGTTTACT | ATGGATTTTGCAGAT |
| | AATAAGACGCTCTCA | TCCCAGATGCCCCTA |
| | ACCCATAAAGTCTTA | CAGAAAGAGGTACAA |
| | TTAGTAAACATATTI | AACATTTATTGGAAT |
| | CAACT | TAATT |
| | (SEQ ID NO: 168) | (SEQ ID NO: 199) |
| Int12 | TTTTTGTATGTTAGT | GTTCGTGGTAACTAT |
| | TGTGTCACTGGGTAG | GGGTGGTACAGGTGC |
| | ACCTAAATAGTGACA | CACATTAGTTGTACC |
| | CAACTGCTATTAAAA | ATTTATGTTTATGTG |
| | TTTAA | GTTAAC |
| | (SEQ ID NO: 169) | (SEQ ID NO: 200) |
| Int13 | CAATAACGGTTGTAT | GCATACATTGTTGTT |
| | TTGTAGAACTTGACC | GTTTTTCCAGATCCA |
| | AGTTGTTTTAGTAAC | GTTGGTCCTGTAAAT |
| | ATAAATACAACTCCG | ATAAGCAATCCATGT |
| | AATA | GAGT |
| | (SEQ ID NO: 170) | (SEQ ID NO: 201) |
| LI | GTTTAGTATCTCGTT | TAACTTTTTCGGATC |
| | ATCTCTCGTTGGAGG | GAGTTATGATGGACG |
| | GAGAAGAAACGGGAT | TAAAGAGGGAACAAA |
| | ACCAAAA | GCATCTA |
| | (SEQ ID NO: 171) | (SEQ ID NO: 202) |
| Peaches | TAGTTTCCAATGTTA | CGGTCTCCATCGGGA |
| | CAGGAACTGCTGGCA | TCTGCTGATCGAGCA |
| | GAATCCAACACATTG | GCATGCCGACCA |
| | GAAGTCG | (SEQ ID NO: 203) |
| | (SEQ ID NO: 172) | |
| Bxz2 | TAACCGCAAGTGTAC | CGGTCTCCATCGGGA |
| | ATCCCTCGGCTGGCC | TCTGCTGATCGAGCA |
| | GAGACAAGTACAGTT | GCATGCCGACCA |
| | GCGACAG | (SEQ ID NO: 203) |
| | (SEQ ID NO: 173) | |
| SV1 | ATGTGGTCCTTTAGA | CATCAGGGCGGTCAG |
| | TCCACTGACGTGGGT | GCCGTAGATGTGGAA |
| | CAGTGTCTCTAAAGG | GAAACGGCAGCACGG |
| | ACTCGCG | CGAGGACG |
| | (SEQ ID NO: 174) | (SEQ ID NO: 204) |
| SPBc2 | GTAGTAAGTATCTTA | CACCCATTGTGTTCA |
| | ATATACAGCTTTATC | CAGGAGATACAGCTT |
| | TGTTTTTTAAGATAC | TATCTGTACTGATAT |
| | TTACTACTTT | TAATGACATGCTGCA |
| | (SEQ ID NO: 247) | CT |
| | | (SEQ ID NO: 263) |

The following is a list of suitable serine resolvase SSR target sequences that may be used, along with their cognate serine resolvase:

| Resolvase | Left Target | Right Target |
|---|---|---|
| Gin | CGTTTCCTGTAA | CGTTTCCTGTAAA |
| | ACCGAGGTTTT | CCGAGGTTTTGGA |
| | GGATAAACA | TAATGG |
| | (SEQ ID NO: 205) | (SEQ ID NO: 210) |
| Cin | GAGTTCTCTTAA | GAGTTCTCTTAAA |
| | ACCAAGGTTTAG | CCAAGGTATTGGA |
| | GATTGAAA | TAACAG |
| | (SEQ ID NO: 206) | (SEQ ID NO: 211) |
| Hin | TGGTTCTTGAAA | AAATTTTCCTTTT |
| | ACCAAGGTTTT | TGGAAGGTTTTTG |
| | TGATAAAGC | ATAACCA |
| | (SEQ ID NO: 207) | (SEQ ID NO: 212) |
| Min | GCCTTCCCCTAA | GCCTTCCCCCAAAC |
| | ACCAACGTTTTT | CAAGGTAATCAA |
| | ATGCCGCC | GAACGC |
| | SEQ ID NO: 208) | (SEQ ID NO: 213) |
| Sin | TTGTGAAATTT | CGTATGATTAGGGT |
| | GGGTACACCCT | GTATATTAATTT |
| | AATCATACAA | (SEQ ID NO: 214) |
| | (SEQ ID NO: 209) | |

The following is a list of suitable tyrosine integrase SSR target sequences that may be used, along with their cognate tyrosine integrase:

| Integrase | attP | attB |
|---|---|---|
| HK022 | CAAATGATTTTATTTTGACTAATAATGACCTACTT | GCACTTTAGGTGAAA |
| | ACATTAATTTACTGATAATTAAAGAGATTTTAAAT | (SEQ ID NO: |
| | AAGGTTATACAACTTATTCACCTAAAGGATGACAA | 219) |
| | AA | |
| | (SEQ ID NO: 215) | |
| P22 | CTAAGTGGTTTGGGACAAAAATGGGACATACAAA | GCAGCGCATTCGTAAT |
| | TCTTTGCATCGGTTTGCAAGGCTTTGCATGTCTTT | GCGAAGGTCGT (SEQ |
| | CGAAGATGGGACGTGTGAGCGCAGGTATGACGTGID | NO: 220) |
| | GTATGTGTTGACTTAAAAGGTAGTTCTTATAATTC | |
| | GTAATGCGAAGGTCGTAGGTTCGACTCCTATTAT | |
| | CGGCACCAGTTAAATCAAATACTTACGTATTATTC | |
| | GTGCCTTCCTTATTTTTACTGTGGGACATATTTGG | |
| | GACAGAAGTACCAAAAA (SEQ ID NO: 217) | |
| L5 | GCGATCCCCATCCGCGACGTGCCAACTAGGTCTC | GAGCGGGCGACGGGA |
| | CTCTCGTCGTGAACAAGGCTACCGGGTTGCAACT | ATCGAACCCGCGTAG |
| | CCTGTGCAACTCTCAGGCTTCAACGCGCTTCTACG | CTAGTTTGGAAGA |
| | ACCTGCAATTTCTTTCCACTTAGAGGATGCAGCCG | (SEQ ID NO: 221) |
| | AGAGGGGTAAAAACCTATCTTGACCGGCCCATAT | |
| | GTGGTCGGCAGACACCCATTCTTCCAAACTAGCT | |
| | ACGCGGGTTCGATTCCCGTCGCCCGCTCCGCTGGT | |
| | CAGAGGGTGTTTTCGCCCTCTGGCCATTTTTCTTT | |
| | CCAGGGGTCTGCAACTCTTGTGCGACTCTTCTGAC | |
| | CTGGGCATACGCGGTTGCAACGCATCCCTGATCT | |
| | GGCTACTTTCGATGCTGACAAACGAATAGAGCCC | |
| | CCCGCCTGCGCGAACAGACGAGGGGCATTCACA | |
| | (SEQ ID NO: 218) | |

The above list of sequences is not intended in any way to limit which recombinase and SSR recognition sequences that may be used in the instant disclosure.

### Methods for Modifying Genome Using Installed SSR Recognition Sequences

In various embodiments, the disclosure provides (i) a prime editor system comprising a prime editor (PE) comprising a nucleic acid programmable DNA binding protein (“napDNAbp”) and a polymerase (e.g., reverse transcriptase) and a prime editing guide RNA (PEgRNA) for targeting the prime editor to a target DNA sequence and (ii) a site-specific recombinase, wherein the PEgRNA comprises (a) a spacer sequence that comprises a region of complementarity to a first strand of a target DNA sequence, (b) an extension arm that comprises a DNA synthesis template and a primer binding site in a 5′ to 3′ orientation, and (c) a gRNA core that associates with the napDNAbp, and wherein the DNA synthesis template comprises one or more site-specific recombinase recognition sequences. Via prime editing, one or more site specific recombinase recognition sequences encoded by the DNA synthesis template become integrated into the target DNA sequence. The integrated one or more site-specific recombinase recognition sequences may then undergo site-specific recombination in the presence of the recombinase. In various embodiments, the disclosure also provides isolated prime editor systems describe herein. In various other embodiments, the disclosure provides complexes comprising the prime editor and a PEgRNA. In still other embodiments, the disclosure provides one or more nucleic acid molecules encoding the prime editor systems, PEgRNAs, and recombinases. In various embodiments, the prime editor systems, PEgRNAs, and recombinase may be encoded on the same nucleic acid molecule, or they may be encoded on different nucleic molecule.

In various embodiments, the disclosure provides (i) a prime editor system (twin PE system) comprising a prime editor comprising a nucleic acid programmable DNA binding protein (“napDNAbp”) and a polymerase (e.g., reverse transcriptase) and a pair of prime editing guide RNAs (PEgRNA) for targeting the prime editor to opposite strands of a target DNA sequence and (ii) a site-specific recombinase. In some embodiments, the twin PE system comprises a first PEgRNA comprising a first spacer, a first gRNA core, and a first DNA synthesis template, wherein the first spacer binds to a first target site in the target DNA and wherein the first DNA synthesis template comprises one or more recombinase recognition sites as compared to the target DNA, and a second PEgRNA comprising a second spacer, a second gRNA core, and a second DNA synthesis template, wherein the second spacer binds to a second target site in the target DNA and wherein the second DNA synthesis template comprises one or more recombinase recognition sites as compared to the target DNA. In some embodiments, a double stranded DNA sequence corresponding to (the first DNA synthesis template not complement to the second DNA synthesis template+the region of complementarity between the first DNA synthesis template+the second DNA synthesis template not complement to the first DNA synthesis template), referred to as the replacement duplex between the first DNA synthesis template and the second DNA synthesis template, comprises one or more recombinase recognition sites. In some embodiments, the region of complementarity between the first DNA synthesis template and the second DNA synthesis template comprises one or more recombinase recognition sites. In operation, once the polymerase component of the prime editing system synthesizes the first and the second 3′ DNA flaps based on the sequence of the DNA synthesis templates, the 3′ DNA flaps are capable of forming the replacement duplex comprising the one or more site-specific recombinase recognition sequences. This duplex then replaces the endogenous and corresponding strands of the target DNA sequence, such that after replacement and then ligation, the one or more recombinase recognition sequences become permanently installed into the target DNA sequence.

In various embodiments, the disclosure provides a prime editor system for installing one site-specific recombinase recognition sequence at a target DNA locus. In some embodiments, the disclosure provides a prime editor system for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one DNA locus or multiple target DNA loci. For example, in some embodiments, a prime editor system comprises two or more PEgRNAs, wherein each of the PEgRNAs comprises a spacer sequence targeting a different target site in the target DNA, and each of the PEgRNAs independently comprises a DNA synthesis template that comprises one or more recombinase recognition sequence. The recombinase recognition sequences in each of the DNA synthesis templates may be the same or may be different.

In some embodiments, the disclosure provides a twin PE system, or a multi-flap PE system, for installing one site-specific recombinase recognition sequence at a target DNA locus. In some embodiments, the disclosure provides a twin PE system, or a multi-flap PE system, for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one DNA locus or multiple target DNA loci. In some embodiments, the disclosure provides multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one DNA locus or multiple target DNA loci. For example, in some embodiments, a prime editor system comprises two or more pairs of PEgRNAs, wherein each pair of the PEgRNAs comprises a pair of spacer sequences targeting a different region of the target DNA, and wherein the replacement duplex between each pair of the two or more pairs of PEgRNAs independently comprises one or more recombinase recognition sequence. The recombinase recognition sequences in each of the replacement duplexes may be the same or may be different.

In various embodiments, the disclosure provides a prime editor system comprising a PE for installing one site-specific recombinase recognition sequence at a target DNA locus, or multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one DNA locus or multiple target DNA loci.

In various embodiments, the disclosure provides a prime editor system comprising a twinPE for installing one site-specific recombinase recognition sequence at a target DNA locus, or multiple prime editor systems for installing at least two, three, four, five, six, seven, eight, nine, ten, eleven, twelve, thirteen, fourteen, fifteen, sixteen, seventeen, eighteen, nineteen, twenty, or more site-specific recombinase recognition sequences at one DNA locus or multiple target DNA loci.

The integrated one or more site-specific recombinase recognition sequences may then undergo site-specific recombination in the presence of the recombinase. In various embodiments, the disclosure also provides isolated prime editor systems describe herein. In various other embodiments, the disclosure provides complexes comprising the prime editor and a PEgRNA. In still other embodiments, the disclosure provides one or more nucleic acid molecules encoding the prime editor systems, PEgRNAs, and recombinases. In various embodiments, the prime editor systems, PEgRNAs, and recombinase may be encoded on the same nucleic acid molecule, or they may be encoded on different nucleic molecule.

In some embodiments, the disclosure provides a prime editor system having a recombinase for introducing a single recombinase recognition site in the target DNA, target gene, target genome, or target cell, and results in an intended edit in the target DNA, target gene, target genome, or target cell.

In some embodiments, a prime editor system with a recombinase component can result in insertion of an exogenous DNA sequence in a target DNA or target gene. In some embodiments, a single installed recombinase recognition site can be used as a landing site for a recombinase mediated reaction between the landing site installed in the target DNA and a second recombinase recognition site in a donor polynucleotide, for example, an exogenous donor DNA.

Insertion of a single recombinase recognition site can be accomplished with either PE having single PEgRNAs (i.e., single flap PE) or twinPE. For example, in some embodiments, a prime editor system comprises a single PEgRNA comprising a DNA synthesis template comprising a single recombinase recognition site, which then directs the prime editor system to introduce the single recombinase recognition sites in a target DNA.

In some embodiments, a prime editor system comprises a pair of PEgRNAs each comprising a DNA synthesis template, wherein the first DNA synthesis template and the second DNA synthesis template comprises a region of complementarity to each other, and wherein the sequence (first DNA synthesis template and second DNA synthesis template have a region of complementarity between one another) comprises a single recombinase recognition site, and the prime editor system introduces the single recombinase recognition site in the target DNA.

For example, in some embodiments, a PEgRNA directs the prime editor system to introduce a recombinase recognition site in a target DNA. In some embodiments, a first PEgRNA and a second PEgRNA having a region of complementarity to each other introduces a recombinase recognition site in a target DNA. In some embodiments, the prime editor system further comprises a donor polynucleotide, e.g., a donor DNA, wherein the donor polynucleotide comprises one or more recombinase recognition site. In some embodiments, the recombinase component of the prime editor system results in recombination between the donor polynucleotide and the target DNA at the recombinase recognition sites, thereby inserting the sequence of the donor polynucleotide in the target DNA.

In some embodiments, the recombinase is a serine recombinase.

In some embodiments, the recombinase is a Bxb1 recombinase. In some embodiments, the recombinase is a phiC31 recombinase. In some embodiments, the recombinase is a serine recombinase as described herein, or any serine recombinase known in the art, or any functional variant thereof. In some embodiments, the recombinase recognition site introduced in the target DNA is an attP sequence, and the second recombinase recognition site in the donor polynucleotide is an attB sequence.

In some embodiments, a prime editor system having a recombinase component introduces two or more recombinase recognition sites in the target DNA, target gene, target genome, or target cell, and results in an intended edit in the target DNA, target gene, target genome, or target cell.

In certain embodiments, insertion of a two or more recombinase recognition sites can be accomplished with either PE having single PEgRNAs (multiplexed single flap PE) or twinPE. For example, in some embodiments, a prime editor system comprising a single PEgRNA directs the prime editor system to introduce two or more recombinase recognition sites in a target DNA. In some embodiments, a prime editor system comprises two or more PEgRNAs, wherein each of the two or more PEgRNAs comprises a DNA synthesis template that independently comprises a recombinase recognition site. For example, in some embodiments, a prime editor system comprises a first PEgRNA and a second PEgRNA, wherein the first PEgRNA comprises a first spacer that is complementary to a first target region in a target DNA, and a first DNA synthesis template that comprises a first recombinase recognition site, and wherein the second PEgRNA comprises a second spacer that is complementary to a second target region in a target DNA, and a second DNA synthesis template that comprises a second recombinase recognition site, and wherein the first target region and the second target region are in different positions in the target DNA.

In some embodiments, a prime editor system comprises a pair of PEgRNAs each comprising a DNA synthesis template, wherein the first DNA synthesis template and the second DNA synthesis template comprises a region of complementarity to each other, and wherein the replacement duplex between the first DNA synthesis template and the second DNA synthesis template comprises two or more recombinase recognition sites.

In some embodiments, a prime editor system comprises at least two pair of PEgRNAs each comprising a DNA synthesis template, wherein the first pair comprises a PEgRNA comprising a first DNA synthesis template and a second PEgRNA comprising a second DNA synthesis template, wherein the first DNA synthesis template and the second DNA synthesis template comprises a region of complementarity to each other, and wherein the replacement duplex between the first DNA synthesis template and the second DNA synthesis template comprises a recombinase recognition sites; and wherein the second pair comprises a third DNA a third PEgRNA comprising a third DNA synthesis template and a fourth PEgRNA comprising a fourth DNA synthesis template, wherein the third and the fourth DNA synthesis template comprise a region of complementarity to each other, and wherein the replacement duplex between the third DNA synthesis template and the fourth DNA synthesis template comprises a recombinase recognition site.

In some embodiments, the recombinase is a tyrosine recombinase. The recombinase is a Cre recombinase. In some embodiments, the recombinase is a Flp recombinase. In some embodiments, the recombinase is a tyrosine recombinase disclosed herein, or any tyrosine recombinase known in the art. In some embodiments, the two or more recombinase recognition sites introduced in the target DNA each comprises a Lox sequence. In some embodiments, the two or more recombinase recognition sites introduced in the target DNA each individually comprises a different (orthogonal) Lox sequence, e.g., a LoxP sequence, a Lox511 sequence, a Lox66 sequence, a Lox71 sequence, or a Lox2272 sequence.

In some embodiments, the recombinase is a serine recombinase. In some embodiments, the recombinase is a Bxb1 recombinase. In some embodiments, the two or more recombinase recognition sites each independently comprises an attB sequence or an attP sequence. In some embodiments, the two or more recombinase recognition sites each independently comprises an attB sequence or an attP sequence, wherein the central dinucleotide of the two recombinase recognition sites are the same, e.g., both recombinase recognition sites have GT central dinucleotide or both recombinase recognition sites have GA central dinucleotide. In some embodiments, the central dinucleotide of the two recombinase recognition sites are different, e.g., a first recombinase recognition site has GT central dinucleotide and a second recombinase recognition site has GA central dinucleotide, or vice versa. In some embodiments, the central dinucleotide of attB sequence or the attB sequence is GT. In some embodiments, the central dinucleotide of attB sequence or the attB sequence is GA. In some embodiments, the central dinucleotide of attB sequence or the attB sequence is GC. In some embodiments, the central dinucleotide of attB sequence or the attB sequence is CT.

Recombinase recognition sites introduced by prime editing can be used to generate an intended edit, including deletions, insertions, integrations, and replacement by donor sequences.

In some embodiments, a prime editor system with a recombinase component can result in deletion of one or more nucleotides in a target DNA or target gene. For example, in some embodiments, a prime editor system can result in integration of a first recombinase recognition site and a second recombinase recognition site in the target DNA, wherein the first and the second recombinase recognition sites are in the same orientation, and wherein the recombinase component mediates recombination between the two recombinase recognition sites, thereby resulting in deletion of the sequence in between the first and the second recombinase recognition sites.

In some embodiments, a prime editor system with a recombinase component can result in replacement of an endogenous sequence in a target DNA or a target gene by an exogenous DNA sequence. For example, in some embodiments, a prime editor system can result in a first recombinase recognition site and a second recombinase recognition site in the target DNA. In some embodiments, the prime editor system further comprises a donor DNA, wherein the donor DNA comprises a third and a forth recombinase recognition sites, and wherein the recombinase component mediates recombination between the first recombinase recognition site and the third recombinase recognition site and recombination between the second recombinase recognition site and the fourth recombinase recognition site, thereby resulting in replacement of the sequence between the first and the second recombinase recognition sites in the target DNA by the sequence between the third and the fourth recombinase recognition sites in the donor DNA.

The replacement of an endogenous sequence by a sequence in a donor DNA can be done with either a serine recombinase or a tyrosine recombinase and corresponding recombinase recognition sequences. In some embodiments, the recombinase is a tyrosine recombinase, e.g., a tyrosine recombinase disclosed herein or any tyrosine recombinase known in the art. In some embodiments, the recombinase is a Cre recombinase. In some embodiments, the recombinase is a Flp recombinase. In some embodiments, the two or more recombinase recognition site introduced by the prime editor system into the target DNA each comprises a Lox sequence. In some embodiments, the two or more recombinase recognition sites introduced in the target DNA each individually comprises a different (orthogonal) Lox sequence, for example, a first recombinase recognition site being a LoxP sequence, and the second one being a Lox2272 sequence.

In some embodiments, the recombinase is a serine recombinase, e.g., a serine recombinase disclosed herein or any serine recombinase known in the art. In some embodiments, the recombinase is a Bxb recombinase, and the two recombinase recognition sites introduced into the target DNA by the prime editor system are orthogonal recombinase recognition sites, e.g., an attB-GT sequence and an attB-GA sequence. In such embodiments, the donor DNA sequence comprises two recombinase recognition sites, e.g., an attP-GT sequence and an attP-GA sequence, that can each individually recombine with to the two recombinase recognition sites introduced into the target DNA, wherein the central dinucleotide (GA or GT) controls the recombination between the attB-GA sequence with the attP-GA sequence and the recombination between the attB-GT sequence and the attP-GA sequence.

In some embodiments, a prime editor system with a recombinase component can result in an inversion of a DNA fragment between two nucleotides in a target DNA or target gene. For example in some embodiments, a prime editor system can result in a first recombinase recognition site and a second recombinase recognition site in a target DNA, wherein the first and the second recombinase recognition sites are in opposite directions, and wherein the recombinase component mediates recombination between the first and the second recombinase recognition sites, thereby resulting in inversion of the sequence in the target DNA between the first and the second recombinase recognition sites.

In some embodiments, a prime editor system with a recombinase component can result in an insertion of a DNA fragment between two nucleotides in a target DNA or target gene. For example, in some embodiments, a prime editor system can result in integration of a first recombinase recognition site, a second recombinase recognition site, and a linker sequence between the first and the second recombinase recognition sites in the target DNA. In some embodiments, the linker sequence is an exogenous DNA sequence, e.g., a expression tag or reporter tag. In some embodiments, the prime editor system further comprises a donor DNA, wherein the donor DNA comprises a third and a forth recombinase recognition sites, and wherein the recombinase component mediates recombination between the first recombinase recognition site and the third recombinase recognition site and recombination between the second recombinase recognition site and the fourth recombinase recognition site, thereby resulting in replacement of the sequence between the first and the second recombinase recognition sites in the target DNA by the sequence between the third and the fourth recombinase recognition sites in the donor DNA. Because the linker sequence is exogenous to the target DNA, the effect of the recombination is insertion of the sequence between the third and the fourth recombinase recognition sites in the target DNA.

In some embodiments, a prime editor system with a recombinase component introduces two or more recombinase recognition sites in the target DNA, target gene, target genome, or target cell, and results in two or more intended edits in the target DNA, target gene, target genome, or target cell.

In some embodiments, the two or more intended edits are in the same gene.

In some embodiments, the two or more intended edits are in different genes.

In some embodiments, the two or more intended edits are insertions, deletions, inversions, replacement by exogenous sequences, or any combination thereof.

In some embodiments, the two or more intended edits is different from each other, and is each independently an insertion, a deletion, an inversion, or a replacement by an exogenous sequence.

The instant disclosure provides constructs, systems, and methodologies that leverage the power of prime editing (PE) or twin prime editing (twinPE) to carry out site-specific and large-scale genetic modification, such as, but not limited to, insertions, deletions, inversions, and chromosomal translocations of whole or partial genes (e.g., whole gene, gene exons and/or introns, and gene regulatory regions).

In some embodiments, a prime editor system with a recombinase component result in an insertion of a DNA sequence in a target DNA, target gene, or a target genome.

The insertion can be at any intended position of the target DNA/target gene. In some embodiments, the insertion is within a sequence of a protein encoding gene. In some embodiments, the insertion interrupts the expression of a protein encoding gene, wherein lack of expression of the protein does not have deleterious impact or have beneficial effect when occurs in an individual (i.e. a “safe harbor” gene).

In some embodiments, the insertion is within a CCR5 gene. In some embodiments, the insertion is within a AAVS1 gene. In some embodiments, the insertion is within a PCSK9 gene. In some embodiments, the insertion is within a Rosa26 gene.

In some embodiments, the insertion interrupts the expression of a protein encoding gene, wherein the interruption of expression confers a therapeutic benefit in a cell. In some embodiments, the insertion interrupts the expression of a protein coding gene, wherein the protein coding gene has deleterious effect to the cell or the subject, for example, a gene having a gain of function mutation. In some embodiments, the insertion is at a genomic location that allows expression of the DNA sequence that is inserted. For example, in some embodiments, the insertion is directly downstream of an endogenous gene promoter.

The endogenous promoter can be downstream of any promoter with appropriate expression. In some embodiments, the promoter is a constitutive promoter. In some embodiments, the promoter is an inducible promoter, for example, a promoter responsive to metabolic or environmental signals. In some embodiments, the promoter is a tissue or organ specific promoter.

In some embodiments, the promoter is a promoter of a highly expressed gene. In some embodiments, the insertion is downstream of an albumin promoter. In some embodiments, the insertion is downstream of a hemoglobin promoter.

In some embodiments, the insertion is within an endogenous gene and is downstream of a portion of the endogenous gene. For example, in some embodiments, a target gene includes a 5′ portion and a 3′ portion, where the 3′ portion comprises a mutation associated with a disease. In some embodiments, the inserted DNA sequence is upstream of the mutation. The inserted DNA sequence can be a cDNA or a genomic DNA. In some embodiments, the inserted DNA sequence can be downstream of the 5′ portion, e.g., at the 3′ end of an endogenous exon, wherein the inserted DNA sequence has a wild type sequence corresponding to the 3′ portion of the gene, thereby restoring wild type sequence of the gene. In some embodiments, the inserted DNA sequence includes a stop codon at the 3′ end thereby preventing expression of the remaining portion of the target gene, which includes the mutation.

In some embodiments, the insertion is within an untranslated region of a gene. In some embodiments, the insertion is within a regulatory sequence of a gene.

In some embodiments, the insertion is in a promoter region of a gene. For example, a DNA sequence can be inserted in the promoter region of a gene to regulate expression of the gene. In some embodiments, a DNA sequence comprising at least 50%, 60%, 70%, 80%, 90% or more CpGs can be inserted in the promoter region of a target gene to repress expression of the target gene.

The insertion can be of any length:

In some embodiments, the insertion is about 10, 20, 50, 100, 200, 300, 500, 1000, 2000 nucleotides in length. In some embodiments, the insertion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 kb in length. In some embodiments, the insertion is at least 50 kb in length.

The inserted DNA sequence can be any sequence desired.

In some embodiments, the inserted sequence encodes a protein, or is a portion of a gene that encodes a protein. For example, in some embodiments, a target gene includes a 5′ portion and a 3′ portion, where the 3′ portion comprises a mutation associated with a disease. In some embodiments, the inserted DNA sequence can be downstream of the 5′ portion, e.g., at the 3′ end of an endogenous exon, wherein the inserted DNA sequence has a wild type sequence corresponding to the 3′ portion of the gene, thereby restoring wild type sequence of the gene. In some embodiments, the inserted DNA sequence includes a stop codon at the 3′ end.

In some embodiments, the inserted sequence encodes a therapeutic protein. In some embodiments, the inserted sequence comprises a therapeutic gene or a portion of the therapeutic gene that encodes a therapeutic protein, wherein the target genome comprises one or more endogenous copy of the same gene that comprises a mutation. In some embodiments, the mutation is a loss of function mutation, and expression of the inserted sequence restores or partially restores wild type expression of the protein. In some embodiments, the therapeutic protein is Factor VIII. In some embodiments, the therapeutic protein is adult hemoglobin or fetal hemoglobin.

In some embodiments, the inserted sequence comprises a therapeutic gene or a portion of the therapeutic gene that encodes a therapeutic protein, wherein expression improves subject's health. In some embodiments, the inserted sequence encodes a steroid. In some embodiments, the inserted sequence encodes a growth factor. In some embodiments, the inserted sequence encodes insulin. In some embodiments, the inserted sequence encodes a neurotransmitter. In some embodiments, the inserted sequence encodes dopamine, norepinephrine, epinephrine, histamine, or serotonin. In some embodiments, the inserted sequence encodes a therapeutic protein, e.g., a checkpoint inhibitor protein.

In some embodiments, the inserted sequence encodes an antibody, an antigen receptor polypeptide, or a cell surface marker recognition polypeptide.

In some embodiments, the inserted sequence encodes an antibody, an antigen receptor polypeptide, or a cell surface marker recognition polypeptide that, when expressed in a cell, directs the cell to a specific tissue

In some embodiments, the inserted sequence encodes an antibody, an antigen receptor polypeptide, or a cell surface marker recognition polypeptide that, when expressed in a cell, directs the cell to a specific target cell type that expresses the specific antigen or cell surface receptor. For example, in some embodiments, the inserted sequence encodes an antibody or antigen recognition polypeptide that specifically binds to a surface marker of a tumor cell. In some embodiments, the inserted sequence encodes two or more antibodies, antigen recognition polypeptides, or cell surface receptor recognition polypeptides each recognizing different cell types or antigens.

In some embodiments, the inserted sequence encodes a non-naturally occurring sequence; for example, a chimeric antigen receptor (CAR), or a T-cell receptor, for example, for expression in a T cell. CARs may be inserted in a safe harbor, or in a T cell receptor gene, e.g., TRAC/TRBC. In some embodiments, a sequence encoding the CAR may be inserted in a gene that disrupts natural surface recognition. For example, in some embodiments, a CAR may be inserted in a gene that disrupts natural surface recognition in a T cell, e.g., in TRAC, TRBC1, TRBC2, CIITA, B2M, PD1, such that the disruption prevents fratricide of the CAR-T cell and/or host versus graft/graft versus host disease that result form administration of the CAR-T cell.

In some embodiments, a sequence encoding the CAR may be inserted in a gene involved in immune response or regulation and disrupt its function, e.g., a gene that negatively regulates immune response.

In some embodiments, the inserted sequence encodes a surface polypeptide or protein that allows tracking of the cell where the inserted sequence is expressed. For example, the inserted sequence can encode a “kill switch” that allows the cell expressing the inserted sequence to be targeted by a “kill switch” molecule, e.g., a small molecule such as rimiducid.

In some embodiments, the inserted sequence encodes a polypeptide or a peptide that is involved in protein processing, e.g., protein degradation. In some embodiments, the inserted sequence encodes a degron. In some embodiments, the inserted sequence encodes a ubiquitin. In some embodiments, the inserted sequence encodes an inducible degron, e.g., an auxin inducible degron.

In some embodiments, the inserted sequence encodes a dimerization domain. In some embodiments, the inserted sequence encodes an inducible dimerization domain.

In some embodiments, the inserted sequence is a regulatory sequence, and the insertion can be in an un-translated region of an endogenous gene, for example, upstream of the coding sequence of an endogenous gene.

In some embodiments, the inserted sequence comprises a regulatory sequence, e.g., promoter that enhances expression of an endogenous gene.

In some embodiments, the inserted sequence comprises a tissue specific promoter.

In some embodiments, the inserted sequence comprises an enhancer.

In some embodiments, the inserted sequence comprises a repressor. In some embodiments, the inserted sequence comprises an insulator, for example, a sequence that is recognized by a cellular CTCF protein.

In some embodiments, the inserted sequence comprises one or more regulatory sequences and one or more sequences encoding one or more polypeptides.

In some embodiments, the inserted sequence comprises an expression cassette comprising a promoter, an open reading frame, and optionally a terminator.

In some embodiments, the inserted sequence comprises two or more expression cassettes each comprising a promoter, an open reading frame, and optionally a terminator.

In some embodiments, the inserted sequence further comprises polynucleotide sequences that encode peptide linkers, nuclear localization signals, T2A sequences, or expression tags.

In some embodiments, a prime editor system with a recombinase component results in replacement of an endogenous sequence in a target DNA by an exogenous DNA sequence, or replacement of a target gene by an exogeneous DNA sequence.

For example, in some embodiments, the target DNA is a target gene that comprises one or more mutations associated with a disease. In some embodiments, a fragment of the target gene that includes the one or more mutations can be replaced with an exogenous DNA sequence that has wild type sequence of the same gene corresponding to the endogenous portion that comprises the one or more disease associated mutations.

In some embodiments, the replaced endogenous fragment is in a coding sequence of the target gene. In some embodiments, the replaced endogenous fragments includes one exon and one or both introns flanking the exon. In some embodiments, the replaced endogenous fragment includes multiple exons and intervening introns thereof. In some embodiments, the replaced endogenous fragment is at a splice site of the target gene, and the replacement restores wild type splicing pattern of the target gene.

In some embodiments, the replaced endogenous fragment comprises 1, 2, 3, 4, 5 or more mutations compared to a wild type gene. In some embodiments, the replaced endogenous fragment corresponds to a fragment of the gene, within which at least 1, 2, 3, 4, 5, or more mutations have been identified in same or different individuals or populations.

In some embodiments, the endogenous fragment is replaced by a cDNA, wherein the cDNA does not include any intron sequence.

In some embodiments, the replaced endogenous fragment is in a non-coding region, e.g., a regulatory region.

In some embodiments, the replaced endogenous fragment comprises both non-coding and coding regions.

In some embodiments, the replacement of a target DNA or target gene requires two recombinase recognition sites. The recombinase recognition sites flank on either side of the region to be replaced.

In some embodiments, for replacement of a target gene, one RRS is upstream of the coding region and one RRS is downstream of the coding region. In some embodiments, for replacement of a target gene, one RRS is upstream of the 5′ regulatory sequences of the target gene and one RRS is downstream of the 3′ UTR.

In some embodiments, an endogenous sequence in a target DNA, such as a portion of a gene, is replaced. The RRSs flanking the region to be replaced may be in exons, introns, upstream of 5′ regulatory sequences, downstream of 3′ UTRs, or any combination thereof.

In some embodiments, a prime editor system with a recombinase component results in deletion of a fragment in a target DNA or a target gene.

The deletion can be of any length.

In some embodiments, the deletion is about 10, 20, 50, 100, 200, 300, 500, 1000, 2000 nucleotides in length.

In some embodiments, the deletion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 kb in length,

In some embodiments, the deletion is at least 50 kb in length.

The deletion can be in any region of a gene or a genome, for example, by placing two recombinase recognition sites on each side of the region to be deleted, wherein the two recombinase recognition sites are in the same orientation.

In some embodiments, the deletion is in a non-coding region, e.g., a regulatory region, of a gene.

In some embodiments, the deletion comprises both non-coding and coding regions of a gene.

In some embodiments, for deletion of a target gene, one recombinase recognition site is upstream of the coding region and one recombinase recognition site is downstream of the coding region. In some embodiments, for deletion of a target gene, one recombinase recognition site is upstream of the 5′ regulatory sequences of the target gene and one recombinase recognition site is downstream of the 3′ UTR.

In some embodiments, a endogenous sequence in a target DNA, such as a portion of a gene, is replaced. The recombinase recognition sites flanking the region to be deleted may be in exons, introns, upstream of 5′ regulatory sequences, downstream of 3′ UTRs, or any combination thereof.

The deleted DNA sequence can be any sequence desired.

In some embodiments, a whole gene or a portion of a gene is deleted.

In some embodiments, an exon or an intron is deleted. In some embodiments, two or more exons and intervening introns are deleted. In some embodiments, an untranslated region, e.g., a regulatory sequence, of a target gene, is deleted.

In some embodiments, a target gene comprises a series of abnormally expanded trinucleotide repeats compared to a wild type gene sequence. Such aberrant expansions in trinucleotide repeats can be deleted by a prime editor system with a recombinase component provided herein.

In some embodiments, deletion of a portion of a target gene, for example, one or more exons and intervening intron sequences, allows for restoration of a wild type reading frame. For example, mutations in the DMD gene can result in frameshifting and premature stop codons in the gene, which leads to lack of functional dystrophin protein and causes Duchenne or Becker forms of muscular dystrophy. In some embodiments, one or more exons, e.g., exon 51, is deleted by a prime editor system with a recombinase component for restoration of the reading frame.

In some embodiments, the deletion comprises a duplicated gene.

In some embodiments, the deletion comprises a transposable element in the target gene, for example a transposable element associated with a disease, including a LINE element or an Alu element.

In some embodiments, the deletion comprises a viral element in the target DNA, e.g., a target genome.

In some embodiments, a prime editor system with a recombinase component results in an inversion of a fragment between two nucleotides in a target DNA or a target gene. For example, in some embodiments, a prime editor system can integrate two recombinase recognition sites at each end of a sequence to be inverted, wherein the two recombinase recognition sites are in opposite directions, and wherein the recombinase component of the prime editor system mediates recombination between the two recombinase recognition sites, thereby resulting in inversion of the sequence in the target DNA between the integrate two recombinase recognition sites.

The inverted fragment can be of any length.

In some embodiments, the inverted fragment is about 10, 20, 50, 100, 200, 300, 500, 1000, 2000 nucleotides in length.

In some embodiments, the inverted fragment is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 kb in length.

In some embodiments, the inverted fragment is at least 50 kb in length.

The inverted DNA sequence or fragment can be any sequence desired.

In some embodiments, a prime editor system having a recombinase component restores a wild-type sequence in a target gene that comprises a mutation due to an inversion. For example, in Factor VIII, inversion of mutations caused by inversion of a portion of a gene, e.g., inversion of F8 intron 22 or intron 1 are major causes of hemophilia A. in some embodiments, inversion of intron 22 or intron 1 of F8 corrects the inversion mutation, and restores wild type sequence of the F8 gene.

In some embodiments, the prime editor system with a recombinase component is a Regulatable system.

Components of the prime editor system, e.g., the prime editor protein, the PEgRNA(s) or pair(s) of PEgRNAs, may be introduced to simultaneously or sequentially to a target DNA or a target cell.

In some embodiments, a prime editor system introduces a regulatory sequence, e.g., a promoter, upstream of a target gene. In some embodiments, the regulatory sequence is flanked by two recombinase recognition sequences. in some embodiments, a corresponding recombinase or a polynucleotide encoding the recombinase is introduced to the target genome or target cell after expression of the target gene and deletes the regulatory sequence, thereby stopping expression of the target gene. in some embodiments, a corresponding recombinase or a polynucleotide encoding the recombinase is introduced to the target genome or target cell after expression of the target gene and inverts the regulatory sequence, thereby stopping expression of the target gene.

In some embodiments, a prime editor system introduces an inverted regulatory sequence, e.g., a promoter, upstream of a target gene. In some embodiments, the inverted regulatory sequence is flanked by two recombinase recognition sequences. in some embodiments, a corresponding recombinase or a polynucleotide encoding the recombinase is later introduced to the target genome or target cell to invert the regulatory sequence to the correct order, thereby activating expression of the target gene.

In one aspect, the present disclosure provides methods for simultaneously editing a first and a second complementary strands of a double-stranded DNA sequence at a target site, said method comprising contacting the double-stranded DNA sequence with a pair of prime editor complexes, said pair comprising:
a. a first prime editor complex, comprising:
i. a first prime editor comprising a first nucleic acid programmable DNA binding protein (napDNAbp) and a first polypeptide comprising an RNA-dependent DNA polymerase activity; and
ii. a first prime editing guide RNA (first PEgRNA) that binds to a first binding site on the first strand of the genomic DNA sequence upstream of the target site;
b. a second prime editor complex, comprising:
i. a second prime editor comprising a second nucleic acid programmable DNA binding protein (second napDNAbp) and a second polypeptide comprising an RNA-dependent DNA polymerase activity; and
ii. a second prime editing guide RNA (second PEgRNA) that binds to a second binding site on the second strand of the genomic DNA sequence downstream of the target site;
wherein the first prime editor complex causes a first nick at a sequence complementary to the first binding site and the subsequent polymerization of a first single-stranded DNA sequence having a 3′-end from the available 5′-end formed by the first nick;
wherein the second prime editor complex causes a second nick at a sequence complementary to the second binding site and the subsequent polymerization of a second single-stranded DNA sequence having a 3′-end from the available 5′-end formed by the second nick;
wherein the first single-stranded DNA sequence and the second single-stranded DNA sequence are reverse complements over at least a region of complementarity and form a duplex comprising an edit; and
wherein the duplex replaces the nicked first and second complementary strands of the double-stranded DNA sequence.

In another aspect, the present disclosure provides methods for simultaneously editing first and second complementary strands of a double-stranded DNA sequence at a target site, the method comprising contacting the double-stranded DNA sequence with a pair of prime editor complexes, the pair comprising:
(a) a first prime editor complex, comprising:
i. a first prime editor comprising a first nucleic acid programmable DNA binding protein (first napDNAbp) and a first polypeptide comprising an RNA-dependent DNA polymerase activity; and
ii. a first prime editing guide RNA (first PEgRNA) that binds to a first target sequence on the first strand of the genomic DNA sequence upstream of the target site;
(b) a second prime editor complex, comprising:
i. a second prime editor comprising a second nucleic acid programmable DNA binding protein (second napDNAbp) and a second polypeptide comprising an RNA-dependent DNA polymerase activity; and
ii. a second prime editing guide RNA (second PEgRNA) that binds to a second target sequence on the second strand of the genomic DNA sequence upstream of the target site;
(c) a third prime editor complex, comprising:
i. a third prime editor comprising a third nucleic acid programmable DNA binding protein (third napDNAbp) and a third polypeptide comprising an RNA-dependent DNA polymerase activity; and
ii. a third prime editing guide RNA (third PEgRNA) that binds to a third target sequence on the first strand of the genomic DNA sequence downstream of the target site;
(d) a fourth prime editor complex, comprising:
i. a fourth prime editor comprising a second nucleic acid programmable DNA binding protein (fourth napDNAbp) and a fourth polypeptide comprising an RNA-dependent DNA polymerase activity; and
ii. a fourth prime editing guide RNA (fourth PEgRNA) that binds to a fourth target sequence on the second strand of the genomic DNA sequence downstream of the target site;
wherein the first prime editor complex causes a first nick at the first target sequence and the subsequent polymerization of a first single-stranded DNA sequence having a 3′-end from the available 5′-end formed by the first nick;
wherein the second prime editor complex causes a second nick at the second target sequence and the subsequent polymerization of a second single-stranded DNA sequence having a 3′-end from the available 5′-end formed by the second nick;
wherein the third prime editor complex causes a third nick at the third target sequence and the subsequent polymerization of a third single-stranded DNA sequence having a 3′-end from the available 5′-end formed by the third nick;
wherein the fourth prime editor complex causes a fourth nick at the fourth target sequence and the subsequent polymerization of a fourth single-stranded DNA sequence having a 3′-end from the available 5′-end formed by the fourth nick;
wherein the first single-stranded DNA sequence and the second single-stranded DNA sequence are reverse complements over at least a region of complementarity and form a duplex, wherein the duplex replaces the nicked first and second complementary strands of the double-stranded DNA sequence; and
wherein the third single-stranded DNA sequence and the fourth single-stranded DNA sequence are reverse complements over at least a region of complementarity and form a duplex, wherein the duplex replaces the nicked first and second complementary strands of the double-stranded DNA sequence.

This Specification describes PE, twinPE, and multi-flap prime editing systems (including, for example, a quadruple-flap prime editing system) that address the challenges associated with flap equilibration and subsequent incorporation of the edit into the non-edited complementary genomic DNA strand by simultaneously editing both DNA strands. In the dual-flap prime editing system, two PEgRNAs are used to target opposite strands of a genomic site and direct the synthesis of two complementary 3′ flaps containing edited DNA sequence. In the quadruple-flap prime editing system, four PEgRNAs are used and direct the synthesis of four 3′ flaps, two of which are complementary to one another and the other two of which are complementary to one another. Unlike classical prime editing, there is no requirement for the pair of edited DNA strands (3′ flaps) to directly compete with 5′ flaps in endogenous genomic DNA, as the complementary edited strand is available for hybridization instead. Since both strands of the duplex are synthesized as edited DNA, the multi-flap prime editing system obviates the need for the replacement of the non-edited complementary DNA strand required by classical prime editing. Instead, cellular DNA repair machinery need only excise the paired 5′ flaps (original genomic DNA) and ligate the paired 3′ flaps (edited DNA) into the locus. Therefore, there is also no need to include sequences homologous to genomic DNA in the newly synthesized DNA strands, allowing selective hybridization of the new strands and facilitating edits that contain minimal genomic homology. Nuclease-active versions of multi-flap prime editors that cut both strands of DNA could also be used to accelerate the removal of the original DNA sequence.

Like classical prime editing, multi-flap prime editing is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the dual prime editors, as described herein, not only search and locate the desired target site to be edited, but at the same time, encode a replacement strand containing a desired edit which is installed in place of the corresponding target site endogenous DNA strand.

In dual-flap prime editing, a double-stranded DNA sequence is contacted at a target site with a first and a second prime editor complex. Each complex comprises a fusion protein and a PEgRNA. In some embodiments, each fusion protein comprises a nucleic acid programmable DNA binding protein (napDNAbp) and a polypeptide having an RNA-dependent DNA polymerase activity (e.g., a reverse transcriptase), and each PEgRNA comprises a spacer sequence, a gRNA core, a DNA synthesis template, and a primer binding site. Each DNA synthesis template can encode a single-stranded DNA sequence, which may comprise an edited portion of one or more nucleotides. The two single-stranded DNA sequences encoded may be complementary to one another and form a duplex, which can integrate into the target site to be edited. The various elements of the prime editor complexes (e.g., fusion proteins, napDNAbp, polymerase, PEgRNAs, etc.) may comprise any of the embodiments of the systems disclosed herein.

In quadruple-flap prime editing, a double-stranded DNA sequence is contacted at a target site with a first, a second, a third, and a fourth prime editor complex. Each complex comprises a fusion protein and a PEgRNA. In some embodiments, each fusion protein comprises a nucleic acid programmable DNA binding protein (napDNAbp) and a polypeptide having an RNA-dependent DNA polymerase activity (e.g., a reverse transcriptase), and each PEgRNA comprises a spacer sequence, a gRNA core, a DNA synthesis template, and a primer binding site. Each DNA synthesis template encodes a single-stranded DNA sequence. The two single-stranded DNA sequences encoded may be complementary to one another and form a duplex, which can integrate into the target site to be edited. The various elements of the prime editor complexes (e.g., fusion proteins, napDNAbp, polymerase, PEgRNAs, etc.) may comprise any of the embodiments of the systems disclosed herein.

The methods for multi-flap prime editing provided herein can be used for numerous applications. For example, they can be used to facilitate the inversion of a target DNA sequence. In this application, a first single-stranded DNA sequence encoded by the DNA synthesis template of the first PEgRNA and a second single-stranded DNA sequence encoded by the DNA synthesis template of the second PEgRNA are on opposite ends of a target DNA sequence, and a third single-stranded DNA sequence encoded by the DNA synthesis template of the third PEgRNA and a fourth single-stranded DNA sequence encoded by the DNA synthesis template of a fourth PEgRNA are on opposite ends of the same target DNA sequence.

In some embodiments, the methods for multi-flap prime editing provided herein further comprise providing a circular DNA donor, part of which can be integrated into a double-stranded nucleic acid at a target site. In this application, a first single-stranded DNA sequence encoded by the DNA synthesis template of the first PEgRNA and a third single-stranded DNA sequence encoded by the DNA synthesis template of the third PEgRNA are on opposite ends of the target DNA sequence, and a second single-stranded DNA sequence encoded by the DNA synthesis template of the second PEgRNA and a fourth single-stranded DNA sequence encoded by the DNA synthesis template of the fourth PEgRNA are on the circular DNA donor. The portion of the circular DNA donor between the second single-stranded DNA sequence and the fourth single-stranded DNA sequence can form a duplex, which replaces the target DNA sequence between the first single-stranded DNA sequence and the third single-stranded DNA sequence.

In another application, the methods for multi-flap prime editing provided herein allow for translocation of a target DNA sequence from a first nucleic acid molecule (e.g., a first chromosome) to a second nucleic acid molecule (e.g., a second chromosome). In this application, a first single-stranded DNA sequence encoded by the DNA synthesis template of the first PEgRNA and a third single-stranded DNA sequence encoded by the DNA synthesis template of the third PEgRNA are on a first nucleic acid molecule, and a second single-stranded DNA sequence encoded by the DNA synthesis template of the second PEgRNA and a fourth single-stranded DNA sequence encoded by the DNA synthesis template of the fourth PEgRNA are on a second nucleic acid molecule. The portion of the first nucleic acid molecule between the first single-stranded DNA sequence and the third single-stranded DNA sequence can be incorporated into the second nucleic acid molecule, and the portion of the second nucleic acid molecule between the second single-stranded DNA sequence and the fourth single-stranded DNA sequence is incorporated into the first nucleic acid molecule.

### Pharmaceutical Compositions

Other aspects of the present disclosure relate to pharmaceutical compositions comprising any of the various components of the PE, twinPE, and multi-flap prime editing systems described herein (e.g., including, but not limited to, the napDNAbps, reverse transcriptases, fusion proteins (e.g., comprising napDNAbps and reverse transcriptases), extended guide RNAs, and complexes comprising fusion proteins and extended guide RNAs, as well as accessory elements, such as second strand nicking components and 5′ endogenous DNA flap removal endonucleases for helping to drive the multi-flap prime editing process towards the edited product formation).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[27bp spacer]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits, Cells, Vectors, and Delivery

### Kits

The compositions of the present disclosure involving the PE, twinPE, and multi-flap PE systems may be assembled into kits. In some embodiments, the kit comprises nucleic acid vectors for the expression of the PE, twinPE, or multi-flap prime editors described herein. In other embodiments, the kit further comprises appropriate guide nucleotide sequences (e.g., PEgRNAs and second-site gRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively, it may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the multi-flap prime editing systems (e.g., dual prime editing and quadruple prime editing systems) described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the multi-flap prime editing process towards the edited product formation). In some embodiments, the nucleotide sequence(s) comprises a heterologous promoter (or more than a single promoter) that drives expression of the multi-flap prime editing system components.

Other aspects of this disclosure provide kits comprising one or more nucleic acid constructs encoding the various components of the multi-flap prime editing systems described herein, e.g., comprising a nucleotide sequence encoding the components of the multi-flap prime editing system capable of modifying a target DNA sequence. In some embodiments, the nucleotide sequence comprises a heterologous promoter that drives expression of the multi-flap prime editing system components.

Some aspects of this disclosure provide kits comprising a nucleic acid construct, comprising (a) a nucleotide sequence encoding a napDNAbp (e.g., a Cas9 domain) fused to a reverse transcriptase and (b) a heterologous promoter that drives expression of the sequence of (a).

### Cells

Cells that may contain any of the PE, twinPE, and/or multi-flap PE compositions described herein include prokaryotic cells and eukaryotic cells. The methods described herein are used to deliver a Cas9 protein or a multi-flap prime editor into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalc1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Vectors

Some aspects of the present disclosure relate to using recombinant virus vectors (e.g., adeno-associated virus vectors, adenovirus vectors, or herpes simplex virus vectors) for the delivery of the PE, twinPE, and/or multi-flap prime editors or components thereof described herein, e.g., the split Cas9 protein or a split nucleobase multi-flap prime editors, into a cell. In the case of a split-PE approach, the N-terminal portion of a PE fusion protein and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or multi-flap prime editors exceeds the packaging limit of various virus vectors, e.g., rAAV (˜4.9 kb).

Thus, in one embodiment, the disclosure contemplates vectors capable of delivering split multi-flap prime editor fusion proteins, or split components thereof. In some embodiments, a composition for delivering the split Cas9 protein or split prime editor into a cell (e.g., a mammalian cell, a human cell) is provided. In some embodiments, the composition of the present disclosure comprises: (i) a first recombinant adeno-associated virus (rAAV) particle comprising a first nucleotide sequence encoding a N-terminal portion of a Cas9 protein or prime editor fused at its C-terminus to an intein-N; and (ii) a second recombinant adeno-associated virus (rAAV) particle comprising a second nucleotide sequence encoding an intein-C fused to the N-terminus of a C-terminal portion of the Cas9 protein or prime editor. The rAAV particles of the present disclosure comprise a rAAV vector (i.e., a recombinant genome of the rAAV) encapsidated in the viral capsid proteins.

In some embodiments, the rAAV vector comprises: (1) a heterologous nucleic acid region comprising the first or second nucleotide sequence encoding the N-terminal portion or C-terminal portion of a split Cas9 protein or a split multi-flap prime editor in any form as described herein, (2) one or more nucleotide sequences comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of a cell. In some embodiments, viral sequences that facilitate integration comprise Inverted Terminal Repeat (ITR) sequences. In some embodiments, the first or second nucleotide sequence encoding the N-terminal portion or C-terminal portion of a split Cas9 protein or a split multi-flap prime editor is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6.

Thus, in some embodiments, the rAAV particles disclosed herein comprise at least one rAAV2 particle, rAAV6 particle, rAAV8 particle, rPHP.B particle, rPHP.eB particle, or rAAV9 particle, or a variant thereof. In particular embodiments, the disclosed rAAV particles are rPHP.B particles, rPHP.eB particles, rAAV9 particles.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators of the bovine growth hormone gene (bGH), human growth hormone gene (hGH), SV40, CW3, ϕ, or combinations thereof. The efficiencies of several transcriptional terminators have been tested to determine their respective effects in the expression level of the split Cas9 protein or the split multi-flap prime editor. In some embodiments, the transcriptional terminator used in the present disclosure is a bGH transcriptional terminator. In some embodiments, the rAAV vector further comprises a Woodchuck Hepatitis Virus Posttranscriptional Regulatory Element (WPRE). In certain embodiments, the WPRE is a truncated WPRE sequence, such as “W3.” In some embodiments, the WPRE is inserted 5′ of the transcriptional terminator. Such sequences, when transcribed, create a tertiary structure which enhances expression, in particular, from viral vectors.

In some embodiments, the vectors used herein may encode the PE fusion proteins, or any of the components thereof (e.g., napDNAbp, linkers, or polymerases). In addition, the vectors used herein may encode the PEgRNAs, and/or the accessory gRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFlα promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the prime editor vectors (e.g., including any vectors encoding the prime editor fusion protein and/or the PEgRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor fusion protein and/or the PEgRNAs, and/or the accessory second strand nicking gRNAs) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the nucleotide sequence encoding the PEgRNA (or any guide RNAs used in connection with multi-flap prime editing) may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript. In some embodiments, the intracellular half-life of the PE fusion protein transcript may be reduced by containing the guide RNA within its 3′ UTR and thereby shortening the length of its 3′ UTR. In additional embodiments, the guide RNA may be within an intron of the PE fusion protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The multi-flap prime editor vector system may comprise one vector, or two vectors, or three vectors, or four vectors, or five vector, or more. In some embodiments, the vector system may comprise one single vector, which encodes both the PE fusion protein and PEgRNA. In other embodiments, the vector system may comprise two vectors, wherein one vector encodes the PE fusion protein and the other encodes the PEgRNA. In additional embodiments, the vector system may comprise three vectors, wherein the third vector encodes the second strand nicking gRNA used in the herein methods.

In some embodiments, the composition comprising the rAAV particle (in any form contemplated herein) further comprises a pharmaceutically acceptable carrier. In some embodiments, the composition is formulated in appropriate pharmaceutical vehicles for administration to human or animal subjects.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Delivery Methods

In some aspects, the invention provides methods comprising delivering one or more polynucleotides encoding the various components of the multi-flap prime editors described herein, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

Exemplary delivery strategies are described herein elsewhere, which include vector-based strategies, PE ribonucleoprotein complex delivery, and delivery of PE by mRNA methods.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electroporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of fusion proteins markedly increases the DNA specificity of base editing. RNP delivery of fusion proteins leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

Additional methods for the delivery of nucleic acids to cells are known to those skilled in the art. See, for example, US 2003/0087817, incorporated herein by reference.

Other aspects of the present disclosure provide methods of delivering the multi-flap prime editor constructs into a cell to form a complete and functional prime editor within a cell. For example, in some embodiments, a cell is contacted with a composition described herein (e.g., compositions comprising nucleotide sequences encoding the split Cas9 or the split prime editor or AAV particles containing nucleic acid vectors comprising such nucleotide sequences). In some embodiments, the contacting results in the delivery of such nucleotide sequences into a cell, wherein the N-terminal portion of the Cas9 protein or the prime editor and the C-terminal portion of the Cas9 protein or the prime editor are expressed in the cell and are joined to form a complete Cas9 protein or a complete prime editor.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a split protein), or transfected (e.g., with a plasmid encoding a split protein) with a nucleic acid molecule that encodes a split protein, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences, for example in delivery of a split Cas9 (e.g., nCas9) protein. In some embodiments, a plasmid expressing a split protein may be introduced into cells through electroporation, transient (e.g., lipofection) and stable genome integration (e.g., piggybac) and viral transduction or other methods known to those of skill in the art.

In certain embodiments, the compositions provided herein comprise a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

The guide RNA sequence may be 15-100 nucleotides in length and comprise a sequence of at least 10, at least 15, or at least 20 contiguous nucleotides that is complementary to a target nucleotide sequence. The guide RNA may comprise a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target nucleotide sequence. The guide RNA may be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the target nucleotide sequence is a DNA sequence in a genome, e.g. a eukaryotic genome. In certain embodiments, the target nucleotide sequence is in a mammalian (e.g. a human) genome.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

Treatment of a disease or disorder includes delaying the development or progression of the disease, or reducing disease severity. Treating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Introduction of Recombinase Target Sites with PE

This Example describes a method to address genetic disease or generate tailor-made animal or plant models by using prime editing (PE) to introduce site-specific recombinase target sites (SSR target sites) in mammalian and other genomes with high specificity and efficiency.

This Example describes use of PE to introduce recombinase recognition sequences at high-value loci in human or other genomes, which, after exposure to site-specific recombinase(s), will direct precise and efficient genomic modifications (FIG. 1). In various embodiments shown in FIG. 1, PE may be used to (b) insert a single SSR target for use as a site for genomic integration of a DNA donor template. (c) shows how a tandem insertion of SSR target sites can be used to delete a portion of the genome. (d) shows how a tandem insertion of SSR target sites can be used to invert a portion of the genome. (e) shows how the insertion of two SSR target sites at two distal chromosomal regions can result in chromosomal translocation. (f) shows how the insertion of two different SSR target sites in the genome can be used to exchange a cassette from a DNA donor template. Each of the types of genome modifications are envisioned by using PE to insert SSR targets, but this list also is not meant to be limiting.

Many large-scale genomic changes, such as gene insertions, deletions, inversions, replacements, or chromosomal translocations, are implicated in genetic disease¹⁻⁷. In addition, custom and targeted manipulation of eukaryotic genomes is important for research into human disease, as well as generation of transgenic plants^{8,9} or other biotechnological products. For example, microdeletions of chromosomes can lead to disease, and replacement of these deletions by insertions of critical DNA elements could lead to a permanent amelioration of disease. In addition, diseases resulting from inversions, gene copy number changes, or chromosomal translocations could be addressed by restoring the previous gene structure in affected cells. Alternatively, in plants or other high value eukaryotic organisms used in industry, introduction of recombinant DNA or targeted genomic rearrangements could lead to improved products, for example, crops which require fewer resources or are resistant to pathogens. Current technologies for effecting large-scale genomic changes rely on random or stochastic processes, for example the use of transposons or retroviruses, while other desired genomic modifications have only been achieved by homologous recombination strategies.

One appealing class of proteins for accomplishing targeted and efficient genomic modification is site-specific recombinases (SSRs). SSRs have a long history of being used as a tool for genomic modification¹⁰⁻¹³. SSRs are considered promising tools for gene therapy because they catalyze the precise cleavage, strand exchange, and rejoining of DNA fragments at defined recombination targets¹⁴ without relying on the endogenous repair of double-strand breaks which can induce indels, translocations, other DNA rearrangements, or p53 activation¹⁵⁻¹⁸ The reactions catalyzed by SSRs can result in the direct replacement, insertion, or deletion of target DNA fragments with efficiencies exceeding those of homology-directed repair¹⁴⁻¹⁹.

Although SSRs offer many advantages, they are not widely used because they have a strong innate preference for their cognate target sequence. The recognition sequences of SSRs are typically ≥20 base pairs and thus unlikely to occur in the genomes of humans or model organisms. Further, the native substrate preferences of SSRs are not easily altered, even with extensive laboratory engineering or evolution²⁰. This limitation is overcome by using PE to directly introduce recombinase targets into the genome, or to modify endogenous genomic sequences which natively resemble recombinase targets. Subsequent exposure of the cell to recombinase protein will permit precise and efficient genomic modification directed by the location and orientation of the recombinase target(s) (FIG. 1).

PE-mediated introduction of recombinase targets could be particularly useful for the treatment of genetic diseases which are caused by large-scale genomic defects, such as gene loss, inversion, or duplication, or chromosomal translocation¹⁻⁷(Table 1). For example, Williams-Beuren syndrome is a developmental disorder caused by a deletion of 24 in chromosome 721. No technology exists currently for the efficient and targeted insertion of multiple entire genes in living cells (the potential of PE to do such a full-length gene insertion is currently being explored but has not yet been established); however, recombinase-mediated integration at a target inserted by PE offers one approach towards a permanent cure for this and other diseases. In addition, targeted introduction of recombinase recognition sequences could be highly enabling for applications including generation of transgenic plants, animal research models, bioproduction of cell lines, or other custom eukaryotic cell lines. For example, recombinase-mediated genomic rearrangement in transgenic plants at PE-specific targets could overcome one of the bottlenecks to generating agricultural crops with improved properties^{8,9.}

A number of SSR family members have been characterized and their target sequences described, including natural and engineered tyrosine recombinases (Table 2), large serine integrases (Table 3), serine resolvases (Table 4), and tyrosine integrases (Table 5). Modified target sequences that demonstrate enhanced rates of genomic integration have also been described for several SSRs²²⁻³⁰. In addition to natural recombinases, programmable recombinases with distinct specificities have been developed³¹⁻⁴⁰. Using PE, one or more of these recognition sequences could be introduced into the genome at a specified location, such as a safe harbor locus⁴¹⁻⁴³, depending on the desired application.

For example, introduction of a single recombinase target in the genome would result in integrative recombination with a DNA donor template (FIG. 1B). Serine integrases, which operate robustly in human cells, may be especially well-suited for gene integration^{44,45}. Additionally, introduction of two recombinase targets could result in deletion of the intervening sequence, inversion of the intervening sequence, chromosomal translocation, or cassette exchange, depending on the identity and orientation of the targets (FIGS. 1C-1F). By choosing endogenous sequences that already closely resemble recombinase targets, the scope of editing required to introduce the complete recombinase target would be reduced.

Finally, several recombinases have been demonstrated to integrate into human or eukaryotic genomes at natively occurring pseudosites⁴⁶⁻⁶⁴. Prime editing could be used to modify these loci to enhance rates of integration at these natural pseudosites, or alternatively, to eliminate pseudosites that may serve as unwanted off-target sequences.

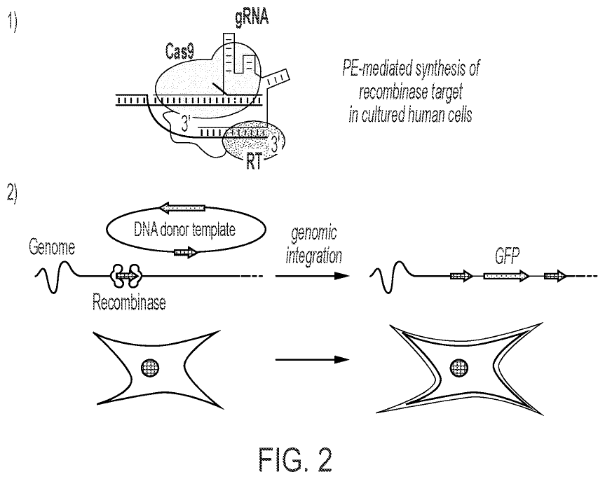

This report describes a general methodology for introducing recombinase target sequences in eukaryotic genomes using PE, the applications of which are nearly limitless. The genome editing reactions are intended for use with “prime editors,” a chimeric fusion of a CRISPR/Cas9 protein and a reverse-transcriptase domain, which utilizes a custom prime editing guide RNA (PEgRNA). By extension, Cas9 tools and homology-directed repair (HDR) pathways may also be exploited to introduce recombinase targets through DNA templates by lowering the rates of indels using several techniques⁶⁵⁻⁶⁷. A proof-of-concept experiment in human cell culture is shown in FIG. 2.

TABLE 1
Examples of genetic diseases linked to
large-scale genomic modifications.

| Disease | Source | Cause |
|---|---|---|
| Trisomy 17p | 68 | Gene duplication |
| Charcot-Marie-Tooth disease type I | 69 | Gene duplication |
| Smith-Magenis syndrome | 70 | Gene deletion |
| Williams-Beuren syndrome | 21 | Gene deletion |
| De la Chapelle syndrome | 71 | Chromosomal |
| | | translocation |
| Down syndrome (some forms) | 72 | Chromosomal |
| | | translocation |
| Hemophilia A | 73 | Gene inversion |
| Hunter syndrome | 74 | Gene inversion |

Table 2. Tyrosine recombinases and SSR target sequences.

+0 TABLE 2
Tyrosine recombinases and SSR target sequences.

| | | | | SEQ ID |
|---|---|---|---|---|
| Recombinase | Source | Recombinase recognition sequence | Name | NO: |
| Cre | 75 | ATAACTTCGTATAGCATACATTATACGAAGTTAT | loxP | 124 |
| Dre | 76 | TAACTTTAAATAATGCCAATTATTTAAAGTTA | rox | 125 |
| VCre | 77 | TCAATTTCTGAGAACTGTCATTCTCGGAAATTGA | loxV | 126 |
| SCre | 77 | CTCGTGTCCGATAACTGTAATTATCGGACATGAT | loxS | 127 |
| Flp | 78 | GAAGTTCCTATTCTCTAGAAAGTATAGGAACTTC | FRT | 128 |
| B2 | 79 | GAGTTTCATTAAGGAATAACTAATTCCCTAATGA | loxB | 129 |
| | | AACTC | | |
| B3 | 79 | GGTTGCTTAAGAATAAGTAATTCTTAAGCAACC | loxB3 | 130 |
| Kw | 80 | ACGAAAAATGGTAAGGAATAGACCATTCCTTAC | | 131 |
| | | CATTTTTGGT | | |
| R | 81 | TTGATGAAAGAATAACGTATTCTTTCATCAA | RS | 132 |
| TD1-40 | 82 | GTGCGTCAAATAATAACGTATTATTTGACACTT | TDRS | 133 |
| Vika | 83 | AATAGGTCTGAGAACGCCCATTCTCAGACGTATT | vox | 134 |
| Nigri | 84 | TGAATGTCCTATAATTACACTTATAGGACATTCA | nox | 135 |
| Panto | 84 | GAAACTTTAAATAATAAGTCTTATTTAAAGTTTC | pox | 136 |
| Kd | 79 | AAACGATATCAGACATTTGTCTGATAATGCTTCA | loxK | 137 |
| | | TTATCAGACAAATGTCTGATATCGTTT | | |
| Fre | 85 | ATATATACGTATATAGACATATATACGTATATAT | loxH | 138 |
| CreALSHG | 86 | ATAACTCTATATAATGTATGCTATATAGAGTTAT | loxM7 | 139 |
| Tre | 87 | ACAACATCCTATTACACCCTATATGCCAACATGG | loxLTR | 140 |
| Brec 1 | 12 | AACCCACTGCTTAAGCCTCAATAAAGCTTGCCTT | loxBTR | 141 |
| Cre-R3M3 | 88 | GATACAACGTATATACCTTTCTATACGTTGTTTA | loxK2 | 142 |

Table 3. Large serine integrases and SSR target sequences.

+0 TABLE 3
Large serine integrases and SSR target sequences.

| | | | SEQ | | SEQ |
|---|---|---|---|---|---|
| | | | ID | | ID |
| Integrase | Source | Left Target | NO: | Right Target | NO: |
| Bxb1 | 89 | GGTTTGTCTGGTCAACCACC | 143 | GGCTTGTCGACGACGGCGG | 175 |
| | | GCGGTCTCAGTGGTGTACGG | | TCTCCGTCGTCAGGATCAT | |
| | | TACAAACC | | | |
| phiC31 | 90 | GTGCCCCAACTGGGGTAACC | 144 | TGCGGGTGCCAGGGCGTGC | 176 |
| | | TTTGAGTTCTCTCAGTTGGG GG | | CCTTGGGCTCCCCGGGCGC | |
| | | | | GTACTCC | |
| R4 | 91 | TGTTCCCCAAAGCGATACCA | 145 | GCATGTTCCCCAAAGCGAT | 177 |
| | | CTTGAAGCAGTGGTACTGCT | | ACCACTTGAAGCAGTGGTA | |
| | | TGTGGGTACA | | CTGCTTGTGGGTACACTCTG | |
| | | | | CGGGTG | |
| phiBT1 | 92 | GGTGCTGGGTTGTTGTCTCT | 146 | CAGGTTTTTGACGAAAGTG | 178 |
| | | GGACAGTGATCCATGGGAA | | ATCCAGATGATCCAG | |
| | | ACTACTCAGCACC | | | |
| MJ1 | 93 | ATTTTAGGTATATGATTTTGT | 147 | CAAAGGATCACTGAATCAA | 179 |
| | | TTATTAGTGTAAATAACACT | | AAGTATTGCTCATCCACGC | |
| | | ATGTACCTAAAAT | | GAAA | |
| MR11 | 94 | TTTGTGCGGAACTACGAACA | 148 | CGAAAATGTATGGAGGCAC | 180 |
| | | GTTCATTAATACGAAGTGTA | | TTGTATCAATATAGGATGTA | |
| | | CAAACTTCCATACAA | | TACCTTCGAAGACACTT | |
| TP901-1 | 95 | GAGTTTTTATTTCGTTTATTT | 149 | ATGCCAACACAATTAACAT | 181 |
| | | CAATTAAGGTAACTAAAAAA | | CTCAATCAAGGTAAATGCT | |
| | | CTCCTTTTAAGG | | TTTTGCTTTTTTTGC | |
| A118 | 96 | TTCCTCGTTTTCTCTCGTTGG | 150 | TTTCGGATCAAGCTATGAA | 182 |
| | | AAGAAGAAGAAACGAGAAA | | GGACGCAAAGAGGGAACTA | |
| | | | | AA | |
| U153 | 97 | TTCCTCGTTTTCTCTCGTTGG | 151 | TTTCGGATCAAGCTATGAA | 182 |
| | | ACGGAAACGAATCGAGAAA | | GGACGCAAAGAGGGAACTA | |
| | | | | AA | |
| phiRV1 | 98 | GTAGTGTATCTCACAGGTCC | 152 | GAAGGTGTTGGTGCGGGGT | 183 |
| | | ACGGTTGGCCGTGGACTGCT | | TGGCCGTGGTCGAGGTGGG | |
| | | GAAGAACATTCC | | GT | |
| phi370.1 | 99 | AAAAAAATACAGCGTTTTTC | 153 | TTGTAAAGGAGACTGATAA | 184 |
| | | ATGTACAACTATACTAGTTG | | TGGCATGTACAACTATACTC | |
| | | TAGTGCCTAAAA | | GTCGGTAAAAAGGCA | |
| TG1 | 100 | TCCAGCCCAACAGTGTTAGT | 154 | GATCAGCTCCGCGGGCAAG | 185 |
| | | CTTTGCTCTTACCCAGTTGG | | ACCTTTCTCCTTCACGGGGT | |
| | | GCGGGA | | GGAAGGTC | |
| WB | 101 | CTAGTTTTAAAGTTGGTTATT | 155 | CGGAAGGTAGCGTCAACGA | 186 |
| | | AGTTACTGTGATATTTATCA | | TAGGTGTAACTGTCGTGTTT | |
| | | CGGTACCCAATAACCAATGA | | GTAACGGTACTTCCAACAG | |
| | | AT | | CTGGCGCCGCCAC | |
| BL3 | 102 | CAATGAAAAACTAGGCATGT | 156 | TTTCCACAGACAACTCACG | 187 |
| | | AGAAGTTGTTTGT | | TGGAGGTAGTCAC | |
| SprA | 103 | TGTAGTAAGTATCTTAATAT | 157 | CACCCATTGTGTTCACAGG | 188 |
| | | ACAGCTTTATCTGTTTTTTA | | AGATACAGCTTTATCTGTAC | |
| | | AGATACTTACTACTTT | | TGATATTAATGACATGCTG | |
| phiJoe | 104 | AGTTGTGGCCATGTGTCCAT | 158 | ATCTGGATGTGGGTGTCCAT | 189 |
| | | CTGGGGGCAGATGGAGACG | | CTGCGGGCAGACGCCGCAG | |
| | | GGGTCACA | | TCGAAGCACGG | |
| phiK38 | 105 | CCCTAATACGCAAGTCGATA | 160 | GAGCGCCGGATCAGGGAGT | 191 |
| | | ACTCTCCTGGGAGCGTTGAC | | GGACGGCCTGGGAGCGCTA | |
| | | AACTTGCGCACCCTGATCTG | | CACGCTGTGGCTGCGGTCG | |
| | | | | GTGC | |
| Int2 | 105 | GCTCATGTATGTGTCTACGC | 161 | GGACGGCGCAGAAGGGGA | 192 |
| | | GAGATTCTCGCCCGAGAACT | | GTAGCTCTTCGCCGGACCG | |
| | | TCTGCAAGGCACTGCTCTTG | | TCGACATACTGCTCAGCTC | |
| | | GCT | | GTC | |
| Int3 | 105 | ATGGATAAAAAAATACAGC | 162 | GTTTGTAAAGGAGACTGAT | 193 |
| | | GTTTTTCATGTACAACTATA | | AATGGCATGTACAACTATA | |
| | | CTAGTTGTAGTGCCTAAATA | | CTCGTCGGTAAAAAGGCAT | |
| | | ATGCTT | | CTTAT | |
| Int4 | 105 | AAAAATTACAAAGTTTTCAA | 163 | TTCCAAAGAGCGCCCAACG | 194 |
| | | CCCTTGATTTGAATTAGCGG | | CGACCTGAAATTTGAATAA | |
| | | TCAAATAATTTGTAATTCGT | | GACTGCTGCTTGTGTAAAG | |
| | | TT | | GCGATGATT | |
| Int7 | 105 | GTGTTATAAACCTGTGTGAG | 164 | AGACGAGAAACGTTCCGTC | 195 |
| | | AGTTAAGTTTACATGCCTAA | | CGTCTGGGTCAGTTGGGCA | |
| | | CCTTAACTTTTACGCAGGTT | | AAGTTGATGACCGGGTCGT | |
| | | CAGCTT | | CCGTT | |
| Int8 | 105 | TTAATAAACTATGGAAGTAT | 165 | CAATCATCAGATAACTATG | 196 |
| | | GTACAGTCTTGCAATGTTGA | | GCGGCACGTGCATTAACCA | |
| | | GTGAACAAACTTCCATAATA | | CGGTTGTATCCCGTCTAAAG | |
| | | AAAT | | TACTCGT | |
| Int9 | 105 | GTGGTTGTTTTTGTTGGAAG | 166 | TTTATATTGCGAAAAATAAT | 197 |
| | | TGTGTATCAGGTATCTGCAT | | TGGCGAACGAGGTAACTGG | |
| | | AGTTATTCCGAACTTCCAAT | | ATACCTCATCCGCCAATTA | |
| | | TA | | AAATTTG | |
| Int10 | 105 | GGAAAATATAAATAATTTTA | 167 | AGCACGCTGATAATCAGCA | 198 |
| | | GTAACCTACATCTCAATCAA | | AGACCACCAACATTTCCAC | |
| | | GGATAGTAAAACTCTCACTC | | CAATGTAAAAGCTTTAACC | |
| | | TT | | TTAGC | |
| Int11 | 105 | GTTTATATGTTTACTAATAA | 168 | ATGGATTTTGCAGATTCCCA | 199 |
| | | GACGCTCTCAACCCATAAAG | | GATGCCCCTACAGAAAGAG | |
| | | TCTTATTAGTAAACATATTTC | | GTACAAAACATTTATTGGA | |
| | | AACT | | ATTAATT | |
| Int12 | 105 | TTTTTGTATGTTAGTTGTGTC | 169 | GTTCGTGGTAACTATGGGT | 200 |
| | | ACTGGGTAGACCTAAATAGT | | GGTACAGGTGCCACATTAG | |
| | | GACACAACTGCTATTAAAAT | | TTGTACCATTTATGTTTATG | |
| | | TTAA | | TGGTTAAC | |
| Int13 | 105 | CAATAACGGTTGTATTTGTA | 170 | GCATACATTGTTGTTGTTTT | 201 |
| | | GAACTTGACCAGTTGTTTTA | | TCCAGATCCAGTTGGTCCTG | |
| | | GTAACATAAATACAACTCCG | | TAAATATAAGCAATCCATG | |
| | | AATA | | TGAGT | |
| LI | 106 | GTTTAGTATCTCGTTATCTCT | 171 | TAACTTTTTCGGATCGAGTT | 202 |
| | | CGTTGGAGGGAGAAGAAAC | | ATGATGGACGTAAAGAGGG | |
| | | GGGATACCAAAA | | AACAAAGCATCTA | |
| Peaches | 107 | TAGTTTCCAATGTTACAGGA | 172 | CGGTCTCCATCGGGATCTG | 203 |
| | | ACTGCTGGCAGAATCCAACA | | CTGATCGAGCAGCATGCCG | |
| | | CATTGGAAGTCG | | ACCA | |
| Bxz2 | 107 | TAACCGCAAGTGTACATCCC | 173 | CGGTCTCCATCGGGATCTG | 203 |
| | | TCGGCTGGCCGAGACAAGTA | | CTGATCGAGCAGCATGCCG | |
| | | CAGTTGCGACAG | | ACCA | |
| SV1 | 108 | ATGTGGTCCTTTAGATCCAC | 174 | CATCAGGGCGGTCAGGCCG | 204 |
| | | TGACGTGGGTCAGTGTCTCT | | TAGATGTGGAAGAAACGGC | |
| | | AAAGGACTCGCG | | AGCACGGCGAGGACG | |

Table 4. Serine resolvases and SSR target sequences.

+0 TABLE 4
Serine resolvases and SSR target sequences.

| Resolvase | Source | Left Target | Right Target |
|---|---|---|---|
| Gin | 109 | CGTTTCCTGTAA | CGTTTCCTGTA |
| | | ACCGAGGTTTT | AACCGAGGTTT |
| | | GGATAAACA | TGGATAATGG |
| | | (SEQ ID | (SEQ ID |
| | | NO: 205) | NO: 210) |
| Cin | 110 | GAGTTCTCTTAA | GAGTTCTCTTA |
| | | ACCAAGGTTTA | AACCAAGGTAT |
| | | GGATTGAAA | TGGATAACAG |
| | | (SEQ ID | (SEQ ID |
| | | NO: 206) | NO: 211) |
| Hin | 111 | TGGTTCTTGAAA | AAATTTTCCTT |
| | | ACCAAGGTTTT | TTTGGAAGGTT |
| | | TGATAAAGC | TTTGATAACCA |
| | | (SEQ ID | (SEQ ID |
| | | NO: 207) | NO: 212) |
| Min | 112 | GCCTTCCCCTAA | GCCTTCCCCCA |
| | | ACCAACGTTTT | AACCAAGGTAA |
| | | TATGCCGCC | TCAAGAACGC |
| | | (SEQ ID | (SEQ ID |
| | | NO: 208) | NO: 213) |
| Sin | 113 | TTGTGAAATTT | CGTATGATTAG |
| | | GGGTACACCCT | GGTGTATATTA |
| | | AATCATACAA | ATTT |
| | | (SEQ ID | (SEQ ID |
| | | NO: 209) | NO: 214) |

Table 5. Tyrosine integrases and target sequences.

+0 TABLE 5
Tyrosine integrases and target sequences.

| Integrase | Source | attP | attB |
|---|---|---|---|
| HK022 | 114 | CAAATGATTTTATTTTGACTAATAATGACCT | GCACTTTAGGTGAA |
| | | ACTTACATTAATTTACTGATAATTAAAGAGA | AAAGGTT (SEQ ID |
| | | TTTTAAATATACAACTTATTCACCTAAAGGA | NO: 219) |
| | | TGACAAAA (SEQ ID NO: 215) | |
| P22 | 115 | CTAAGTGGTTTGGGACAAAAATGGGACATA | GCAGCGCATTCGTAA |
| | | CAAATCTTTGCATCGGTTTGCAAGGCTTTGC | TGCGAAGGTCGT |
| | | ATGTCTTTCGAAGATGGGACGTGTGAGCGCA | (SEQ ID NO: 220) |
| | | GGTATGACGTGGTATGTGTTGACTTAAAAGG | |
| | | TAGTTCTTATAATTCGTAATGCGAAGGTCGT | |
| | | AGGTTCGACTCCTATTATCGGCACCAGTTAA | |
| | | ATCAAATACTTACGTATTATTCGTGCCTTCCT | |
| | | TATTTTTACTGTGGGACATATTTGGGACAGA | |
| | | AGTACCAAAAA (SEQ ID NO: 217) | |
| L5 | 116 | GCGATCCCCATCCGCGACGTGCCAACTAGGT | GAGCGGGCGACGGG |
| | | CTCCTCTCGTCGTGAACAAGGCTACCGGGTT | AATCGAACCCGCGTA |
| | | GCAACTCCTGTGCAACTCTCAGGCTTCAACG | GCTAGTTTGGAAGA |
| | | CGCTTCTACGACCTGCAATTTCTTTCCACTTA | (SEQ ID NO: 221) |
| | | GAGGATGCAGCCGAGAGGGGTAAAAACCTA | |
| | | TCTTGACCGGCCCATATGTGGTCGGCAGACA | |
| | | CCCATTCTTCCAAACTAGCTACGCGGGTTCG | |
| | | ATTCCCGTCGCCCGCTCCGCTGGTCAGAGGG | |
| | | TGTTTTCGCCCTCTGGCCATTTTTCTTTCCAG | |
| | | GGGTCTGCAACTCTTGTGCGACTCTTCTGAC | |
| | | CTGGGCATACGCGGTTGCAACGCATCCCTGA | |
| | | TCTGGCTACTTTCGATGCTGACAAACGAATA | |
| | | GAGCCCCCCGCCTGCGCGAACAGACGAGGG | |
| | | GCATTCACA (SEQ ID NO: 218) | |

### REFERENCES CITED IN EXAMPLE 1

Each of the following references are cited in Example 1, each of which are incorporated herein by reference.
1. Feuk, L. Inversion variants in the human genome: role in disease and genome architecture. Genome Med 2, 11 (2010).
2. Zhang, F., Gu, W., Hurles, M. E. & Lupski, J. R. Copy number variation in human health, disease, and evolution. Annu Rev Genomics Hum Genet 10, 451-481 (2009).
3. Shaw, C. J. & Lupski, J. R. Implications of human genome architecture for rearrangement-based disorders: the genomic basis of disease. Hum Mol Genet 13 Spec No 1, R57-64 (2004).
4. Carvalho, C. M., Zhang, F. & Lupski, J. R. Evolution in health and medicine Sackler colloquium: Genomic disorders: a window into human gene and genome evolution. Proc Natl Acad Sci USA 107 Suppl 1, 1765-1771 (2010).
5. Rowley, J. D. Chromosome translocations: dangerous liaisons revisited. Nat Rev Cancer 1, 245-250 (2001).
6. Aplan, P. D. Causes of oncogenic chromosomal translocation. Trends Genet 22, 46-55 (2006).
7. McCarroll, S. A. & Altshuler, D. M. Copy-number variation and association studies of human disease. Nat Genet 39, S37-42 (2007).
8. Wijnker, E. & de Jong, H. Managing meiotic recombination in plant breeding. Trends Plant Sci 13, 640-646 (2008).
9. Petolino, J. F., Srivastava, V. & Daniell, H. Editing Plant Genomes: a new era of crop improvement. Plant Biotechnol J 14, 435-436 (2016).
10. Smith, M. C. M. Phage-encoded Serine Integrases and Other Large Serine Recombinases. Microbiol Spectr 3 (2015).
11. Meinke, G., Bohm, A., Hauber, J., Pisabarro, M. T. & Buchholz, F. Cre Recombinase and Other Tyrosine Recombinases. Chem Rev 116, 12785-12820 (2016).
12. Karpinski, J. et al. Directed evolution of a recombinase that excises the provirus of most HIV-1 primary isolates with high specificity. Nat Biotechnol 34, 401-409 (2016).
13. Olorunniji, F. J., Rosser, S. J. & Stark, W. M. Site-specific recombinases: molecular machines for the Genetic Revolution. Biochem J 473, 673-684 (2016).
14. Grindley, N.D., Whiteson, K. L. & Rice, P. A. Mechanisms of site-specific recombination. Annu Rev Biochem 75, 567-605 (2006).
15. Lukacsovich, T., Yang, D. & Waldman, A. S. Repair of a specific double-strand break generated within a mammalian chromosome by yeast endonuclease I-SceI. Nucleic Acids Res 22, 5649-5657 (1994).
16. Rouet, P., Smih, F. & Jasin, M. Introduction of double-strand breaks into the genome of mouse cells by expression of a rare-cutting endonuclease. Mol Cell Biol 14, 8096-8106 (1994).
17. Jeggo, P. A. DNA breakage and repair. Adv Genet 38, 185-218 (1998).
18. Haapaniemi, E., Botla, S., Persson, J., Schmierer, B. & Taipale, J. CRISPR-Cas9 genome editing induces a p53-mediated DNA damage response. Nat Med 24, 927-930 (2018).
19. Wang, B. et al. Highly efficient CRISPR/HDR-mediated knock-in for mouse embryonic stem cells and zygotes. Biotechniques 59, 201-202, 204, 206-208 (2015).
20. Bogdanove, A. J., Bohm, A., Miller, J. C., Morgan, R.D. & Stoddard, B. L. Engineering altered protein-DNA recognition specificity. Nucleic Acids Res 46, 4845-4871 (2018).
21. Tassabehji, M. Williams-Beuren syndrome: a challenge for genotype-phenotype correlations. Hum Mol Genet 12 Spec No 2, R229-237 (2003).
22. Araki, K., Araki, M. & Yamamura, K. Targeted integration of DNA using mutant lox sites in embryonic stem cells. Nucleic Acids Res 25, 868-872 (1997).
23. Araki, K., Okada, Y., Araki, M. & Yamamura, K. Comparative analysis of right element mutant lox sites on recombination efficiency in embryonic stem cells. BMC Biotechnol 10, 29 (2010).
24. Thomson, J. G., Rucker, E. B., 3rd & Piedrahita, J. A. Mutational analysis of loxP sites for efficient Cre-mediated insertion into genomic DNA. Genesis 36, 162-167 (2003).
25. Jusiak, B. et al. Comparison of Integrases Identifies Bxb1-GA Mutant as the Most Efficient Site-Specific Integrase System in Mammalian Cells. ACS Synth Biol 8, 16-24 (2019).
26. Xie, F. et al. Adjusting the attB site in donor plasmid improves the efficiency of PhiC31 integrase system. DNA Cell Biol 31, 1335-1340 (2012).
27. Gupta, M., Till, R. & Smith, M. C. Sequences in attB that affect the ability of phiC31 integrase to synapse and to activate DNA cleavage. Nucleic Acids Res 35, 3407-3419 (2007).
28. Kolot, M., Malchin, N., Elias, A., Gritsenko, N. & Yagil, E. Site promiscuity of coliphage HK022 integrase as tool for gene therapy. Gene Ther 22, 602 (2015).
29. Gaj, T., Mercer, A. C., Sirk, S. J., Smith, H. L. & Barbas, C. F., 3rd A comprehensive approach to zinc-finger recombinase customization enables genomic targeting in human cells. Nucleic Acids Res 41, 3937-3946 (2013).
30. Chuang, K., Nguyen, E., Sergeev, Y. & Badea, T. C. Novel Heterotypic Rox Sites for Combinatorial Dre Recombination Strategies. G3 (Bethesda) 6, 559-571 (2015).
31. Chaikind, B., Bessen, J. L., Thompson, D. B., Hu, J. H. & Liu, D. R. A programmable Cas9-serine recombinase fusion protein that operates on DNA sequences in mammalian cells. Nucleic Acids Res 44, 9758-9770 (2016).
32. Gaj, T., Mercer, A. C., Gersbach, C. A., Gordley, R. M. & Barbas, C. F. Structure-guided reprogramming of serine recombinase DNA sequence specificity. P Natl Acad Sci USA 108, 498-503 (2011).
33. Gaj, T., Sirk, S. J. & Barbas, C. F., 3rd Expanding the scope of site-specific recombinases for genetic and metabolic engineering. Biotechnol Bioeng 111, 1-15 (2014).
34. Akopian, A., He, J., Boocock, M. R. & Stark, W. M. Chimeric recombinases with designed DNA sequence recognition. Proc Natl Acad Sci USA 100, 8688-8691 (2003).
35. Prorocic, M. M. et al. Zinc-finger recombinase activities in vitro. Nucleic Acids Research 39, 9316-9328 (2011).
36. Gersbach, C. A., Gaj, T., Gordley, R. M., Mercer, A. C. & Barbas, C. F. Targeted plasmid integration into the human genome by an engineered zinc-finger recombinase. Nucleic Acids Research 39, 7868-7878 (2011).
37. Sirk, S. J., Gaj, T., Jonsson, A., Mercer, A. C. & Barbas, C. F. Expanding the zinc-finger recombinase repertoire: directed evolution and mutational analysis of serine recombinase specificity determinants. Nucleic Acids Research 42, 4755-4766 (2014).
38. Gaj, T. & Barbas, C. F., 3rd Genome engineering with custom recombinases. Methods Enzymol 546, 79-91 (2014).
39. Olorunniji, F. J., Rosser, S. J. & Marshall Stark, W. Purification and In Vitro Characterization of Zinc Finger Recombinases. Methods Mol Biol 1642, 229-245 (2017).
40. Proudfoot, C., McPherson, A. L., Kolb, A. F. & Stark, W. M. Zinc finger recombinases with adaptable DNA sequence specificity. PLoS One 6, e19537 (2011).
41. Irion, S. et al. Identification and targeting of the ROSA26 locus in human embryonic stem cells. Nat Biotechnol 25, 1477-1482 (2007).
42. Sadelain, M., Papapetrou, E. P. & Bushman, F. D. Safe harbours for the integration of new DNA in the human genome. Nat Rev Cancer 12, 51-58 (2012).
43. Pellenz, S. et al. New human chromosomal safe harbor sites for genome engineering with CRISPR/Cas9, TAL effector and homing endonucleases. bioRxiv (2019).
44. Brown, W. R., Lee, N. C., Xu, Z. & Smith, M. C. Serine recombinases as tools for genome engineering. Methods 53, 372-379 (2011).
45. Xu, Z. et al. Accuracy and efficiency define Bxb1 integrase as the best of fifteen candidate serine recombinases for the integration of DNA into the human genome. BMC Biotechnol 13, 87 (2013).
46. Thyagarajan, B., Guimaraes, M. J., Groth, A. C. & Calos, M. P. Mammalian genomes contain active recombinase recognition sites. Gene 244, 47-54 (2000).
47. Shultz, J. L., Voziyanova, E., Konieczka, J. H. & Voziyanov, Y. A genome-wide analysis of FRT-like sequences in the human genome. PLoS One 6, e18077 (2011).
48. Thyagarajan, B., Olivares, E. C., Hollis, R. P., Ginsburg, D. S. & Calos, M. P. Site-specific genomic integration in mammalian cells mediated by phage phiC31 integrase. Mol Cell Biol 21, 3926-3934 (2001).
49. Sivalingam, J. et al. Biosafety assessment of site-directed transgene integration in human umbilical cord-lining cells. Mol Ther 18, 1346-1356 (2010).
50. Ortiz-Urda, S. et al. Stable nonviral genetic correction of inherited human skin disease. Nat Med 8, 1166-1170 (2002).
51. Chalberg, T. W. et al. Integration specificity of phage phiC31 integrase in the human genome. J Mol Biol 357, 28-48 (2006).
52. Thyagarajan, B. et al. Creation of engineered human embryonic stem cell lines using phiC31 integrase. Stem Cells 26, 119-126 (2008).
53. Olivares, E. C. et al. Site-specific genomic integration produces therapeutic Factor IX levels in mice. Nat Biotechnol 20, 1124-1128 (2002).
54. Hollis, R. P. et al. Phage integrases for the construction and manipulation of transgenic mammals. Reprod Biol Endocrinol 1, 79 (2003).
55. Held, P. K. et al. In vivo correction of murine hereditary tyrosinemia type I by phiC31 integrase-mediated gene delivery. Mol Ther 11, 399-408 (2005).
56. Ma, H. et al. PhiC31 integrase induces efficient site-specific recombination in the Capra hircus genome. DNA Cell Biol 33, 484-491 (2014).
57. Bi, Y. et al. Pseudo attP sites in favor of transgene integration and expression in cultured porcine cells identified by Streptomyces phage phiC31 integrase. BMC Mol Biol 14, 20 (2013).
58. Ma, Q. W. et al. Identification of pseudo attP sites for phage phiC31 integrase in bovine genome. Biochem Biophys Res Commun 345, 984-988 (2006).
59. Qu, L. et al. Global mapping of binding sites for phiC31 integrase in transgenic madendarby bovine kidney cells using ChIP-seq. Hereditas 156, 3 (2019).
60. Ghahfarokhi, M. K., Dormiani, K., Mohammadi, A., Jafarpour, F. & Nasr-Esfahani, M. H. Blastocyst Formation Rate and Transgene Expression are Associated with Gene Insertion into Safe and Non-Safe Harbors in the Cattle Genome. Sci Rep 7, 15432 (2017).
61. Groth, A. C., Fish, M., Nusse, R. & Calos, M. P. Construction of transgenic Drosophila by using the site-specific integrase from phage phiC31. Genetics 166, 1775-1782 (2004).
62. Chalberg, T. W., Genise, H. L., Vollrath, D. & Calos, M. P. phiC31 integrase confers genomic integration and long-term transgene expression in rat retina. Invest Ophthalmol Vis Sci 46, 2140-2146 (2005).
63. Keravala, A. et al. A diversity of serine phage integrases mediate site-specific recombination in mammalian cells. Mol Genet Genomics 276, 135-146 (2006).
64. Lei, X., Wang, L., Zhao, G. & Ding, X. Site-specificity of serine integrase demonstrated by the attB sequence preference of BT1 integrase. FEBS Lett 592, 1389-1399 (2018).
65. Chu, V. T. et al. Increasing the efficiency of homology-directed repair for CRISPR-Cas9-induced precise gene editing in mammalian cells. Nat Biotechnol 33, 543-548 (2015).
66. Yu, C. et al. Small molecules enhance CRISPR genome editing in pluripotent stem cells. Cell Stem Cell 16, 142-147 (2015).
67. Paquet, D. et al. Efficient introduction of specific homozygous and heterozygous mutations using CRISPR/Cas9. Nature 533, 125 (2016).
68. Martsolf, J. T. et al. Complete trisomy 17p a relatively new syndrome. Ann Genet 31, 172-174 (1988).
69. Bird, T. D. in GeneReviews((R)). (eds. M. P. Adam et al.) (Seattle (WA); 1993).
70. Smith, A. C. M. et al. in GeneReviews((R)). (eds. M. P. Adam et al.) (Seattle (WA); 1993).
71. Dupuy, O. et al. [De La Chapelle syndrome]. Presse Med 30, 369-372 (2001).
72. Jyothy, A. et al. Translocation Down syndrome. Indian J Med Sci 56, 122-126 (2002).
73. Lakich, D., Kazazian, H. H., Jr., Antonarakis, S. E. & Gitschier, J. Inversions disrupting the factor VIII gene are a common cause of severe haemophilia A. Nat Genet 5, 236-241 (1993).
74. Bondeson, M. L. et al. Inversion of the IDS gene resulting from recombination with IDS-related sequences is a common cause of the Hunter syndrome. Hum Mol Genet 4, 615-621 (1995).
75. Abremski, K. & Hoess, R. Bacteriophage P1 site-specific recombination. Purification and properties of the Cre recombinase protein. J Biol Chem 259, 1509-1514 (1984).
76. Sauer, B. & McDermott, J. DNA recombination with a heterospecific Cre homolog identified from comparison of the pac-c1 regions of P1-related phages. Nucleic Acids Res 32, 6086-6095 (2004).
77. Suzuki, E. & Nakayama, M. VCre/VloxP and SCre/SloxP: new site-specific recombination systems for genome engineering. Nucleic Acids Res 39, e49 (2011).
78. Sadowski, P. D. The Flp recombinase of the 2-microns plasmid of Saccharomyces cerevisiae. Prog Nucleic Acid Res Mol Biol 51, 53-91 (1995).
79. Nern, A., Pfeiffer, B. D., Svoboda, K. & Rubin, G. M. Multiple new site-specific recombinases for use in manipulating animal genomes. Proc Natl Acad Sci USA 108, 14198-14203 (2011).
80. Ringrose, L., Angrand, P. O. & Stewart, A. F. The Kw recombinase, an integrase from Kluyveromyces waltii. Eur J Biochem 248, 903-912 (1997).
81. Araki, H. et al. Site-specific recombinase, R, encoded by yeast plasmid pSR1. J Mol Biol 225, 25-37 (1992).
82. Blaisonneau, J., Sor, F., Cheret, G., Yarrow, D. & Fukuhara, H. A circular plasmid from the yeast Torulaspora delbrueckii. Plasmid 38, 202-209 (1997).
83. Karimova, M. et al. Vika/vox, a novel efficient and specific Cre/loxP-like site-specific recombination system. Nucleic Acids Res 41, e37 (2013).
84. Karimova, M., Splith, V., Karpinski, J., Pisabarro, M. T. & Buchholz, F. Discovery of Nigri/nox and Panto/pox site-specific recombinase systems facilitates advanced genome engineering. Sci Rep 6, 30130 (2016).
85. Buchholz, F. & Stewart, A. F. Alteration of Cre recombinase site specificity by substrate-linked protein evolution. Nat Biotechnol 19, 1047-1052 (2001).
86. Santoro, S. W. & Schultz, P. G. Directed evolution of the site specificity of Cre recombinase. Proc Natl Acad Sci USA 99, 4185-4190 (2002).
87. Sarkar, I., Hauber, I., Hauber, J. & Buchholz, F. HIV-1 proviral DNA excision using an evolved recombinase. Science 316, 1912-1915 (2007).
88. Rufer, A. W. & Sauer, B. Non-contact positions impose site selectivity on Cre recombinase. Nucleic Acids Res 30, 2764-2771 (2002).
89. Kim, A. I. et al. Mycobacteriophage Bxb1 integrates into the Mycobacterium smegmatis groEL1 gene. Mol Microbiol 50, 463-473 (2003).
90. Brown, D. P., Idler, K. B. & Katz, L. Characterization of the genetic elements required for site-specific integration of plasmid pSE211 in Saccharopolyspora erythraea. J Bacteriol 172, 1877-1888 (1990).
91. Matsuura, M. et al. A GENE ESSENTIAL FOR THE SITE-SPECIFIC EXCISION OF ACTINOPHAGE R4 PROPHAGE GENOME FROM THE CHROMOSOME OF A LYSOGEN. The Journal of General and Applied Microbiology 41, 53-61 (1995).
92. Gregory, M. A., Till, R. & Smith, M. C. Integration site for Streptomyces phage phiBT1 and development of site-specific integrating vectors. J Bacteriol 185, 5320-5323 (2003).
93. Yang, H. Y., Kim, Y. W. & Chang, H. I. Construction of an integration-proficient vector based on the site-specific recombination mechanism of enterococcal temperate phage phiFC1. J Bacteriol 184, 1859-1864 (2002).
94. Rashel, M. et al. A novel site-specific recombination system derived from bacteriophage phiMR11. Biochem Biophys Res Commun 368, 192-198 (2008).
95. Christiansen, B., Johnsen, M. G., Stenby, E., Vogensen, F. K. & Hammer, K. Characterization of the lactococcal temperate phage TP901-1 and its site-specific integration. J Bacteriol 176, 1069-1076 (1994).
96. Loessner, M. J., Inman, R. B., Lauer, P. & Calendar, R. Complete nucleotide sequence, molecular analysis and genome structure of bacteriophage A118 of Listeria monocytogenes: implications for phage evolution. Mol Microbiol 35, 324-340 (2000).
97. Lauer, P., Chow, M. Y., Loessner, M. J., Portnoy, D. A. & Calendar, R. Construction, characterization, and use of two Listeria monocytogenes site-specific phage integration vectors. J Bacteriol 184, 4177-4186 (2002).
98. Bibb, L. A., Hancox, M. I. & Hatfull, G. F. Integration and excision by the large serine recombinase phiRv1 integrase. Mol Microbiol 55, 1896-1910 (2005).
99. Canchaya, C. et al. Genome analysis of an inducible prophage and prophage remnants integrated in the Streptococcus pyogenes strain SF370. Virology 302, 245-258 (2002).
100. Morita, K. et al. The site-specific recombination system of actinophage TG1. FEMS Microbiol Lett 297, 234-240 (2009).
101. Fouts, D. E. et al. Sequencing Bacillus anthracis typing phages gamma and cherry reveals a common ancestry. J Bacteriol 188, 3402-3408 (2006).
102. Kilcher, S., Loessner, M. J. & Klumpp, J. Brochothrix thermosphacta bacteriophages feature heterogeneous and highly mosaic genomes and utilize unique prophage insertion sites. J Bacteriol 192, 5441-5453 (2010).
103. Lazarevic, V. et al. Nucleotide sequence of the Bacillus subtilis temperate bacteriophage SPbetac2. Microbiology 145 (Pt 5), 1055-1067 (1999).
104. Fogg, P. C. M., Haley, J. A., Stark, W. M. & Smith, M. C. M. Genome Integration and Excision by a New Streptomyces Bacteriophage, varphiJoe. Appl Environ Microbiol 83 (2017).
105. Yang, L. et al. Permanent genetic memory with >1-byte capacity. Nat Methods 11, 1261-1266 (2014).
106. Rutherford, K., Yuan, P., Perry, K., Sharp, R. & Van Duyne, G. D. Attachment site recognition and regulation of directionality by the serine integrases. Nucleic Acids Res 41, 8341-8356 (2013).
107. Singh, S., Rockenbach, K., Dedrick, R. M., VanDemark, A. P. & Hatfull, G. F. Cross-talk between diverse serine integrases. J Mol Biol 426, 318-331 (2014).
108. Gupta, N. et al. Cross-talk between cognate and noncognate RpoE sigma factors and Zn(2+)-binding anti-sigma factors regulates photooxidative stress response in Azospirillum brasilense. Antioxid Redox Signal 20, 42-59 (2014).
109. Kahmann, R., Rudt, F., Koch, C. & Mertens, G. G inversion in bacteriophage Mu DNA is stimulated by a site within the invertase gene and a host factor. Cell 41, 771-780 (1985).
110. Iida, S., Meyer, J., Kennedy, K. E. & Arber, W. A site-specific, conservative recombination system carried by bacteriophage P1. Mapping the recombinase gene cin and the cross-over sites cix for the inversion of the C segment. EMBO J 1, 1445-1453 (1982).
111. Glasgow, A. C., Bruist, M. F. & Simon, M. I. DNA-binding properties of the Hin recombinase. J Biol Chem 264, 10072-10082 (1989).
112. Iida, S. et al. The Min DNA inversion enzyme of plasmid p15B of Escherichia coli 15T-: a new member of the Din family of site-specific recombinases. Mol Microbiol 4, 991-997 (1990).
113. Rowland, S. J., Stark, W. M. & Boocock, M. R. Sin recombinase from Staphylococcus aureus: synaptic complex architecture and transposon targeting. Mol Microbiol 44, 607-619 (2002).
114. Kolot, M., Silberstein, N. & Yagil, E. Site-specific recombination in mammalian cells expressing the Int recombinase of bacteriophage HK022. Mol Biol Rep 26, 207-213 (1999).
115. Cho, E. H., Nam, C. E., Alcaraz, R., Jr. & Gardner, J. F. Site-specific recombination of bacteriophage P22 does not require integration host factor. J Bacteriol 181, 4245-4249 (1999).
116. Lee, M. H., Pascopella, L., Jacobs, W. R., Jr. & Hatfull, G. F. Site-specific integration of mycobacteriophage L5: integration-proficient vectors for Mycobacterium smegmatis, Mycobacterium tuberculosis, and bacille Calmette-Guerin. Proc Natl Acad Sci USA 88, 3111-3115 (1991).

### Example 2: Targeted Integration of Recombinase Recognition Sites in a Target Gene

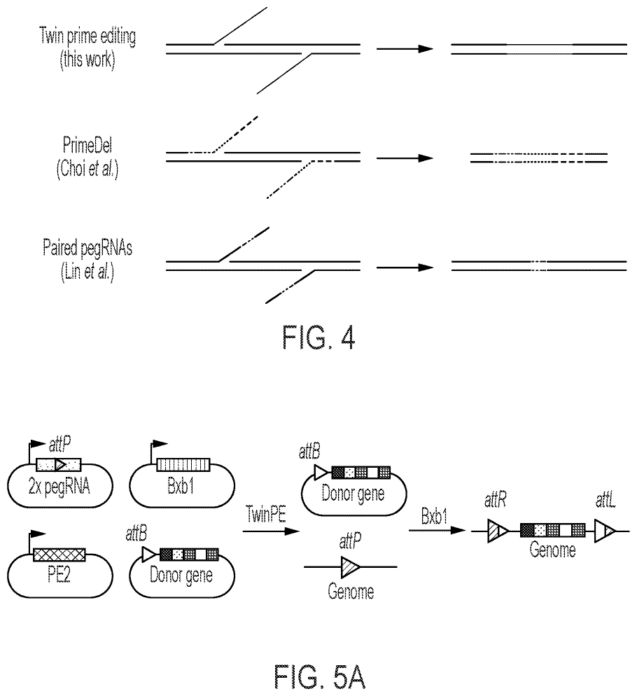

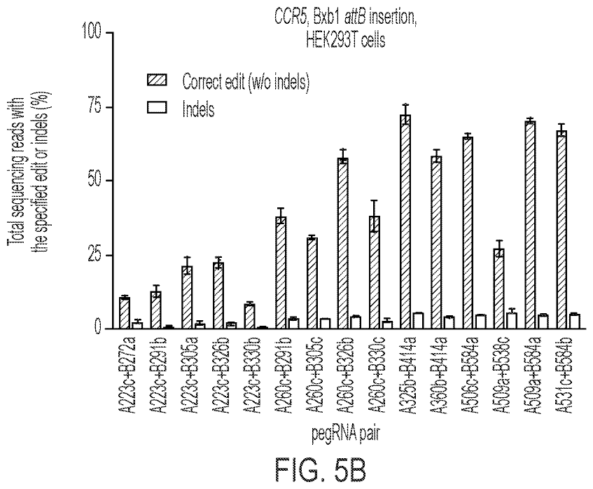

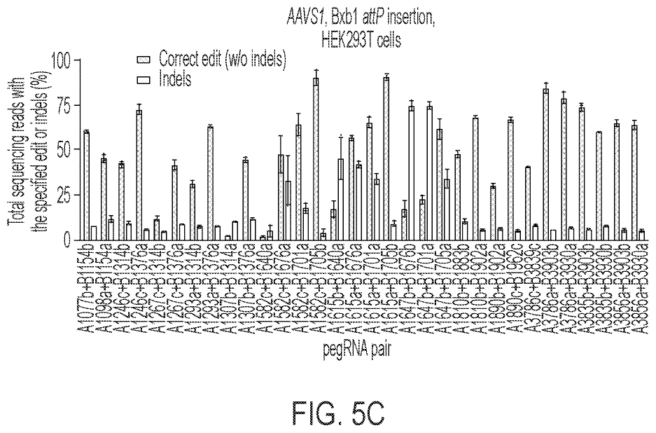

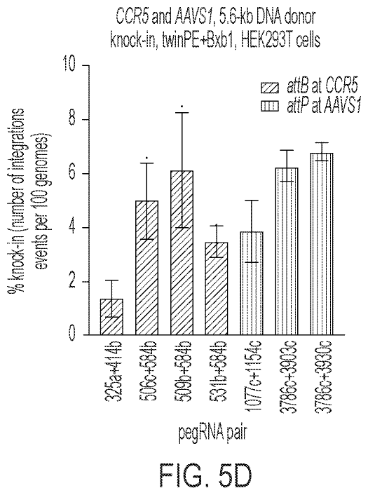

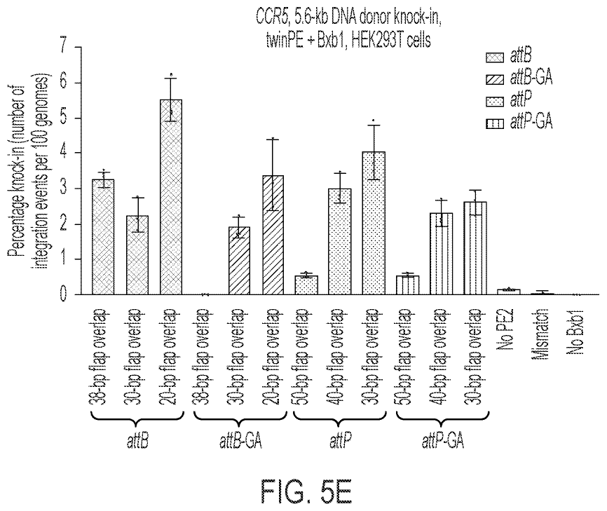

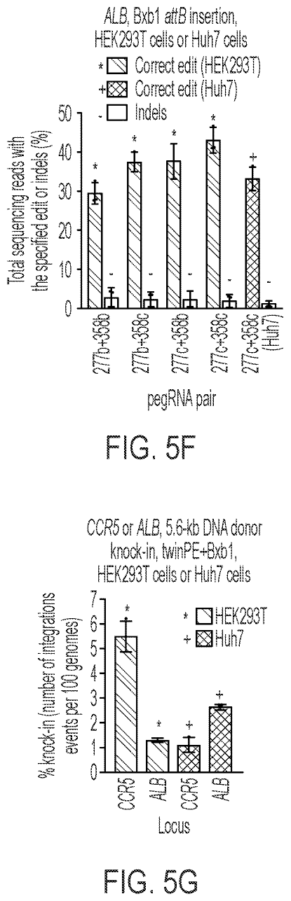

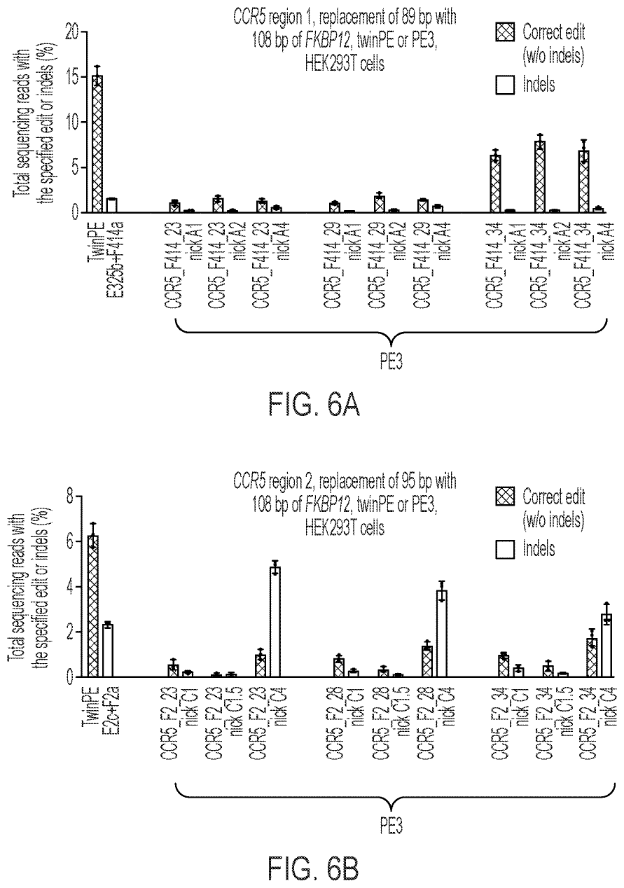

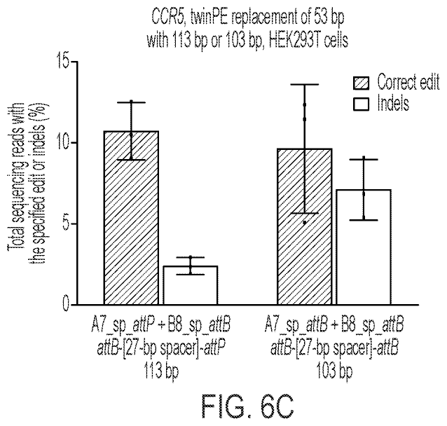

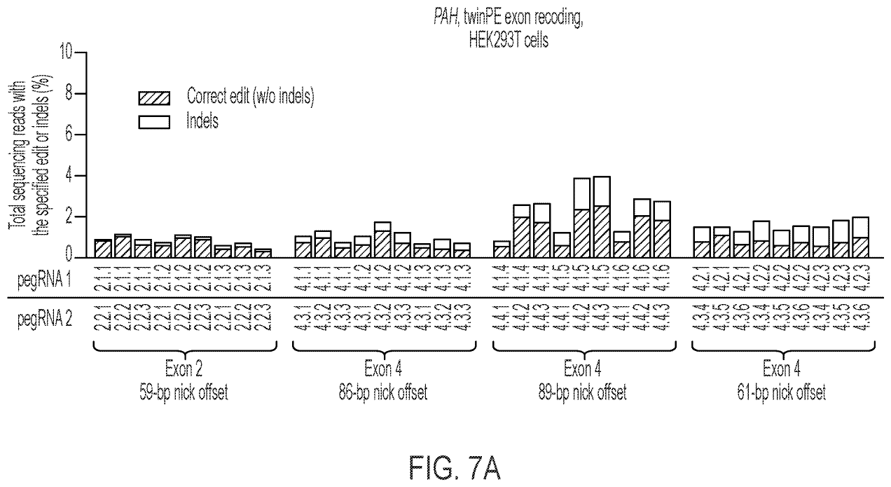

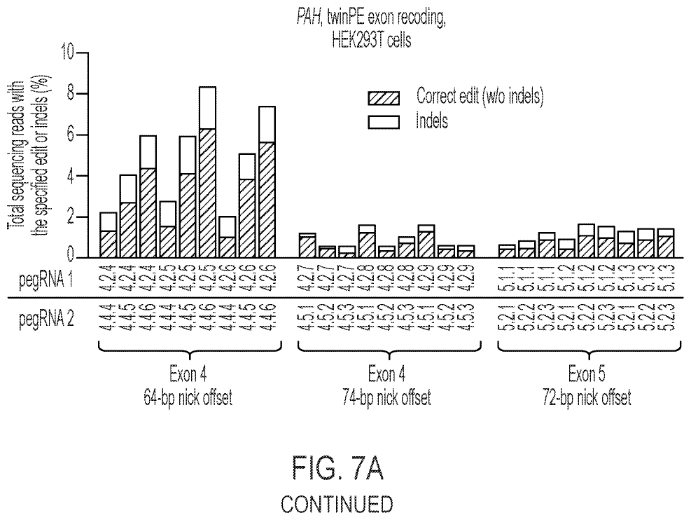

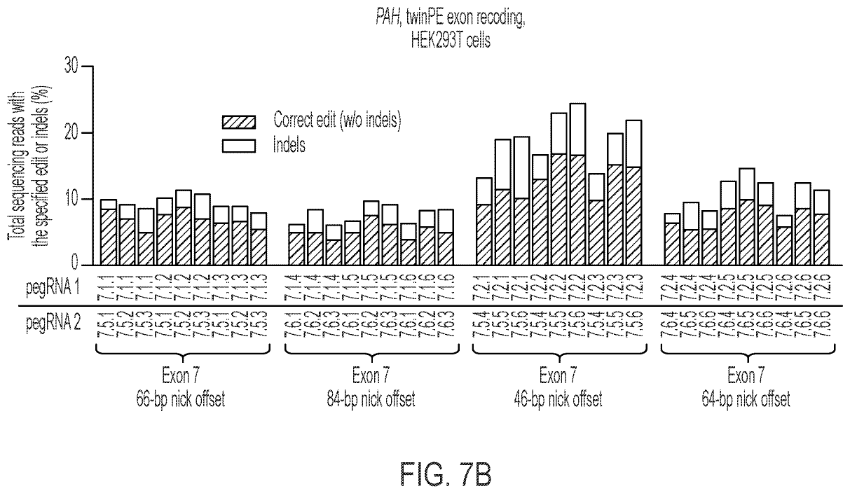

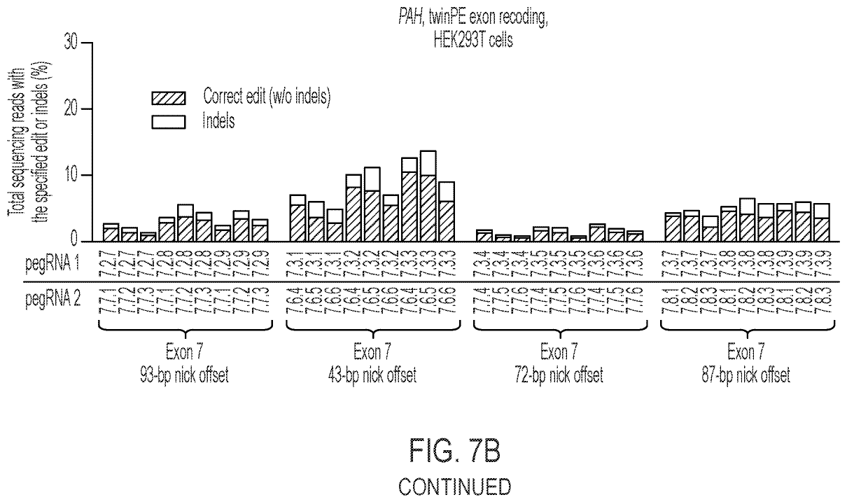

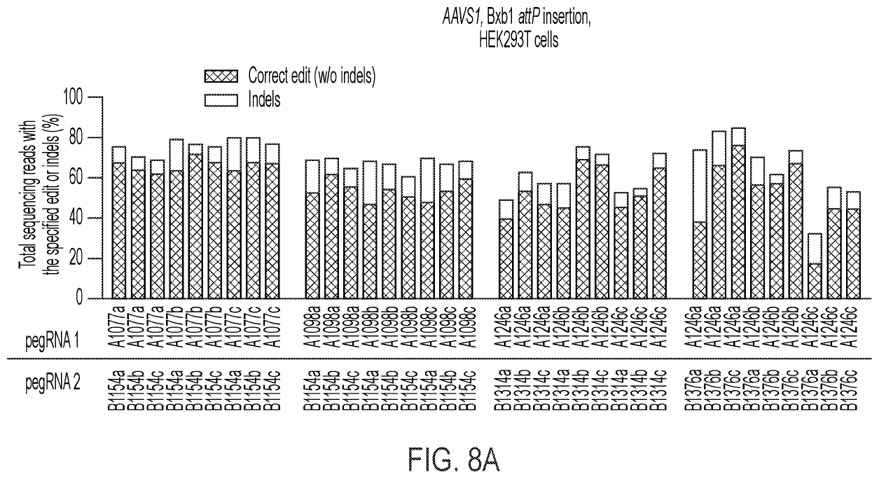

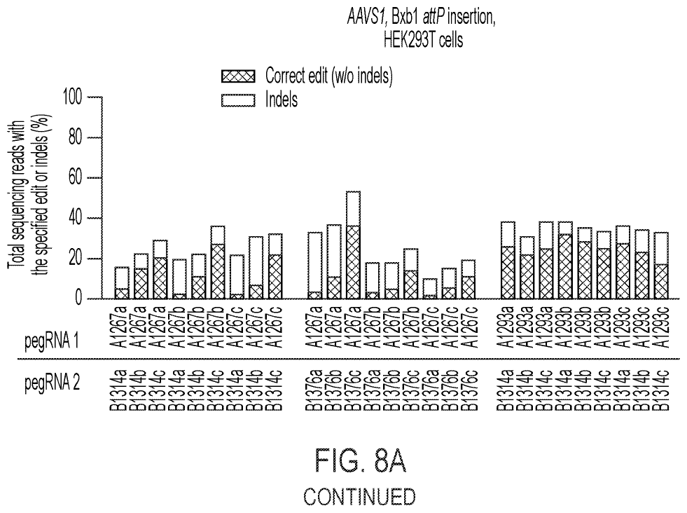

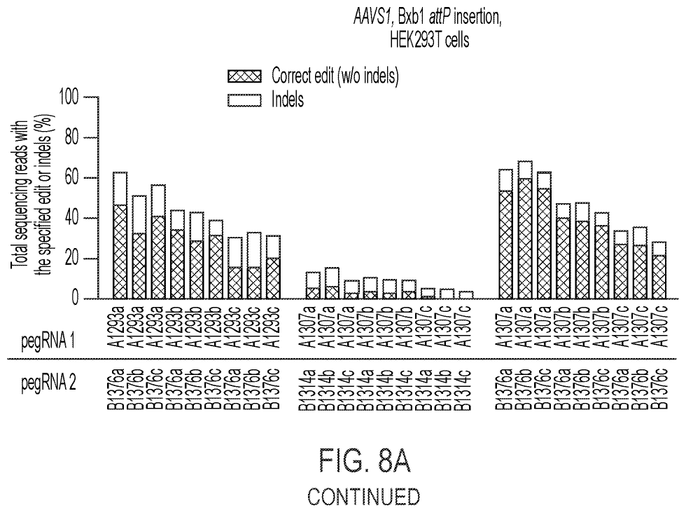

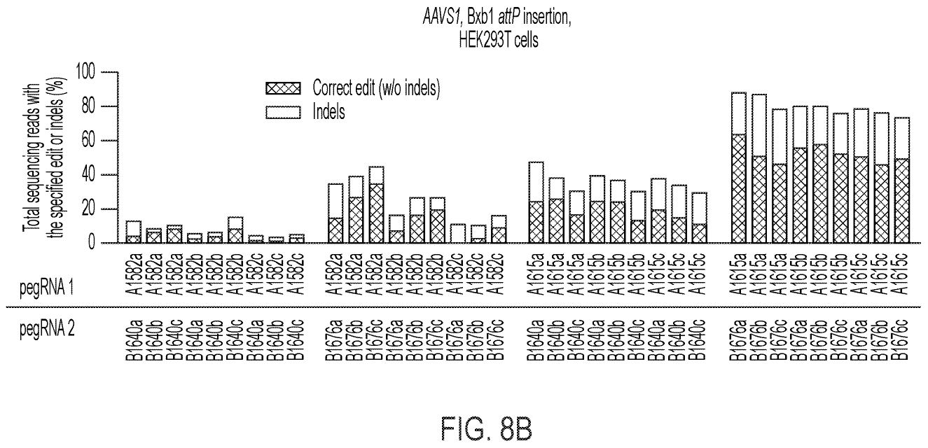

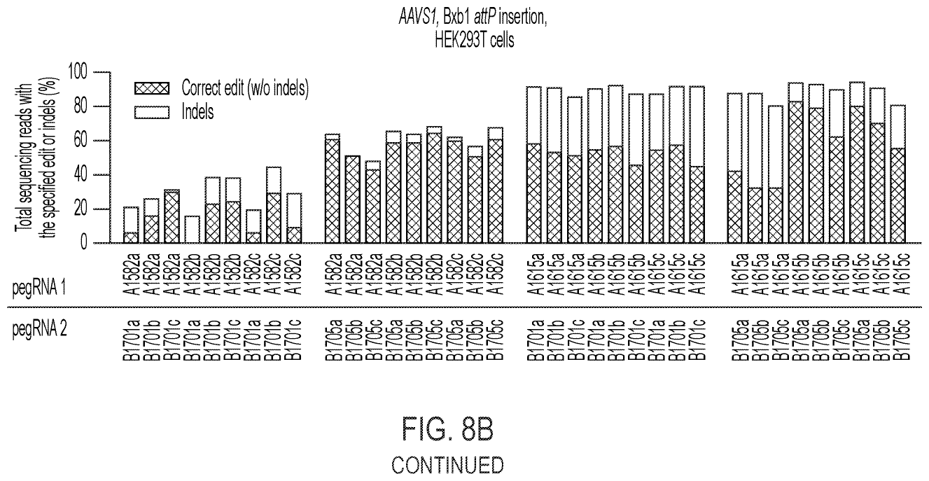

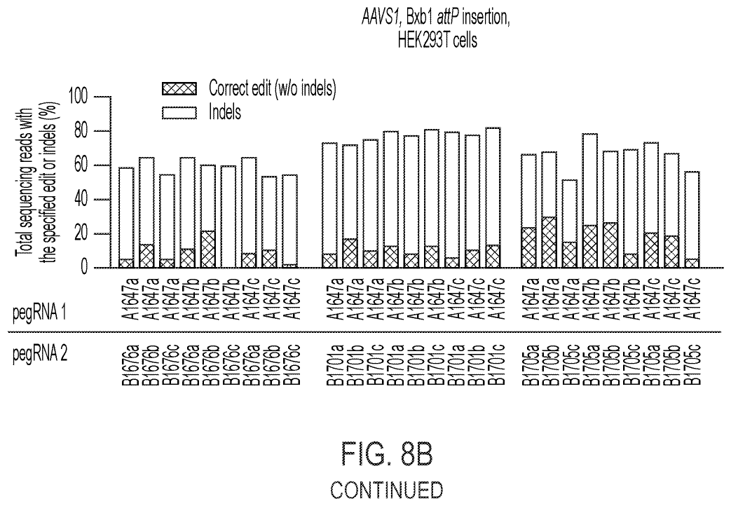

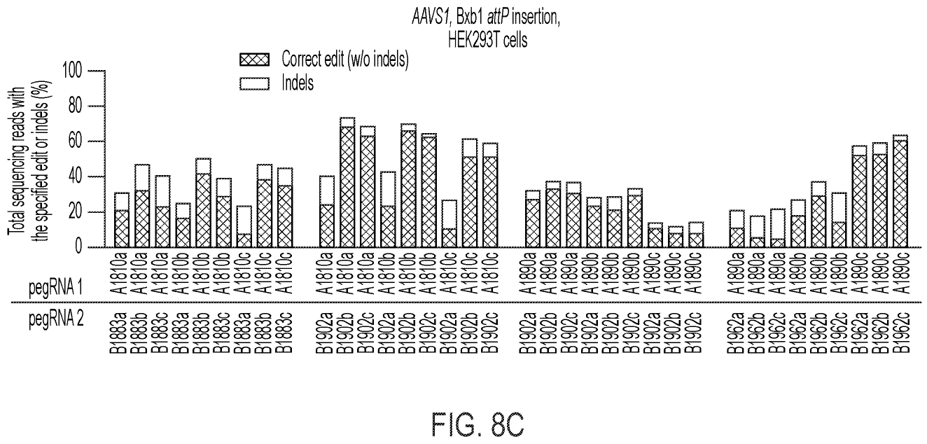

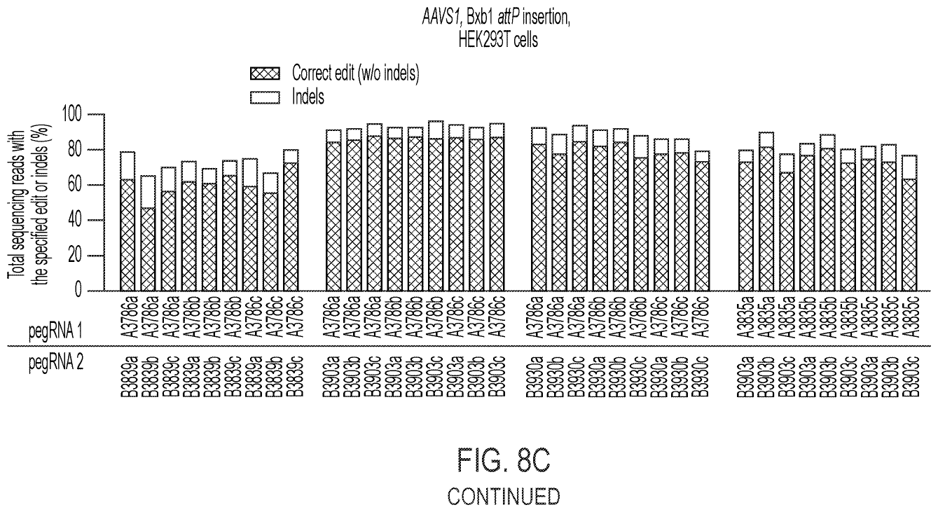

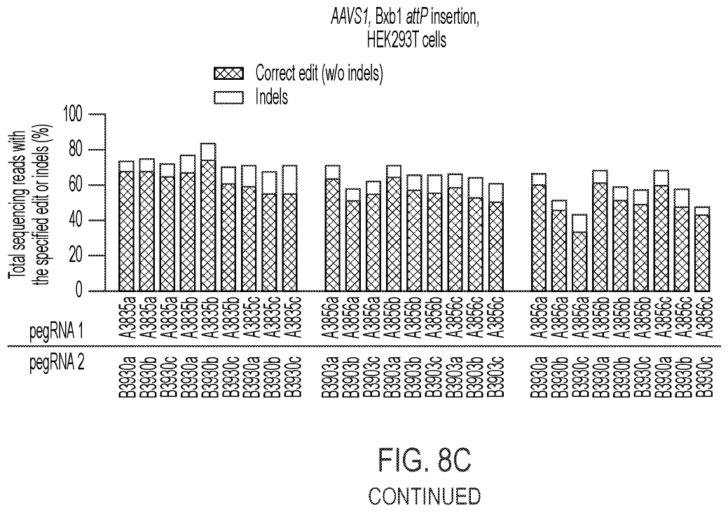

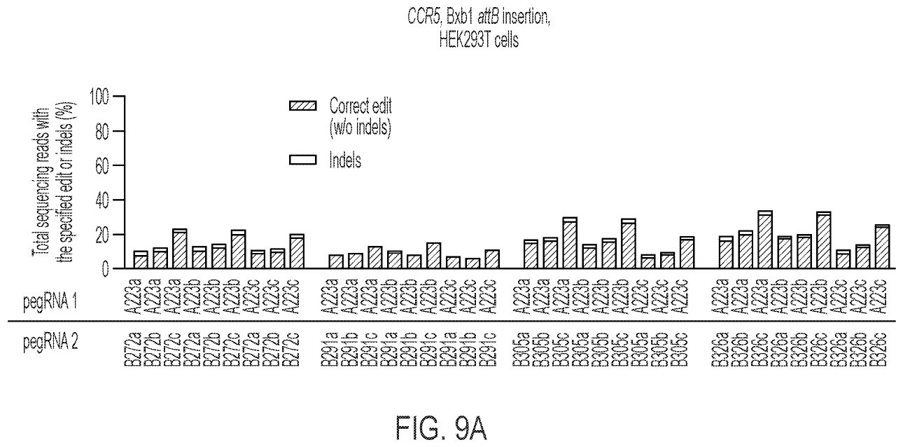

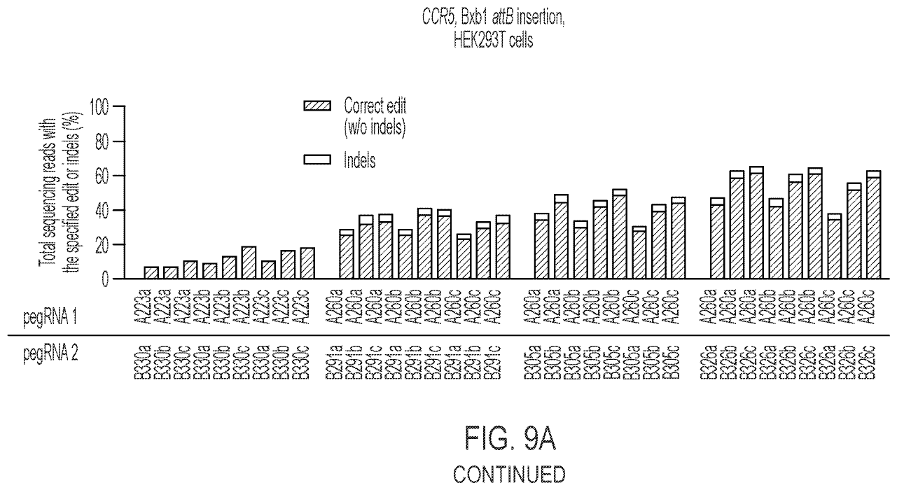

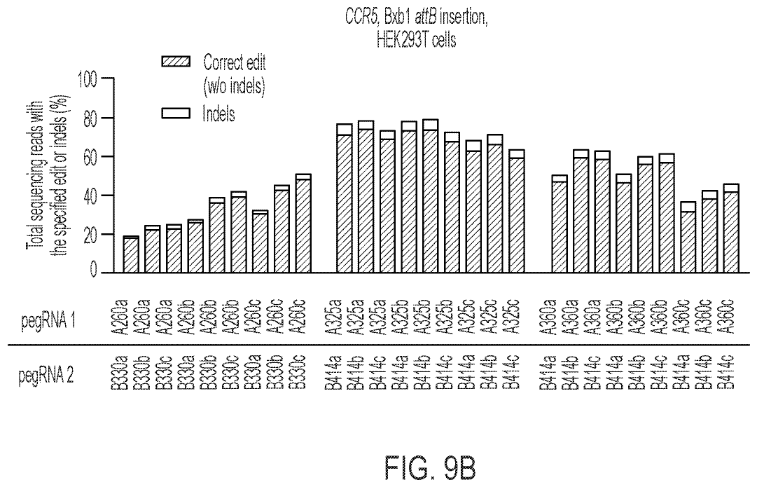

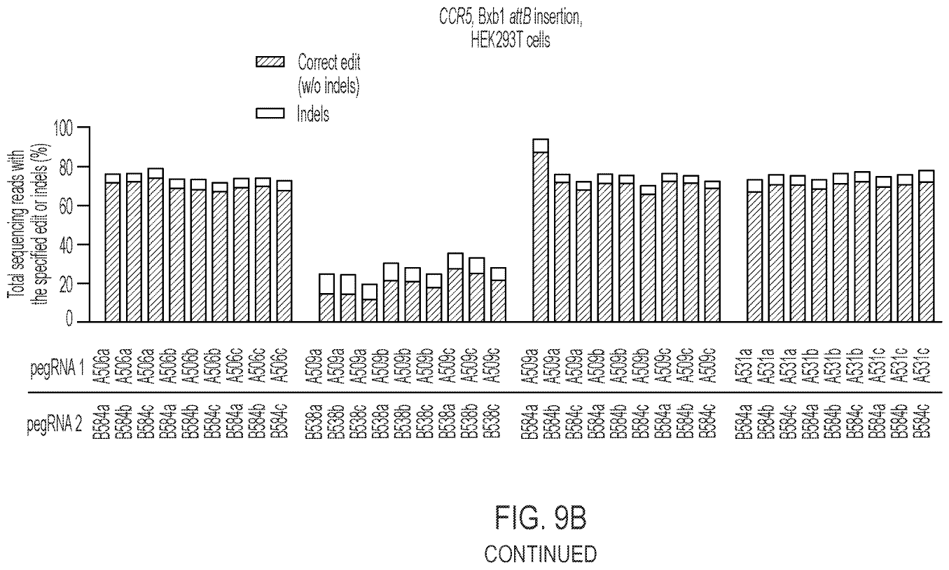

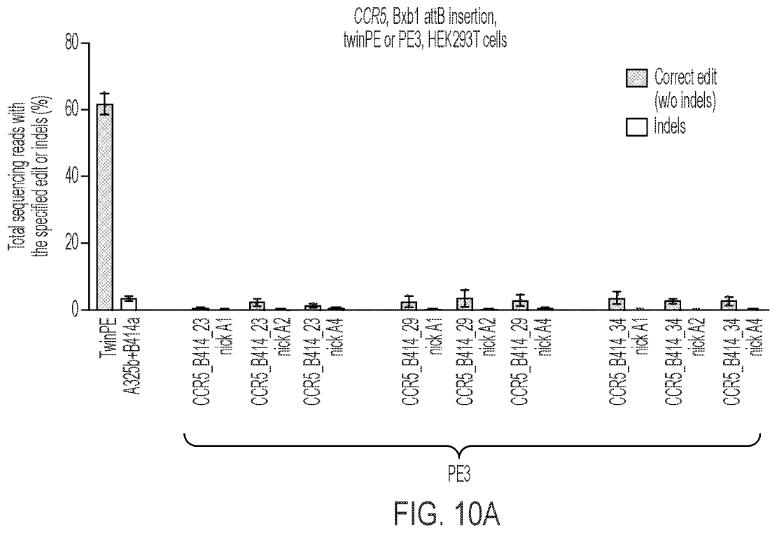

Although twinPE is able to perform large insertion edits, it remains difficult to integrate gene-sized DNA fragments of thousands of base pairs. Having successfully inserted Bxb1 recombinase substrate sequences into endogenous human genomic sites with high efficiency using twinPE, twinPE (FIGS. 3 and 4) was combined with serine recombinases for the site-specific integration of DNA cargo (FIG. 5). Researchers have previously used Bxb1recombinase to integrate exogenous DNA ranging in size from single genes to entire genetic circuits into genomically-integrated attB and attP DNA sequences in a variety of cultured mammalian cells (Xu, Z. et al. BMC Biotechnology 13, 87 (2013); Duportet, X. et al. Nucleic Acids Research 42, 13440-13451 (2014)) and in Drosophila (Voutev, R. and Mann, R. S. Biotechniques 62, 37-38 (2017)). To identify sites for DNA cargo integration, twinPE-mediated insertion of Bxb1 attB and attP attachment sequences was first tested at established human genome safe harbor loci in HEK293T cells. 19 spacer pairs targeting the CCR5 locus were screened for insertion of the 38-bp Bxb1 attB sequence (FIGS. 8A-8C). Optimal PEgRNAs for six spacer pairs achieved >50% editing efficiency of perfectly edited alleles with 3.9-5.4% indel byproducts. Likewise, 32 PEgRNA pairs targeting the AAVS1 locus were screened for insertion of the 50-bp Bxb1 attP sequence (FIGS. 9A-9B), of which 17 optimal spacer combinations achieved >50% efficiency of perfectly edited alleles with a median of 6.6% indels. Notably, twinPE outperformed PE3 for the replacement of endogenous sequence at CCR5 in HEK293T cells with Bxb1 recombinase attB sites. TwinPE yielded 62% perfectly edited alleles with 3.3% indels at one CCR5 site, and 67% perfectly edited alleles with 4.3% indels at a second CCR5 site, compared to 3.3% perfectly edited alleles with 0.1% indels at the first site and 25% perfectly edited alleles with 1.8% indels at the second site by PE3 (FIGS. 10A-10B). These results demonstrate that twinPE can be used to insert recombinase substrate sequences at safe harbor loci in human cells with high efficiency.

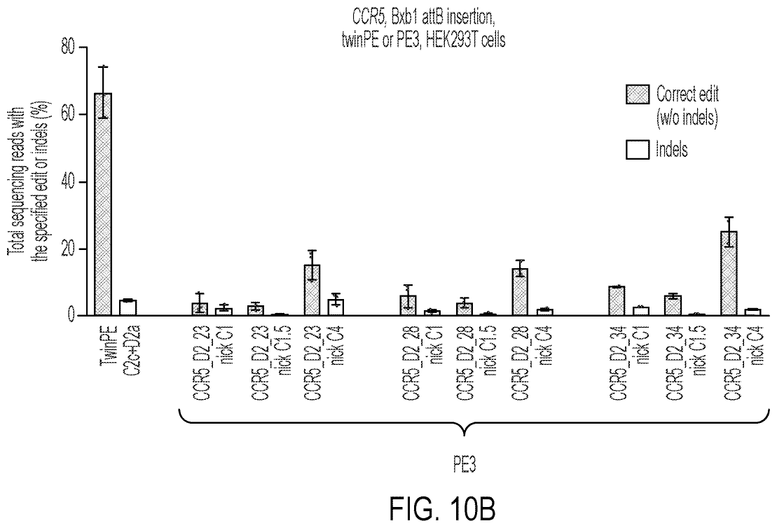

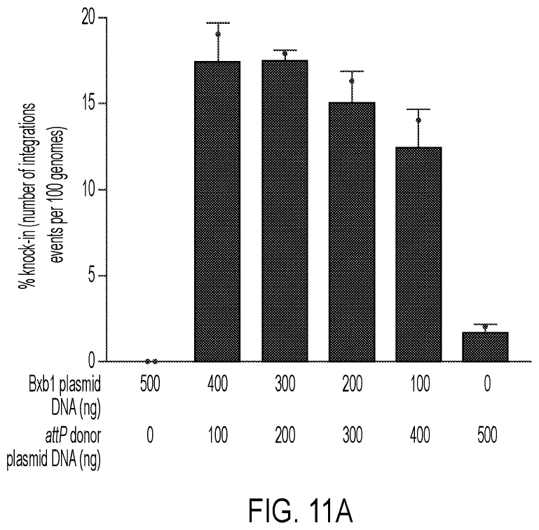

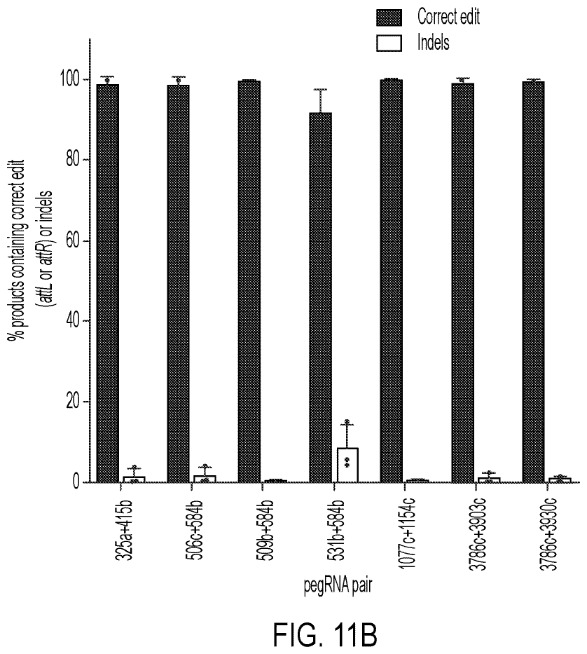

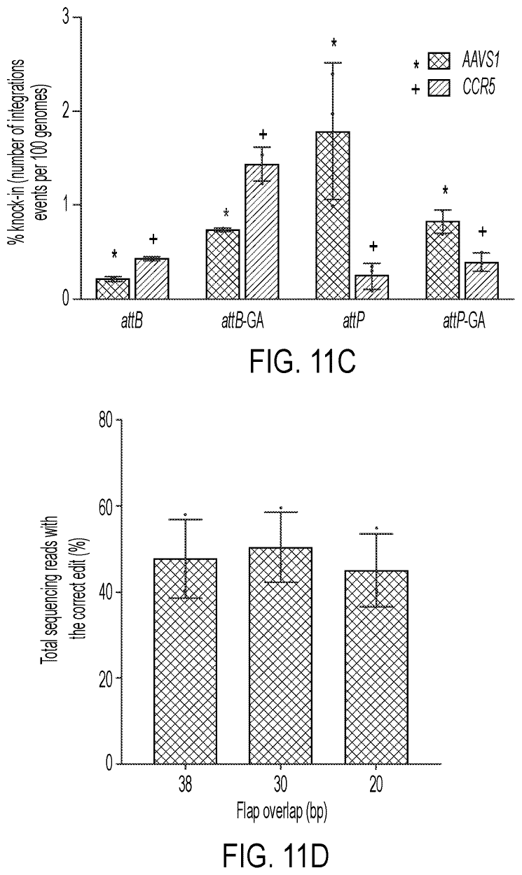

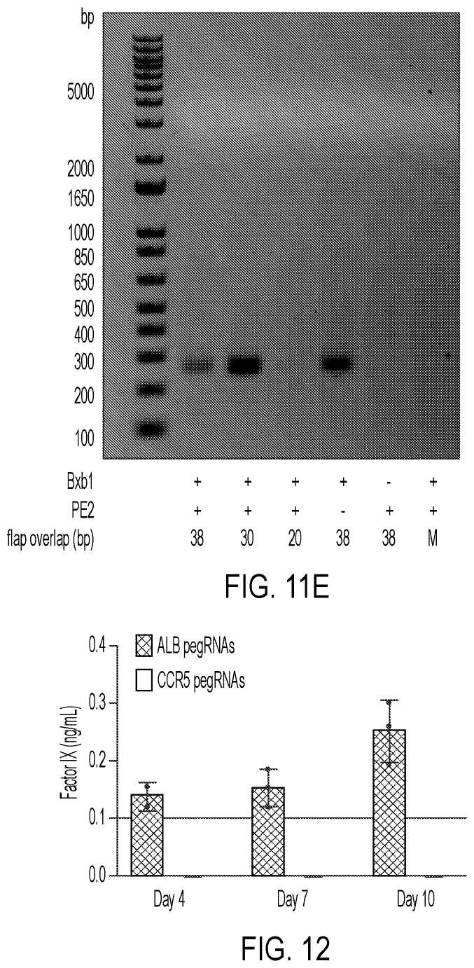

Next, it was examined whether twinPE-incorporated attB or attP sequences could serve as target substrates for the BxB1-mediated integration of DNA plasmids containing partner attP or attB sequences. First, twinPE was used to generate single-cell HEK293T clones bearing homozygous attB site insertions at the CCR5 locus. Transfection of this clonal HEK293T cell line with a plasmid expressing Bxb1 recombinase and a 5.6-kB attP-containing donor DNA plasmid yielded an average of 12-17% integration events per genome of the 5.6-kB plasmid at the target CCR5 site as measured by ddPCR and comparison with an ACTB reference (FIGS. 11A-11C). This efficiency is consistent with previously reported Bxb1-mediated plasmid integration efficiencies in mammalian cells (Voutev, R. and Mann, R. S. Biotechniques 62, 37-38 (2017)).

It was next explored whether twinPE-mediated recombinase site insertion and Bxb1recombinase-mediated DNA donor integration could be achieved by delivering all necessary components in a single transfection step. Initial efforts to transfect HEK293T cells with plasmids encoding PE2, both PEgRNAs, BxB1, and a 5.6-kB donor plasmid resulted in 1.4-6.8% knock-in efficiency as measured by ddPCR. The anticipated junction sequences containing the expected attL and attR recombination products were confirmed by amplicon sequencing, with very high product purities ranging from 91.6-99.8% average attL or attR junctions without indels (median=99.0%) (FIGS. 11A-11C).

### Example 3: Targeted Insertion of Exogenous DNA Sequence in a Target Gene with twinPE

Huh7 cells were transfected with plasmids encoding Bxb1, PE2, an attP-containing donor harboring a splice acceptor followed by the cDNA for human factor IX (hFIX) exons 2-8, and PEgRNAs programming the insertion of Bxb1 attB at intron 1 of ALB. This led to detectable levels of hFIX in conditioned media, whereas no hFIX was detected when the PEgRNAs targeted CCR5 instead of ALB (FIG. 12). Collectively, these results establish a new method for the insertion of gene-sized DNA sequences into targeted genomic loci in previously unmodified human cells without double strand breaks or HDR.

### Example 4: Targeted Inversion of Endogenous DNA Sequence in a Target Gene with twinPE

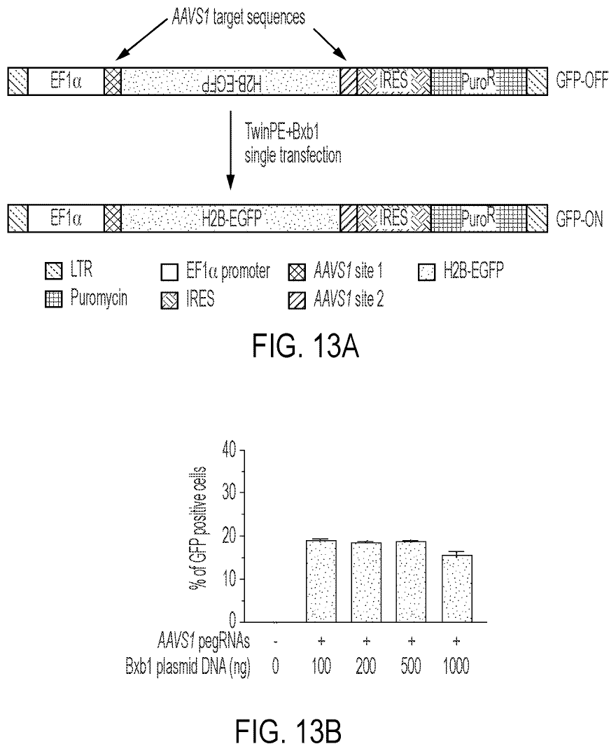

Large structural variants are found in many human pathogenic alleles, such as the large 600-kb inversion at the F8 locus that causes ˜50% of severe hemophilia A cases 54. Inspired by the high efficiency of recombinase attachments site insertions using twinPE, it was reasoned that multiplexing the insertion of both attB and attP Bxb1 attachment sites could be used to correct more complex genetic variants by unidirectional deletion or inversion of the intervening DNA sequence. It was first tested whether twinPE and Bxb1 can revert an inverted H2B-EGFP coding sequence that is stably integrated into the HEK293T genome via lentivirus transduction (FIGS. 13A-13B). After transfection of the reporter cells with twinPE and Bxb1, up to 19% GFP positive cells were observed by flow cytometry, indicating successful inversion (FIG. 13A-13B).

To carry out one-step twinPE and Bxb1-mediated inversion and circumvent unwanted recombination between PEgRNA plasmid DNA, all-RNA components comprising PE2 mRNA, synthetic PEgRNAs from set 1, and Bxb1 mRNA were nucleofected. Using amplicon sequencing, the expected inverted allele junctions containing attR and attL sequences were captured (FIGS. 14A-14B). A reverse primer was designed that can bind to an identical sequence in both the non-inverted and inverted alleles and therefore amplify both edits using the same primer pair, and the inversion efficiency was quantified (FIG. 14A-14B). Collectively, these data suggest that combining twinPE with site-specific serine recombinases can be used to correct a common 39-kb inversion found in Hunter syndrome alleles and may eventually serve as a therapeutic strategy for correcting other large or complex pathogenic gene variants.

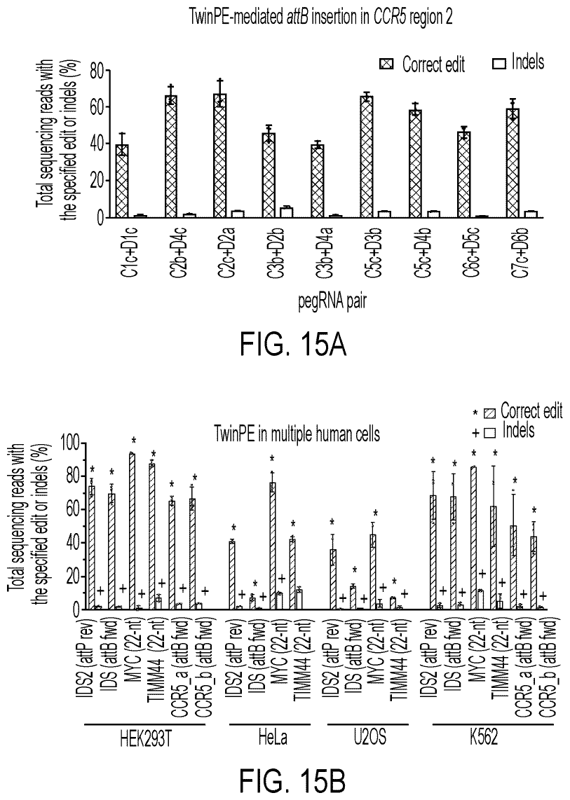

Overall, the Examples provided herein show that twinPE can efficiently replace endogenous genomic DNA sequences with exogenous sequences containing Bxb1 attB or attP recombination sites with observed editing efficiencies of >80% at HEK3 and >40% in four different genomic loci in HeLa, U2OS, and K562 cells (FIGS. 15A-15B).

### Example 5: Targeted Insertion of Exogenous DNA Sequence in a Target Gene with Prime Editor System Having a Single PEgRNA

A single PEgRNA may be designed with a DNA synthesis template comprising an attP sequence, and the PEgRNA may be designed for insertion in a CCR5 safe harbor locus. A donor DNA containing an attB sequence may be designed and co-transfected with the single PEgRNA and the sequence encoding PE2 fusion protein. Plasmid encoding the Bxb1recombinase may be used for recombination between the attB and attP sequences.

### Example 6: Targeted Insertion of Exogenous DNA Sequence in a Target Gene with a Prime Editor System Having twinPE PEgRNAs

A pair of PEgRNAs may be designed with DNA synthesis templates having complementarity to each other, where the region of complementarity includes an attP sequence, and spacers are designed for integration in a CCR5 safe harbor locus. A donor DNA containing an attB sequence may be designed and co-transfected with the single PEgRNA and the sequence encoding PE2 fusion protein. Plasmid encoding the Bxb1recombinase may be used for recombination between the attB and attP sequences.

### Example 7: Targeted Inversion of Endogenous DNA Sequence in a Target Gene with Multiplexed Single PEgRNAs Targeting Different Locations

Two single PEgRNAs may be designed, each comprising a DNA synthesis template including an attB or an attP sequence, for integration of an attB and an attP sequence flanking each end of the region to be inverted in the endogenous target DNA. The PEgRNAs, a plasmid encoding the PE2 prime editor fusion protein, and a plasmid encoding the Bxb1 recombinase may be co-delivered.

### Example 8: Targeted Inversion of Endogenous DNA Sequence in a Target Gene with Multiplexed twinPE

Two pairs of PEgRNAs are designed for integration of an attB sequence and an attP sequence in the target DNA, the attB and an attP sequence flanking each end of the region to be inverted. The two pairs of PEgRNAs, the sequences encoding the PE2 prime editor fusion protein, and the Bxb1 recombinase may be co-delivered.

### Example 9: Targeted Replacement of an Endogenous DNA Sequence with an Exogenous DNA Sequence, Using a Prime Editor System Having Multiplexed Single PEgRNAs Targeting Different Locations

Two single PEgRNAs may be designed and introduced to the target cell to integrate two orthogonal recombinase recognition sites (e.g., a loxP and a lox2272 for Cre and other tyrosine recombinases, or two attB sites for Bxb1 and other serine recombinases) flanking a region to be replaced. Then, a donor DNA with the DNA sequence of interest flanked by a corresponding pair of orthogonal recombinase recognition sites may be supplied with the recombinase (for LoxP and Lox2272, the donor DNA has a LoxP and Lox2272 sequence flanking the sequence of interest; for attB and attB, the donor DNA has attP and attP flanking the sequence of interest). The region between the recombinase sites in the donor may replace the region between the recombinase sites in the genome.

### Example 10: Targeted Replacement of an Endogenous DNA Sequence with an Exogenous DNA Sequence, Using a Prime Editor System Having a Single PEgRNA

A single PEgRNA may be designed and introduced to the target cell to integrate two orthogonal recombinase recognition sites (e.g., a loxP and a lox2272 for Cre and other tyrosine recombinases, or two attB sites for Bxb1 and other serine recombinases) flanking a region to be replaced. Then, a donor DNA with the DNA sequence of interest flanked by a corresponding pair of orthogonal recombinase recognition sites may be supplied with the recombinase (for LoxP and Lox2272, the donor DNA has a LoxP and Lox2272 sequence flanking the sequence of interest; for attB and attB, the donor DNA has attP and attP flanking the sequence of interest). The region between the recombinase sites in the donor may replace the region between the recombinase sites in the genome.

### Example 11: Targeted Replacement of an Endogenous DNA Sequence with an Exogenous DNA Sequence, Using a twinPE System

Two pairs of PEgRNAs may be designed and introduced to the target cell to integrate two orthogonal recombinase recognition sites (e.g., a loxP and a lox2272 for Cre and other tyrosine recombinases, or an attB-GT and an attB-GA for Bxb1 and other serine recombinases) flanking a region to be replaced. Then, a donor DNA with the DNA sequence of interest flanked by a corresponding pair of orthogonal recombinase recognition sites may be supplied with the recombinase (for LoxP and Lox2272, the donor DNA has a LoxP and Lox2272 sequence flanking the sequence of interest; for attB-GT and attB-GA, the donor DNA has attP-GT and attP-GA flanking the sequence of interest). The region between the recombinase sites in the donor may replace the region between the recombinase sites in the genome.

### Example 12: Targeted Deletion of an Endogenous DNA Sequence with a Prime Editor System Having a Single PEgRNA

A single PEgRNA may be designed for integration of an attB sequence and an attP sequence flanking the region to be deleted. The attB and the attP sequences are in the same orientation. The PEgRNA, the sequence encoding PE2 fusion protein, and the Bxb1recombinase may be introduced into the cell.

### Example 13: Targeted Deletion of an Endogenous DNA Sequence with a Prime Editor System Having twinPE PEgRNAs

A pair of PEgRNAs may be designed for integration of an attB sequence and an attP sequence flanking the region to be deleted. The attB and the attP sequences may be in the same orientation. The PEgRNAs, the sequence encoding PE2 fusion protein, and the Bxb1recombinase may be introduced into the cell.

### Example 14: Targeted Installation or Correction of Structural Variants Using twinPE and Bxb1 Recombinase

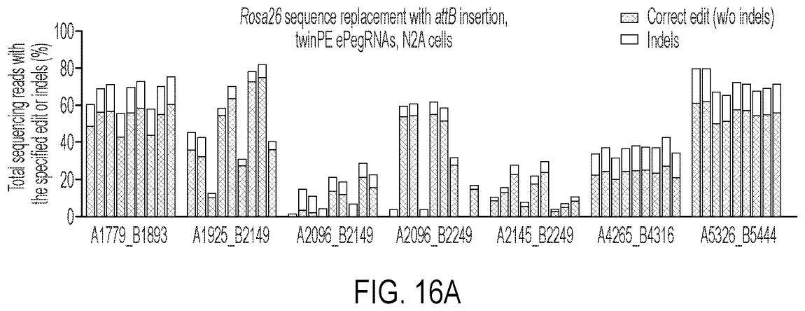

This Example corresponds to the data in FIG. 16A through FIG. 20B.

The invention describes the use of twin prime editing (twinPE)+Bxb1 and multi-flap prime editing for targeted installation or correction of the structural variants (deletion, insertion, sequence replacement, inversion, and translocation) and particularly targeted integration of gene-sized DNA cargo at a specific location in the human genome and other mammalian genome. The latter could enable gene augmentation therapeutic strategies and other biotechnologies and basic research tools (e.g. mouse zygote engineering and transgenic mice models).

The serine integrase phiC31 and fusions of zinc fingers, TALEs, or dCas9 to the catalytic domain of Gin recombinase have been used to integrate at endogenous pseudo-sites in the human genome, but the efficiency of these sequence manipulations has generally been low and, more importantly, the extensive sequence preferences inherent to these recombinases limit the number of targetable loci to a minute fraction of pseudo-sites in the genome. Methods that use HDR can enable control of sequence insertion and can achieve efficiencies on the order of 5-10% without drug selection or suppression of NHEJ, but HDR is less efficient than NHEJ in most cell types and typically requires DSBs, which are highly toxic and can cause genome rearrangements.

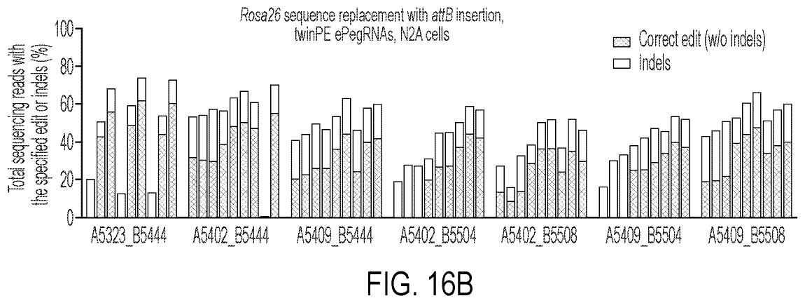

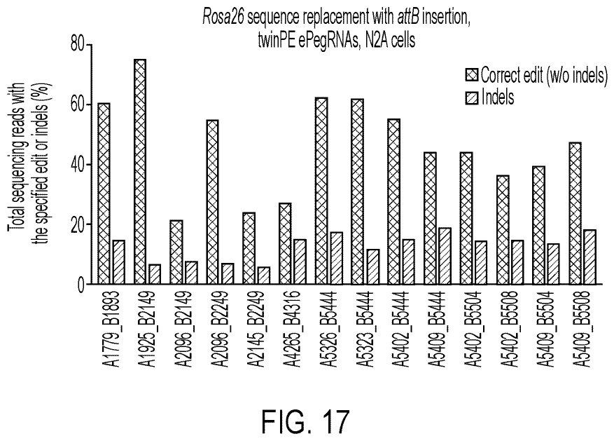

To achieve the programmable integration of attachment site and overcome the other challenges, the inventors applied twinPE and enabled efficient insertion of Bxb1 recombinase attP (50 bp) and attB (38 bp) sites into human and mouse genomic loci, including safe harbor loci. Safe harbor locus (e.g., Rosa26 in the mouse genome and CCR5 in the human genome) is considered a desirable location to perform targeted gene integration for therapeutic and other applications. The inventors have performed dual PEgRNA screen using different spacer pairs with varied primer binding sites targeting Rosa26 locus in the mouse N2A cells. It was found that twinPE-mediated sequence replacement and insertion of attB site can achieve up to 75% efficiency (FIG. 16A-16B). Among 6 of 18 tested spacer pairs, twinPE-mediated attB insertion efficiency is above 50% (FIG. 17). This demonstrated the programmable Bxb1 recombination site insertion via twinPE and thus allow subsequent Bxb1 mediated donor integration or one-pot editing with all the required components (PE2, dual PEgRNAs, Bxb1, and DNA donor).

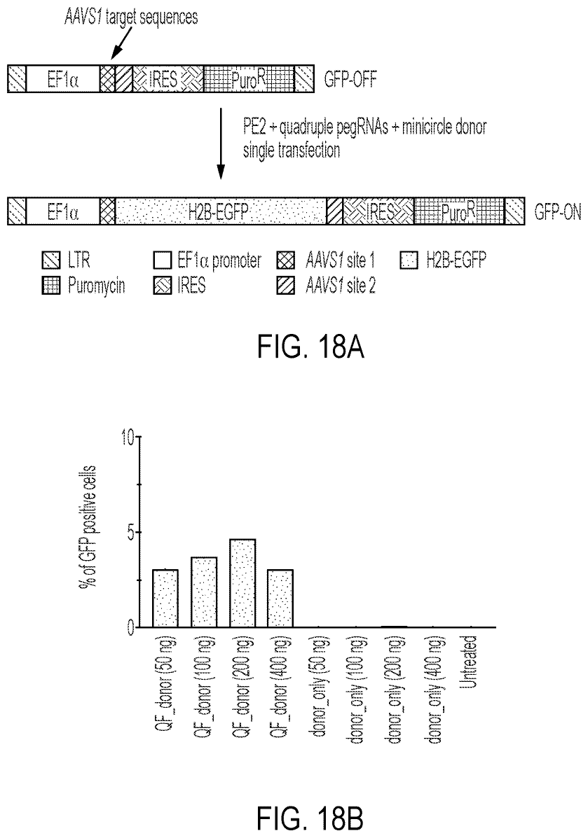

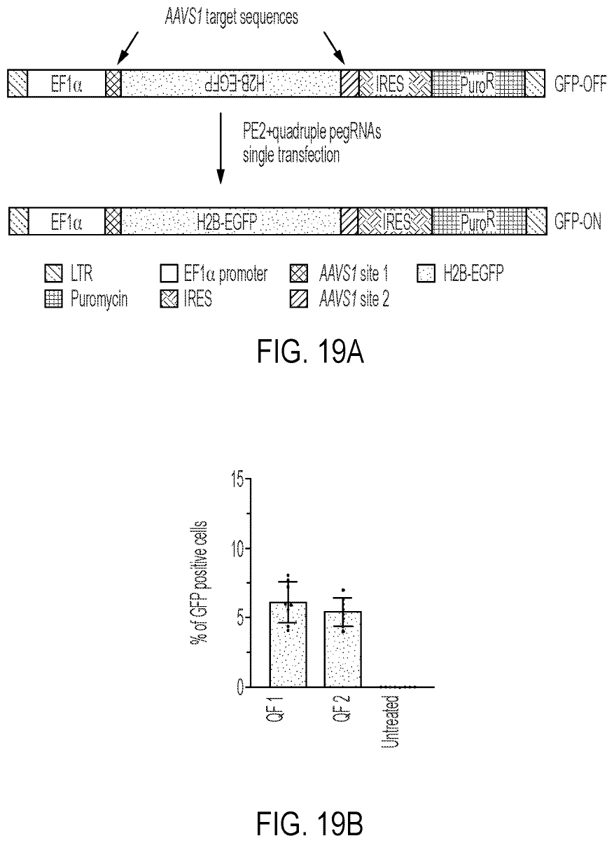

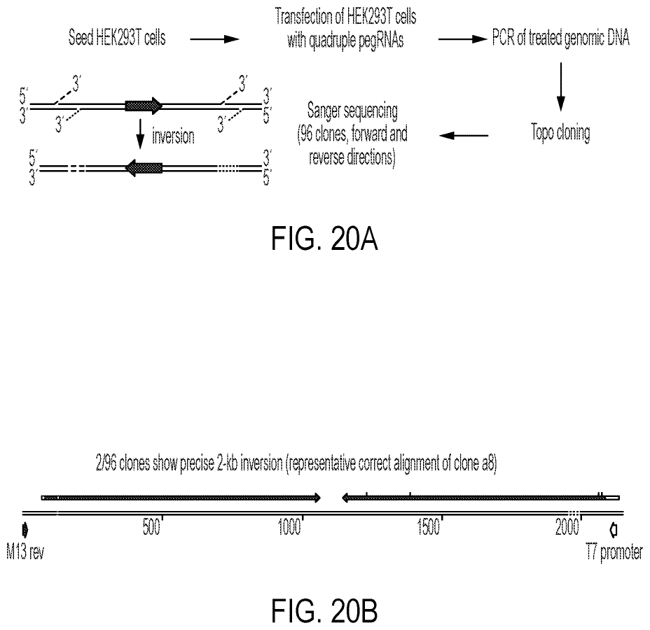

Furthermore, to test whether quadruple PEgRNAs and PE2 editor (quad-flap PE) can achieve DNA donor integration and inversion at the targeted locus (concepts mentioned in the previous patent provision), the inventors constructed two HEK293T GFP reporter cell lines (shown in FIG. 18A and FIG. 19A) and carried out experiments in these cells for assessing quad-flap PE mediated donor integration and sequence inversion. In FIG. 18B, ˜5% GFP+ cells were observed after transfecting reporter cells with quadruple PEgRNAs, PE2 and donor and 0% GFP+ cells with donor alone, suggesting successful promoter-less GFP integration. Quad-flap PE can also efficiently revert inverted H2B-EGFP sequence in the GFP reporter with 6.1% efficiency (FIG. 19B). The inventors also applied our quad-flap PE strategy to target CCR5 in HEK293T cells and successfully invert 2-kb DNA sequences with 2.1% efficiency (FIG. 20A-20B).

Collectively, these results demonstrate twinPE+Bxb1 and quad-flap PE strategies can achieve programmable large DNA donor integration and sequence inversion, promising gene augmentation therapeutic strategies and other biotechnologies and basic research tools.

### Example 15: Installation of Recombinase Sites in iPSCs Using Twin Prime Editing

Induced pulripotent stell cell (iPSC) colonies at 70%-80% confluency were washed once with DPBS and dissociated in pre-warmed Accutase. Next, iPS cells were gently triturated, moved into a sterile 15 mL conical tube, then combined with an equal volume of DMEM/F12 (Thermo Fisher Scientific) to quench dissociation enzyme activity. Cells were pelleted at 300 g for 3 min and resuspended in StemFlex medium supplemented with 10 μM Y-27632. For electroporation using the NEON Transfection System 10 μL kit (Thermo Fisher Scientific), 0.2 million iPS cells were pelleted at 300 g for 3 min and resuspended in 9 μL NEON Buffer R. The cell solution was combined with a 3 μL mixture of 1 mg PE2 mRNA and 75 pmol of each synthetic pegRNA (Integrated DNA Technologies). Mock control electroporations were performed with 3 μL NEON Buffer R without any RNA added. Directly prior to electroporation, rhLa-minin-521 was aspirated and immediately replaced with 250 μL pre-warmed StemFlex medium supplemented with 10 μM Y-27632 per 24-well. Next, 10 μL of the combined cell and RNA mixture was electroporated using the NEON Transfection System (Thermo FisherScientific) with the following parameters: 1400 V, 20 ms, and one pulse. Cells were seeded immediately into rhLaminin-521-coated 24-well plates with 250 mL StemFlex medium supplemented with 10 mM Y-27632 per well. Media was changed the following day with 500 mL StemFlex medium supplemented with 5 mM Y-27632. 72 h following electroporation, media was changed to 500 μL StemFlex medium per well. Genomic DNA was extracted, and PCR amplified for Illumina sequencing.

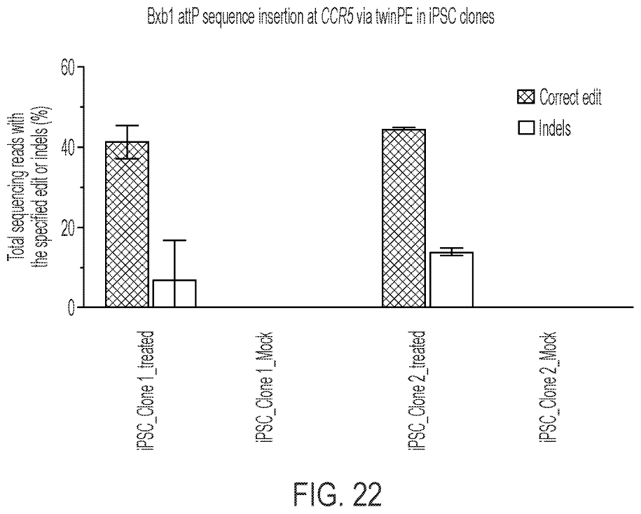

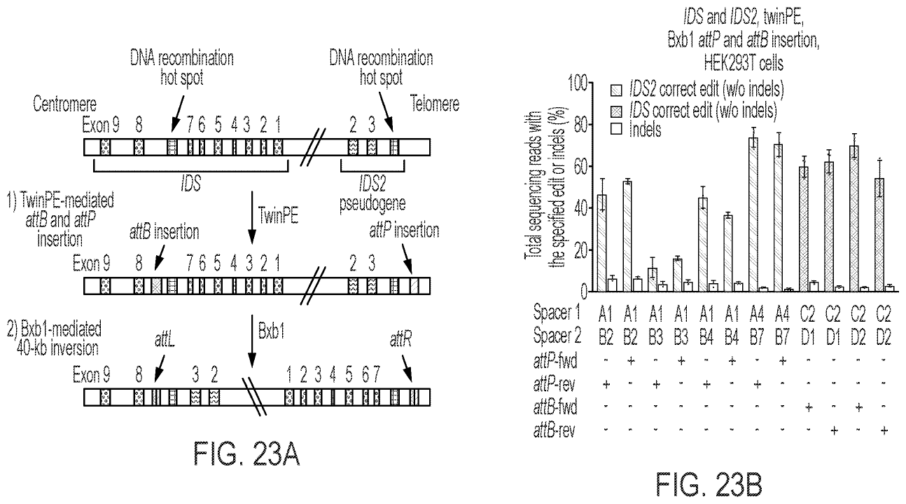

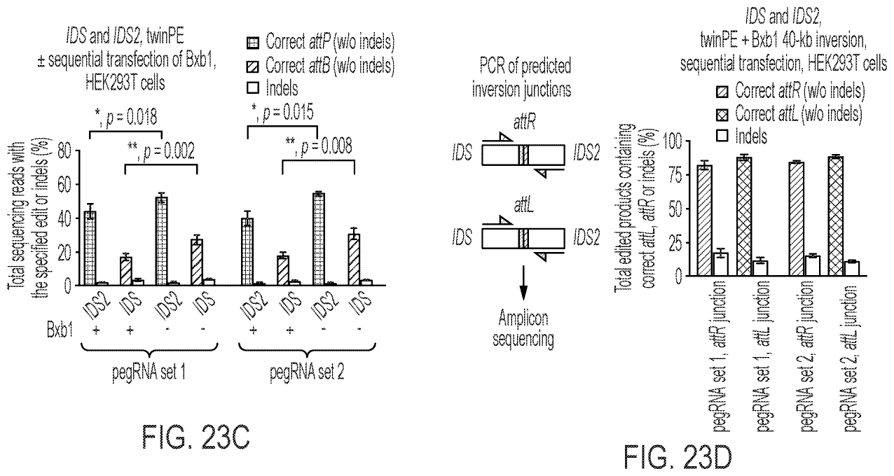

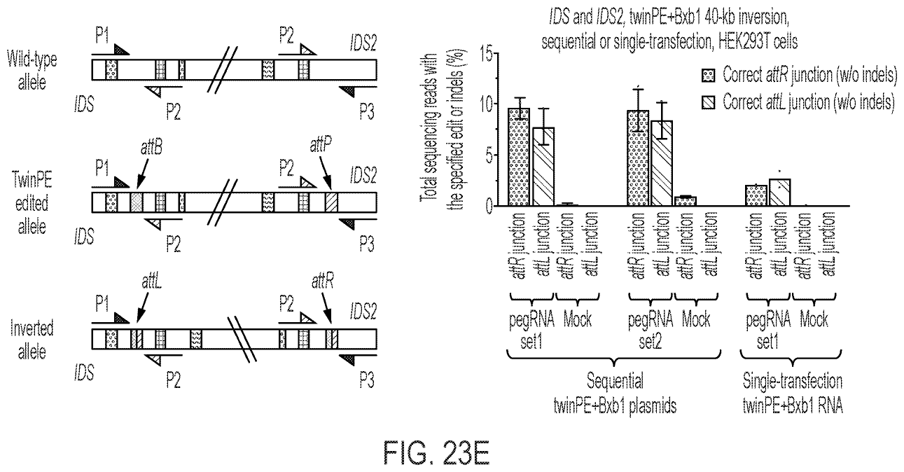

Overall, TwinPE (with PE2 mRNA and dual synthetic pegRNAs) was used to successfully insert 38 bp Bxb1 at the CCR5 safe harbor locus with up to 45% editing efficiency in bulk iPSCs without any selection (FIG. 22).

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method for editing a target DNA, comprising contacting the target DNA with a prime editor system comprising:
(i) a prime editor comprising a napDNAbp and a DNA polymerase, or a polynucleotide encoding the napDNAbp and/or the DNA polymerase; and
(ii) a prime editing guide RNA (PEgRNA), or a polynucleotide encoding the PEgRNA, wherein the PEgRNA comprises a spacer, a gRNA core, and a DNA synthesis template, wherein the DNA synthesis template comprises one or more recombinase recognition site as compared to the target DNA;
wherein the prime editor system introduces the one or more recombinase recognition sites in the target DNA and results in an intended edit in the target DNA.

2. A method for editing a target DNA, comprising contacting the target DNA with a prime editor system comprising:
(i) a prime editor comprising a napDNAbp and a DNA polymerase, or a polynucleotide encoding the napDNAbp and/or the DNA polymerase;
(ii) a first prime editing guide RNA (PEgRNA) or a polynucleotide encoding the first PEgRNA, wherein the first PEgRNA comprises a first spacer, a first gRNA core, and a first DNA synthesis template, wherein the first spacer binds to a first sequence in the target DNA and wherein the first DNA synthesis template comprises one or more recombinase recognition sites as compared to the target DNA,
(iii) a second prime editing guide RNA (PEgRNA) or a polynucleotide encoding the second PEgRNA, wherein the second PEgRNA comprises a second spacer, a second gRNA core, and a second DNA synthesis template, wherein the second spacer binds to a second sequence in the target DNA and wherein the second DNA synthesis template comprises one or more recombinase recognition sites as compared to the target DNA;
wherein the prime editor system introduces two or more recombinase recognition sites in the target DNA and results in an intended edit in the target DNA.

3. A method for editing a target DNA, comprising contacting the target DNA with a prime editor system comprising:
(i) a prime editor comprising a napDNAbp and a DNA polymerase or a polynucleotide encoding the napDNAbp and/or the DNA polymerase;
(ii) a first prime editing guide RNA (PEgRNA) or a polynucleotide encoding the first PEgRNA, wherein the first PEgRNA comprises a first spacer, a first gRNA core, and a first DNA synthesis template; and
(iii) a second prime editing guide RNA (PEgRNA) or a polynucleotide encoding the second PEgRNA, wherein the second PEgRNA comprises a second spacer, a second gRNA core, and a second DNA synthesis template;
wherein the first DNA synthesis template and the second DNA synthesis template comprises a region of complementarity to each other,
wherein the sequence (the first DNA synthesis template not complementary to the second DNA synthesis template+the region of complementarity between the first DNA synthesis template+the second DNA synthesis template not complementary to the first DNA synthesis template) comprises a recombinase recognition site as compared to the target DNA,
and wherein the prime editor system introduces the recombinase recognition site in the target DNA and results in an intended edit in the target DNA.

4. A method for editing a target DNA, comprising contacting the target DNA with a prime editor system comprising:
(i) a prime editor comprising a napDNAbp and a DNA polymerase or a polynucleotide encoding the napDNAbp and/or the DNA polymerase;
(ii) a first prime editing guide RNA (PEgRNA) or a polynucleotide encoding the first PEgRNA, wherein the first PEgRNA comprises a first spacer, a first gRNA core, and a first DNA synthesis template;
(iii) a second prime editing guide RNA (PEgRNA) or a polynucleotide encoding the second PEgRNA, wherein the second PEgRNA comprises a second spacer, a second gRNA core, and a second DNA synthesis template;
(iv) a third prime editing guide RNA (PEgRNA) or a polynucleotide encoding the third PEgRNA, wherein the third PEgRNA comprises a third spacer, a third gRNA core, and a third DNA synthesis template; and
(v) a fourth prime editing guide RNA (PEgRNA) or a polynucleotide encoding the fourth PEgRNA, wherein the fourth PEgRNA comprises a fourth spacer, a fourth gRNA core, and a fourth DNA synthesis template;
wherein the first DNA synthesis template and the second DNA synthesis template comprises a region of complementarity to each other,
wherein the sequence (the first DNA synthesis template not complementary to the second DNA synthesis template+the region of complementarity between the first DNA synthesis template+the second DNA synthesis template not complementary to the first DNA synthesis template) comprises a first recombinase recognition site as compared to the target DNA,
wherein the third DNA synthesis template and the fourth DNA synthesis template comprises a region of complementarity to each other,
wherein the sequence (the third DNA synthesis template not complementary to the second DNA synthesis template+the region of complementarity between the first DNA synthesis template+the fourth DNA synthesis template not complementary to the first DNA synthesis template) comprises a second recombinase recognition site as compared to the target DNA,
and wherein the prime editor system introduces the first and the second recombinase recognition site in the target DNA and results in an intended edit in the target DNA.

5. The method of claim 3, wherein the region of complementarity between the first and the second DNA synthesis template comprises the recombinase recognition site.

6. The method of claim 4, wherein the region of complementarity between the first and the second DNA synthesis template comprises the first recombinase recognition site, and/or wherein the region of complementarity between the third and the fourth DNA synthesis template comprises the second recombinase recognition site.

7. The method of any one of claims 1-6, further comprising contacting the target DNA with a recombinase or a polynucleotide encoding the recombinase.

8. The method of any one of claims 1-5, further comprising contacting the target DNA with a donor DNA, wherein the donor DNA comprises a recombinase recognition site.

9. The method of any one of claims 1-8, wherein the prime editor system introduces a single recombinase recognition site to the target DNA.

10. The method of any one of claims 1-8, wherein the prime editor system introduces at least two recombinase recognition sites to the target DNA.

11. The method of claim 9, wherein the intended edit is an insertion of an exogenous DNA sequence.

12. The method of claim 11, wherein the exogenous DNA sequence is from a donor comprising a recombinase recognition site.

13. The method of claim 11, wherein the insertion is in a non-coding region of a gene.

14. The method of claim 11, wherein the insertion is in a coding region of a gene.

15. The method of claim 14, wherein the gene is CCR5, AAVS1, Rosa26, or PCSK9.

16. The method of claim 14, wherein the gene is IDS or IDS2.

17. The method of claim 11, wherein the insertion is downstream of a promoter of a gene.

18. The method of any one of claims 11-17, wherein the exogenous DNA sequence encodes a protein or a portion thereof.

19. The method of claim 18, wherein the protein is a therapeutic protein.

20. The method of claim 10, wherein the intended edit is a deletion of an endogenous sequence from the target DNA.

21. The method of claim 20, wherein the deletion is in a non-coding region of a gene.

22. The method of claim 20, wherein the deletion is in a coding region of a gene.

23. The method of claim 21 or 22, wherein the deletion is in a disease associated gene, and wherein the deleted sequence comprises a mutation associated with the disease.

24. The method of claim 23, wherein the deleted sequence comprises a trinucleotide repeat.

25. The method of claim 10, wherein the intended edit is replacement of an endogenous sequence of the target DNA by an exogenous DNA sequence.

26. The method of claim 14, wherein the exogenous DNA sequence is from a donor comprising a recombinase recognition site.

27. The method of claim 25 or 26, wherein the endogenous sequence is in a disease associated gene and comprises a mutation associated with the disease.

28. The method of claim 27, wherein the exogenous DNA sequence comprises a wild-type sequence of the disease associated gene.

29. The method of claim 10, wherein the intended edit is an inversion of an endogenous sequence of the target DNA.

30. The method of claim 29, wherein the endogenous sequence is in a coding region of a gene.

31. The method of claim 29, wherein the endogenous sequence is in a non-coding region of a gene.

32. The method of claim 30 or 31, wherein the endogenous sequence is in a disease associated gene, and wherein the inversion restores a wild-type sequence of the gene.

33. The method of any one of claims 11-20, wherein the recombinase is a serine recombinase.

34. The method of any one of claims 11-20, wherein the recombinase is a tyrosine recombinase.

35. The method of any one of claims 21-32, wherein the recombinase is a serine recombinase.

36. The method of any one of claims 1-35, wherein the recombinase recognition site comprises a Bxb1 recombinase recognition site.

37. The method of any one of claims 1-36, wherein the method is performed in a cell.

38. The method of claim 37, wherein the cell is an induced pluripotent stem cell (iPSC).


SEQUENCE LISTING (263 sequences)

SEQ ID NO: 1 - DNA, 20 bp, synthetic construct
  source 1..20: mol_type = other DNA
ttgaaaaagtggcaccgagt

SEQ ID NO: 2 - DNA, 17 bp, synthetic construct
  source 1..17: mol_type = other DNA
ctcccactcatgatcta

SEQ ID NO: 3 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 4 - protein, 19 aa, synthetic construct
MKRTADGSEFESPKKKRKV

SEQ ID NO: 5 - protein, 21 aa, synthetic construct
SGGSKRTADGSEFEPKKKRKV

SEQ ID NO: 6 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 33 aa, synthetic construct
SGGSSGGSSGSETPGTSESATPESSGGSSGGSS

SEQ ID NO: 8 - protein, 677 aa, unidentified
  source 1..677: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 9 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 10 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 11 - protein, 1943 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDNSRLINSGGSKRTADGSEFEPKKKRKV

SEQ ID NO: 12 - protein, 503 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDNSRLIN

SEQ ID NO: 13 - protein, 1368 aa, Streptococcus pyogenes [sequence omitted]

SEQ ID NO: 14 - protein, 560 aa, unidentified
  source 1..560: note = Moloney murine leukemia virus
AFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLGRLEDLKSKTLGDLVREAEKIFNKRETPEEREERIRRETEEKEERRRTVDEQKEKERDRRRHREMSKLLATVVIGQEQDRQEGERKRPQLDKDQCAYCKEKGHWAKDCPKKPRGPRGPRPQTSLLTLGDXGGQGQDPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVSLGFTWLSDFPQAWAESGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFR

SEQ ID NO: 15 - protein, 669 aa, Feline leukemia virus
TLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLPVKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTLFDEALHSDLADFRVRYPALVLLQYVDDLLLAAATRTECLEGTKALLETLGNKGYRASAKKAQICLQEVTYLGYSLKDGQRWLTKARKEAILSIPVPKNSRQVREFLGTAGYCRLWIPGFAELAAPLYPLTRPGTLFQWGTEQQLAFEDIKKALLSSPALGLPDITKPFELFIDENSGFAKGVLVQKLGPWKRPVAYLSKKLDTVASGWPPCLRMVAAIAILVKDAGKLTLGQPLTILTSHPVEALVRQPPNKWLSNARMTHYQAMLLDAERVHFGPTVSLNPATLLPLPSGGNHHDCLQILAETHGTRPDLTDQPLPDADLTWYTDGSSFIRNGEREAGAAVTTESEVIWAAPLPPGTSAQRAELIALTQALKMAEGKKLTVYTDSRYAFATTHVHGEIYRRRGLLTSEGKEIKNKNEILALLEALFLPKRLSIIHCPGHQKGDSPQAKGNRLADDTAKKAATETHSSLTVL

SEQ ID NO: 16 - protein, 559 aa, Human immunodeficiency virus 1
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLXKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKV

SEQ ID NO: 17 - protein, 440 aa, Human immunodeficiency virus 1
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 18 - protein, 858 aa, Rous sarcoma virus
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQKEPGVQYLGYKLGSTYAAPVGLVAEPRIATLWDVQKLVGSLQWLRPALGIPPRLRGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFRDELPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATVIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFA

SEQ ID NO: 19 - protein, 675 aa, Cauliflower mosaic virus
MMDHLLQKTQIQNQTEQVMNITNPNSIYIKGRLYFKGYKKIELHCFVDTGASLCIASKFVIPEEHWINAERPIMVKIADGSSITINKVCRDIDLIIAGEIFHIPTVYQQESGIDFIIGNNFCQLYEPFIQFTDRVIFTKDRTYPVHIAKLTRAVRVGTEGFLESMKKRSKTQQPEPVNISTNKIAILSEGRRLSEEKLFITQQRMQKIEELLEKVCSENPLDPNKTKQWMKASIKLSDPSKAIKVKPMKYSPMDREEFDKQIKELLDLKVIKPSKSPHMAPAFLVNNEAEKRRGKKRMVVNYKAMNKATVGDAYNLPNKDELLTLIRGKKIFSSFDCKSGFWQVLLDQDSRPLTAFTCPQGHYEWNVVPFGLKQAPSIFQRHMDEAFRVFRKFCCVYVDDILVFSNNEEDHLLHVAMILQKCNQHGIILSKKKAQLFKKKINFLGLEIDEGTHKPQGHILEHINKFPDTLEDKKQLQRFLGILTYASDYIPKLAQIRKPLQAKLKENVPWKWTKEDTLYMQKVKKNLQGFPPLHHPLPEEKLIIETDASDDYWGGMLKAIKINEGTNTELICRYASGSFKAAEKNYHSNDKETLAVINTIKKFSIYLTPVHFLIRTDNTHFKSFVNLNYKGDSKLGRNIRWQAWLSHYSFDVEHIKGTDNHFADFLSREFNRVNS

SEQ ID NO: 20 - protein, 366 aa, Klebsiella pneumoniae
MKEKISKIDKNFYTDIFIKTSFQNEFEAGGVIPPIAKNQVSTISNKNKTFYSLAHSSPHYSIQTRIEKFLLKNIPLSASSFAFRKERSYLHYLEPHTQNVKYCHLDIVSFFHSIDVNIVRDTFSVYFSDEFLVKEKQSLLDAFMASVTLTAELDGVEKTFIPMGFKSSPSISNIIFRKIDILIQKFCDKNKITYTRYADDLLFSTKKENNILSSTFFINEISSILSINKFKLNKSKYLYKEGTISLGGYVIENILKDNSSGNIRLSSSKLNPLYKALYEIKKGSSSKHICIKVFNLKLKRFIYKKNKEKFEAKFYSSQLKNKLLGYRSYLLSFVIFHKKYKCINPIFLEKCVFLISEIESIMNRKF

SEQ ID NO: 21 - protein, 263 aa, Escherichia coli
MKITSNNVTAVINGKGWHSINWKKCHQHVKTIQTRIAKAACNQQWRTVGRLQRLLVRSFSARALAVKRVTENSGRKTPGVDGQIWSTPESKWEAIFKLRRKGYKPLPLKRVFIPKSNGKKRPLGIPVMLDRAMQALHLLGLEPVSETNADHNSYGFRPARCTADAIQQVCNMYSSRNASKWVLEGDIKGCFEHISHEWLLENIPMDKQILRNWLKAGIIEKSIFSKTLSGTPQGGIISPVLANMALDGLERLLQNRFGRNRLI

SEQ ID NO: 22 - protein, 438 aa, Bacillus subtilis
MSKIKINYEKYHIKPFPHFDQRIKVNKKVKENLQNPFYIAAHSFYPFIHYKKISYKFKNGTLSSPKERDIFYSGHMDGYIYKHYGEILNHKYNNTCIGKGIDHVSLAYRNNKMGKSNIHFAAEVINFISEQQQAFIFVSDFSSYFDSLDHAILKEKLIEVLEEQDKLSKDWWNVFKHITRYNWVEKEEVISDLECTKEKIARDKKSRERYYTPAEFREFRKRVNIKSNDTGVGIPQGTAISAVLANVYAIDLDQKLNQYALKYGGIYRRYSDDIIMVLPMTSDGQDPSNDHVSFIKSVVKRNKVTMGDSKTSVLYYANNNIYEDYQRKRESKMDYLGFSFDGMTVKIREKSLFKYYHRTYKKINSINWASVKKEKKVGRKKLYLLYSHLGRNYKGHGNFISYCKKAHAVFEGNKKIESLINQQIKRHWKKIQKRLVDV

SEQ ID NO: 23 - protein, 426 aa, Eubacterium rectale
DTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 24 - protein, 412 aa, Geobacillus stearothermophilus
ALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQR

SEQ ID NO: 25 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 26 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 27 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 28 - protein, 677 aa, synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 29 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 30 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 31 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 32 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 33 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 34 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 35 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 36 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 37 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 38 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 39 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 40 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 41 - protein, 2117 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 42 - protein, 2144 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSSGGSSGSETPGTSESATPESAGSYPYDVPDYAGSAAPAAKKKKLDGSGSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 43 - protein, 60 aa, synthetic construct
SGGSSGGSSGSETPGTSESATPESAGSYPYDVPDYAGSAAPAAKKKKLDGSGSGGSSGGS

SEQ ID NO: 44 - protein, 2529 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGKSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 45 - protein, 379 aa, synthetic construct
GIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 46 - protein, 1803 aa, Homo sapiens
MKRTADGSEFESPKKKRKVGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 47 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 48 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 49 - protein, 150 aa, synthetic construct
  SITE 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 50 - protein, 30 aa, synthetic construct
  SITE 2..30: note = may be absent
GGGGGGGGGGGGGGGGGGGGGGGGGGGGGG

SEQ ID NO: 51 - protein, 150 aa, synthetic construct
  SITE 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 52 - protein, 90 aa, synthetic construct
  SITE 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 53 - protein, 120 aa, synthetic construct
  SITE 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 54 - protein, 60 aa, synthetic construct
  SITE 3..60: note = may be absent
XPXPXPXPXPXPXPXPXPXPXPXPXPXPXPXPXPXPXPXPXPXPXPXPXPXPXPXPXPXP

SEQ ID NO: 55 - protein, 16 aa, synthetic construct
SGSETPGTSESATPES

SEQ ID NO: 56 - protein, 32 aa, synthetic construct
SGGSSGGSSGSETPGTSESATPESSGGSSGGS

SEQ ID NO: 57 - protein, 10 aa, synthetic construct
SGGSGGSGGS

SEQ ID NO: 58 - protein, 4 aa, synthetic construct
SGGS

SEQ ID NO: 59 - skipped

SEQ ID NO: 60 - protein, 6 aa, synthetic construct
GGSGGS

SEQ ID NO: 61 - protein, 9 aa, synthetic construct
GGSGGSGGS

SEQ ID NO: 62 - protein, 7 aa, synthetic construct
PKKKRKV

SEQ ID NO: 63 - protein, 30 aa, synthetic construct
MDSLLMNRRKFLYQFKNVRWAKGRRETYLC

SEQ ID NO: 64 - protein, 20 aa, synthetic construct
AVKRPAATKKAGQAKKKKLD

SEQ ID NO: 65 - protein, 25 aa, synthetic construct
MSRRRKANPTKLSENAKKLAKEVEN

SEQ ID NO: 66 - protein, 9 aa, synthetic construct
PAAKRVKLD

SEQ ID NO: 67 - protein, 9 aa, synthetic construct
KLKIKRPVK

SEQ ID NO: 68 - protein, 8 aa, synthetic construct
VSRKRPRP

SEQ ID NO: 69 - protein, 10 aa, synthetic construct
EGAPPAKRAR

SEQ ID NO: 70 - protein, 12 aa, synthetic construct
PPQPKKKPLDGE

SEQ ID NO: 71 - protein, 18 aa, synthetic construct
KRTADGSEFESPKKKRKV

SEQ ID NO: 72 - protein, 17 aa, synthetic construct
KRTADGSEFEPKKKRKV

SEQ ID NO: 73 - protein, 19 aa, synthetic construct
NLSKRPAAIKKAGQAKKKK

SEQ ID NO: 74 - protein, 11 aa, synthetic construct
RQRRNELKRSF

SEQ ID NO: 75 - protein, 38 aa, synthetic construct
NQSSNFGPMKGGNFGGRSSGPYGGGGQYFAKPRNQGGY

SEQ ID NO: 76 - protein, 16 aa, unidentified
  source 1..16: note = Xenopus nucleoplasmin
KRXXXXXXXXXXKKKL

SEQ ID NO: 77 - protein, 380 aa, Homo sapiens
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 78 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVRAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 79 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGAPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 80 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSARRKEPEPKGSTKKKAKTGAAGKFKRGK

SEQ ID NO: 81 - protein, 908 aa, synthetic construct
MGVNDLWQILEPVKQHIPLRNLGGKTIAVDLSLWVCEAQTVKKMMGSVMKPHLRNLFFRISYLTQMDVKLVFVMEGEPPKLKADVISKRNQSRYGSSGKSWSQKTGRSHFKSVLRECLHMLECLGIPWVQAAGEAEAMCAYLNAGGHVDGCLTNDGDTFLYGAQTVYRNFTMNTKDPHVDCYTMSSIKSKLGLDRDALVGLAILLGCDYLPKGVPGVGKEQALKLIQILKGQSLLQRFNRWNETSCNSSPQLLVTKKLAHCSVCSHPGSPKDHERNGCRLCKSDKYCEPHDYEYCCPCEWHRTEHDRQLSEVENNIKKKACCCEGFPFHEVIQEFLLNKDKLVKVIRYQRPDLLLFQRFTLEKMEWPNHYACEKLLVLLTHYDMIERKLGSRNSNQLQPIRIVKTRIRNGVHCFEIEWEKPEHYAMEDKQHGEFALLTIEEESLFEAAYPEIVAVYQKQKLEIKGKKQKRIKPKENNLPEPDEVMSFQSHMTLKPTCEIFHKQNSKLNSGISPDPTLPQESISASLNSLLLPKNTPCLNAQEQFMSSLRPLAIQQIKAVSKSLISESSQPNTSSHNISVIADLHLSTIDWEGTSFSNSPAIQRNTFSHDLKSEVESELSAIPDGFENIPEQLSCESERYTANIKKVLDEDSDGISPEEHLLSGITDLCLQDLPLKERIFTKLSYPQDNLQPDVNLKTLSILSVKESCIANSGSDCTSHLSKDLPGIPLQNESRDSKILKGDQLLQEDYKVNTSVPYSVSNTVVKTCNVRPPNTALDHSRKVDMQTTRKILMKKSVCLDRHSSDEQSAPVFGKAKYTTQRMKHSSQKHNSSHFKESGHNKLSSPKIHIKETEQCVRSYETAENEESCFPDSTKSSLSSLQCHKKENNSGTCLDSPLPLRQRLKLRFQST

SEQ ID NO: 82 - protein, 1186 aa, synthetic construct
MGVQGLWKLLECSGRQVSPEALEGKILAVDISIWLNQALKGVRDRHGNSIENPHLLTLFHRLCKLLFFRIRPIFVFDGDAPLLKKQTLVKRRQRKDLASSDSRKTTEKLLKTFLKRQAIKTAFRSKRDEALPSLTQVRRENDLYVLPPLQEEEKHSSEEEDEKEWQERMNQKQALQEEFFHNPQAIDIESEDFSSLPPEVKHEILTDMKEFTKRRRTLFEAMPEESDDFSQYQLKGLLKKNYLNQHIEHVQKEMNQQHSGHIRRQYEDEGGFLKEVESRRVVSEDTSHYILIKGIQAKTVAEVDSESLPSSSKMHGMSFDVKSSPCEKLKTEKEPDATPPSPRTLLAMQAALLGSSSEEELESENRRQARGRNAPAAVDEGSISPRTLSAIKRALDDDEDVKVCAGDDVQTGGPGAEEMRINSSTENSDEGLKVRDGKGIPFTATLASSSVNSAEEHVASTNEGREPTDSVPKEQMSLVHVGTEAFPISDESMIKDRKDRLPLESAVVRHSDAPGLPNGRELTPASPTCTNSVSKNETHAEVLEQQNELCPYESKFDSSLLSSDDETKCKPNSASEVIGPVSLQETSSIVSVPSEAVDNVENVVSFNAKEHENFLETIQEQQTTESAGQDLISIPKAVEPMEIDSEESESDGSFIEVQSVISDEELQAEFPETSKPPSEQGEEELVGTREGEAPAESESLLRDNSERDDVDGEPQEAEKDAEDSLHEWQDINLEELETLESNLLAQQNSLKAQKQQQERIAATVTGQMFLESQELLRLFGIPYIQAPMEAEAQCAILDLTDQTSGTITDDSDIWLFGARHVYRNFFNKNKFVEYYQYVDFHNQLGLDRNKLINLAYLLGSDYTEGIPTVGCVTAMEILNEFPGHGLEPLLKFSEWWHEAQKNPKIRPNPHDTKVKKKLRTLQLTPGFPNPAVAEAYLKPVVDDSKGSFLWGKPDLDKIREFCQRYFGWNRTKTDESLFPVLKQLDAQQTQLRIDSFFRLAQQEKEDAKRIKSQRLNRAVTCMLRKEKEAAASEIEAVSVAMEKEFELLDKAKRKTQKRGITNTLEESSSLKRKRLSDSKRKNTCGGFLGETCLSESSDGSSSEDAESSSLMNVQRRTAAKEPKTSASDSQNSVKEAPVKNGGATTSSSSDSDDDGGKEKMVLVTARSVFGKKRRKLRRARGRKRKT

SEQ ID NO: 83 - protein, 236 aa, Homo sapiens
MSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 84 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 85 - protein, 236 aa, Rattus norvegicus
MSEPLRAETFVFLDLEATGLPNMDPEIAEISLFAVHRSSLENPERDDSGSLVLPRVLDKLTLCMCPERPFTAKASEITGLSSEGLMNCRKAAFNDAVVRTLQGFLSRQEGPICLVAHNGFDYDFPLLCTELQRLGAHLPRDTVCLDTLPALRGLDRVHSHGTRAQGRKSYSLASLFHRYFQAEPSAAHSAEGDVNTLLLIFLHRAPELLAWADEQARSWAHIEPMYVPPDGPSLEA

SEQ ID NO: 86 - protein, 803 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 87 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 88 - protein, 845 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 89 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 90 - protein, 413 aa, synthetic construct
CLAEGTRIFDPVTGTTHRIEDVVDGRKPIHVVAVAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYTNVVPLYDLLLEMLDAHRLHAGGSGASRVQAFADALDDKFLHDMLAEELRYSVIREVLPTRRARTFDLEVEELHTLVAEGVVVHNC

SEQ ID NO: 91 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 92 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 93 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 94 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYNPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 95 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 96 - protein, 413 aa, synthetic construct
CLAEGTRIFDPVTGTTHRIEDVVDGRKPIHVVAVAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 97 - protein, 33 aa, synthetic construct
GCCAACATGAGGATCACCCATGTCTGCAGGGCC

SEQ ID NO: 98 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 99 - protein, 84 aa, synthetic construct
MTNLSDIIEKETGKQLVIQESILMLPEEVEEVIGNKPESDILVHTAYDESTDENVMLLTSDAPEYKPWALVIQDSNGENKIKML

SEQ ID NO: 100 - protein, 345 aa, Homo sapiens
MPARALLPRRMGHRTLASTPALWASIPCPRSELRLDLVLPSGQSFRWREQSPAHWSGVLADQVWTLTQTEEQLHCTVYRGDKSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVLFSADLRQSRHAQEPPAKRRKGSKGPEG

SEQ ID NO: 101 - protein, 298 aa, Homo sapiens
MVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 102 - protein, 580 aa, Homo sapiens
MGTTGLESLSLGDRGAAPTVTSSERLVPDPPNDLRKEDVAMELERVGEDEEQMMIKRSSECNPLLQEPIASAQFGATAGTECRKSVPCGWERVVKQRLFGKTAGRFDVYFISPQGLKFRSKSSLANYLHKNGETSLKPEDFDFTVLSKRGIKSRYKDCSMAALTSHLQNQSNNSNWNLRTRSKCKKDVFMPPSSSSELQESRGLSNFTSTHLLLKEDEGVDDVNFRKVRKPKGKVTILKGIPIKKTKKGCRKSCSGFVQSDSKRESVCNKADAESEPVAQKSQLDRTVCISDAGACGETLSVTSEENSLVKKKERSLSSGSNFCSEQKTSGIINKFCSAKDSEHNEKYEDTFLESEEIGTKVEVVERKEHLHTDILKRGSEMDNNCSPTRKDFTGEKIFQEDTIPRTQIERRKTSLYFSSKYNKEALSPPRRKAFKKWTPPRSPFNLVQETLFHDPWKLLIATIFLNRTSGKMAIPVLWKFLEKYPSAEVARTADWRDVSELLKPLGLYDLRAKTIVKFSDEYLTKQWKYPIELHGIGKYGNDSYRIFCVNEWKQVHPEDHKLNKYHDWLWENHEKLSLS

SEQ ID NO: 103 - protein, 410 aa, Homo sapiens
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 104 - DNA, 125 bp, synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 105 - DNA, 121 bp, synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 106 - DNA, 109 bp, synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 107 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 108 - DNA, 88 bp, synthetic construct
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 109 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 110 - RNA, 82 nt, synthetic construct
  source 1..82: mol_type = other RNA
gttttagagctagaaatagcaagttaaaataaaggctagtccgttatcaacttgaaaaagtggcaccgagtcggtgcttttt

SEQ ID NO: 111 - DNA, 905 bp, synthetic construct
  source 1..905: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgact

SEQ ID NO: 112 - DNA, 942 bp, synthetic construct
  source 1..942: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 113 - DNA, 1370 bp, synthetic construct
  source 1..1370: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaaacacactgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaatctctctgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 114 - DNA, 807 bp, synthetic construct
  source 1..807: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttgtttcaaaagtagactgtacgctaagggtcatatctttttttgtttggtttgtgtcttggttggcgtcttaaa

SEQ ID NO: 115 - DNA, 743 bp, synthetic construct
  source 1..743: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcagttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttcagcaagttcagagaaatctgaacttgctggatttttggagcagggagatggaataggagcttgctccgtccactccacgcatcgacctggtattgcagtacctccaggaacggtgcacccactttctggagtttcaaaagtagactgtacgctaagggtcatatctttttttgtttggtttgtgtcttggttggcgtcttaaa

SEQ ID NO: 116 - DNA, 10 bp, synthetic construct
  source 1..10: mol_type = other DNA
gttttaaaac

SEQ ID NO: 117 - DNA, 141 bp, synthetic construct
  source 1..141: mol_type = other DNA
ggcccagactgagcacgtgagttttagagctagctcatgaaaatgagctagcaagttaaaataaggctagtccgttatcaacttgaaaaagtgggaccgagtcggtcctctgccatcaaagcgtgctcagtctgttttttt

SEQ ID NO: 118 - DNA, 129 bp, synthetic construct
  source 1..129: mol_type = other DNA
ggcccagactgagcacgtgagtttgagagctagaaatagcaagtttaaataaggctagtccgttatcaacttgaaaaagtgggaccgagtcggtcctctgccatcaaagcgtgctcagtctgttttttt

SEQ ID NO: 119 - DNA, 197 bp, synthetic construct
  source 1..197: mol_type = other DNA
ggcccagactgagcacgtgagttttagagctagaaatagcaagttaaaataaggctagtccgttatcaacttgaaaaagtgggaccgagtcggtcctctgccatcaaagcgtgctcagtctgggccggcatggtcccagcctcctcgctggcgccggctgggcaacatgcttcggcatggcgaatgggacttttttt

SEQ ID NO: 120 - DNA, 171 bp, synthetic construct
  source 1..171: mol_type = other DNA
ggtgggagacgtcccaccggcccagactgagcacgtgagttttagagctagaaatagcaagttaaaataaggctagtccgttatcaacttgaaaaagtgggaccgagtcggtcctctgccatcaaagcttcgaccgtgctcagtctggtgggagacgtcccaccttttttt

SEQ ID NO: 121 - DNA, 180 bp, synthetic construct
  source 1..180: mol_type = other DNA
gagcagcatggcgtcgctgctcacggcccagactgagcacgtgagttttagagctagaaatagcaagttaaaataaggctagtccgttatcaacttgaaaaagtgggaccgagtcggtcctctgccatcaaagcttcgaccgtgctcagtctccatcagttgacaccctgaggttttttt

SEQ ID NO: 122 - DNA, 188 bp, synthetic construct
  source 1..188: mol_type = other DNA
  misc_feature 15: note = RNA
  misc_feature 61: note = RNA
  misc_feature 101: note = RNA
gcagacctaagtggtgacatatggtctgggcccagactgagcacgtgagttttagagctatacgtagcaagttaaaataaggctagtccgttatcaactttacgaagtgggaccgagtcggtcctctgccatcaaagcttcgaccgtgctcagtctgcatgcgattagaaataatcgcatgttttttt

SEQ ID NO: 123 - DNA, 125 bp, synthetic construct
  source 1..125: mol_type = other DNA
tctgccatcaaagctgcgaccgtgctcagtctggtgggagacgtcccaccggccggcatggtcccagcctcctcgctggcgccggctgggcaacatgcttcggcatggcgaatgggacttttttt

SEQ ID NO: 124 - DNA, 34 bp, synthetic construct
  source 1..34: mol_type = other DNA
ataacttcgtatagcatacattatacgaagttat

SEQ ID NO: 125 - DNA, 32 bp, synthetic construct
  source 1..32: mol_type = other DNA
taactttaaataatgccaattatttaaagtta

SEQ ID NO: 126 - DNA, 34 bp, synthetic construct
  source 1..34: mol_type = other DNA
tcaatttctgagaactgtcattctcggaaattga

SEQ ID NO: 127 - DNA, 34 bp, synthetic construct
  source 1..34: mol_type = other DNA
ctcgtgtccgataactgtaattatcggacatgat

SEQ ID NO: 128 - DNA, 34 bp, synthetic construct
  source 1..34: mol_type = other DNA
gaagttcctattctctagaaagtataggaacttc

SEQ ID NO: 129 - DNA, 39 bp, synthetic construct
  source 1..39: mol_type = other DNA
gagtttcattaaggaataactaattccctaatgaaactc

SEQ ID NO: 130 - DNA, 33 bp, synthetic construct
  source 1..33: mol_type = other DNA
ggttgcttaagaataagtaattcttaagcaacc

SEQ ID NO: 131 - DNA, 43 bp, synthetic construct
  source 1..43: mol_type = other DNA
acgaaaaatggtaaggaatagaccattccttaccatttttggt

SEQ ID NO: 132 - DNA, 31 bp, synthetic construct
  source 1..31: mol_type = other DNA
ttgatgaaagaataacgtattctttcatcaa

SEQ ID NO: 133 - DNA, 33 bp, synthetic construct
  source 1..33: mol_type = other DNA
gtgcgtcaaataataacgtattatttgacactt

SEQ ID NO: 134 - DNA, 34 bp, synthetic construct
  source 1..34: mol_type = other DNA
aataggtctgagaacgcccattctcagacgtatt

SEQ ID NO: 135 - DNA, 34 bp, synthetic construct
  source 1..34: mol_type = other DNA
tgaatgtcctataattacacttataggacattca

SEQ ID NO: 136 - DNA, 34 bp, synthetic construct
  source 1..34: mol_type = other DNA
gaaactttaaataataagtcttatttaaagtttc

SEQ ID NO: 137 - DNA, 61 bp, synthetic construct
  source 1..61: mol_type = other DNA
aaacgatatcagacatttgtctgataatgcttcattatcagacaaatgtctgatatcgttt

SEQ ID NO: 138 - DNA, 34 bp, synthetic construct
  source 1..34: mol_type = other DNA
atatatacgtatatagacatatatacgtatatat

SEQ ID NO: 139 - DNA, 34 bp, synthetic construct
  source 1..34: mol_type = other DNA
ataactctatataatgtatgctatatagagttat

SEQ ID NO: 140 - DNA, 34 bp, synthetic construct
  source 1..34: mol_type = other DNA
acaacatcctattacaccctatatgccaacatgg

SEQ ID NO: 141 - DNA, 34 bp, synthetic construct
  source 1..34: mol_type = other DNA
aacccactgcttaagcctcaataaagcttgcctt

SEQ ID NO: 142 - DNA, 34 bp, synthetic construct
  source 1..34: mol_type = other DNA
gatacaacgtatatacctttctatacgttgttta

SEQ ID NO: 143 - DNA, 48 bp, synthetic construct
  source 1..48: mol_type = other DNA
ggtttgtctggtcaaccaccgcggtctcagtggtgtacggtacaaacc

SEQ ID NO: 144 - DNA, 42 bp, synthetic construct
  source 1..42: mol_type = other DNA
gtgccccaactggggtaacctttgagttctctcagttggggg

SEQ ID NO: 145 - DNA, 50 bp, synthetic construct
  source 1..50: mol_type = other DNA
tgttccccaaagcgataccacttgaagcagtggtactgcttgtgggtaca

SEQ ID NO: 146 - DNA, 52 bp, synthetic construct
  source 1..52: mol_type = other DNA
ggtgctgggttgttgtctctggacagtgatccatgggaaactactcagcacc

SEQ ID NO: 147 - DNA, 54 bp, synthetic construct
  source 1..54: mol_type = other DNA
attttaggtatatgattttgtttattagtgtaaataacactatgtacctaaaat

SEQ ID NO: 148 - DNA, 55 bp, synthetic construct
  source 1..55: mol_type = other DNA
tttgtgcggaactacgaacagttcattaatacgaagtgtacaaacttccatacaa

SEQ ID NO: 149 - DNA, 53 bp, synthetic construct
  source 1..53: mol_type = other DNA
gagtttttatttcgtttatttcaattaaggtaactaaaaaactccttttaagg

SEQ ID NO: 150 - DNA, 40 bp, synthetic construct
  source 1..40: mol_type = other DNA
ttcctcgttttctctcgttggaagaagaagaaacgagaaa

SEQ ID NO: 151 - DNA, 40 bp, synthetic construct
  source 1..40: mol_type = other DNA
ttcctcgttttctctcgttggacggaaacgaatcgagaaa

SEQ ID NO: 152 - DNA, 52 bp, synthetic construct
  source 1..52: mol_type = other DNA
gtagtgtatctcacaggtccacggttggccgtggactgctgaagaacattcc

SEQ ID NO: 153 - DNA, 52 bp, synthetic construct
  source 1..52: mol_type = other DNA
aaaaaaatacagcgtttttcatgtacaactatactagttgtagtgcctaaaa

SEQ ID NO: 154 - DNA, 46 bp, synthetic construct
  source 1..46: mol_type = other DNA
tccagcccaacagtgttagtctttgctcttacccagttgggcggga

SEQ ID NO: 155 - DNA, 63 bp, synthetic construct
  source 1..63: mol_type = other DNA
ctagttttaaagttggttattagttactgtgatatttatcacggtacccaataaccaatgaat

SEQ ID NO: 156 - DNA, 33 bp, synthetic construct
  source 1..33: mol_type = other DNA
caatgaaaaactaggcatgtagaagttgtttgt

SEQ ID NO: 157 - DNA, 56 bp, synthetic construct
  source 1..56: mol_type = other DNA
tgtagtaagtatcttaatatacagctttatctgttttttaagatacttactacttt

SEQ ID NO: 158 - DNA, 47 bp, synthetic construct
  source 1..47: mol_type = other DNA
agttgtggccatgtgtccatctgggggcagatggagacggggtcaca

SEQ ID NO: 159 - protein, 240 aa, synthetic construct
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPV

SEQ ID NO: 160 - DNA, 60 bp, synthetic construct
  source 1..60: mol_type = other DNA
ccctaatacgcaagtcgataactctcctgggagcgttgacaacttgcgcaccctgatctg

SEQ ID NO: 161 - DNA, 63 bp, synthetic construct
  source 1..63: mol_type = other DNA
gctcatgtatgtgtctacgcgagattctcgcccgagaacttctgcaaggcactgctcttggct

SEQ ID NO: 162 - DNA, 65 bp, synthetic construct
  source 1..65: mol_type = other DNA
atggataaaaaaatacagcgtttttcatgtacaactatactagttgtagtgcctaaataatgctt

SEQ ID NO: 163 - DNA, 62 bp, synthetic construct
  source 1..62: mol_type = other DNA
aaaaattacaaagttttcaacccttgatttgaattagcggtcaaataatttgtaattcgttt

SEQ ID NO: 164 - DNA, 66 bp, synthetic construct
  source 1..66: mol_type = other DNA
gtgttataaacctgtgtgagagttaagtttacatgcctaaccttaacttttacgcaggttcagctt

SEQ ID NO: 165 - DNA, 64 bp, synthetic construct
  source 1..64: mol_type = other DNA
ttaataaactatggaagtatgtacagtcttgcaatgttgagtgaacaaacttccataataaaat

SEQ ID NO: 166 - DNA, 62 bp, synthetic construct
  source 1..62: mol_type = other DNA
gtggttgtttttgttggaagtgtgtatcaggtatctgcatagttattccgaacttccaatta

SEQ ID NO: 167 - DNA, 62 bp, synthetic construct
  source 1..62: mol_type = other DNA
ggaaaatataaataattttagtaacctacatctcaatcaaggatagtaaaactctcactctt

SEQ ID NO: 168 - DNA, 65 bp, synthetic construct
  source 1..65: mol_type = other DNA
gtttatatgtttactaataagacgctctcaacccataaagtcttattagtaaacatatttcaact

SEQ ID NO: 169 - DNA, 65 bp, synthetic construct
  source 1..65: mol_type = other DNA
tttttgtatgttagttgtgtcactgggtagacctaaatagtgacacaactgctattaaaatttaa

SEQ ID NO: 170 - DNA, 64 bp, synthetic construct
  source 1..64: mol_type = other DNA
caataacggttgtatttgtagaacttgaccagttgttttagtaacataaatacaactccgaata

SEQ ID NO: 171 - DNA, 52 bp, synthetic construct
  source 1..52: mol_type = other DNA
gtttagtatctcgttatctctcgttggagggagaagaaacgggataccaaaa

SEQ ID NO: 172 - DNA, 52 bp, synthetic construct
  source 1..52: mol_type = other DNA
tagtttccaatgttacaggaactgctggcagaatccaacacattggaagtcg

SEQ ID NO: 173 - DNA, 52 bp, synthetic construct
  source 1..52: mol_type = other DNA
taaccgcaagtgtacatccctcggctggccgagacaagtacagttgcgacag

SEQ ID NO: 174 - DNA, 52 bp, synthetic construct
  source 1..52: mol_type = other DNA
atgtggtcctttagatccactgacgtgggtcagtgtctctaaaggactcgcg

SEQ ID NO: 175 - DNA, 38 bp, synthetic construct
  source 1..38: mol_type = other DNA
ggcttgtcgacgacggcggtctccgtcgtcaggatcat

SEQ ID NO: 176 - DNA, 45 bp, synthetic construct
  source 1..45: mol_type = other DNA
tgcgggtgccagggcgtgcccttgggctccccgggcgcgtactcc

SEQ ID NO: 177 - DNA, 64 bp, synthetic construct
  source 1..64: mol_type = other DNA
gcatgttccccaaagcgataccacttgaagcagtggtactgcttgtgggtacactctgcgggtg

SEQ ID NO: 178 - DNA, 34 bp, synthetic construct
  source 1..34: mol_type = other DNA
caggtttttgacgaaagtgatccagatgatccag

SEQ ID NO: 179 - DNA, 42 bp, synthetic construct
  source 1..42: mol_type = other DNA
caaaggatcactgaatcaaaagtattgctcatccacgcgaaa

SEQ ID NO: 180 - DNA, 56 bp, synthetic construct
  source 1..56: mol_type = other DNA
cgaaaatgtatggaggcacttgtatcaatataggatgtataccttcgaagacactt

SEQ ID NO: 181 - DNA, 53 bp, synthetic construct
  source 1..53: mol_type = other DNA
atgccaacacaattaacatctcaatcaaggtaaatgctttttgctttttttgc

SEQ ID NO: 182 - DNA, 40 bp, synthetic construct
  source 1..40: mol_type = other DNA
tttcggatcaagctatgaaggacgcaaagagggaactaaa

SEQ ID NO: 183 - DNA, 40 bp, synthetic construct
  source 1..40: mol_type = other DNA
gaaggtgttggtgcggggttggccgtggtcgaggtggggt

SEQ ID NO: 184 - DNA, 54 bp, synthetic construct
  source 1..54: mol_type = other DNA
ttgtaaaggagactgataatggcatgtacaactatactcgtcggtaaaaaggca

SEQ ID NO: 185 - DNA, 47 bp, synthetic construct
  source 1..47: mol_type = other DNA
gatcagctccgcgggcaagacctttctccttcacggggtggaaggtc

SEQ ID NO: 186 - DNA, 71 bp, synthetic construct
  source 1..71: mol_type = other DNA
cggaaggtagcgtcaacgataggtgtaactgtcgtgtttgtaacggtacttccaacagctggcgccgccac

SEQ ID NO: 187 - DNA, 32 bp, synthetic construct
  source 1..32: mol_type = other DNA
tttccacagacaactcacgtggaggtagtcac

SEQ ID NO: 188 - DNA, 58 bp, synthetic construct
  source 1..58: mol_type = other DNA
cacccattgtgttcacaggagatacagctttatctgtactgatattaatgacatgctg

SEQ ID NO: 189 - DNA, 50 bp, synthetic construct
  source 1..50: mol_type = other DNA
atctggatgtgggtgtccatctgcgggcagacgccgcagtcgaagcacgg

SEQ ID NO: 190 - DNA, 50 bp, synthetic construct
  source 1..50: mol_type = other DNA
accttgatctcggtgtccatcgccgggcagacgccgcagtcgaagcacgg

SEQ ID NO: 191 - DNA, 61 bp, synthetic construct
  source 1..61: mol_type = other DNA
gagcgccggatcagggagtggacggcctgggagcgctacacgctgtggctgcggtcggtgc

SEQ ID NO: 192 - DNA, 59 bp, synthetic construct
  source 1..59: mol_type = other DNA
ggacggcgcagaaggggagtagctcttcgccggaccgtcgacatactgctcagctcgtc

SEQ ID NO: 193 - DNA, 62 bp, synthetic construct
  source 1..62: mol_type = other DNA
gtttgtaaaggagactgataatggcatgtacaactatactcgtcggtaaaaaggcatcttat

SEQ ID NO: 194 - DNA, 66 bp, synthetic construct
  source 1..66: mol_type = other DNA
ttccaaagagcgcccaacgcgacctgaaatttgaataagactgctgcttgtgtaaaggcgatgatt

SEQ ID NO: 195 - DNA, 62 bp, synthetic construct
  source 1..62: mol_type = other DNA
agacgagaaacgttccgtccgtctgggtcagttgggcaaagttgatgaccgggtcgtccgtt

SEQ ID NO: 196 - DNA, 65 bp, synthetic construct
  source 1..65: mol_type = other DNA
caatcatcagataactatggcggcacgtgcattaaccacggttgtatcccgtctaaagtactcgt

SEQ ID NO: 197 - DNA, 65 bp, synthetic construct
  source 1..65: mol_type = other DNA
tttatattgcgaaaaataattggcgaacgaggtaactggatacctcatccgccaattaaaatttg

SEQ ID NO: 198 - DNA, 62 bp, synthetic construct
  source 1..62: mol_type = other DNA
agcacgctgataatcagcaagaccaccaacatttccaccaatgtaaaagctttaaccttagc

SEQ ID NO: 199 - DNA, 65 bp, synthetic construct
  source 1..65: mol_type = other DNA
atggattttgcagattcccagatgcccctacagaaagaggtacaaaacatttattggaattaatt

SEQ ID NO: 200 - DNA, 66 bp, synthetic construct
  source 1..66: mol_type = other DNA
gttcgtggtaactatgggtggtacaggtgccacattagttgtaccatttatgtttatgtggttaac

SEQ ID NO: 201 - DNA, 64 bp, synthetic construct
  source 1..64: mol_type = other DNA
gcatacattgttgttgtttttccagatccagttggtcctgtaaatataagcaatccatgtgagt

SEQ ID NO: 202 - DNA, 52 bp, synthetic construct
  source 1..52: mol_type = other DNA
taactttttcggatcgagttatgatggacgtaaagagggaacaaagcatcta

SEQ ID NO: 203 - DNA, 42 bp, synthetic construct
  source 1..42: mol_type = other DNA
cggtctccatcgggatctgctgatcgagcagcatgccgacca

SEQ ID NO: 204 - DNA, 53 bp, synthetic construct
  source 1..53: mol_type = other DNA
catcagggcggtcaggccgtagatgtggaagaaacggcagcacggcgaggacg

SEQ ID NO: 205 - DNA, 32 bp, synthetic construct
  source 1..32: mol_type = other DNA
cgtttcctgtaaaccgaggttttggataaaca

SEQ ID NO: 206 - DNA, 32 bp, synthetic construct
  source 1..32: mol_type = other DNA
gagttctcttaaaccaaggtttaggattgaaa

SEQ ID NO: 207 - DNA, 32 bp, synthetic construct
  source 1..32: mol_type = other DNA
tggttcttgaaaaccaaggtttttgataaagc

SEQ ID NO: 208 - DNA, 32 bp, synthetic construct
  source 1..32: mol_type = other DNA
gccttcccctaaaccaacgtttttatgccgcc

SEQ ID NO: 209 - DNA, 32 bp, synthetic construct
  source 1..32: mol_type = other DNA
ttgtgaaatttgggtacaccctaatcatacaa

SEQ ID NO: 210 - DNA, 32 bp, synthetic construct
  source 1..32: mol_type = other DNA
cgtttcctgtaaaccgaggttttggataatgg

SEQ ID NO: 211 - DNA, 32 bp, synthetic construct
  source 1..32: mol_type = other DNA
gagttctcttaaaccaaggtattggataacag

SEQ ID NO: 212 - DNA, 33 bp, synthetic construct
  source 1..33: mol_type = other DNA
aaattttcctttttggaaggtttttgataacca

SEQ ID NO: 213 - DNA, 32 bp, synthetic construct
  source 1..32: mol_type = other DNA
gccttcccccaaaccaaggtaatcaagaacgc

SEQ ID NO: 214 - DNA, 26 bp, synthetic construct
  source 1..26: mol_type = other DNA
cgtatgattagggtgtatattaattt

SEQ ID NO: 215 - DNA, 101 bp, synthetic construct
  source 1..101: mol_type = other DNA
caaatgattttattttgactaataatgacctacttacattaatttactgataattaaagagattttaaatatacaacttattcacctaaaggatgacaaaa

SEQ ID NO: 216 - DNA, 126 bp, synthetic construct
  source 1..126: mol_type = other DNA
taacattaatcacttaaaaatcatcgcattacactaatctgtggttaaatgatagactacataatgcgacaaaacgcaacatatccagtcactatgaatcaactacttagatagtattagtgacct

SEQ ID NO: 217 - DNA, 259 bp, synthetic construct
  source 1..259: mol_type = other DNA
ctaagtggtttgggacaaaaatgggacatacaaatctttgcatcggtttgcaaggctttgcatgtctttcgaagatgggacgtgtgagcgcaggtatgacgtggtatgtgttgacttaaaaggtagttcttataattcgtaatgcgaaggtcgtaggttcgactcctattatcggcaccagttaaatcaaatacttacgtattattcgtgccttccttatttttactgtgggacatatttgggacagaagtaccaaaaa

SEQ ID NO: 218 - DNA, 412 bp, synthetic construct
  source 1..412: mol_type = other DNA
gcgatccccatccgcgacgtgccaactaggtctcctctcgtcgtgaacaaggctaccgggttgcaactcctgtgcaactctcaggcttcaacgcgcttctacgacctgcaatttctttccacttagaggatgcagccgagaggggtaaaaacctatcttgaccggcccatatgtggtcggcagacacccattcttccaaactagctacgcgggttcgattcccgtcgcccgctccgctggtcagagggtgttttcgccctctggccatttttctttccaggggtctgcaactcttgtgcgactcttctgacctgggcatacgcggttgcaacgcatccctgatctggctactttcgatgctgacaaacgaatagagccccccgcctgcgcgaacagacgaggggcattcaca

SEQ ID NO: 219 - DNA, 21 bp, synthetic construct
  source 1..21: mol_type = other DNA
gcactttaggtgaaaaaggtt

SEQ ID NO: 220 - DNA, 27 bp, synthetic construct
  source 1..27: mol_type = other DNA
gcagcgcattcgtaatgcgaaggtcgt

SEQ ID NO: 221 - DNA, 43 bp, synthetic construct
  source 1..43: mol_type = other DNA
gagcgggcgacgggaatcgaacccgcgtagctagtttggaaga

SEQ ID NO: 222 - DNA, 34 bp, synthetic construct
  source 1..34: mol_type = other DNA
ataacttcgtataaagtatcctatacgaagttat

SEQ ID NO: 223 - DNA, 34 bp, synthetic construct
  source 1..34: mol_type = other DNA
ataacttcgtatagtataccttatacgaagttat

SEQ ID NO: 224 - DNA, 34 bp, synthetic construct
  source 1..34: mol_type = other DNA
ataacttcgtataatgtatactatacgaagttat

SEQ ID NO: 225 - DNA, 34 bp, synthetic construct
  source 1..34: mol_type = other DNA
ataacttcgtataatgtgtactatacgaagttat

SEQ ID NO: 226 - DNA, 34 bp, synthetic construct
  source 1..34: mol_type = other DNA
ataacttcgtataagaaaccatatacgaagttat

SEQ ID NO: 227 - DNA, 34 bp, synthetic construct
  source 1..34: mol_type = other DNA
ataacttcgtatataataccatatacgaagttat

SEQ ID NO: 228 - DNA, 34 bp, synthetic construct
  source 1..34: mol_type = other DNA
ataacttcgtataagatagaatatacgaagttat

SEQ ID NO: 229 - DNA, 34 bp, synthetic construct
  source 1..34: mol_type = other DNA
ataacttcgtatacgataccatatacgaagttat

SEQ ID NO: 230 - DNA, 34 bp, synthetic construct
  source 1..34: mol_type = other DNA
taccgttcgtatagcatacattatacgaagttat

SEQ ID NO: 231 - DNA, 34 bp, synthetic construct
  source 1..34: mol_type = other DNA
ataacttcgtatagcatacattatacgaacggta

SEQ ID NO: 232 - DNA, 48 bp, synthetic construct
  source 1..48: mol_type = other DNA
ggtttgtctggtcaaccaccgcggactcagtggtgtacggtacaaacc

SEQ ID NO: 233 - DNA, 48 bp, synthetic construct
  source 1..48: mol_type = other DNA
ggtttgtctggtcaaccaccgcggcctcagtggtgtacggtacaaacc

SEQ ID NO: 234 - DNA, 48 bp, synthetic construct
  source 1..48: mol_type = other DNA
ggtttgtctggtcaaccaccgcgggctcagtggtgtacggtacaaacc

SEQ ID NO: 235 - DNA, 48 bp, synthetic construct
  source 1..48: mol_type = other DNA
ggtttgtctggtcaaccaccgcgaactcagtggtgtacggtacaaacc

SEQ ID NO: 236 - DNA, 48 bp, synthetic construct
  source 1..48: mol_type = other DNA
ggtttgtctggtcaaccaccgcgacctcagtggtgtacggtacaaacc

SEQ ID NO: 237 - DNA, 48 bp, synthetic construct
  source 1..48: mol_type = other DNA
ggtttgtctggtcaaccaccgcgagctcagtggtgtacggtacaaacc

SEQ ID NO: 238 - DNA, 48 bp, synthetic construct
  source 1..48: mol_type = other DNA
ggtttgtctggtcaaccaccgcgatctcagtggtgtacggtacaaacc

SEQ ID NO: 239 - DNA, 48 bp, synthetic construct
  source 1..48: mol_type = other DNA
ggtttgtctggtcaaccaccgcgcactcagtggtgtacggtacaaacc

SEQ ID NO: 240 - DNA, 48 bp, synthetic construct
  source 1..48: mol_type = other DNA
ggtttgtctggtcaaccaccgcgccctcagtggtgtacggtacaaacc

SEQ ID NO: 241 - DNA, 48 bp, synthetic construct
  source 1..48: mol_type = other DNA
ggtttgtctggtcaaccaccgcgcgctcagtggtgtacggtacaaacc

SEQ ID NO: 242 - DNA, 48 bp, synthetic construct
  source 1..48: mol_type = other DNA
ggtttgtctggtcaaccaccgcgctctcagtggtgtacggtacaaacc

SEQ ID NO: 243 - DNA, 48 bp, synthetic construct
  source 1..48: mol_type = other DNA
ggtttgtctggtcaaccaccgcgtactcagtggtgtacggtacaaacc

SEQ ID NO: 244 - DNA, 48 bp, synthetic construct
  source 1..48: mol_type = other DNA
ggtttgtctggtcaaccaccgcgtcctcagtggtgtacggtacaaacc

SEQ ID NO: 245 - DNA, 48 bp, synthetic construct
  source 1..48: mol_type = other DNA
ggtttgtctggtcaaccaccgcgtgctcagtggtgtacggtacaaacc

SEQ ID NO: 246 - DNA, 48 bp, synthetic construct
  source 1..48: mol_type = other DNA
ggtttgtctggtcaaccaccgcgttctcagtggtgtacggtacaaacc

SEQ ID NO: 247 - DNA, 55 bp, synthetic construct
  source 1..55: mol_type = other DNA
gtagtaagtatcttaatatacagctttatctgttttttaagatacttactacttt

SEQ ID NO: 248 - DNA, 38 bp, synthetic construct
  source 1..38: mol_type = other DNA
ggcttgtcgacgacggcggactccgtcgtcaggatcat

SEQ ID NO: 249 - DNA, 38 bp, synthetic construct
  source 1..38: mol_type = other DNA
ggcttgtcgacgacggcggcctccgtcgtcaggatcat

SEQ ID NO: 250 - DNA, 38 bp, synthetic construct
  source 1..38: mol_type = other DNA
ggcttgtcgacgacggcgggctccgtcgtcaggatcat

SEQ ID NO: 251 - DNA, 38 bp, synthetic construct
  source 1..38: mol_type = other DNA
ggcttgtcgacgacggcgaactccgtcgtcaggatcat

SEQ ID NO: 252 - DNA, 38 bp, synthetic construct
  source 1..38: mol_type = other DNA
ggcttgtcgacgacggcgacctccgtcgtcaggatcat

SEQ ID NO: 253 - DNA, 38 bp, synthetic construct
  source 1..38: mol_type = other DNA
ggcttgtcgacgacggcgagctccgtcgtcaggatcat

SEQ ID NO: 254 - DNA, 38 bp, synthetic construct
  source 1..38: mol_type = other DNA
ggcttgtcgacgacggcgatctccgtcgtcaggatcat

SEQ ID NO: 255 - DNA, 38 bp, synthetic construct
  source 1..38: mol_type = other DNA
ggcttgtcgacgacggcgcactccgtcgtcaggatcat

SEQ ID NO: 256 - DNA, 38 bp, synthetic construct
  source 1..38: mol_type = other DNA
ggcttgtcgacgacggcgccctccgtcgtcaggatcat

SEQ ID NO: 257 - DNA, 38 bp, synthetic construct
  source 1..38: mol_type = other DNA
ggcttgtcgacgacggcgcgctccgtcgtcaggatcat

SEQ ID NO: 258 - DNA, 38 bp, synthetic construct
  source 1..38: mol_type = other DNA
ggcttgtcgacgacggcgctctccgtcgtcaggatcat

SEQ ID NO: 259 - DNA, 38 bp, synthetic construct
  source 1..38: mol_type = other DNA
ggcttgtcgacgacggcgtactccgtcgtcaggatcat

SEQ ID NO: 260 - DNA, 38 bp, synthetic construct
  source 1..38: mol_type = other DNA
ggcttgtcgacgacggcgtcctccgtcgtcaggatcat

SEQ ID NO: 261 - DNA, 38 bp, synthetic construct
  source 1..38: mol_type = other DNA
ggcttgtcgacgacggcgtgctccgtcgtcaggatcat

SEQ ID NO: 262 - DNA, 38 bp, synthetic construct
  source 1..38: mol_type = other DNA
ggcttgtcgacgacggcgttctccgtcgtcaggatcat

SEQ ID NO: 263 - DNA, 62 bp, synthetic construct
  source 1..62: mol_type = other DNA
cacccattgtgttcacaggagatacagctttatctgtactgatattaatgacatgctgcact